SEQUENCE LISTING (152 sequences)

SEQ ID NO: 1 - protein, 174 aa, Artificial sequence
  Synthetic sequence
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 175 aa, Artificial sequence
  Synthetic sequence
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 167 aa, Artificial sequence
  Synthetic sequence
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 4 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 174 aa, Artificial sequence
  Synthetic sequence
  misc_feature (47)..(47): Xaa can be any naturally occurring amino acid other than a lysine
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 174 aa, Artificial sequence
  Synthetic sequence
PAGLLDLRQGMFAQLVAQNVLLIDGPLSWYSDPGLAGVSLTGGLSYAEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 7 - protein, 174 aa, Artificial sequence
  Synthetic sequence
  misc_feature (147)..(147): Xaa can be any naturally occurring amino acid other than a glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 8 - protein, 174 aa, Artificial sequence
  Synthetic sequence
  misc_feature (11)..(11): Xaa can be any naturally occurring amino acid other than a methionine
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 9 - protein, 174 aa, Artificial sequence
  Synthetic sequence
  misc_feature (12)..(12): Xaa can be any naturally occurring amino acid other than a phenylalanine
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 174 aa, Artificial sequence
  Synthetic sequence
  misc_feature (14)..(14): Xaa can be any naturally occurring amino acid other than a glutamine
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 174 aa, Artificial sequence
  Synthetic sequence
  misc_feature (15)..(15): Xaa can be any naturally occurring amino acid other than a leucine
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 174 aa, Artificial sequence
  Synthetic sequence
  misc_feature (16)..(16): Xaa can be any naturally occurring amino acid other than a valine
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 174 aa, Artificial sequence
  Synthetic sequence
  misc_feature (18)..(18): Xaa can be any naturally occurring amino acid other than a glutamine
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 14 - protein, 174 aa, Artificial sequence
  Synthetic sequence
  misc_feature (19)..(19): Xaa can be any naturally occurring amino acid other than a asparagine
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 15 - protein, 174 aa, Artificial sequence
  Synthetic sequence
  misc_feature (20)..(20): Xaa can be any naturally occurring amino acid other than a valine
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 16 - protein, 174 aa, Artificial sequence
  Synthetic sequence
  misc_feature (21)..(21): Xaa can be any naturally occurring amino acid other than a leucine
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 17 - protein, 174 aa, Artificial sequence
  Synthetic sequence
  misc_feature (22)..(22): Xaa can be any naturally occurring amino acid other than a leucine
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 18 - protein, 174 aa, Artificial sequence
  Synthetic sequence
  misc_feature (23)..(23): Xaa can be any naturally occurring amino acid other than a isoleucine
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 19 - protein, 174 aa, Artificial sequence
  Synthetic sequence
  misc_feature (24)..(24): Xaa can be any naturally occurring amino acid other than a aspartic acid
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 20 - protein, 174 aa, Artificial sequence
  Synthetic sequence
  misc_feature (25)..(25): Xaa can be any naturally occurring amino acid other than a glycine
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 21 - protein, 174 aa, Artificial sequence
  Synthetic sequence
  misc_feature (26)..(26): Xaa can be any naturally occurring amino acid other than a proline
PAGLLDLRQGMFAQLVAQNVLLIDGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 22 - protein, 174 aa, Artificial sequence
  Synthetic sequence
  misc_feature (27)..(27): Xaa can be any naturally occurring amino acid other than a leucine
PAGLLDLRQGMFAQLVAQNVLLIDGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 23 - protein, 174 aa, Artificial sequence
  Synthetic sequence
  misc_feature (28)..(28): Xaa can be any naturally occurring amino acid other than a serine
PAGLLDLRQGMFAQLVAQNVLLIDGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 24 - protein, 174 aa, Artificial sequence
  Synthetic sequence
  misc_feature (29)..(29): Xaa can be any naturally occurring amino acid other than a Tryptophan
PAGLLDLRQGMFAQLVAQNVLLIDGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - protein, 174 aa, Artificial sequence
  Synthetic sequence
  misc_feature (30)..(30): Xaa can be any naturally occurring amino acid other than a tyrosine
PAGLLDLRQGMFAQLVAQNVLLIDGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 26 - protein, 174 aa, Artificial sequence
  Synthetic sequence
  misc_feature (31)..(31): Xaa can be any naturally occurring amino acid other than a Serine
PAGLLDLRQGMFAQLVAQNVLLIDGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 27 - protein, 174 aa, Artificial sequence
  Synthetic sequence
  misc_feature (32)..(32): Xaa can be any naturally occurring amino acid other than a Aspartic acid
PAGLLDLRQGMFAQLVAQNVLLIDGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 28 - protein, 174 aa, Artificial sequence
  Synthetic sequence
  misc_feature (33)..(33): Xaa can be any naturally occurring amino acid other than a Proline
PAGLLDLRQGMFAQLVAQNVLLIDGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 174 aa, Artificial sequence
  Synthetic sequence
  misc_feature (34)..(34): Xaa can be any naturally occurring amino acid other than a glycine
PAGLLDLRQGMFAQLVAQNVLLIDGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 30 - protein, 174 aa, Artificial sequence
  Synthetic sequence
  misc_feature (35)..(35): Xaa can be any naturally occurring amino acid other than a Leucine
PAGLLDLRQGMFAQLVAQNVLLIDGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 31 - protein, 174 aa, Artificial sequence
  Synthetic sequence
  misc_feature (37)..(37): Xaa can be any naturally occurring amino acid other than a Glycine
PAGLLDLRQGMFAQLVAQNVLLIDGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 32 - protein, 174 aa, Artificial sequence
  Synthetic sequence
  misc_feature (38)..(38): Xaa can be any naturally occurring amino acid other than a Valine
PAGLLDLRQGMFAQLVAQNVLLIDGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 33 - protein, 174 aa, Artificial sequence
  Synthetic sequence
  misc_feature (39)..(39): Xaa can be any naturally occurring amino acid other than a Serine
PAGLLDLRQGMFAQLVAQNVLLIDGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 34 - protein, 174 aa, Artificial sequence
  Synthetic sequence
  misc_feature (40)..(40): Xaa can be any naturally occurring amino acid other than a Leucine
PAGLLDLRQGMFAQLVAQNVLLIDGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 35 - protein, 174 aa, Artificial sequence
  Synthetic sequence
  misc_feature (41)..(41): Xaa can be any naturally occurring amino acid other than a Threonine
PAGLLDLRQGMFAQLVAQNVLLIDGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 36 - protein, 174 aa, Artificial sequence
  Synthetic sequence
  misc_feature (42)..(42): Xaa can be any naturally occurring amino acid other than a Glycine
PAGLLDLRQGMFAQLVAQNVLLIDGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 37 - protein, 174 aa, Artificial sequence
  Synthetic sequence
  misc_feature (43)..(43): Xaa can be any naturally occurring amino acid other than a Glycine
PAGLLDLRQGMFAQLVAQNVLLIDGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 38 - protein, 174 aa, Artificial sequence
  Synthetic sequence
  misc_feature (44)..(44): Xaa can be any naturally occurring amino acid other than a Leucine
PAGLLDLRQGMFAQLVAQNVLLIDGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 39 - protein, 174 aa, Artificial sequence
  Synthetic sequence
  misc_feature (45)..(45): Xaa can be any naturally occurring amino acid other than a Serine
PAGLLDLRQGMFAQLVAQNVLLIDGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 40 - protein, 174 aa, Artificial sequence
  Synthetic sequence
  misc_feature (46)..(46): Xaa can be any naturally occurring amino acid other than a Tyrosine
PAGLLDLRQGMFAQLVAQNVLLIDGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 41 - protein, 174 aa, Artificial sequence
  Synthetic sequence
  misc_feature (48)..(48): Xaa can be any naturally occurring amino acid other than a Glutamic Acid
PAGLLDLRQGMFAQLVAQNVLLIDGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 42 - protein, 174 aa, Artificial sequence
  Synthetic sequence
  misc_feature (49)..(49): Xaa can be any naturally occurring amino acid other than a Aspartic acid
PAGLLDLRQGMFAQLVAQNVLLIDGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 43 - protein, 174 aa, Artificial sequence
  Synthetic sequence
  misc_feature (50)..(50): Xaa can be any naturally occurring amino acid other than a Threonine
PAGLLDLRQGMFAQLVAQNVLLIDGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 44 - protein, 174 aa, Artificial sequence
  Synthetic sequence
  misc_feature (51)..(51): Xaa can be any naturally occurring amino acid other than a Lysine
PAGLLDLRQGMFAQLVAQNVLLIDGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 45 - protein, 174 aa, Artificial sequence
  Synthetic sequence
  misc_feature (52)..(52): Xaa can be any naturally occurring amino acid other than a Glutamic acid
PAGLLDLRQGMFAQLVAQNVLLIDGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 46 - protein, 174 aa, Artificial sequence
  Synthetic sequence
  misc_feature (64)..(64): Xaa can be any naturally occurring amino acid other than a Phenylalanine
PAGLLDLRQGMFAQLVAQNVLLIDGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 47 - protein, 174 aa, Artificial sequence
  Synthetic sequence
  misc_feature (65)..(65): Xaa can be any naturally occurring amino acid other than a Phenylalanine
PAGLLDLRQGMFAQLVAQNVLLIDGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 174 aa, Artificial sequence
  Synthetic sequence
  misc_feature (66)..(66): Xaa can be any naturally occurring amino acid other than a Glutamine
PAGLLDLRQGMFAQLVAQNVLLIDGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 49 - protein, 174 aa, Artificial sequence
  Synthetic sequence
  misc_feature (67)..(67): Xaa can be any naturally occurring amino acid other than a Leucine
PAGLLDLRQGMFAQLVAQNVLLIDGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 50 - protein, 174 aa, Artificial sequence
  Synthetic sequence
  misc_feature (68)..(68): Xaa can be any naturally occurring amino acid other than a Glutamic acid
PAGLLDLRQGMFAQLVAQNVLLIDGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 51 - protein, 174 aa, Artificial sequence
  Synthetic sequence
  misc_feature (69)..(69): Xaa can be any naturally occurring amino acid other than a Leucine
PAGLLDLRQGMFAQLVAQNVLLIDGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 52 - protein, 174 aa, Artificial sequence
  Synthetic sequence
  misc_feature (70)..(70): Xaa can be any naturally occurring amino acid other than a Arginine
PAGLLDLRQGMFAQLVAQNVLLIDGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 53 - protein, 174 aa, Artificial sequence
  Synthetic sequence
  misc_feature (71)..(71): Xaa can be any naturally occurring amino acid other than a Arginine
PAGLLDLRQGMFAQLVAQNVLLIDGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 54 - protein, 174 aa, Artificial sequence
  Synthetic sequence
  misc_feature (72)..(72): Xaa can be any naturally occurring amino acid other than a Valine
PAGLLDLRQGMFAQLVAQNVLLIDGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 55 - protein, 174 aa, Artificial sequence
  Synthetic sequence
  misc_feature (73)..(73): Xaa can be any naturally occurring amino acid other than a Valine
PAGLLDLRQGMFAQLVAQNVLLIDGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 56 - protein, 174 aa, Artificial sequence
  Synthetic sequence
  misc_feature (75)..(75): Xaa can be any naturally occurring amino acid other than a Glycine
PAGLLDLRQGMFAQLVAQNVLLIDGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 57 - protein, 174 aa, Artificial sequence
  Synthetic sequence
  misc_feature (76)..(76): Xaa can be any naturally occurring amino acid other than a Glutamic acid
PAGLLDLRQGMFAQLVAQNVLLIDGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - protein, 174 aa, Artificial sequence
  Synthetic sequence
  misc_feature (77)..(77): Xaa can be any naturally occurring amino acid other than a Glycine
PAGLLDLRQGMFAQLVAQNVLLIDGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 59 - protein, 174 aa, Artificial sequence
  Synthetic sequence
  misc_feature (78)..(78): Xaa can be any naturally occurring amino acid other than a Serine
PAGLLDLRQGMFAQLVAQNVLLIDGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - protein, 174 aa, Artificial sequence
  Synthetic sequence
  misc_feature (104)..(104): Xaa can be any naturally occurring amino acid other than a Aspartic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 61 - protein, 174 aa, Artificial sequence
  Synthetic sequence
  misc_feature (105)..(105): Xaa can be any naturally occurring amino acid other than a Leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 62 - protein, 174 aa, Artificial sequence
  Synthetic sequence
  misc_feature (106)..(106): Xaa can be any naturally occurring amino acid other than a Proline
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 63 - protein, 174 aa, Artificial sequence
  Synthetic sequence
  misc_feature (107)..(107): Xaa can be any naturally occurring amino acid other than a Proline
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPXASSEARNSAFGFQGRLLHLSAGQRLGVHLHTEARARHAWQLTQGATVLGLFRVTPEIPAGLPSPRSE

SEQ ID NO: 64 - protein, 174 aa, Artificial sequence
  Synthetic sequence
  misc_feature (109)..(109): Xaa can be any naturally occurring amino acid other than a Serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 65 - protein, 174 aa, Artificial sequence
  Synthetic sequence
  misc_feature (110)..(110): Xaa can be any naturally occurring amino acid other than a Serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 66 - protein, 174 aa, Artificial sequence
  Synthetic sequence
  misc_feature (111)..(111): Xaa can be any naturally occurring amino acid other than a Glutamic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 67 - protein, 174 aa, Artificial sequence
  Synthetic sequence
  misc_feature (113)..(113): Xaa can be any naturally occurring amino acid other than a Arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 68 - protein, 174 aa, Artificial sequence
  Synthetic sequence
  misc_feature (114)..(114): Xaa can be any naturally occurring amino acid other than a Asparagine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 69 - protein, 174 aa, Artificial sequence
  Synthetic sequence
  misc_feature (115)..(115): Xaa can be any naturally occurring amino acid other than a Serine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 70 - protein, 174 aa, Artificial sequence
  Synthetic sequence
  misc_feature (117)..(117): Xaa can be any naturally occurring amino acid other than a Phenylalanine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 71 - protein, 174 aa, Artificial sequence
  Synthetic sequence
  misc_feature (130)..(130): Xaa can be any naturally occurring amino acid other than a Glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 72 - protein, 174 aa, Artificial sequence
  Synthetic sequence
  misc_feature (131)..(131): Xaa can be any naturally occurring amino acid other than a Asparagine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 73 - protein, 174 aa, Artificial sequence
  Synthetic sequence
  misc_feature (132)..(132): Xaa can be any naturally occurring amino acid other than a Leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 74 - protein, 174 aa, Artificial sequence
  Synthetic sequence
  misc_feature (133)..(133): Xaa can be any naturally occurring amino acid other than a Glycine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 75 - protein, 174 aa, Artificial sequence
  Synthetic sequence
  misc_feature (134)..(134): Xaa can be any naturally occurring amino acid other than a Valine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 76 - protein, 174 aa, Artificial sequence
  Synthetic sequence
  misc_feature (135)..(135): Xaa can be any naturally occurring amino acid other than a Histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 77 - protein, 174 aa, Artificial sequence
  Synthetic sequence
  misc_feature (136)..(136): Xaa can be any naturally occurring amino acid other than a Leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 78 - protein, 174 aa, Artificial sequence
  Synthetic sequence
  misc_feature (137)..(137): Xaa can be any naturally occurring amino acid other than a Histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 79 - protein, 174 aa, Artificial sequence
  Synthetic sequence
  misc_feature (138)..(138): Xaa can be any naturally occurring amino acid other than a Threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 80 - protein, 174 aa, Artificial sequence
  Synthetic sequence
  misc_feature (139)..(139): Xaa can be any naturally occurring amino acid other than a Glutamic acid
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 81 - protein, 174 aa, Artificial sequence
  Synthetic sequence
  misc_feature (141)..(141): Xaa can be any naturally occurring amino acid other than Arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 82 - protein, 174 aa, Artificial sequence
  Synthetic sequence
  misc_feature (143)..(143): Xaa can be any naturally occurring amino acid other than a Arginine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 83 - protein, 174 aa, Artificial sequence
  Synthetic sequence
  misc_feature (144)..(144): Xaa can be any naturally occurring amino acid other than a Histidine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 84 - protein, 174 aa, Artificial sequence
  Synthetic sequence
  misc_feature (146)..(146): Xaa can be any naturally occurring amino acid other than a Tryptophan
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 85 - protein, 174 aa, Artificial sequence
  Synthetic sequence
  misc_feature (148)..(148): Xaa can be any naturally occurring amino acid other than a  Leucine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 86 - protein, 174 aa, Artificial sequence
  Synthetic sequence
  misc_feature (149)..(149): Xaa can be any naturally occurring amino acid other than a Threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 87 - protein, 174 aa, Artificial sequence
  Synthetic sequence
  misc_feature (150)..(150): Xaa can be any naturally occurring amino acid other than a Glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 88 - protein, 174 aa, Artificial sequence
  Synthetic sequence
  misc_feature (151)..(151): Xaa can be any naturally occurring amino acid other than a Glycine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 89 - protein, 174 aa, Artificial sequence
  Synthetic sequence
  misc_feature (153)..(153): Xaa can be any naturally occurring amino acid other than a Threonine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRSE

SEQ ID NO: 90 - protein, 174 aa, Artificial sequence
  Synthetic sequence
  misc_feature (154)..(154): Xaa can be any naturally occurring amino acid other than a Valine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 91 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 92 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 94 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 96 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 97 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 98 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 99 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 101 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 102 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 103 - protein, 119 aa, homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 104 - protein, 119 aa, Pan troglodytes; identical to SEQ ID NO: 103

SEQ ID NO: 105 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 106 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 107 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 108 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 109 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 110 - protein, 208 aa, Artificial sequence [sequence omitted]

SEQ ID NO: 111 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 112 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 113 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 114 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 115 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 116 - protein, 8 aa, Artificial sequence
  Synthetic sequence
LEVLFQGP

SEQ ID NO: 117 - protein, 7 aa, Artificial sequence
  Synthetic sequence
ENLYTQS

SEQ ID NO: 118 - protein, 4 aa, Artificial sequence
  Synthetic sequence
LVPR

SEQ ID NO: 119 - protein, 22 aa, Artificial sequence
  Synthetic sequence
GSGATNFSLLKQAGDVEENPGP

SEQ ID NO: 120 - protein, 21 aa, Artificial sequence
  Synthetic sequence
GSGEGRGSLLTCGDVEENPGP

SEQ ID NO: 121 - protein, 23 aa, Artificial sequence
  Synthetic sequence
GSGQCTNYALLKLAGDVESNPGP

SEQ ID NO: 122 - protein, 25 aa, Artificial sequence
  Synthetic sequence
GSGVKQTLNFDLLKLAGDVESNPGP

SEQ ID NO: 123 - protein, 5 aa, Artificial sequence
  Synthetic sequence
  Misc_Feat (1)..(5): This stretch of residues may be repeated.
GSGGS

SEQ ID NO: 124 - protein, 4 aa, Artificial sequence
  Synthetic sequence
  Misc_feat (1)..(4): This stretch of residues may be repeated.
GGGS

SEQ ID NO: 125 - protein, 4 aa, Artificial sequence
  Synthetic sequence
GGSG

SEQ ID NO: 126 - protein, 5 aa, Artificial sequence
  Synthetic sequence
GGSGG

SEQ ID NO: 127 - protein, 5 aa, Artificial sequence
  Synthetic sequence
GSGSG

SEQ ID NO: 128 - protein, 5 aa, Artificial sequence
  Synthetic sequence
GSGGG

SEQ ID NO: 129 - protein, 5 aa, Artificial sequence
  Synthetic sequence
GGGSG

SEQ ID NO: 130 - protein, 5 aa, Artificial sequence
  Synthetic sequence
GSSSG

SEQ ID NO: 131 - protein, 5 aa, Artificial sequence
  Synthetic sequence
  Misc_feat (1)..(5): This stretch of residues may be repeated.
GSSSS

SEQ ID NO: 132 - protein, 5 aa, Artificial sequence
  Synthetic sequence
  Misc_feat (1)..(5): This stretch of residues may be repeated.
GGGGS

SEQ ID NO: 133 - protein, 15 aa, Artificial sequence
  Synthetic sequence
GCGASGGGGSGGGGS

SEQ ID NO: 134 - protein, 276 aa, Artificial sequence [sequence omitted]

SEQ ID NO: 135 - protein, 274 aa, Artificial sequence
  Synthetic sequence
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 136 - protein, 276 aa, Artificial sequence [sequence omitted]

SEQ ID NO: 137 - protein, 276 aa, Artificial sequence
  Synthetic sequence
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 138 - protein, 99 aa, Artificial sequence
  Synthetic sequence
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 139 - protein, 276 aa, Artificial sequence; identical to SEQ ID NO: 137
  Synthetic sequence

SEQ ID NO: 140 - protein, 9 aa, Artificial sequence
  Synthetic sequence
YPYDVPDYA

SEQ ID NO: 141 - protein, 8 aa, Artificial sequence
  Synthetic sequence
DYKDDDDK

SEQ ID NO: 142 - protein, 10 aa, Artificial sequence
  Synthetic sequence
EQKLISEEDL

SEQ ID NO: 143 - protein, 5 aa, Artificial sequence
  Synthetic sequence
HHHHH

SEQ ID NO: 144 - protein, 6 aa, Artificial sequence
  Synthetic sequence
HHHHHH

SEQ ID NO: 145 - protein, 10 aa, Artificial sequence
  Synthetic sequence
EQKLISEEDL

SEQ ID NO: 146 - protein, 8 aa, Artificial sequence
  Synthetic sequence
DYKDDDDK

SEQ ID NO: 147 - protein, 8 aa, Artificial sequence
  Synthetic sequence
WSHPQFEK

SEQ ID NO: 148 - protein, 9 aa, Artificial sequence
  Synthetic sequence
YPYDVPDYA

SEQ ID NO: 149 - protein, 5 aa, Artificial sequence
  Synthetic sequence
RYIRS

SEQ ID NO: 150 - protein, 4 aa, Artificial sequence
  Synthetic sequence
FHHT

SEQ ID NO: 151 - protein, 17 aa, Artificial sequence
  Synthetic sequence
WEAAAREACCRECCARA

SEQ ID NO: 152 - protein, 5 aa, Artificial sequence
  Synthetic sequence
DDDDK

(19) United States
(12) Patent Application Publication
(10) Pub. No.: US 2019/0046648 A1
(43) Pub. Date: Feb. 14, 2019

(54) T-CELL MODULATORY MULTIMERIC POLYPEPTIDES AND METHODS OF USE THEREOF

(71) Applicant: Cue Biopharma, Inc., Cambridge, MA (US)

(72) Inventors: Ronald D. Seidel, III, Cambridge, MA (US); Rodolfo Chaparro, Cambridge, MA (US)

(21) Appl. No.: 16/077,303

(22) PCT Filed: Mar. 2, 2017
(86) PCT No.: PCT/US17/20480

§ 371 Date: Aug. 10, 2018

Related U.S. Application Data
(60) Provisional application No. 62/303,268, filed on Mar. 3, 2016.

(51) Int. Cl. A61K 47/42; A61K 38/17; C07K 14/74; C12N 15/62; A61K 47/65; A61K 35/17; A61P 31/12; A61P 31/04

(52) U.S. Cl. CPC A61K 47/42; A61K 38/1774; C07K 14/70539; A61P 31/04; A61K 47/65; A61K 35/17; A61P 31/12; C12N 15/62

(57) ABSTRACT

The present disclosure provides variant immunomodulatory polypeptides, and fusion polypeptides comprising the variant immunomodulatory peptides. The present disclosure provides T-cell modulatory multimeric polypeptides, and compositions comprising same, where the T-cell modulatory multimeric polypeptides comprise a variant immunomodulatory polypeptide of the present disclosure. The present disclosure provides nucleic acids comprising nucleotide sequences encoding the T-cell modulatory multimeric polypeptides, and host cells comprising the nucleic acids. The present disclosure provides methods of modulating the activity of a T cell; the methods comprise contacting the T cell with a T-cell modulatory multimeric polypeptide of the present disclosure.

### CROSS-REFERENCE

This application claims the benefit of U.S. Provisional Patent Application No. 62/303,268, filed Mar. 3, 2016, which application is incorporated herein by reference in its entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides variant immunomodulatory polypeptides, and fusion polypeptides comprising the variant immunomodulatory peptides. The present disclosure provides T-cell modulatory multimeric polypeptides, and compositions comprising same, where the T-cell modulatory multimeric polypeptides comprise a variant immunomodulatory polypeptide of the present disclosure. The present disclosure provides nucleic acids comprising nucleotide sequences encoding the T-cell modulatory multimeric polypeptides, and host cells comprising the nucleic acids. The present disclosure provides methods of modulating the activity of a T cell; the methods comprise contacting the T cell with a T-cell modulatory multimeric polypeptide of the present disclosure.

The present disclosure provides a variant 4-1BBL immunomodulatory polypeptide comprising an amino acid sequence having at least 85%, at least 90%, at least 95%, at least 98%, or at least 99%, amino acid sequence identity to the 4-1BBL amino acid sequence depicted in FIG. 2A or to the 4-1BBL amino acid sequence set forth in one of SEQ ID NOs:1-3, wherein the variant 4-1BBL immunomodulatory polypeptide has one or more (e.g., 1, 2, 3, 4, 5, 6, 7, 8, 9, or 10, or more than 10) amino acid substitutions relative to the 4-1BBL amino acid sequence depicted in FIG. 2A or to the 4-1BBL amino acid sequence set forth in one of SEQ ID NOs:1-3; and wherein the variant 4-1BBL immunomodulatory polypeptide: i) exhibits reduced binding affinity to a 4-1BB polypeptide having an amino acid sequence depicted in FIG. 3 and set forth in SEQ ID NO:91, compared to the binding affinity of the 4-1BBL amino acid sequence depicted in FIG. 2A or set forth in one of SEQ ID NOs:1-3 for the 4-1BB polypeptide; and/or ii) wherein the variant 4-1BBL immunomodulatory polypeptide exhibits increased production levels by a mammalian cell, compared to the production levels of the 4-1BBL amino acid sequence depicted in FIG. 2A or set forth in one of SEQ ID NOs:1-3. In some cases, the polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A. In some cases, the variant immunomodulatory polypeptide exhibits less than 50% of binding affinity exhibited by to the 4-1BBL amino acid sequence depicted in FIG. 2A, or as set forth in one of SEQ ID NOs:1-3, for the 4-1BB polypeptide.

The present disclosure provides a multimeric polypeptide comprising: a) a first polypeptide comprising, in order from N-terminus to C-terminus: i) an epitope; ii) a first major histocompatibility complex (MHC) polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the multimeric polypeptide comprises one or more immunomodulatory domains, wherein the one or more immunomodulatory domain is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, wherein the immunomodulatory domain is a variant 4-1BBL immunomodulatory polypeptide as described above or elsewhere herein; and wherein: i) the multimeric polypeptide exhibits reduced binding affinity to a 4-1BB polypeptide having an amino acid sequence depicted in FIG. 3 and set forth in SEQ ID NO:91, compared to the binding affinity of a control multimeric polypeptide comprising an immunomodulatory domain comprising the 4-1BBL amino acid sequence depicted in FIG. 2A or set forth in one of SEQ ID NOs:1-3 for the 4-1BB polypeptide; and/or ii) wherein the multimeric polypeptide exhibits increased production levels by a mammalian cell, compared to the production levels of a control multimeric polypeptide comprising an immunomodulatory domain comprising the 4-1BBL amino acid sequence depicted in FIG. 2A or set forth in one of SEQ ID NOs:1-3. In some cases, the multimeric polypeptide comprises: a) a first polypeptide comprising, in order from N-terminus to C-terminus: i) an epitope; ii) a first MHC polypeptide; and iii) an immunomodulatory domain; and b) a second polypeptide comprising, in order from N-terminus to C-terminus: i) a second MHC polypeptide; and ii) an Ig Fc polypeptide. In some cases, the multimeric polypeptide comprises: a) a first polypeptide comprising, in order from N-terminus to C-terminus: i) an epitope; and ii) a first MHC polypeptide; and b) a second polypeptide comprising, in order from N-terminus to C-terminus: i) an immunomodulatory domain; iii) a second MHC polypeptide; and iv) an immunoglobulin (Ig) Fc polypeptide. In some cases, the multimeric polypeptide comprises: a) a first polypeptide comprising, in order from N-terminus to C-terminus: i) an epitope; and ii) a first MHC polypeptide; and b) a second polypeptide comprising, in order from N-terminus to C-terminus: i) a second MHC polypeptide; and ii) an Ig Fc polypeptide; and iii) an immunomodulatory domain. In some cases, the multimeric polypeptide comprises: a) a first polypeptide comprising, in order from N-terminus to C-terminus: i) an epitope; and ii) a first MHC polypeptide; and b) a second polypeptide comprising, in order from N-terminus to C-terminus: i) a second MHC polypeptide; and ii) an immunomodulatory domain. In some cases, the multimeric polypeptide comprises: a) a first polypeptide comprising, in order from N-terminus to C-terminus: i) an epitope; and ii) a first MHC polypeptide; and b) a second polypeptide comprising, in order from N-terminus to C-terminus: i) an immunomodulatory domain; and ii) a second MHC polypeptide. In some cases, the multimeric polypeptide comprises: a) a first polypeptide comprising, in order from N-terminus to C-terminus: i) an epitope; ii) a first MHC polypeptide; and iii) an immunomodulatory domain; and b) a second polypeptide comprising, in order from N-terminus to C-terminus: i) a second MHC polypeptide. In some cases, the non-Ig scaffold is an XTEN polypeptide, a transferrin polypeptide, an elastin-like polypeptide, a silk-like polypeptide, or a silk-elastin-like polypeptide. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to any one of the amino acid sequences set forth in SEQ ID NO:6. In some cases, the MHC class I heavy chain polypeptide is an HLA-A, an HLA-B, or an HLA-C heavy chain. In some cases, the MHC class I heavy chain polypeptide comprises an amino acid sequence having at least 85% amino acid sequence identity to the amino acid sequence depicted in any one of FIG. 5A-5C. In some cases, the first MHC polypeptide is an MHC Class II alpha chain polypeptide; and wherein the second MHC polypeptide is an MHC class II beta chain polypeptide. In some cases, the epitope is a T-cell epitope. In some cases, the multimeric polypeptide comprises an Fc polypeptide, and wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIG. 4A-4C. In some cases, the first polypeptide and the second polypeptide are non-covalently associated. In some cases, the first polypeptide and the second polypeptide are covalently linked. In some cases, the covalent linkage is via a disulfide bond. In some cases, the first MHC polypeptide or a linker between the epitope and the first MHC polypeptide comprises an amino acid substitution to provide a first Cys residue, and the second MHC polypeptide comprises an amino acid substitution to provide a second Cys residue, and wherein the disulfide linkage is between the first and the second Cys residues. In some cases, the multimeric polypeptide comprises a first linker interposed between the epitope and the first MHC polypeptide. In some cases, the variant 4-1BBL immunomodulatory polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A. In some cases, the multimeric polypeptide comprises 2 or more immunomodulatory polypeptides. In some cases, the 2 or more immunomodulatory polypeptides are in tandem. In some cases, the multimeric polypeptide comprises a third polypeptide, wherein the third polypeptide comprises an immunomodulatory polypeptide comprising an amino acid sequence having at least 90% amino acid sequence identity to the immunomodulatory polypeptide of the first polypeptide or the second polypeptide. In some cases, the third polypeptide is covalently linked to the first polypeptide. In some cases, the second polypeptide comprises, in order from N-terminus to C-terminus: i) the second MHC polypeptide; ii) the Ig Fc polypeptide; and iii) an affinity tag.

The present disclosure provides a nucleic acid comprising a nucleotide sequence encoding a recombinant polypeptide, i) wherein the recombinant polypeptide comprises, in order from N-terminus to C-terminus: a) an epitope; b) a first major histocompatibility complex (MHC) polypeptide; c) an immunomodulatory polypeptide; d) a proteolytically cleavable linker or a ribosome skipping signal; e) a second MHC polypeptide; and f) an immunoglobulin (Ig) Fc polypeptide; wherein the immunomodulatory polypeptide is a variant immunomodulatory polypeptide as described above or elsewhere herein; or ii) wherein the recombinant polypeptide comprises, in order from N-terminus to C-terminus: a) an epitope; b) a first MHC polypeptide;

c) a proteolytically cleavable linker or a ribosome skipping signal; d) an immunomodulatory polypeptide; e) a second MHC polypeptide; and f) an Ig Fc polypeptide, wherein the immunomodulatory polypeptide is a variant immunomodulatory polypeptide as described above or elsewhere herein. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to any one of the amino acid sequences depicted in FIG. 6. In some cases, the MHC class I heavy chain polypeptide is an HLA-A, HLA-B, or HLA-C heavy chain. In some cases, the MHC class I heavy chain polypeptide comprises an amino acid sequence having at least 85% amino acid sequence identity to the amino acid sequence depicted in any one of FIG. 5A-5C. In some cases, the first MHC polypeptide is an MHC Class II alpha chain polypeptide; and wherein the second MHC polypeptide is an MHC class II beta chain polypeptide. In some cases, the epitope is a T-cell epitope. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIGS. 4A-4C. In some cases, the variant 4-1BBL immunomodulatory polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A. In some cases, the multimeric polypeptide comprises a second immunomodulatory polypeptide selected from a CD7, CD30L, CD40, CD70, CD83, HLA-G, MICA, MICB, HVEM, lymphotoxin beta receptor, 3/TR6, ILT3, ILT4, and HVEM. In some cases, the proteolytically cleavable linker or ribosome skipping signal comprises an amino acid sequence selected from: a) LEVLFQGP (SEQ ID NO:116); b) ENLYTQS (SEQ ID NO:117); c) a furin cleavage site; d) LVPR (SEQ ID NO:118); e) GSGATNFSLLKQAGDVEENPGP (SEQ ID NO:119); f) GSGEGRGSLLTCGDVEENPGP (SEQ ID NO:120); g) GSGQCTNYALLKLAGDVESNPGP (SEQ ID NO:121); and h) GSGVKQTLNFDLLKLAGDVESNPGP (SEQ ID NO:122). In some cases, the recombinant polypeptide comprises, in order from N-terminus to C-terminus: a) a first leader peptide; b) the epitope; c) the first MHC polypeptide; d) the immunomodulatory polypeptide; e) the proteolytically cleavable linker or ribosome skipping signal; f) a second leader peptide; g) the second MHC polypeptide; and h) the immunoglobulin (Ig) Fc polypeptide. In some cases, the first leader peptide and the second leader peptide is a β2-M leader peptide. In some cases, the nucleotide sequence is operably linked to a transcriptional control element. In some cases, the transcriptional control element is a promoter that is functional in a eukaryotic cell. In some cases, the first MHC polypeptide or a linker between the epitope and the first MHC polypeptide comprises an amino acid substitution to provide a first Cys residue, and the second MHC polypeptide comprises an amino acid substitution to provide a second Cys residue, and wherein the first and the second Cys residues provide for a disulfide linkage between the first MHC polypeptide and the second MHC polypeptide. The present disclosure provides a recombinant expression vector comprising a nucleic acid as described above or elsewhere herein. In some cases, the vector is a viral vector or a non-viral vector. The present disclosure provides a host cell genetically modified with a recombinant expression vector as described above or elsewhere herein. In some cases, the host cell is in vitro. In some cases, the host cell is genetically modified such that the cell does not produce an endogenous MHC β2-microglobulin polypeptide. In some cases, the host cell is a T lymphocyte.

The present disclosure provides a composition comprising: a) a first nucleic acid comprising a nucleotide sequence encoding a first polypeptide comprising, in order from N-terminus to C-terminus: i) an epitope; ii) a first MHC polypeptide; and iii) an immunomodulatory domain, wherein the immunomodulatory domain is a variant immunomodulatory polypeptide as described above or elsewhere herein; and b) a first nucleic acid comprising a nucleotide sequence encoding a second polypeptide comprising, in order from N-terminus to C-terminus: i) a second MHC polypeptide; and ii) an Ig Fc polypeptide. The present disclosure provides a host cell genetically modified with a nucleic acid composition as described above or elsewhere herein.

The present disclosure provides a composition comprising: a) a first nucleic acid comprising a nucleotide sequence encoding a first polypeptide comprising, in order from N-terminus to C-terminus: i) an epitope; and ii) a first MHC polypeptide; and b) a first nucleic acid comprising a nucleotide sequence encoding a second polypeptide comprising, in order from N-terminus to C-terminus: i) an immunomodulatory domain, wherein the immunomodulatory domain is a variant immunomodulatory polypeptide as described above or elsewhere herein; ii) a second MHC polypeptide; and iii) an Ig Fc polypeptide. In some cases, the first and/or the second nucleic acid is present in a recombinant expression vector.

The present disclosure provides a host cell genetically modified with a nucleic acid composition as described above or elsewhere herein.

The present disclosure provides a method of producing a multimeric polypeptide as described above or elsewhere herein, the method comprising: a) culturing a host cell as described above or elsewhere herein in vitro in a culture medium under conditions such that the host cell synthesizes the multimeric polypeptide; and b) isolating the multimeric polypeptide from the host cell and/or from the culture medium. In some cases, the second polypeptide comprises an affinity tag, and wherein said isolating comprises contacting the multimeric polypeptide produced by the cell with a binding partner for the affinity tag, wherein the binding partner is immobilized, thereby immobilizing the multimeric polypeptide. In some cases, the method comprises eluting the immobilized multimeric polypeptide.

The present disclosure provides a method of selectively modulating the activity of an epitope-specific T cell, the method comprising contacting the T cell with a multimeric polypeptide as described above or elsewhere herein, wherein said contacting selectively modulates the activity of the epitope-specific T cell. In some cases, the immunomodulatory polypeptide is an activating polypeptide, and wherein the multimeric polypeptide activates the epitope-specific T cell. In some cases, the immunomodulatory polypeptide is an inhibiting polypeptide, and wherein the multimeric polypeptide inhibits the epitope-specific T cell. In some cases, said contacting is in vitro. In some cases, said contacting is in vivo.

The present disclosure provides a method of selectively modulating the activity of an epitope-specific T cell in an individual, the method comprising administering to the individual an effective amount of a multimeric polypeptide as described above or elsewhere herein effective to selectively modulate the activity of an epitope-specific T cell in an individual. In some cases, the immunomodulatory polypeptide is an activating polypeptide, and wherein the multimeric polypeptide activates the epitope-specific T cell. In some cases, the epitope is a cancer-associated epitope, and wherein said administering selectively increases the activity of a T cell specific for the cancer-associate epitope. In some cases, the immunomodulatory polypeptide is an inhibitory polypeptide, and wherein the multimeric polypeptide inhibits activity of the epitope-specific T cell. In some cases, the epitope is a self-epitope, and wherein said administering selectively inhibits the activity of a T cell specific for the self-epitope.

The present disclosure provides a method of treating an infection in an individual, the method comprising administering to the individual an effective amount of a) a multimeric polypeptide as described above or elsewhere herein; or b) one or more recombinant expression vectors comprising nucleotide sequences encoding a multimeric polypeptide as described above or elsewhere herein; or c) one or more mRNAs comprising nucleotide sequences encoding a multimeric polypeptide as described above or elsewhere herein, wherein the epitope is a pathogen-associated epitope, wherein the immunomodulatory polypeptide is an activating polypeptide, and wherein said administering effective to selectively modulate the activity of a pathogen-associated epitope-specific T cell in an individual. In some cases, the pathogen is a virus, a bacterium, or a protozoan. In some cases, said administering is subcutaneous. In some cases, said administering is intravenous. In some cases, said administering is intramuscular. In some cases, said administering is systemic. In some cases, said administering is distal to a treatment site. In some cases, said administering is local. In some cases, said administering is at or near a treatment site.

The present disclosure provides a composition comprising: a) a multimeric polypeptide as described above or elsewhere herein; and b) a pharmaceutically acceptable excipient.

The present disclosure provides a composition comprising: a) a nucleic acid as described above or elsewhere herein or a recombinant expression vector as described above or elsewhere herein; and b) a pharmaceutically acceptable excipient.

### BRIEF DESCRIPTION OF THE DRAWINGS

FIG. 1A-1D schematically depict various embodiments of a T-cell modulatory multimeric polypeptide of the present disclosure. In these embodiments, disulfide bonds are formed between MHC (e.g., HLA) polypeptides present in separate polypeptides.

FIG. 2A-2IIII provide an amino acid sequence of a 4-1BBL (FIG. 2A) and examples of variant 4-1BBL polypeptides (FIG. 2B-2IIII).

FIG. 3 provides an amino acid sequence of 4-1BB (FIG. 3A-3C).

FIG. 4A-4C provide amino acid sequences of immunoglobulin Fc polypeptides.

FIG. 5A-5C provide amino acid sequences of human leukocyte antigen (HLA) Class I heavy chain polypeptides. Signal sequences are underlined.

FIG. 6 provides a multiple amino acid sequence alignment of beta-2 microglobulin (β2M) precursors (i.e., including the leader sequence) from Homo sapiens (NP_004039.1; SEQ ID NO:103), Pan troglodytes (NP_001009066.1; SEQ ID NO:104), Macaca mulatta (NP_001040602.1; SEQ ID NO:105), Bos Taurus (NP_776318.1; SEQ ID NO:106) and Mus musculus (NP_033865.2; SEQ ID NO:107). Amino acids 1-20 are a signal peptide.

FIG. 7A-7B provide amino acid sequences of PD-L1 polypeptides.

FIG. 8 provides an amino acid sequence of a CD80 polypeptide.

FIG. 9 provides an amino acid sequence of an ICOS-L polypeptide.

FIG. 10 provides an amino acid sequence of an OX40L polypeptide.

FIG. 11 provides an amino acid sequence of a PD-L2 polypeptide.

FIG. 12 provides an amino acid sequence of a CD86 (B7-2) polypeptide.

FIG. 13 provides an amino acid sequence of a Fas ligand (FAS-L) polypeptide.

FIG. 14A-14B depicts interferon-gamma (IFN-γ) secretion by target cells contacted with a synTac polypeptide for 3 days (FIG. 14A) or 5 days (FIG. 14B) according to an embodiment of the present disclosure.

FIG. 15A-15B depicts interleukin-2 (IL-2) secretion by target cells contacted with a synTac polypeptide for 3 days (FIG. 15A) or 5 days (FIG. 15B) according to an embodiment of the present disclosure.

FIG. 16A-16B depicts interleukin-6 (IL-6) secretion by target cells contacted with a synTac polypeptide for 3 days (FIG. 16A) or 5 days (FIG. 16B) according to an embodiment of the present disclosure.

FIG. 17A-17B depicts tumor necrosis factor-alpha (TNFα) secretion by target cells contacted with a synTac polypeptide for 3 days (FIG. 17A) or 5 days (FIG. 17B) according to an embodiment of the present disclosure.

FIG. 18A-18B depicts interleukin-10 (IL-10) secretion by target cells contacted with a synTac polypeptide for 3 days (FIG. 18A) or 5 days (FIG. 18B) according to an embodiment of the present disclosure.

FIG. 19A-19B depicts interleukin-17A (IL-17A) secretion by target cells contacted with a synTac polypeptide for 3 days (FIG. 19A) or 5 days (FIG. 19B) according to an embodiment of the present disclosure.

FIG. 20A-20B depicts interleukin-4 (IL-4) secretion by target cells contacted with a synTac polypeptide for 3 days (FIG. 20A) or 5 days (FIG. 20B) according to an embodiment of the present disclosure.

FIG. 21 depicts proliferation of target cells contacted with a synTac polypeptide according to an embodiment of the present disclosure.

FIG. 22 depicts viability of target cells contacted with a synTac polypeptide according to an embodiment of the present disclosure.

FIG. 23 depicts expression levels of various synTac polypeptides produced in CHO cells.

FIG. 24 depicts the in vivo effect of a synTac polypeptide of the present disclosure on tumor volume.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“Binding” as used herein (e.g. with reference to binding of a T-cell modulatory multimeric polypeptide of the present disclosure to a polypeptide (e.g., a T-cell receptor) on a T cell) refers to a non-covalent interaction between. Binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

“Co-stimulatory polypeptide,” as the term is used herein, includes a polypeptide on an antigen presenting cell (APC) (e.g., a dendritic cell, a B cell, and the like) that specifically binds a cognate co-stimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

A “modulatory domain” of a T-cell modulatory multimeric polypeptide of the present disclosure comprises a co-stimulatory polypeptide.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells, for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a modulatory domain” includes a plurality of such modulatory domains and reference to “the HLA polypeptide” includes reference to one or more HLA polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides variant immunomodulatory polypeptides, and fusion polypeptides comprising the variant immunomodulatory peptides. The present disclosure provides T-cell modulatory multimeric polypeptides, and compositions comprising same, where the T-cell modulatory multimeric polypeptides comprise a variant immunomodulatory polypeptide of the present disclosure. The present disclosure provides nucleic acids comprising nucleotide sequences encoding the T-cell modulatory multimeric polypeptides, and host cells comprising the nucleic acids. The present disclosure provides methods of modulating the activity of a T cell; the methods comprise contacting the T cell with a T-cell modulatory multimeric polypeptide of the present disclosure.

A T-cell modulatory multimeric polypeptide of the present disclosure is also referred to as a “synTac polypeptide.” A synTac polypeptide of the present disclosure comprises a variant modulatory domain, where the variant modulatory domain exhibits reduced binding affinity to an immunomodulatory polypeptide. A synTac polypeptide of the present disclosure can modulate the activity of a target T-cell. A synTac polypeptide of the present disclosure provides for enhanced target cell specificity.

### Variant Immunomodulatory Polypeptides

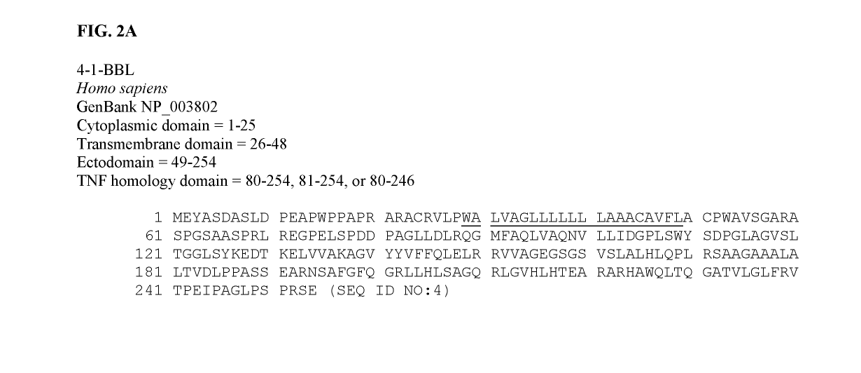

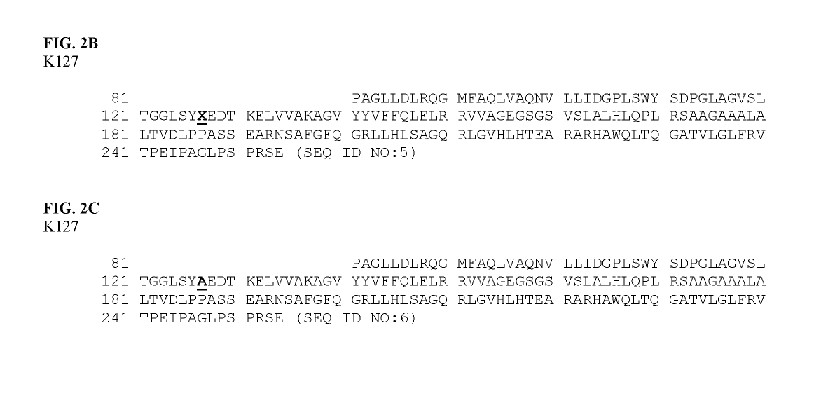

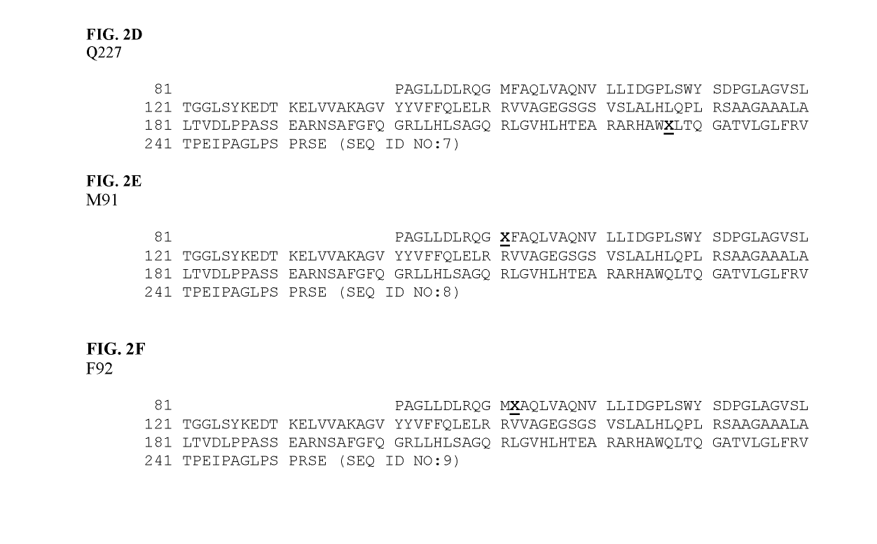

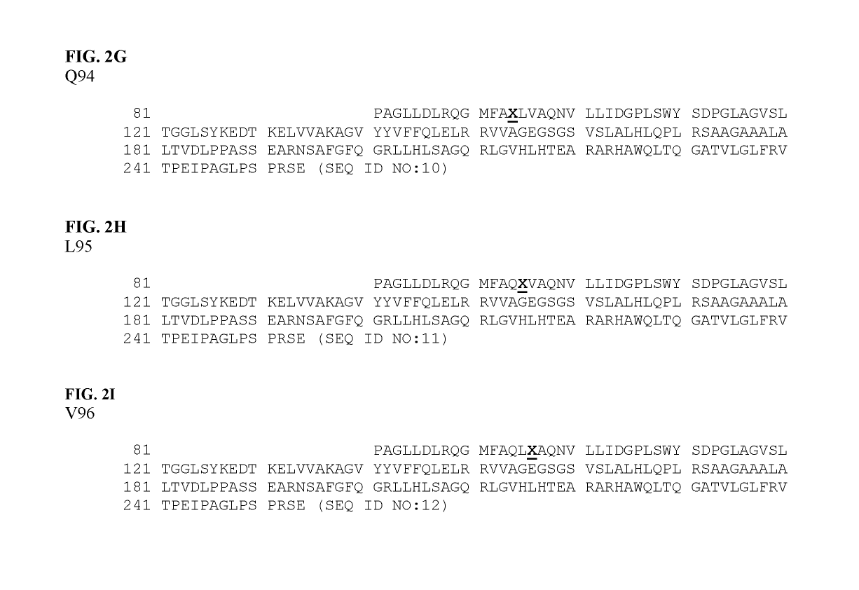

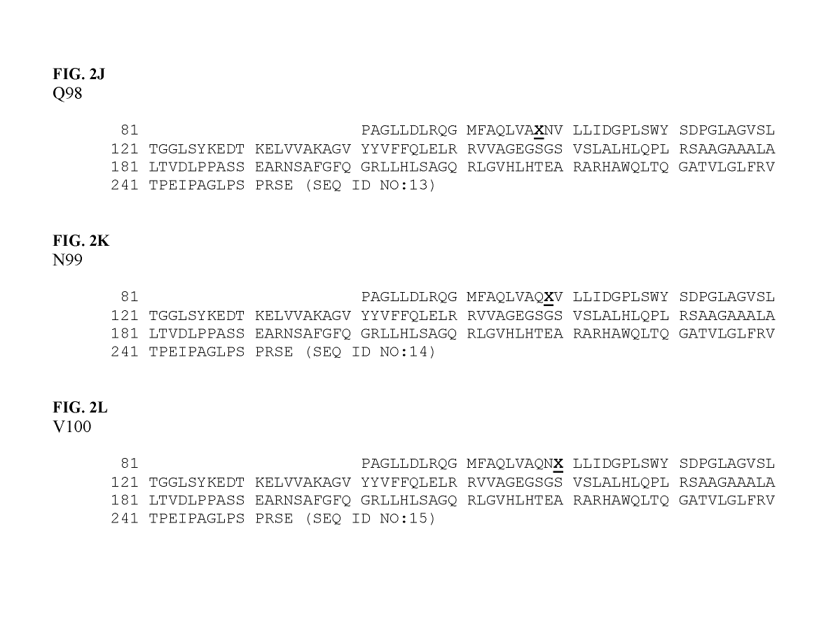

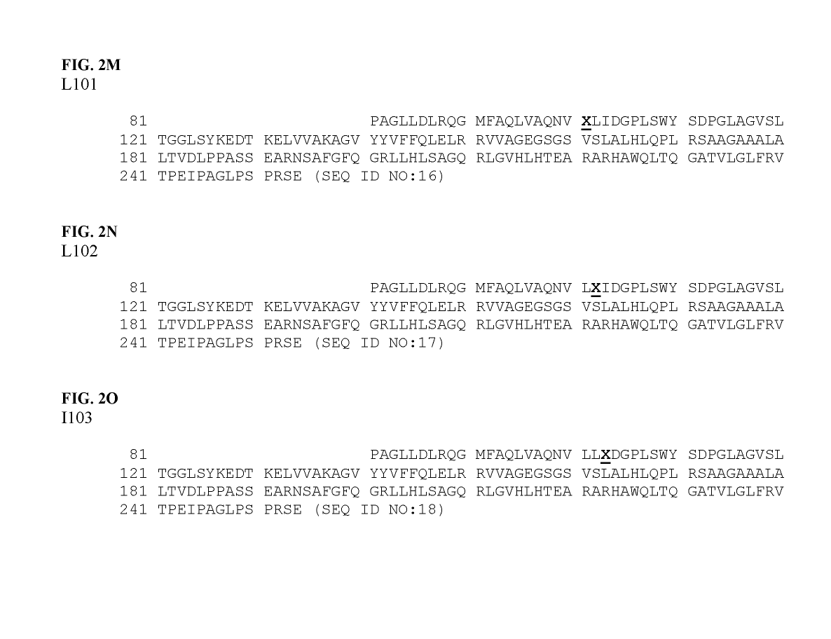

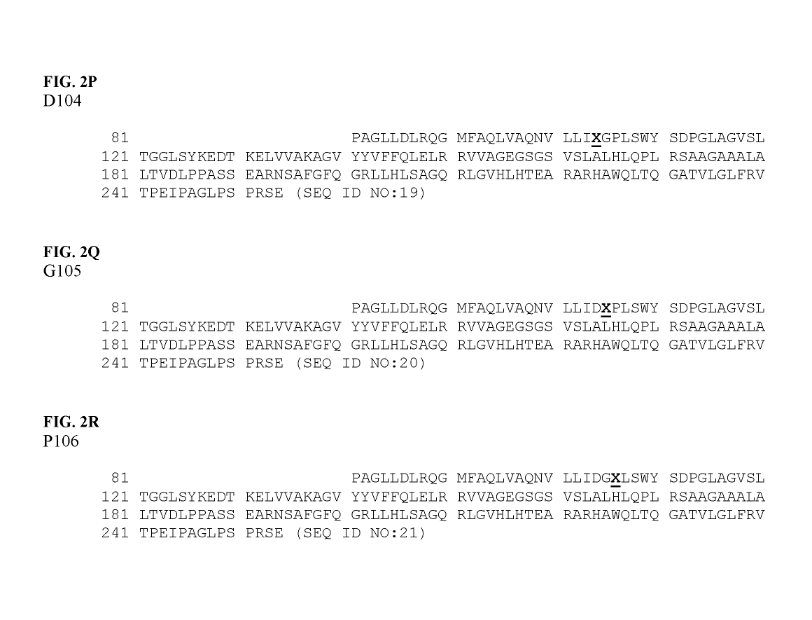

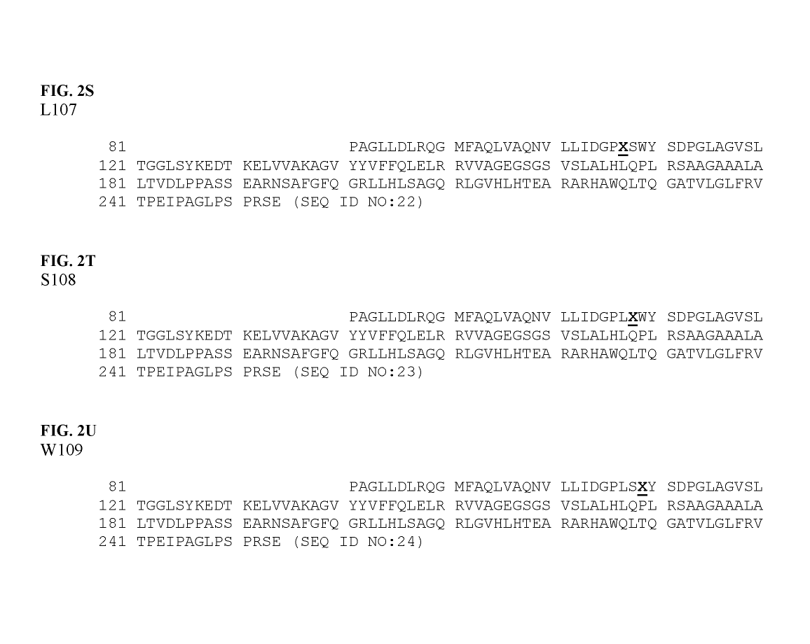

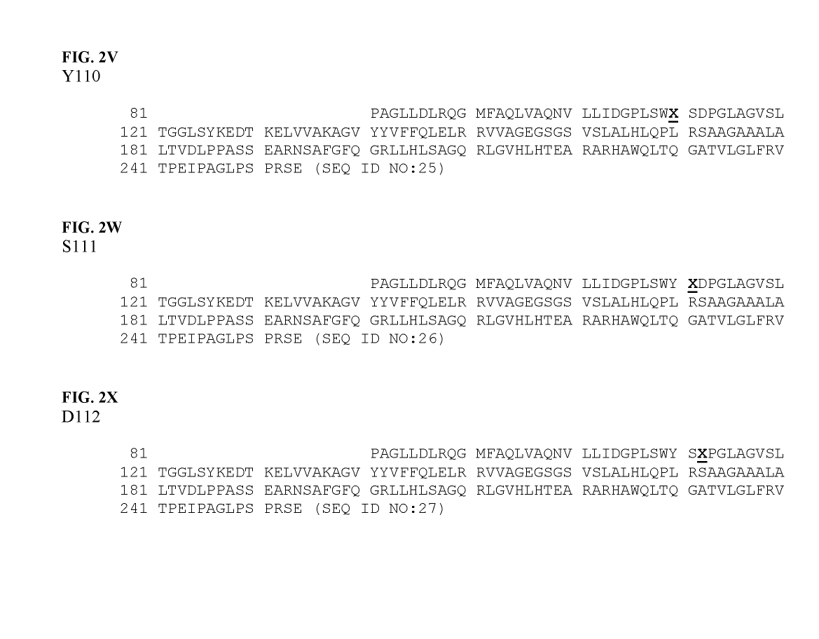

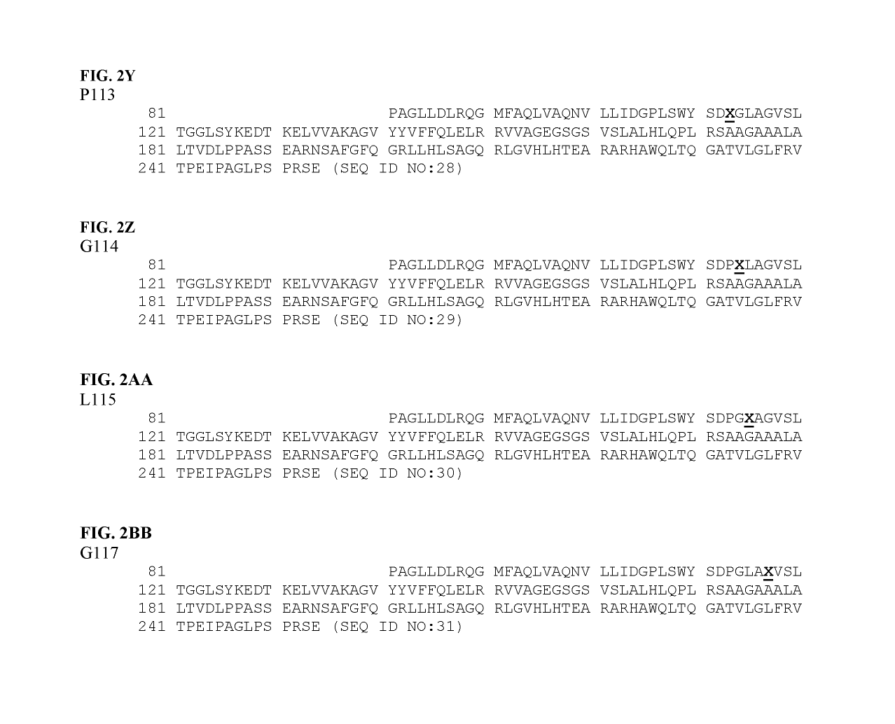

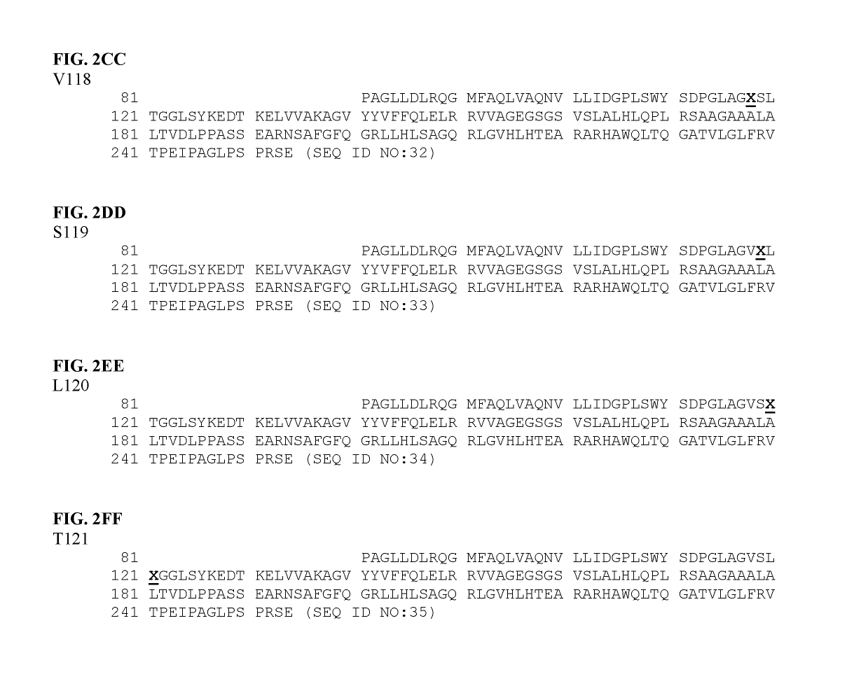

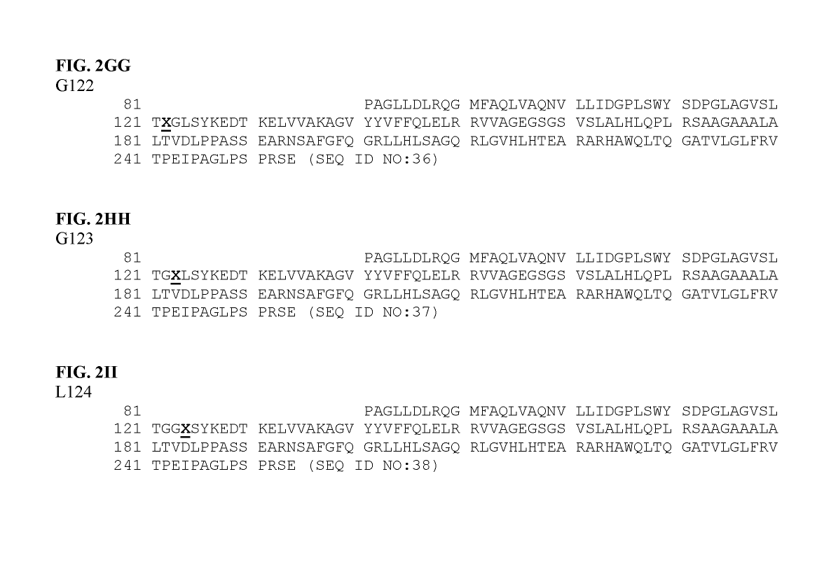

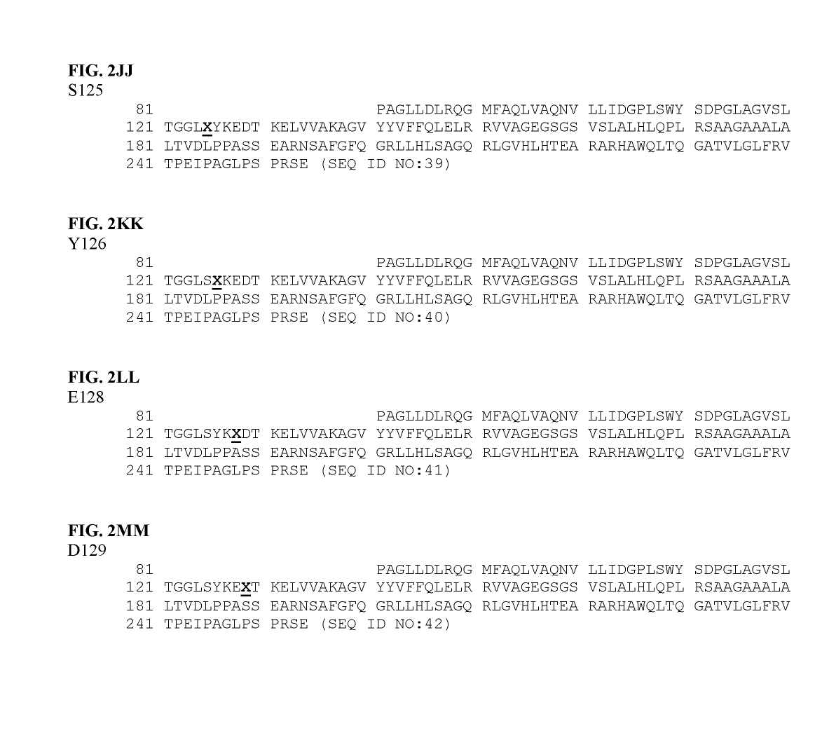

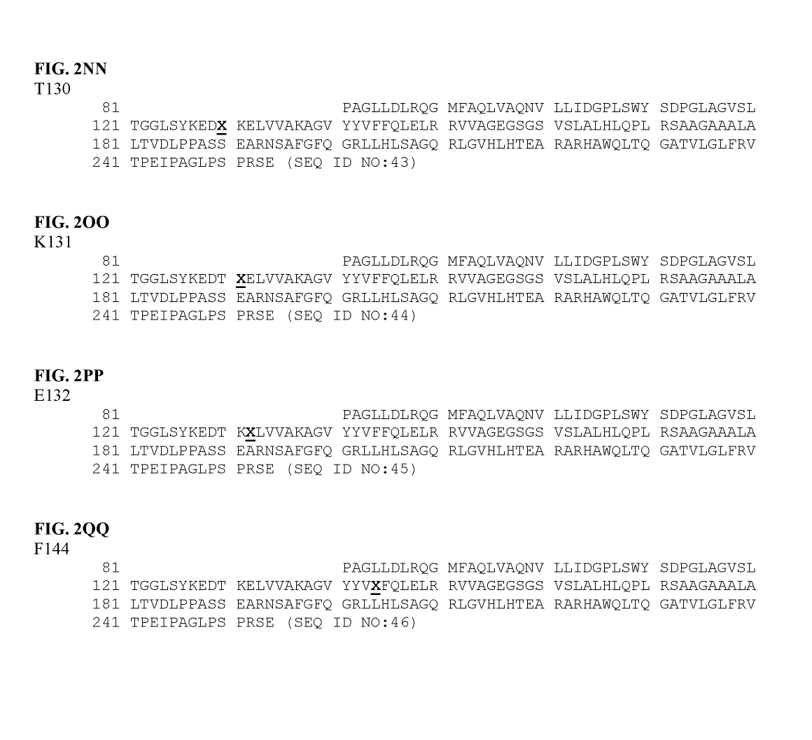

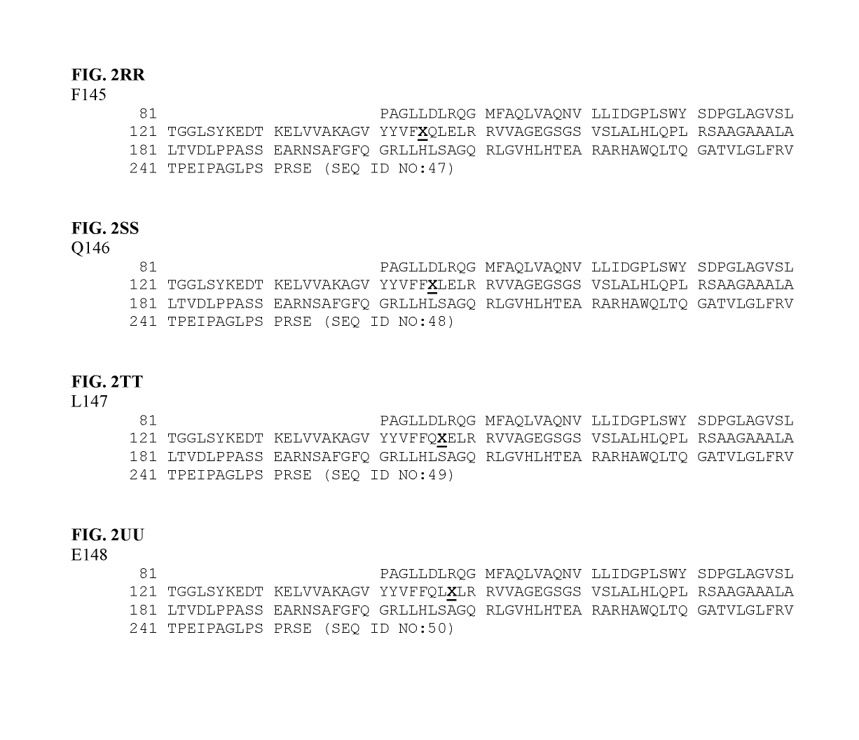

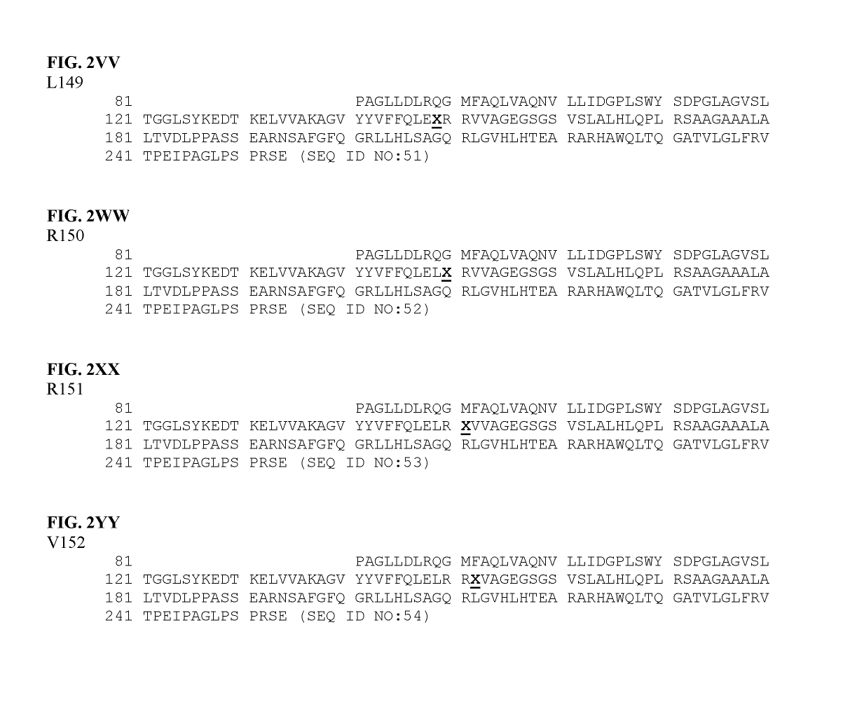

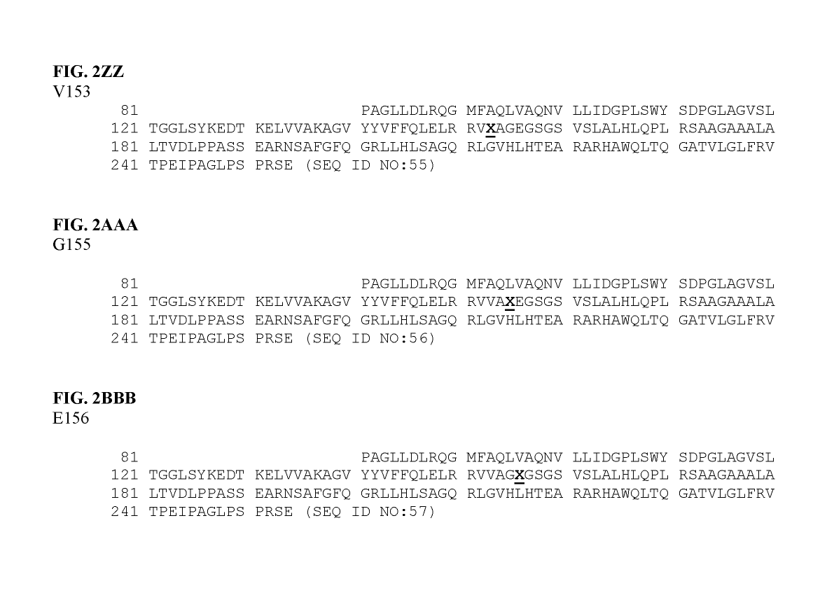

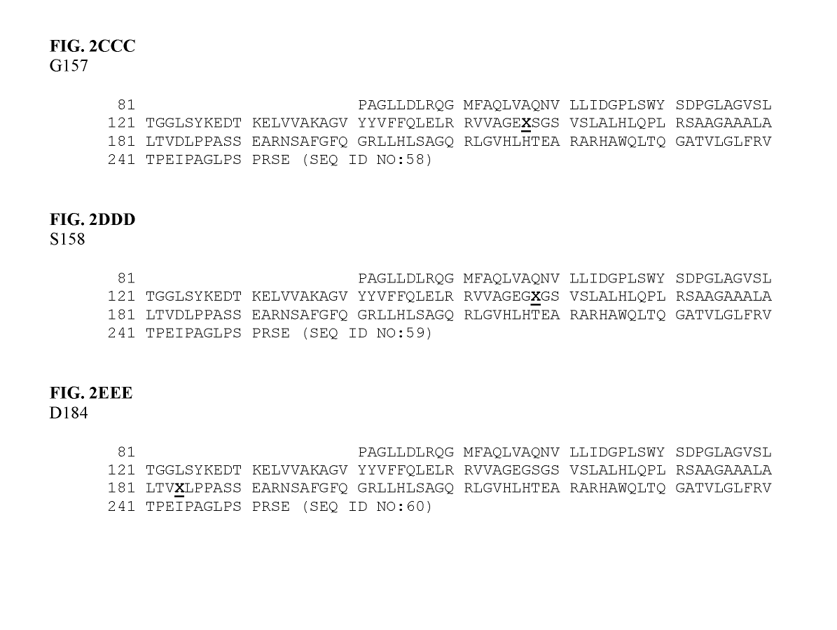

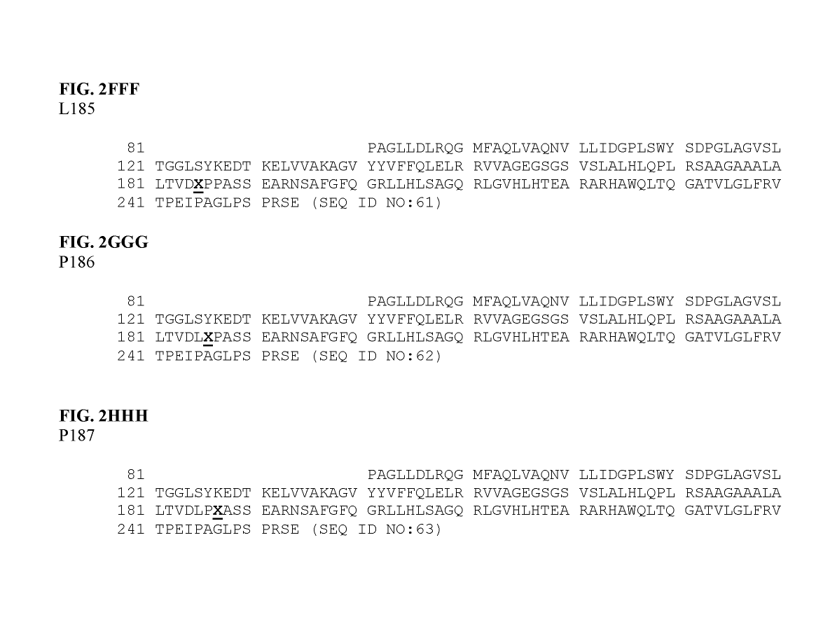

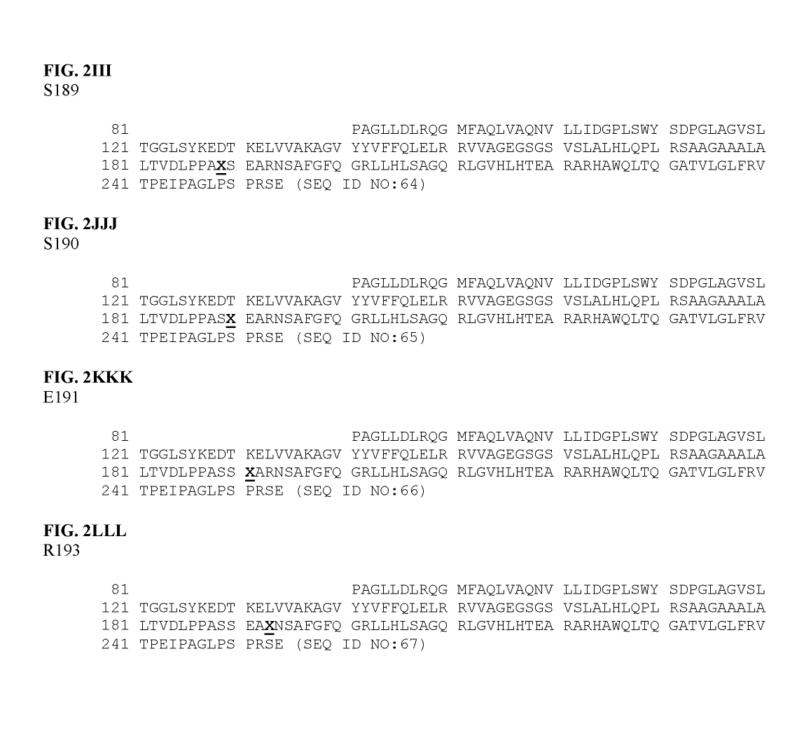

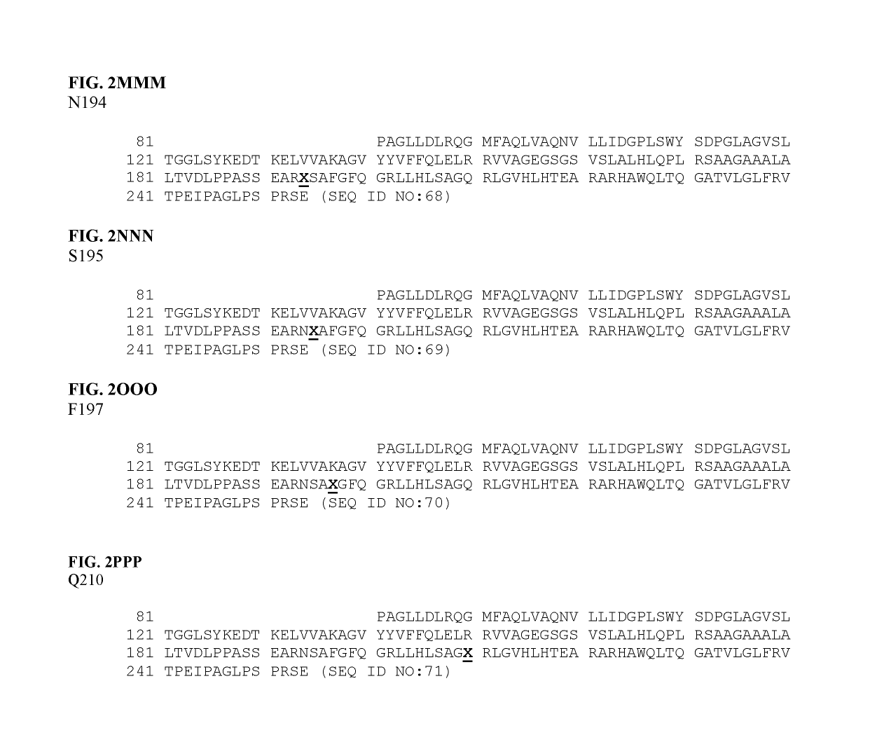

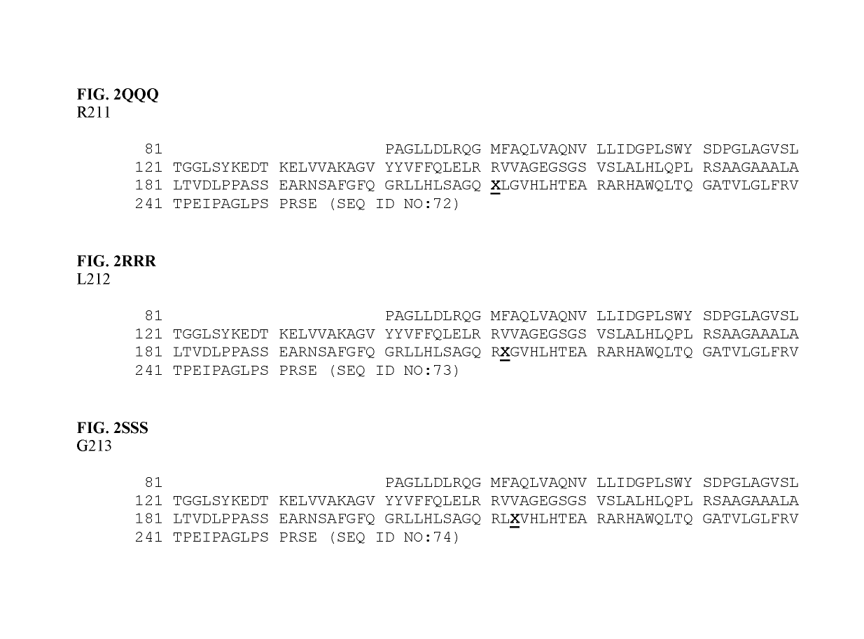

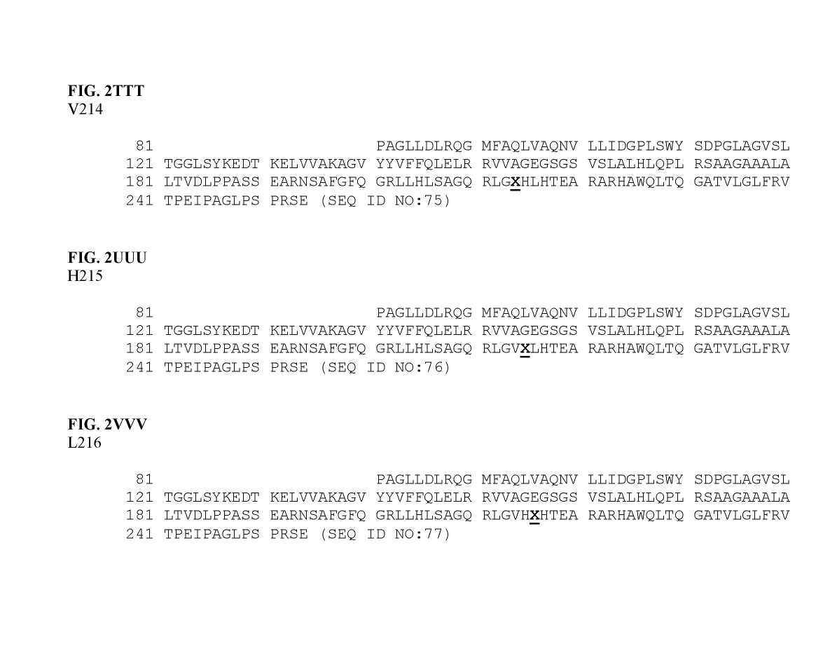

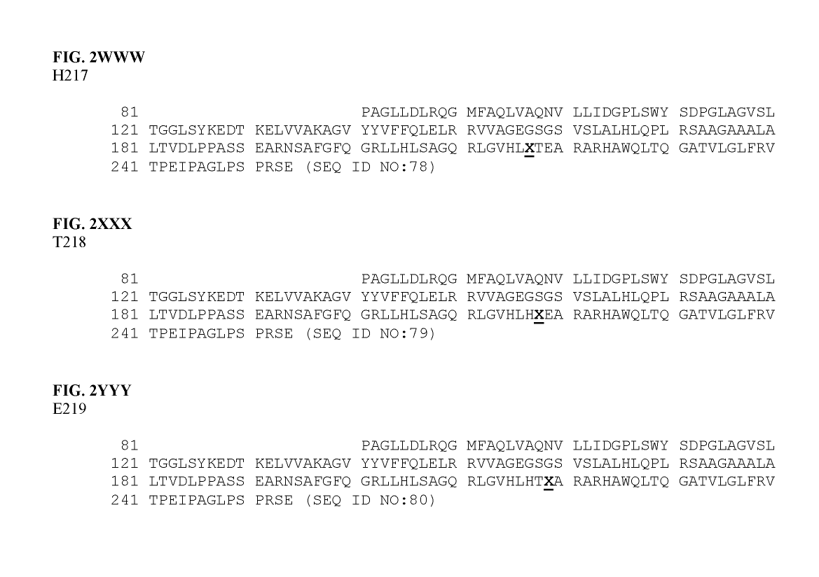

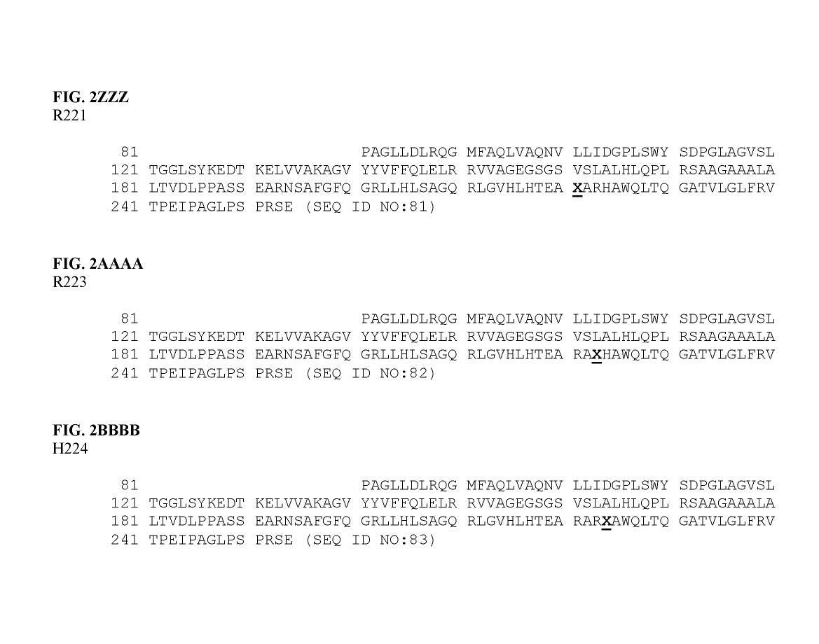

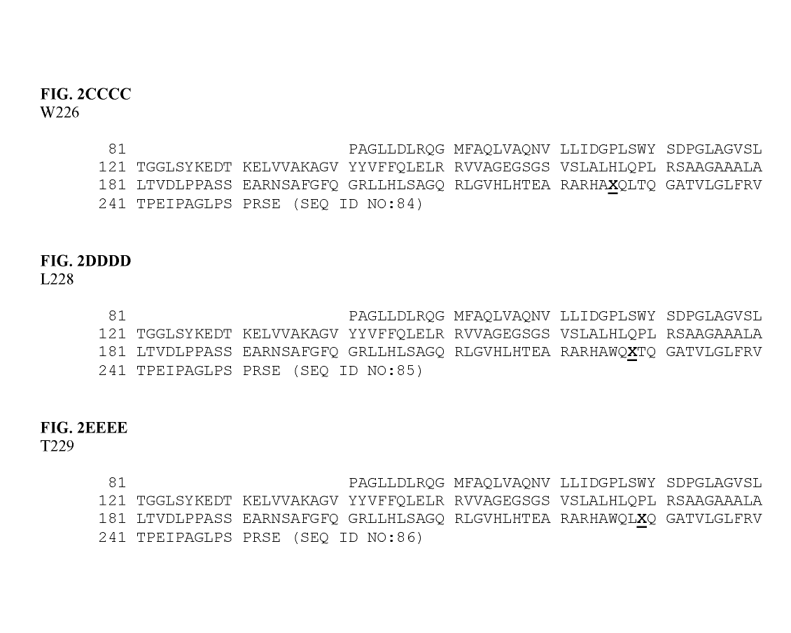

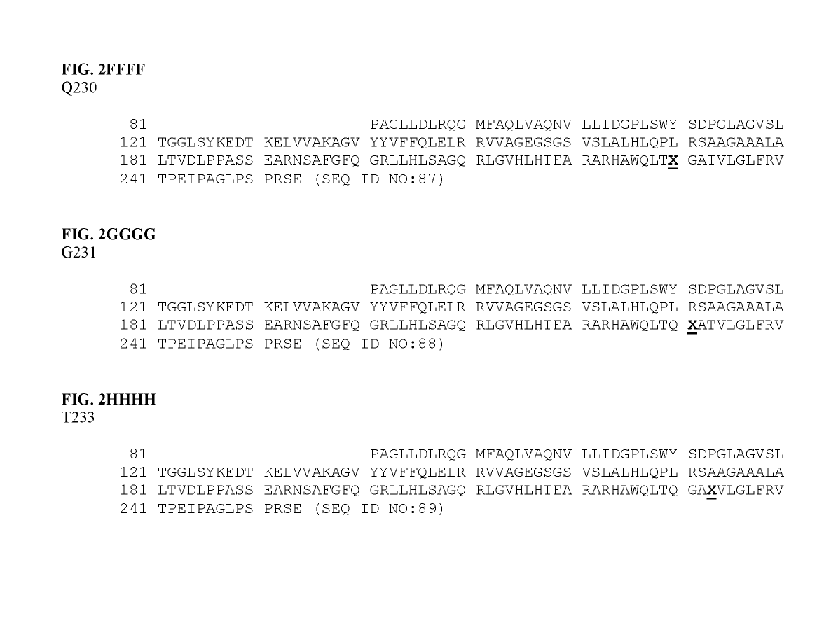

The present disclosure provides variant 4-1BBL modulatory polypeptides. A wild-type human 4-1BBL amino acid sequence is provided in FIG. 2A. The tumor necrosis factor (TNF) homology domain (THD) of human 4-1BBL comprises amino acids 81-254, amino acids 80-254, or amino acids 80-246 of the amino acid sequence depicted in FIG. 2A. Thus, a wild-type amino acid sequence of the THD of human 4-1BBL can be, e.g., one of SEQ ID NOs:1-3, as follows:

(SEQ ID NO: 1)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 2)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 3)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

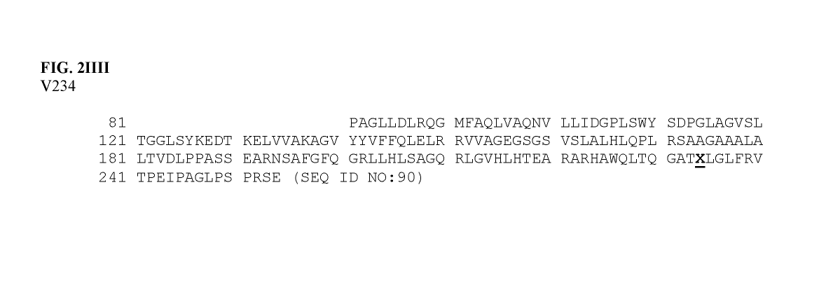

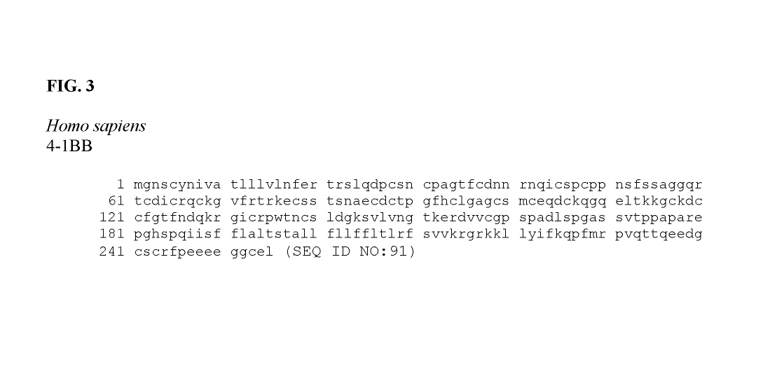

Wild-type 4-1BBL binds to 4-1BB (CD137). An amino acid sequences of 4-1BB is provided in FIG. 3. A variant 4-1BBL polypeptide of the present disclosure binds to 4-1BB with reduced affinity compared to binding of wild-type 4-1BBL to 4-1BB.

In some cases, a variant 4-1BBL polypeptide of the present disclosure exhibits reduced binding affinity to 4-1BB, compared to the binding affinity of a 4-1BBL polypeptide comprising the amino acid sequence depicted in FIG. 2A. For example, in some cases, a variant 4-1BBL polypeptide of the present disclosure binds 4-1BB with a binding affinity that less than the binding affinity of a 4-1BBL polypeptide comprising the amino acid sequence depicted in FIG. 2A for a 4-1BB polypeptide comprising the amino acid sequence depicted in FIG. 3. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence depicted in FIG. 2A for a 4-1BB (e.g., a 4-1BB polypeptide comprising the amino acid sequence depicted in FIG. 3), when assayed under the same conditions.

In some cases, a variant 4-1BBL polypeptide of the present disclosure exhibits reduced binding affinity to 4-1BB, compared to the binding affinity of a 4-1BBL polypeptide comprising the amino acid sequence set forth in SEQ ID NO:1. For example, in some cases, a variant 4-1BBL polypeptide of the present disclosure binds 4-1BB with a binding affinity that less than the binding affinity of a 4-1BBL polypeptide comprising the amino acid sequence set forth in SEQ ID NO:1 for a 4-1BB polypeptide comprising the amino acid sequence depicted in FIG. 3. For example, in some cases, a variant 4-1BBL polypeptide of the present disclosure binds 4-1BB with a binding affinity that is is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in SEQ ID NO:1 for a 4-1BB polypeptide (e.g., a 4-1BB polypeptide comprising the amino acid sequence depicted in FIG. 3), when assayed under the same conditions.

In some cases, a variant 4-1BBL polypeptide of the present disclosure has a binding affinity to 4-1BB that is from 100 nM to 100 μM. As another example, in some cases, a variant 4-1BBL polypeptide of the present disclosure has a binding affinity for 4-1BB (e.g., a 4-1BB polypeptide comprising the amino acid sequence depicted in FIG. 3) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure exhibits increased production in a mammalian host cell, compared to the production in the same mammalian host cell of a wild-type 4-1BBL polypeptide (e.g., a 4-1BBL polypeptide comprising the amino acid sequence depicted in FIG. 2A or as set forth in SEQ ID NO:1). For example, in some cases, a variant 4-1BBL polypeptide of the present disclosure, when expressed in a mammalian host cell, is produced in an amount that is from 25% higher to about 50% higher, from about 50% higher to about 75% higher, from about 75% higher to about 2-fold higher, from about 2-fold higher to about 5-fold higher, from about 5-fold higher to about 10-fold higher, from about 10-fold higher to about 20-fold higher, from about 20-fold higher to about 30-fold higher, from about 30-fold higher to about 40-fold higher, from about 40-fold higher to about 50-fold higher, from about 50-fold higher to about 75-fold higher, from about 75-fold higher to about 100-fold higher, or more than 100-fold higher, than the amount of a wild-type 4-1BBL polypeptide (e.g., a 4-1BBL polypeptide comprising the amino acid sequence depicted in FIG. 2A or as set forth in SEQ ID NO: 1) produced in the same mammalian host cell.

In some cases, a variant 4-1BBL polypeptide of the present disclosure is produced in a mammalian host cell in an amount of from about 50 mg/L to about 75 mg/L, from about 75 mg/L to about 100 mg/L, from about 100 mg/L to about 150 mg/L, from about 150 mg/L to about 200 mg/L, from about 200 mg/L to about 250 mg/L, from about 250 mg/L to about 500 mg/L, or more than 500 mg/L. In some cases, a variant 4-1BBL polypeptide of the present disclosure is produced in a mammalian host cell in an amount of from about 10 mg/L to about 15 mg/L, from about 15 mg/L to about 20 mg/L, from about 20 mg/L to about 25 mg/L, from about 25 mg/L to about 30 mg/L, from about 35 mg/L to about 40 mg/L, from about 40 mg/L to about 45 mg/L, or from about 45 mg/L to about 50 mg/L.

A variant 4-1BBL polypeptide of the present disclosure can have a single amino acid substitution relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide of the present disclosure has from 2 to 10 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 2 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 3 amino acid substitutions relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide of the present disclosure has 4 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 5 amino acid substitutions relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide of the present disclosure has 6 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 7 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 8 amino acid substitutions relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide of the present disclosure has 9 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 10 amino acid substitutions relative to a wild-type 4-1BBL polypeptide (e.g., a 4-1BBL polypeptide comprising the amino acid sequence depicted in FIG. 2A or as set forth in SEQ ID NO: 1).

In some cases, a variant 4-1BBL polypeptide of the present disclosure has from 11 to 50 amino acid substitutions relative to a wild-type 4-1BBL polypeptide (e.g., a 4-1BBL polypeptide comprising the amino acid sequence depicted in FIG. 2A or as set forth in SEQ ID NO:1). For example, in some cases, a variant 4-1BBL polypeptide of the present disclosure has from 11 to 15, from 15 to 20, from 20 to 25, from 25 to 30, from 30 to 35, from 35 to 40, from 40 to 45, or from 45 to 50, amino acid substitutions relative to a wild-type 4-1BBL polypeptide (e.g., a 4-1BBL polypeptide comprising the amino acid sequence depicted in FIG. 2A or as set forth in SEQ ID NO:1).

A variant 4-1BBL polypeptide of the present disclosure can have a length of from 200 amino acids to 254 amino acids. A variant 4-1BBL polypeptide of the present disclosure can have a length of from 150 amino acids to 200 amino acids. For example, in some cases, a variant 4-1BBL polypeptide of the present disclosure has a length of from 150 amino acids to 160 amino acids, from 160 amino acids to 170 amino acids, from 170 amino acids to 180 amino acids, from 180 amino acids to 190 amino acids, or from 190 amino acids to 200 amino acids. In some cases, a variant 4-1BBL polypeptide of the present disclosure has a length of from 160 amino acids to 175 amino acids. In some cases, a variant 4-1BBL polypeptide of the present disclosure has a length of 162 amino acids. In some cases, a variant 4-1BBL polypeptide of the present disclosure has a length of 167 amino acids. In some cases, a variant 4-1BBL polypeptide of the present disclosure has a length of 174 amino acids. In some cases, a variant 4-1BBL polypeptide of the present disclosure has a length of 175 amino acids.

Variant 4-1BBL polypeptides are described below in relation to the amino acid sequences depicted in FIG. 2A-2IIII, and in relation to the amino acid sequence depicted in SEQ ID NO: 1. However, for each variant 4-1BBL polypeptide described below, a corresponding amino acid in SEQ ID NO:2 or SEQ ID NO:3 could be substituted. For example, in some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at K48. In some cases, a variant 4-1BBL polypeptide of the present disclosure comprises the amino acid sequence set forth in SEQ ID NO:2, with an amino acid substitution at K48. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at K48. In some cases, a variant 4-1BBL polypeptide of the present disclosure comprises the amino acid sequence set forth in SEQ ID NO:3, with an amino acid substitution at K48.

### K127 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu. The amino acid numbering in FIG. 2B retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Glu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2B, where amino acid 127 is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at K127. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at K47. In some cases, a variant 4-1BBL polypeptide of the present disclosure comprises the amino acid sequence set forth in SEQ ID NO:1, with an amino acid substitution at K47. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at K48. In some cases, a variant 4-1BBL polypeptide of the present disclosure comprises the amino acid sequence set forth in SEQ ID NO:2, with an amino acid substitution at K48. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at K48. In some cases, a variant 4-1BBL polypeptide of the present disclosure comprises the amino acid sequence set forth in SEQ ID NO:3, with an amino acid substitution at K48.

For example, in some cases, a variant 4-1BBL polypeptide of the present disclosure comprises the amino acid sequence set forth in FIG. 2B, where “x” is any amino acid other than lysine; for example, “x” can be Gly, Ala, Val, Leu, Ile, Pro, Phe, Tyr, Trp, Ser, Thr, Cys, Met, Asn, Gln, Arg, His, Asp, or Glu. In some cases, a variant 4-1BBL polypeptide of the present disclosure comprises the amino acid sequence set forth in FIG. 2B, where “x” is Phe. In some cases, a variant 4-1BBL polypeptide of the present disclosure comprises the amino acid sequence set forth in FIG. 2B, where “x” is Leu. In some cases, a variant 4-1BBL polypeptide of the present disclosure comprises the amino acid sequence set forth in FIG. 2B, where “x” is Ile. In some cases, a variant 4-1BBL polypeptide of the present disclosure comprises the amino acid sequence set forth in FIG. 2B, where “x” is Met. In some cases, a variant 4-1BBL polypeptide of the present disclosure comprises the amino acid sequence set forth in FIG. 2B, where “x” is Val. In some cases, a variant 4-1BBL polypeptide of the present disclosure comprises the amino acid sequence set forth in FIG. 2B, where “x” is Ser. In some cases, a variant 4-1BBL polypeptide of the present disclosure comprises the amino acid sequence set forth in FIG. 2B, where “x” is Pro. In some cases, a variant 4-1BBL polypeptide of the present disclosure comprises the amino acid sequence set forth in FIG. 2B, where “x” is Thr. In some cases, a variant 4-1BBL polypeptide of the present disclosure comprises the amino acid sequence set forth in FIG. 2B, where “x” is Ala. In some cases, a variant 4-1BBL polypeptide of the present disclosure comprises the amino acid sequence set forth in FIG. 2B, where “x” is Tyr. In some cases, a variant 4-1BBL polypeptide of the present disclosure comprises the amino acid sequence set forth in FIG. 2B, where “x” is His. In some cases, a variant 4-1BBL polypeptide of the present disclosure comprises the amino acid sequence set forth in FIG. 2B, where “x” is Gln. In some cases, a variant 4-1BBL polypeptide of the present disclosure comprises the amino acid sequence set forth in FIG. 2B, where “x” is Asn. In some cases, a variant 4-1BBL polypeptide of the present disclosure comprises the amino acid sequence set forth in FIG. 2B, where “x” is Asp. In some cases, a variant 4-1BBL polypeptide of the present disclosure comprises the amino acid sequence set forth in FIG. 2B, where “x” is Glu. In some cases, a variant 4-1BBL polypeptide of the present disclosure comprises the amino acid sequence set forth in FIG. 2B, where “x” is Cys. In some cases, a variant 4-1BBL polypeptide of the present disclosure comprises the amino acid sequence set forth in FIG. 2B, where “x” is Trp. In some cases, a variant 4-1BBL polypeptide of the present disclosure comprises the amino acid sequence set forth in FIG. 2B, where “x” is Arg. In some cases, a variant 4-1BBL polypeptide of the present disclosure comprises the amino acid sequence set forth in FIG. 2B, where “x” is Gly. For example, in some cases, a variant 4-1BBL polypeptide of the present disclosure comprises the amino acid sequence set forth in FIG. 2C. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q227 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2D retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Glu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2D, where amino acid 227 is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q227. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at Q147. In some cases, a variant 4-1BBL polypeptide of the present disclosure comprises the amino acid sequence set forth in SEQ ID NO:1, with an amino acid substitution at Q147. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q148. In some cases, a variant 4-1BBL polypeptide of the present disclosure comprises the amino acid sequence set forth in SEQ ID NO:2, with an amino acid substitution at Q148. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q148. In some cases, a variant 4-1BBL polypeptide of the present disclosure comprises the amino acid sequence set forth in SEQ ID NO:3, with an amino acid substitution at Q148.

For example, in some cases, a variant 4-1BBL polypeptide of the present disclosure comprises the amino acid sequence set forth in FIG. 2D, where “x” is any amino acid other than glutamine; for example, “x” can be Gly, Ala, Val, Leu, Ile, Pro, Phe, Tyr, Trp, Ser, Thr, Cys, Met, Asn, Gln, Arg, His, Asp, or Glu. In some cases, a variant 4-1BBL polypeptide of the present disclosure comprises the amino acid sequence set forth in FIG. 2D, where “x” is Phe. In some cases, a variant 4-1BBL polypeptide of the present disclosure comprises the amino acid sequence set forth in FIG. 2D, where “x” is Leu. In some cases, a variant 4-1BBL polypeptide of the present disclosure comprises the amino acid sequence set forth in FIG. 2D, where “x” is Ile. In some cases, a variant 4-1BBL polypeptide of the present disclosure comprises the amino acid sequence set forth in FIG. 2D, where “x” is Met. In some cases, a variant 4-1BBL polypeptide of the present disclosure comprises the amino acid sequence set forth in FIG. 2D, where “x” is Val. In some cases, a variant 4-1BBL polypeptide of the present disclosure comprises the amino acid sequence set forth in FIG. 2D, where “x” is Ser. In some cases, a variant 4-1BBL polypeptide of the present disclosure comprises the amino acid sequence set forth in FIG. 2D, where “x” is Pro. In some cases, a variant 4-1BBL polypeptide of the present disclosure comprises the amino acid sequence set forth in FIG. 2D, where “x” is Thr. In some cases, a variant 4-1BBL polypeptide of the present disclosure comprises the amino acid sequence set forth in FIG. 2D, where “x” is Ala. In some cases, a variant 4-1BBL polypeptide of the present disclosure comprises the amino acid sequence set forth in FIG. 2D, where “x” is Tyr. In some cases, a variant 4-1BBL polypeptide of the present disclosure comprises the amino acid sequence set forth in FIG. 2D, where “x” is His. In some cases, a variant 4-1BBL polypeptide of the present disclosure comprises the amino acid sequence set forth in FIG. 2D, where “x” is Asn. In some cases, a variant 4-1BBL polypeptide of the present disclosure comprises the amino acid sequence set forth in FIG. 2D, where “x” is Lys. In some cases, a variant 4-1BBL polypeptide of the present disclosure comprises the amino acid sequence set forth in FIG. 2D, where “x” is Asp. In some cases, a variant 4-1BBL polypeptide of the present disclosure comprises the amino acid sequence set forth in FIG. 2D, where “x” is Glu. In some cases, a variant 4-1BBL polypeptide of the present disclosure comprises the amino acid sequence set forth in FIG. 2D, where “x” is Cys. In some cases, a variant 4-1BBL polypeptide of the present disclosure comprises the amino acid sequence set forth in FIG. 2D, where “x” is Trp. In some cases, a variant 4-1BBL polypeptide of the present disclosure comprises the amino acid sequence set forth in FIG. 2D, where “x” is Arg. In some cases, a variant 4-1BBL polypeptide of the present disclosure comprises the amino acid sequence set forth in FIG. 2D, where “x” is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### M91 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 (indicated by an “x”) is an amino acid other than a methionine, e.g., where amino acid 91 is Gly, Ala, Val, Leu, Ile, Pro, Phe, Tyr, Trp, Ser, Thr, Cys, Asn, Gln, Lys, Arg, His, Asp, or Glu. The amino acid numbering in FIG. 2E retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 91 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at M91. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at M11. In some cases, a variant 4-1BBL polypeptide of the present disclosure comprises the amino acid sequence set forth in SEQ ID NO:1, with an amino acid substitution at M11. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at M12. In some cases, a variant 4-1BBL polypeptide of the present disclosure comprises the amino acid sequence set forth in SEQ ID NO:2, with an amino acid substitution at M12. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at M12. In some cases, a variant 4-1BBL polypeptide of the present disclosure comprises the amino acid sequence set forth in SEQ ID NO:3, with an amino acid substitution at M12.

For example, in some cases, a variant 4-1BBL polypeptide of the present disclosure comprises the amino acid sequence set forth in FIG. 2E, where “x” is any amino acid other than methionine; for example, “x” can be Gly, Ala, Val, Leu, Ile, Pro, Phe, Tyr, Trp, Ser, Thr, Cys, Asn, Gln, Lys, Arg, His, Asp, or Glu. In some cases, a variant 4-1BBL polypeptide of the present disclosure comprises the amino acid sequence set forth in FIG. 2E, where “x” is Gly. In some cases, a variant 4-1BBL polypeptide of the present disclosure comprises the amino acid sequence set forth in FIG. 2E, where “x” is Ala. In some cases, a variant 4-1BBL polypeptide of the present disclosure comprises the amino acid sequence set forth in FIG. 2E, where “x” is Val. In some cases, a variant 4-1BBL polypeptide of the present disclosure comprises the amino acid sequence set forth in FIG. 2E, where “x” is Leu. In some cases, a variant 4-1BBL polypeptide of the present disclosure comprises the amino acid sequence set forth in FIG. 2E, where “x” is Ile. In some cases, a variant 4-1BBL polypeptide of the present disclosure comprises the amino acid sequence set forth in FIG. 2E, where “x” is Pro. In some cases, a variant 4-1BBL polypeptide of the present disclosure comprises the amino acid sequence set forth in FIG. 2E, where “x” is Phe. In some cases, a variant 4-1BBL polypeptide of the present disclosure comprises the amino acid sequence set forth in FIG. 2E, where “x” is Tyr. In some cases, a variant 4-1BBL polypeptide of the present disclosure comprises the amino acid sequence set forth in FIG. 2E, where “x” is Trp. In some cases, a variant 4-1BBL polypeptide of the present disclosure comprises the amino acid sequence set forth in FIG. 2E, where “x” is Ser. In some cases, a variant 4-1BBL polypeptide of the present disclosure comprises the amino acid sequence set forth in FIG. 2E, where “x” is Thr. In some cases, a variant 4-1BBL polypeptide of the present disclosure comprises the amino acid sequence set forth in FIG. 2E, where “x” is Cys. In some cases, a variant 4-1BBL polypeptide of the present disclosure comprises the amino acid sequence set forth in FIG. 2E, where “x” is Asn. In some cases, a variant 4-1BBL polypeptide of the present disclosure comprises the amino acid sequence set forth in FIG. 2E, where “x” is Gln. In some cases, a variant 4-1BBL polypeptide of the present disclosure comprises the amino acid sequence set forth in FIG. 2E, where “x” is Lys. In some cases, a variant 4-1BBL polypeptide of the present disclosure comprises the amino acid sequence set forth in FIG. 2E, where “x” is Arg. In some cases, a variant 4-1BBL polypeptide of the present disclosure comprises the amino acid sequence set forth in FIG. 2E, where “x” is His. In some cases, a variant 4-1BBL polypeptide of the present disclosure comprises the amino acid sequence set forth in FIG. 2E, where “x” is Asp. In some cases, a variant 4-1BBL polypeptide of the present disclosure comprises the amino acid sequence set forth in FIG. 2E, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### F92 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 (indicated by an “x”) is an amino acid other than a phenylalanine, e.g., where amino acid 92 is Gly, Ala, Val, Leu, Ile, Pro, Tyr, Trp, Ser, Thr, Cys, Met, Asn, Gln, Lys, Arg, His, Asp, or Glu. The amino acid numbering in FIG. 2F retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2F, where amino acid 92 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at F92. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at F12. In some cases, a variant 4-1BBL polypeptide of the present disclosure comprises the amino acid sequence set forth in SEQ ID NO:1, with an amino acid substitution at F12. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F13. In some cases, a variant 4-1BBL polypeptide of the present disclosure comprises the amino acid sequence set forth in SEQ ID NO:2, with an amino acid substitution at F13. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F13. In some cases, a variant 4-1BBL polypeptide of the present disclosure comprises the amino acid sequence set forth in SEQ ID NO:3, with an amino acid substitution at F13.

For example, in some cases, a variant 4-1BBL polypeptide of the present disclosure comprises the amino acid sequence set forth in FIG. 2F, where “x” is any amino acid other than phenylalanine; for example, “x” can be Gly, Ala, Val, Leu, Ile, Pro, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2F, where “x” is Gly. In some cases, a variant 4-1BBL polypeptide of the present disclosure comprises the amino acid sequence set forth in FIG. 2F, where “x” is Ala. In some cases, a variant 4-1BBL polypeptide of the present disclosure comprises the amino acid sequence set forth in FIG. 2F, where “x” is Val. In some cases, a variant 4-1BBL polypeptide of the present disclosure comprises the amino acid sequence set forth in FIG. 2F, where “x” is Leu. In some cases, a variant 4-1BBL polypeptide of the present disclosure comprises the amino acid sequence set forth in FIG. 2F, where “x” is Ile. In some cases, a variant 4-1BBL polypeptide of the present disclosure comprises the amino acid sequence set forth in FIG. 2F, where “x” is Pro. In some cases, a variant 4-1BBL polypeptide of the present disclosure comprises the amino acid sequence set forth in FIG. 2F, where “x” is Tyr. In some cases, a variant 4-1BBL polypeptide of the present disclosure comprises the amino acid sequence set forth in FIG. 2F, where “x” is Trp. In some cases, a variant 4-1BBL polypeptide of the present disclosure comprises the amino acid sequence set forth in FIG. 2F, where “x” is Ser. In some cases, a variant 4-1BBL polypeptide of the present disclosure comprises the amino acid sequence set forth in FIG. 2F, where “x” is Thr. In some cases, a variant 4-1BBL polypeptide of the present disclosure comprises the amino acid sequence set forth in FIG. 2F, where “x” is Cys. In some cases, a variant 4-1BBL polypeptide of the present disclosure comprises the amino acid sequence set forth in FIG. 2F, where “x” is Met. In some cases, a variant 4-1BBL polypeptide of the present disclosure comprises the amino acid sequence set forth in FIG. 2F, where “x” is Asn. In some cases, a variant 4-1BBL polypeptide of the present disclosure comprises the amino acid sequence set forth in FIG. 2F, where “x” is Gln. In some cases, a variant 4-1BBL polypeptide of the present disclosure comprises the amino acid sequence set forth in FIG. 2F, where “x” is Lys. In some cases, a variant 4-1BBL polypeptide of the present disclosure comprises the amino acid sequence set forth in FIG. 2F, where “x” is Arg. In some cases, a variant 4-1BBL polypeptide of the present disclosure comprises the amino acid sequence set forth in FIG. 2F, where “x” is His. In some cases, a variant 4-1BBL polypeptide of the present disclosure comprises the amino acid sequence set forth in FIG. 2F, where “x” is Asp. In some cases, a variant 4-1BBL polypeptide of the present disclosure comprises the amino acid sequence set forth in FIG. 2F, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q94 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 (indicated by an “x”) is an amino acid other than a glutamine, e.g., where amino acid 94 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2G retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2G, where amino acid 94 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q94. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at Q14. In some cases, a variant 4-1BBL polypeptide of the present disclosure comprises the amino acid sequence set forth in SEQ ID NO:1, with an amino acid substitution at Q14. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q15. In some cases, a variant 4-1BBL polypeptide of the present disclosure comprises the amino acid sequence set forth in SEQ ID NO:2, with an amino acid substitution at Q15. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q15. In some cases, a variant 4-1BBL polypeptide of the present disclosure comprises the amino acid sequence set forth in SEQ ID NO:3, with an amino acid substitution at Q15.

For example, in some cases, a variant 4-1BBL polypeptide of the present disclosure comprises the amino acid sequence set forth in FIG. 2G, where “x” is any amino acid other than methionine; for example, “x” can be Gly, Ala, Val, Leu, Ile, Pro, Phe, Tyr, Trp, Ser, Thr, Cys, Met, Asn, Lys, Arg, His, Asp, or Glu. In some cases, a variant 4-1BBL polypeptide of the present disclosure comprises the amino acid sequence set forth in FIG. 2G, where “x” is Gly. In some cases, a variant 4-1BBL polypeptide of the present disclosure comprises the amino acid sequence set forth in FIG. 2G, where “x” is Ala. In some cases, a variant 4-1BBL polypeptide of the present disclosure comprises the amino acid sequence set forth in FIG. 2G, where “x” is Val. In some cases, a variant 4-1BBL polypeptide of the present disclosure comprises the amino acid sequence set forth in FIG. 2G, where “x” is Leu. In some cases, a variant 4-1BBL polypeptide of the present disclosure comprises the amino acid sequence set forth in FIG. 2G, where “x” is Ile. In some cases, a variant 4-1BBL polypeptide of the present disclosure comprises the amino acid sequence set forth in FIG. 2G, where “x” is Pro. In some cases, a variant 4-1BBL polypeptide of the present disclosure comprises the amino acid sequence set forth in FIG. 2G, where “x” is Phe. In some cases, a variant 4-1BBL polypeptide of the present disclosure comprises the amino acid sequence set forth in FIG. 2G, where “x” is Tyr. In some cases, a variant 4-1BBL polypeptide of the present disclosure comprises the amino acid sequence set forth in FIG. 2G, where “x” is Trp. In some cases, a variant 4-1BBL polypeptide of the present disclosure comprises the amino acid sequence set forth in FIG. 2G, where “x” is Ser. In some cases, a variant 4-1BBL polypeptide of the present disclosure comprises the amino acid sequence set forth in FIG. 2G, where “x” is Thr. In some cases, a variant 4-1BBL polypeptide of the present disclosure comprises the amino acid sequence set forth in FIG. 2G, where “x” is Cys. In some cases, a variant 4-1BBL polypeptide of the present disclosure comprises the amino acid sequence set forth in FIG. 2G, where “x” is Met. In some cases, a variant 4-1BBL polypeptide of the present disclosure comprises the amino acid sequence set forth in FIG. 2G, where “x” is Asn. In some cases, a variant 4-1BBL polypeptide of the present disclosure comprises the amino acid sequence set forth in FIG. 2G, where “x” is Lys. In some cases, a variant 4-1BBL polypeptide of the present disclosure comprises the amino acid sequence set forth in FIG. 2G, where “x” is Arg. In some cases, a variant 4-1BBL polypeptide of the present disclosure comprises the amino acid sequence set forth in FIG. 2G, where “x” is His. In some cases, a variant 4-1BBL polypeptide of the present disclosure comprises the amino acid sequence set forth in FIG. 2G, where “x” is Asp. In some cases, a variant 4-1BBL polypeptide of the present disclosure comprises the amino acid sequence set forth in FIG. 2G, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L95 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 (indicated by an “x”) is an amino acid other than a leucine, e.g., where amino acid 95 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2H retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2H, where amino acid 95 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L95. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L15. In some cases, a variant 4-1BBL polypeptide of the present disclosure comprises the amino acid sequence set forth in SEQ ID NO:1, with an amino acid substitution at L15. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L16. In some cases, a variant 4-1BBL polypeptide of the present disclosure comprises the amino acid sequence set forth in SEQ ID NO:2, with an amino acid substitution at L16. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L16. In some cases, a variant 4-1BBL polypeptide of the present disclosure comprises the amino acid sequence set forth in SEQ ID NO:3, with an amino acid substitution at L16.

For example, in some cases, a variant 4-1BBL polypeptide of the present disclosure comprises the amino acid sequence set forth in FIG. 2H, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2H, where “x” is Gly. In some cases, a variant 4-1BBL polypeptide of the present disclosure comprises the amino acid sequence set forth in FIG. 2H, where “x” is Ala. In some cases, a variant 4-1BBL polypeptide of the present disclosure comprises the amino acid sequence set forth in FIG. 2H, where “x” is Val. In some cases, a variant 4-1BBL polypeptide of the present disclosure comprises the amino acid sequence set forth in FIG. 2H, where “x” is Ile. In some cases, a variant 4-1BBL polypeptide of the present disclosure comprises the amino acid sequence set forth in FIG. 2H, where “x” is Pro. In some cases, a variant 4-1BBL polypeptide of the present disclosure comprises the amino acid sequence set forth in FIG. 2H, where “x” is Phe. In some cases, a variant 4-1BBL polypeptide of the present disclosure comprises the amino acid sequence set forth in FIG. 2H, where “x” is Tyr. In some cases, a variant 4-1BBL polypeptide of the present disclosure comprises the amino acid sequence set forth in FIG. 2H, where “x” is Trp. In some cases, a variant 4-1BBL polypeptide of the present disclosure comprises the amino acid sequence set forth in FIG. 2H, where “x” is Ser. In some cases, a variant 4-1BBL polypeptide of the present disclosure comprises the amino acid sequence set forth in FIG. 2H, where “x” is Thr. In some cases, a variant 4-1BBL polypeptide of the present disclosure comprises the amino acid sequence set forth in FIG. 2H, where “x” is Cys. In some cases, a variant 4-1BBL polypeptide of the present disclosure comprises the amino acid sequence set forth in FIG. 2H, where “x” is Met. In some cases, a variant 4-1BBL polypeptide of the present disclosure comprises the amino acid sequence set forth in FIG. v, where “x” is Asn. In some cases, a variant 4-1BBL polypeptide of the present disclosure comprises the amino acid sequence set forth in FIG. 2H, where “x” is Gln. In some cases, a variant 4-1BBL polypeptide of the present disclosure comprises the amino acid sequence set forth in FIG. 2H, where “x” is Lys. In some cases, a variant 4-1BBL polypeptide of the present disclosure comprises the amino acid sequence set forth in FIG. 2H, where “x” is Arg. In some cases, a variant 4-1BBL polypeptide of the present disclosure comprises the amino acid sequence set forth in FIG. 2H, where “x” is His. In some cases, a variant 4-1BBL polypeptide of the present disclosure comprises the amino acid sequence set forth in FIG. 2H, where “x” is Asp. In some cases, a variant 4-1BBL polypeptide of the present disclosure comprises the amino acid sequence set forth in FIG. 2H, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V96 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 (indicated by an “x”) is an amino acid other than a valine, e.g., where amino acid 96 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2I retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2I, where amino acid 96 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V96. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at V16. In some cases, a variant 4-1BBL polypeptide of the present disclosure comprises the amino acid sequence set forth in SEQ ID NO:1, with an amino acid substitution at V16. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V17. In some cases, a variant 4-1BBL polypeptide of the present disclosure comprises the amino acid sequence set forth in SEQ ID NO:2, with an amino acid substitution at V17. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V17. In some cases, a variant 4-1BBL polypeptide of the present disclosure comprises the amino acid sequence set forth in SEQ ID NO:3, with an amino acid substitution at V17.

For example, in some cases, a variant 4-1BBL polypeptide of the present disclosure comprises the amino acid sequence set forth in FIG. 2I, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2I, where “x” is Gly. In some cases, a variant 4-1BBL polypeptide of the present disclosure comprises the amino acid sequence set forth in FIG. 2I, where “x” is Ala. In some cases, a variant 4-1BBL polypeptide of the present disclosure comprises the amino acid sequence set forth in FIG. 2I, where “x” is Leu. In some cases, a variant 4-1BBL polypeptide of the present disclosure comprises the amino acid sequence set forth in FIG. 2I, where “x” is Ile. In some cases, a variant 4-1BBL polypeptide of the present disclosure comprises the amino acid sequence set forth in FIG. 2I, where “x” is Pro. In some cases, a variant 4-1BBL polypeptide of the present disclosure comprises the amino acid sequence set forth in FIG. 2I, where “x” is Phe. In some cases, a variant 4-1BBL polypeptide of the present disclosure comprises the amino acid sequence set forth in FIG. 2I, where “x” is Tyr. In some cases, a variant 4-1BBL polypeptide of the present disclosure comprises the amino acid sequence set forth in FIG. 2I, where “x” is Trp. In some cases, a variant 4-1BBL polypeptide of the present disclosure comprises the amino acid sequence set forth in FIG. 2I, where “x” is Ser. In some cases, a variant 4-1BBL polypeptide of the present disclosure comprises the amino acid sequence set forth in FIG. 2I, where “x” is Thr. In some cases, a variant 4-1BBL polypeptide of the present disclosure comprises the amino acid sequence set forth in FIG. 2I, where “x” is Cys. In some cases, a variant 4-1BBL polypeptide of the present disclosure comprises the amino acid sequence set forth in FIG. 2I, where “x” is Met. In some cases, a variant 4-1BBL polypeptide of the present disclosure comprises the amino acid sequence set forth in FIG. 2I, where “x” is Asn. In some cases, a variant 4-1BBL polypeptide of the present disclosure comprises the amino acid sequence set forth in FIG. 2I, where “x” is Gln. In some cases, a variant 4-1BBL polypeptide of the present disclosure comprises the amino acid sequence set forth in FIG. 2I, where “x” is Lys. In some cases, a variant 4-1BBL polypeptide of the present disclosure comprises the amino acid sequence set forth in FIG. 2I, where “x” is Arg. In some cases, a variant 4-1BBL polypeptide of the present disclosure comprises the amino acid sequence set forth in FIG. 2I, where “x” is His. In some cases, a variant 4-1BBL polypeptide of the present disclosure comprises the amino acid sequence set forth in FIG. 2I, where “x” is Asp. In some cases, a variant 4-1BBL polypeptide of the present disclosure comprises the amino acid sequence set forth in FIG. 2I, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q98 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 (indicated by an “x”) is an amino acid other than a glutamine, e.g., where amino acid 98 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2J retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2J, where amino acid 98 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q98. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at Q18. In some cases, a variant 4-1BBL polypeptide of the present disclosure comprises the amino acid sequence set forth in SEQ ID NO:1, with an amino acid substitution at Q18. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q19. In some cases, a variant 4-1BBL polypeptide of the present disclosure comprises the amino acid sequence set forth in SEQ ID NO:2, with an amino acid substitution at Q19. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q19. In some cases, a variant 4-1BBL polypeptide of the present disclosure comprises the amino acid sequence set forth in SEQ ID NO:3, with an amino acid substitution at Q19.

For example, in some cases, a variant 4-1BBL polypeptide of the present disclosure comprises the amino acid sequence set forth in FIG. 2J, where “x” is any amino acid other than glutamine; for example, “x” can be Gly, Ala, Val, Leu, Ile, Pro, Phe, Tyr, Trp, Ser, Thr, Cys, Met, Asn, Lys, Arg, His, Asp, or Glu. In some cases, a variant 4-1BBL polypeptide of the present disclosure comprises the amino acid sequence set forth in FIG. 2J, where “x” is Gly. In some cases, a variant 4-1BBL polypeptide of the present disclosure comprises the amino acid sequence set forth in FIG. 2J, where “x” is Ala. In some cases, a variant 4-1BBL polypeptide of the present disclosure comprises the amino acid sequence set forth in FIG. 2J, where “x” is Val. In some cases, a variant 4-1BBL polypeptide of the present disclosure comprises the amino acid sequence set forth in FIG. 2J, where “x” is Leu. In some cases, a variant 4-1BBL polypeptide of the present disclosure comprises the amino acid sequence set forth in FIG. 2J, where “x” is Ile. In some cases, a variant 4-1BBL polypeptide of the present disclosure comprises the amino acid sequence set forth in FIG. 2J, where “x” is Pro. In some cases, a variant 4-1BBL polypeptide of the present disclosure comprises the amino acid sequence set forth in FIG. 2J, where “x” is Phe. In some cases, a variant 4-1BBL polypeptide of the present disclosure comprises the amino acid sequence set forth in FIG. 2J, where “x” is Tyr. In some cases, a variant 4-1BBL polypeptide of the present disclosure comprises the amino acid sequence set forth in FIG. 2J, where “x” is Trp. In some cases, a variant 4-1BBL polypeptide of the present disclosure comprises the amino acid sequence set forth in FIG. 2J, where “x” is Ser. In some cases, a variant 4-1BBL polypeptide of the present disclosure comprises the amino acid sequence set forth in FIG. 2J, where “x” is Thr. In some cases, a variant 4-1BBL polypeptide of the present disclosure comprises the amino acid sequence set forth in FIG. 2J, where “x” is Cys. In some cases, a variant 4-1BBL polypeptide of the present disclosure comprises the amino acid sequence set forth in FIG. 2J, where “x” is Met. In some cases, a variant 4-1BBL polypeptide of the present disclosure comprises the amino acid sequence set forth in FIG. 2J, where “x” is Asn. In some cases, a variant 4-1BBL polypeptide of the present disclosure comprises the amino acid sequence set forth in FIG. 2J, where “x” is Lys. In some cases, a variant 4-1BBL polypeptide of the present disclosure comprises the amino acid sequence set forth in FIG. 2J, where “x” is Arg. In some cases, a variant 4-1BBL polypeptide of the present disclosure comprises the amino acid sequence set forth in FIG. 2J, where “x” is His. In some cases, a variant 4-1BBL polypeptide of the present disclosure comprises the amino acid sequence set forth in FIG. 2J, where “x” is Asp. In some cases, a variant 4-1BBL polypeptide of the present disclosure comprises the amino acid sequence set forth in FIG. 2J, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### N99 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 (indicated by an “x”) is an amino acid other than an asparagine, e.g., where amino acid 99 is Gly, Ala, Val, Leu, Ile, Pro, Phe, Tyr, Trp, Ser, Thr, Cys, Met, Gln, Lys, Arg, His, Asp, or Glu. The amino acid numbering in FIG. 2K retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2K, where amino acid 99 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at N99. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at N19. In some cases, a variant 4-1BBL polypeptide of the present disclosure comprises the amino acid sequence set forth in SEQ ID NO:1, with an amino acid substitution at N19. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at N20. In some cases, a variant 4-1BBL polypeptide of the present disclosure comprises the amino acid sequence set forth in SEQ ID NO:2, with an amino acid substitution at N20. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at N20. In some cases, a variant 4-1BBL polypeptide of the present disclosure comprises the amino acid sequence set forth in SEQ ID NO:3, with an amino acid substitution at N20.

For example, in some cases, a variant 4-1BBL polypeptide of the present disclosure comprises the amino acid sequence set forth in FIG. 2K, where “x” is any amino acid other than asparagine; for example, “x” can be Gly, Ala, Val, Leu, Ile, Pro, Phe, Tyr, Trp, Ser, Thr, Cys, Met, Gln, Lys, Arg, His, Asp, or Glu. In some cases, a variant 4-1BBL polypeptide of the present disclosure comprises the amino acid sequence set forth in FIG. 2K, where “x” is Gly. In some cases, a variant 4-1BBL polypeptide of the present disclosure comprises the amino acid sequence set forth in FIG. 2K, where “x” is Ala. In some cases, a variant 4-1BBL polypeptide of the present disclosure comprises the amino acid sequence set forth in FIG. 2K, where “x” is Val. In some cases, a variant 4-1BBL polypeptide of the present disclosure comprises the amino acid sequence set forth in FIG. 2K, where “x” is Leu. In some cases, a variant 4-1BBL polypeptide of the present disclosure comprises the amino acid sequence set forth in FIG. 2K, where “x” is Ile. In some cases, a variant 4-1BBL polypeptide of the present disclosure comprises the amino acid sequence set forth in FIG. 2K, where “x” is Pro. In some cases, a variant 4-1BBL polypeptide of the present disclosure comprises the amino acid sequence set forth in FIG. 2K, where “x” is Phe. In some cases, a variant 4-1BBL polypeptide of the present disclosure comprises the amino acid sequence set forth in FIG. 2K, where “x” is Tyr. In some cases, a variant 4-1BBL polypeptide of the present disclosure comprises the amino acid sequence set forth in FIG. 2K, where “x” is Trp. In some cases, a variant 4-1BBL polypeptide of the present disclosure comprises the amino acid sequence set forth in FIG. 2K, where “x” is Ser. In some cases, a variant 4-1BBL polypeptide of the present disclosure comprises the amino acid sequence set forth in FIG. 2K, where “x” is Thr. In some cases, a variant 4-1BBL polypeptide of the present disclosure comprises the amino acid sequence set forth in FIG. 2K, where “x” is Cys. In some cases, a variant 4-1BBL polypeptide of the present disclosure comprises the amino acid sequence set forth in FIG. 2K, where “x” is Met. In some cases, a variant 4-1BBL polypeptide of the present disclosure comprises the amino acid sequence set forth in FIG. 2K, where “x” is Gln. In some cases, a variant 4-1BBL polypeptide of the present disclosure comprises the amino acid sequence set forth in FIG. 2K, where “x” is Lys. In some cases, a variant 4-1BBL polypeptide of the present disclosure comprises the amino acid sequence set forth in FIG. 2K, where “x” is Arg. In some cases, a variant 4-1BBL polypeptide of the present disclosure comprises the amino acid sequence set forth in FIG. 2K, where “x” is His. In some cases, a variant 4-1BBL polypeptide of the present disclosure comprises the amino acid sequence set forth in FIG. 2K, where “x” is Asp. In some cases, a variant 4-1BBL polypeptide of the present disclosure comprises the amino acid sequence set forth in FIG. 2K, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V100 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 (indicated by an “x”) is an amino acid other than a valine, e.g., where amino acid 100 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2L retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2L, where amino acid 100 is Glu. In some cases, the variant 4-1BBL polypeptide has a binding affinity to CD137 that is from about from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V100. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at V20. In some cases, a variant 4-1BBL polypeptide of the present disclosure comprises the amino acid sequence set forth in SEQ ID NO: 1, with an amino acid substitution at V20. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V21. In some cases, a variant 4-1BBL polypeptide of the present disclosure comprises the amino acid sequence set forth in SEQ ID NO:2, with an amino acid substitution at V21. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V21. In some cases, a variant 4-1BBL polypeptide of the present disclosure comprises the amino acid sequence set forth in SEQ ID NO:3, with an amino acid substitution at V21.

For example, in some cases, a variant 4-1BBL polypeptide of the present disclosure comprises the amino acid sequence set forth in FIG. 2L, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2L, where “x” is Gly. In some cases, a variant 4-1BBL polypeptide of the present disclosure comprises the amino acid sequence set forth in FIG. 2L, where “x” is Ala. In some cases, a variant 4-1BBL polypeptide of the present disclosure comprises the amino acid sequence set forth in FIG. 2L, where “x” is Leu. In some cases, a variant 4-1BBL polypeptide of the present disclosure comprises the amino acid sequence set forth in FIG. 2L, where “x” is Ile. In some cases, a variant 4-1BBL polypeptide of the present disclosure comprises the amino acid sequence set forth in FIG. 2L, where “x” is Pro. In some cases, a variant 4-1BBL polypeptide of the present disclosure comprises the amino acid sequence set forth in FIG. 2L, where “x” is Phe. In some cases, a variant 4-1BBL polypeptide of the present disclosure comprises the amino acid sequence set forth in FIG. 2L, where “x” is Tyr. In some cases, a variant 4-1BBL polypeptide of the present disclosure comprises the amino acid sequence set forth in FIG. 2L, where “x” is Trp. In some cases, a variant 4-1BBL polypeptide of the present disclosure comprises the amino acid sequence set forth in FIG. 2L, where “x” is Ser. In some cases, a variant 4-1BBL polypeptide of the present disclosure comprises the amino acid sequence set forth in FIG. 2L, where “x” is Thr. In some cases, a variant 4-1BBL polypeptide of the present disclosure comprises the amino acid sequence set forth in FIG. 2L, where “x” is Cys. In some cases, a variant 4-1BBL polypeptide of the present disclosure comprises the amino acid sequence set forth in FIG. 2L, where “x” is Met. In some cases, a variant 4-1BBL polypeptide of the present disclosure comprises the amino acid sequence set forth in FIG. 2L, where “x” is Asn. In some cases, a variant 4-1BBL polypeptide of the present disclosure comprises the amino acid sequence set forth in FIG. 2L, where “x” is Gln. In some cases, a variant 4-1BBL polypeptide of the present disclosure comprises the amino acid sequence set forth in FIG. 2L, where “x” is Lys. In some cases, a variant 4-1BBL polypeptide of the present disclosure comprises the amino acid sequence set forth in FIG. 2L, where “x” is Arg. In some cases, a variant 4-1BBL polypeptide of the present disclosure comprises the amino acid sequence set forth in FIG. 2L, where “x” is His. In some cases, a variant 4-1BBL polypeptide of the present disclosure comprises the amino acid sequence set forth in FIG. 2L, where “x” is Asp. In some cases, a variant 4-1BBL polypeptide of the present disclosure comprises the amino acid sequence set forth in FIG. 2L, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L101 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 (indicated by an “x”) is an amino acid other than a leucine, e.g., where amino acid 101 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2M retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2M, where amino acid 101 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L101. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L21. In some cases, a variant 4-1BBL polypeptide of the present disclosure comprises the amino acid sequence set forth in SEQ ID NO:1, with an amino acid substitution at L21. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L22. In some cases, a variant 4-1BBL polypeptide of the present disclosure comprises the amino acid sequence set forth in SEQ ID NO:2, with an amino acid substitution at L22. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L22. In some cases, a variant 4-1BBL polypeptide of the present disclosure comprises the amino acid sequence set forth in SEQ ID NO:3, with an amino acid substitution at L22.

For example, in some cases, a variant 4-1BBL polypeptide of the present disclosure comprises the amino acid sequence set forth in FIG. 2M, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2M, where “x” is Gly. In some cases, a variant 4-1BBL polypeptide of the present disclosure comprises the amino acid sequence set forth in FIG. 2M, where “x” is Ala. In some cases, a variant 4-1BBL polypeptide of the present disclosure comprises the amino acid sequence set forth in FIG. 2M, where “x” is Val. In some cases, a variant 4-1BBL polypeptide of the present disclosure comprises the amino acid sequence set forth in FIG. 2M, where “x” is Ile. In some cases, a variant 4-1BBL polypeptide of the present disclosure comprises the amino acid sequence set forth in FIG. 2M, where “x” is Pro. In some cases, a variant 4-1BBL polypeptide of the present disclosure comprises the amino acid sequence set forth in FIG. 2M, where “x” is Phe. In some cases, a variant 4-1BBL polypeptide of the present disclosure comprises the amino acid sequence set forth in FIG. 2M, where “x” is Tyr. In some cases, a variant 4-1BBL polypeptide of the present disclosure comprises the amino acid sequence set forth in FIG. 2M, where “x” is Trp. In some cases, a variant 4-1BBL polypeptide of the present disclosure comprises the amino acid sequence set forth in FIG. 2M, where “x” is Ser. In some cases, a variant 4-1BBL polypeptide of the present disclosure comprises the amino acid sequence set forth in FIG. 2M, where “x” is Thr. In some cases, a variant 4-1BBL polypeptide of the present disclosure comprises the amino acid sequence set forth in FIG. 2M, where “x” is Cys. In some cases, a variant 4-1BBL polypeptide of the present disclosure comprises the amino acid sequence set forth in FIG. 2M, where “x” is Met. In some cases, a variant 4-1BBL polypeptide of the present disclosure comprises the amino acid sequence set forth in FIG. 2M, where “x” is Asn. In some cases, a variant 4-1BBL polypeptide of the present disclosure comprises the amino acid sequence set forth in FIG. 2M, where “x” is Gln. In some cases, a variant 4-1BBL polypeptide of the present disclosure comprises the amino acid sequence set forth in FIG. 2M, where “x” is Lys. In some cases, a variant 4-1BBL polypeptide of the present disclosure comprises the amino acid sequence set forth in FIG. 2M, where “x” is Arg. In some cases, a variant 4-1BBL polypeptide of the present disclosure comprises the amino acid sequence set forth in FIG. 2M, where “x” is His. In some cases, a variant 4-1BBL polypeptide of the present disclosure comprises the amino acid sequence set forth in FIG. 2M, where “x” is Asp. In some cases, a variant 4-1BBL polypeptide of the present disclosure comprises the amino acid sequence set forth in FIG. 2M, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L102 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 (indicated by an “x”) is an amino acid other than a leucine, e.g., where amino acid 102 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2N retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2N, where amino acid 102 is Glu. In some cases, the variant 4-1BBL polypeptide has a binding affinity to CD137 that is from about from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L102. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L22. In some cases, a variant 4-1BBL polypeptide of the present disclosure comprises the amino acid sequence set forth in SEQ ID NO: 1, with an amino acid substitution at L22. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L23. In some cases, a variant 4-1BBL polypeptide of the present disclosure comprises the amino acid sequence set forth in SEQ ID NO:2, with an amino acid substitution at L23. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L23. In some cases, a variant 4-1BBL polypeptide of the present disclosure comprises the amino acid sequence set forth in SEQ ID NO:3, with an amino acid substitution at L23.

For example, in some cases, a variant 4-1BBL polypeptide of the present disclosure comprises the amino acid sequence set forth in FIG. 2N, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2N, where “x” is Gly. In some cases, a variant 4-1BBL polypeptide of the present disclosure comprises the amino acid sequence set forth in FIG. 2N, where “x” is Ala. In some cases, a variant 4-1BBL polypeptide of the present disclosure comprises the amino acid sequence set forth in FIG. 2N, where “x” is Val. In some cases, a variant 4-1BBL polypeptide of the present disclosure comprises the amino acid sequence set forth in FIG. 2N, where “x” is Ile. In some cases, a variant 4-1BBL polypeptide of the present disclosure comprises the amino acid sequence set forth in FIG. 2N, where “x” is Pro. In some cases, a variant 4-1BBL polypeptide of the present disclosure comprises the amino acid sequence set forth in FIG. 2N, where “x” is Phe. In some cases, a variant 4-1BBL polypeptide of the present disclosure comprises the amino acid sequence set forth in FIG. 2N, where “x” is Tyr. In some cases, a variant 4-1BBL polypeptide of the present disclosure comprises the amino acid sequence set forth in FIG. 2N, where “x” is Trp. In some cases, a variant 4-1BBL polypeptide of the present disclosure comprises the amino acid sequence set forth in FIG. 2N, where “x” is Ser. In some cases, a variant 4-1BBL polypeptide of the present disclosure comprises the amino acid sequence set forth in FIG. 2N, where “x” is Thr. In some cases, a variant 4-1BBL polypeptide of the present disclosure comprises the amino acid sequence set forth in FIG. 2N, where “x” is Cys. In some cases, a variant 4-1BBL polypeptide of the present disclosure comprises the amino acid sequence set forth in FIG. 2N, where “x” is Met. In some cases, a variant 4-1BBL polypeptide of the present disclosure comprises the amino acid sequence set forth in FIG. 2N, where “x” is Asn. In some cases, a variant 4-1BBL polypeptide of the present disclosure comprises the amino acid sequence set forth in FIG. 2N, where “x” is Gln. In some cases, a variant 4-1BBL polypeptide of the present disclosure comprises the amino acid sequence set forth in FIG. 2N, where “x” is Lys. In some cases, a variant 4-1BBL polypeptide of the present disclosure comprises the amino acid sequence set forth in FIG. 2N, where “x” is Arg. In some cases, a variant 4-1BBL polypeptide of the present disclosure comprises the amino acid sequence set forth in FIG. 2N, where “x” is His. In some cases, a variant 4-1BBL polypeptide of the present disclosure comprises the amino acid sequence set forth in FIG. 2N, where “x” is Asp. In some cases, a variant 4-1BBL polypeptide of the present disclosure comprises the amino acid sequence set forth in FIG. 2N, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### I103 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 (indicated by an “x”) is an amino acid other than an isoleucine, e.g., where amino acid 103 is Gly, Ala, Val, Leu, Pro, Phe, Tyr, Trp, Ser, Thr, Cys, Met, Asn, Gln, Lys, Arg, His, Asp, or Glu. The amino acid numbering in FIG. 2O retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2O, where amino acid 103 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at I103. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at 123. In some cases, a variant 4-1BBL polypeptide of the present disclosure comprises the amino acid sequence set forth in SEQ ID NO:1, with an amino acid substitution at 123. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at 124. In some cases, a variant 4-1BBL polypeptide of the present disclosure comprises the amino acid sequence set forth in SEQ ID NO:2, with an amino acid substitution at 124. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at 124. In some cases, a variant 4-1BBL polypeptide of the present disclosure comprises the amino acid sequence set forth in SEQ ID NO:3, with an amino acid substitution at 124.

For example, in some cases, a variant 4-1BBL polypeptide of the present disclosure comprises the amino acid sequence set forth in FIG. 2O, where “x” is any amino acid other than isoleucine; for example, “x” can be Gly, Ala, Val, Leu, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2O, where “x” is Gly. In some cases, a variant 4-1BBL polypeptide of the present disclosure comprises the amino acid sequence set forth in FIG. 2O, where “x” is Ala. In some cases, a variant 4-1BBL polypeptide of the present disclosure comprises the amino acid sequence set forth in FIG. 2O, where “x” is Val. In some cases, a variant 4-1BBL polypeptide of the present disclosure comprises the amino acid sequence set forth in FIG. 2O, where “x” is Leu. In some cases, a variant 4-1BBL polypeptide of the present disclosure comprises the amino acid sequence set forth in FIG. 2O, where “x” is Pro. In some cases, a variant 4-1BBL polypeptide of the present disclosure comprises the amino acid sequence set forth in FIG. 2O, where “x” is Phe. In some cases, a variant 4-1BBL polypeptide of the present disclosure comprises the amino acid sequence set forth in FIG. 2O, where “x” is Tyr. In some cases, a variant 4-1BBL polypeptide of the present disclosure comprises the amino acid sequence set forth in FIG. 2O, where “x” is Trp. In some cases, a variant 4-1BBL polypeptide of the present disclosure comprises the amino acid sequence set forth in FIG. 2O, where “x” is Ser. In some cases, a variant 4-1BBL polypeptide of the present disclosure comprises the amino acid sequence set forth in FIG. 2O, where “x” is Thr. In some cases, a variant 4-1BBL polypeptide of the present disclosure comprises the amino acid sequence set forth in FIG. 2O, where “x” is Cys. In some cases, a variant 4-1BBL polypeptide of the present disclosure comprises the amino acid sequence set forth in FIG. 2O, where “x” is Met. In some cases, a variant 4-1BBL polypeptide of the present disclosure comprises the amino acid sequence set forth in FIG. 2O, where “x” is Asn. In some cases, a variant 4-1BBL polypeptide of the present disclosure comprises the amino acid sequence set forth in FIG. 2O, where “x” is Gln. In some cases, a variant 4-1BBL polypeptide of the present disclosure comprises the amino acid sequence set forth in FIG. 2O, where “x” is Lys. In some cases, a variant 4-1BBL polypeptide of the present disclosure comprises the amino acid sequence set forth in FIG. 2O, where “x” is Arg. In some cases, a variant 4-1BBL polypeptide of the present disclosure comprises the amino acid sequence set forth in FIG. 2O, where “x” is His. In some cases, a variant 4-1BBL polypeptide of the present disclosure comprises the amino acid sequence set forth in FIG. 2O, where “x” is Asp. In some cases, a variant 4-1BBL polypeptide of the present disclosure comprises the amino acid sequence set forth in FIG. 2O, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### D104 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 (indicated by an “x”) is an amino acid other than an aspartic acid, e.g., where amino acid 104 is Gly, Ala, Val, Leu, Ile, Pro, Phe, Tyr, Trp, Ser, Thr, Cys, Met, Asn, Gln, Lys, Arg, His, or Glu. The amino acid numbering in FIG. 2P retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2P, where amino acid 104 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at D104. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at D24. In some cases, a variant 4-1BBL polypeptide of the present disclosure comprises the amino acid sequence set forth in SEQ ID NO: 1, with an amino acid substitution at D24. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D25. In some cases, a variant 4-1BBL polypeptide of the present disclosure comprises the amino acid sequence set forth in SEQ ID NO:2, with an amino acid substitution at D25. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D25. In some cases, a variant 4-1BBL polypeptide of the present disclosure comprises the amino acid sequence set forth in SEQ ID NO:3, with an amino acid substitution at D25.

For example, in some cases, a variant 4-1BBL polypeptide of the present disclosure comprises the amino acid sequence set forth in FIG. 2P, where “x” is any amino acid other than aspartic acid; for example, “x” can be Gly, Ala, Val, Leu, Ile, Pro, Phe, Tyr, Trp, Ser, Thr, Cys, Met, Asn, Gln, Lys, Arg, His, or Glu. In some cases, a variant 4-1BBL polypeptide of the present disclosure comprises the amino acid sequence set forth in FIG. 2P, where “x” is Gly. In some cases, a variant 4-1BBL polypeptide of the present disclosure comprises the amino acid sequence set forth in FIG. 2P, where “x” is Ala. In some cases, a variant 4-1BBL polypeptide of the present disclosure comprises the amino acid sequence set forth in FIG. 2P, where “x” is Val. In some cases, a variant 4-1BBL polypeptide of the present disclosure comprises the amino acid sequence set forth in FIG. 2P, where “x” is Leu. In some cases, a variant 4-1BBL polypeptide of the present disclosure comprises the amino acid sequence set forth in FIG. 2P, where “x” is Ile. In some cases, a variant 4-1BBL polypeptide of the present disclosure comprises the amino acid sequence set forth in FIG. 2P, where “x” is Pro. In some cases, a variant 4-1BBL polypeptide of the present disclosure comprises the amino acid sequence set forth in FIG. 2P, where “x” is Phe. In some cases, a variant 4-1BBL polypeptide of the present disclosure comprises the amino acid sequence set forth in FIG. 2P, where “x” is Tyr. In some cases, a variant 4-1BBL polypeptide of the present disclosure comprises the amino acid sequence set forth in FIG. 2P, where “x” is Trp. In some cases, a variant 4-1BBL polypeptide of the present disclosure comprises the amino acid sequence set forth in FIG. 2P, where “x” is Ser. In some cases, a variant 4-1BBL polypeptide of the present disclosure comprises the amino acid sequence set forth in FIG. 2P, where “x” is Thr. In some cases, a variant 4-1BBL polypeptide of the present disclosure comprises the amino acid sequence set forth in FIG. 2P, where “x” is Cys. In some cases, a variant 4-1BBL polypeptide of the present disclosure comprises the amino acid sequence set forth in FIG. 2P, where “x” is Met. In some cases, a variant 4-1BBL polypeptide of the present disclosure comprises the amino acid sequence set forth in FIG. 2P, where “x” is Asn. In some cases, a variant 4-1BBL polypeptide of the present disclosure comprises the amino acid sequence set forth in FIG. 2P, where “x” is Gln. In some cases, a variant 4-1BBL polypeptide of the present disclosure comprises the amino acid sequence set forth in FIG. 2P, where “x” is Lys. In some cases, a variant 4-1BBL polypeptide of the present disclosure comprises the amino acid sequence set forth in FIG. 2P, where “x” is Arg. In some cases, a variant 4-1BBL polypeptide of the present disclosure comprises the amino acid sequence set forth in FIG. 2P where “x” is His. In some cases, a variant 4-1BBL polypeptide of the present disclosure comprises the amino acid sequence set forth in FIG. 2P, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G105 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 (indicated by an “x”) is an amino acid other than a glycine, e.g., where amino acid 105 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2Q retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Q, where amino acid 105 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G105. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at G25. In some cases, a variant 4-1BBL polypeptide of the present disclosure comprises the amino acid sequence set forth in SEQ ID NO:1, with an amino acid substitution at G25. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G26. In some cases, a variant 4-1BBL polypeptide of the present disclosure comprises the amino acid sequence set forth in SEQ ID NO:2, with an amino acid substitution at G26. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G26. In some cases, a variant 4-1BBL polypeptide of the present disclosure comprises the amino acid sequence set forth in SEQ ID NO:3, with an amino acid substitution at G26.

For example, in some cases, a variant 4-1BBL polypeptide of the present disclosure comprises the amino acid sequence set forth in FIG. 2Q, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2Q, where “x” is Ala. In some cases, a variant 4-1BBL polypeptide of the present disclosure comprises the amino acid sequence set forth in FIG. 2Q, where “x” is Val. In some cases, a variant 4-1BBL polypeptide of the present disclosure comprises the amino acid sequence set forth in FIG. 2Q, where “x” is Leu. In some cases, a variant 4-1BBL polypeptide of the present disclosure comprises the amino acid sequence set forth in FIG. 2Q, where “x” is Ile. In some cases, a variant 4-1BBL polypeptide of the present disclosure comprises the amino acid sequence set forth in FIG. 2Q, where “x” is Pro. In some cases, a variant 4-1BBL polypeptide of the present disclosure comprises the amino acid sequence set forth in FIG. 2Q, where “x” is Phe. In some cases, a variant 4-1BBL polypeptide of the present disclosure comprises the amino acid sequence set forth in FIG. 2Q, where “x” is Tyr. In some cases, a variant 4-1BBL polypeptide of the present disclosure comprises the amino acid sequence set forth in FIG. 2Q, where “x” is Trp. In some cases, a variant 4-1BBL polypeptide of the present disclosure comprises the amino acid sequence set forth in FIG. 2Q, where “x” is Ser. In some cases, a variant 4-1BBL polypeptide of the present disclosure comprises the amino acid sequence set forth in FIG. 2Q, where “x” is Thr. In some cases, a variant 4-1BBL polypeptide of the present disclosure comprises the amino acid sequence set forth in FIG. 2Q, where “x” is Cys. In some cases, a variant 4-1BBL polypeptide of the present disclosure comprises the amino acid sequence set forth in FIG. 2Q, where “x” is Met. In some cases, a variant 4-1BBL polypeptide of the present disclosure comprises the amino acid sequence set forth in FIG. 2Q, where “x” is Asn. In some cases, a variant 4-1BBL polypeptide of the present disclosure comprises the amino acid sequence set forth in FIG. 2Q, where “x” is Gln. In some cases, a variant 4-1BBL polypeptide of the present disclosure comprises the amino acid sequence set forth in FIG. 2Q, where “x” is Lys. In some cases, a variant 4-1BBL polypeptide of the present disclosure comprises the amino acid sequence set forth in FIG. 2Q, where “x” is Arg. In some cases, a variant 4-1BBL polypeptide of the present disclosure comprises the amino acid sequence set forth in FIG. 2Q, where “x” is His. In some cases, a variant 4-1BBL polypeptide of the present disclosure comprises the amino acid sequence set forth in FIG. 2Q, where “x” is Asp. In some cases, a variant 4-1BBL polypeptide of the present disclosure comprises the amino acid sequence set forth in FIG. 2Q, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### P106 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 (indicated by an “x”) is an amino acid other than a proline, e.g., where amino acid 106 is Gly, Ala, Val, Leu, Ile, Phe, Tyr, Trp, Ser, Thr, Cys, Met, Asn, Gln, Lys, Arg, His, Asp, or Glu. The amino acid numbering in FIG. 2R retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 106 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2R, where amino acid 1106 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at P106. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at P26. In some cases, a variant 4-1BBL polypeptide of the present disclosure comprises the amino acid sequence set forth in SEQ ID NO: 1, with an amino acid substitution at P26. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P27. In some cases, a variant 4-1BBL polypeptide of the present disclosure comprises the amino acid sequence set forth in SEQ ID NO:2, with an amino acid substitution at P27. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P27. In some cases, a variant 4-1BBL polypeptide of the present disclosure comprises the amino acid sequence set forth in SEQ ID NO:3, with an amino acid substitution at P27.

For example, in some cases, a variant 4-1BBL polypeptide of the present disclosure comprises the amino acid sequence set forth in FIG. 2R, where “x” is any amino acid other than proline; for example, “x” can be Gly, Ala, Val, Leu, Ile, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2R, where “x” is Gly. In some cases, a variant 4-1BBL polypeptide of the present disclosure comprises the amino acid sequence set forth in FIG. 2R, where “x” is Ala. In some cases, a variant 4-1BBL polypeptide of the present disclosure comprises the amino acid sequence set forth in FIG. 2R, where “x” is Val. In some cases, a variant 4-1BBL polypeptide of the present disclosure comprises the amino acid sequence set forth in FIG. 2R, where “x” is Leu. In some cases, a variant 4-1BBL polypeptide of the present disclosure comprises the amino acid sequence set forth in FIG. 2R, where “x” is Ile. In some cases, a variant 4-1BBL polypeptide of the present disclosure comprises the amino acid sequence set forth in FIG. 2R, where “x” is Phe. In some cases, a variant 4-1BBL polypeptide of the present disclosure comprises the amino acid sequence set forth in FIG. 2R, where “x” is Tyr. In some cases, a variant 4-1BBL polypeptide of the present disclosure comprises the amino acid sequence set forth in FIG. 2R, where “x” is Trp. In some cases, a variant 4-1BBL polypeptide of the present disclosure comprises the amino acid sequence set forth in FIG. 2R, where “x” is Ser. In some cases, a variant 4-1BBL polypeptide of the present disclosure comprises the amino acid sequence set forth in FIG. 2R, where “x” is Thr. In some cases, a variant 4-1BBL polypeptide of the present disclosure comprises the amino acid sequence set forth in FIG. 2R, where “x” is Cys. In some cases, a variant 4-1BBL polypeptide of the present disclosure comprises the amino acid sequence set forth in FIG. 2R, where “x” is Met. In some cases, a variant 4-1BBL polypeptide of the present disclosure comprises the amino acid sequence set forth in FIG. 2R, where “x” is Asn. In some cases, a variant 4-1BBL polypeptide of the present disclosure comprises the amino acid sequence set forth in FIG. 2R, where “x” is Gln. In some cases, a variant 4-1BBL polypeptide of the present disclosure comprises the amino acid sequence set forth in FIG. 2R, where “x” is Lys. In some cases, a variant 4-1BBL polypeptide of the present disclosure comprises the amino acid sequence set forth in FIG. 2R, where “x” is Arg. In some cases, a variant 4-1BBL polypeptide of the present disclosure comprises the amino acid sequence set forth in FIG. 2R, where “x” is His. In some cases, a variant 4-1BBL polypeptide of the present disclosure comprises the amino acid sequence set forth in FIG. 2R, where “x” is Asp. In some cases, a variant 4-1BBL polypeptide of the present disclosure comprises the amino acid sequence set forth in FIG. 2R, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L107 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 (indicated by an “x”) is an amino acid other than a leucine, e.g., where amino acid 107 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2S retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2S, where amino acid 107 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L107. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L27. In some cases, a variant 4-1BBL polypeptide of the present disclosure comprises the amino acid sequence set forth in SEQ ID NO: 1, with an amino acid substitution at L27. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L28. In some cases, a variant 4-1BBL polypeptide of the present disclosure comprises the amino acid sequence set forth in SEQ ID NO:2, with an amino acid substitution at L28. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L28. In some cases, a variant 4-1BBL polypeptide of the present disclosure comprises the amino acid sequence set forth in SEQ ID NO:3, with an amino acid substitution at L28.

For example, in some cases, a variant 4-1BBL polypeptide of the present disclosure comprises the amino acid sequence set forth in FIG. 2S, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2S, where “x” is Gly. In some cases, a variant 4-1BBL polypeptide of the present disclosure comprises the amino acid sequence set forth in FIG. 2S, where “x” is Ala. In some cases, a variant 4-1BBL polypeptide of the present disclosure comprises the amino acid sequence set forth in FIG. 2S, where “x” is Val. In some cases, a variant 4-1BBL polypeptide of the present disclosure comprises the amino acid sequence set forth in FIG. 2S, where “x” is Ile. In some cases, a variant 4-1BBL polypeptide of the present disclosure comprises the amino acid sequence set forth in FIG. 2S, where “x” is Pro. In some cases, a variant 4-1BBL polypeptide of the present disclosure comprises the amino acid sequence set forth in FIG. 2S, where “x” is Phe. In some cases, a variant 4-1BBL polypeptide of the present disclosure comprises the amino acid sequence set forth in FIG. 2S, where “x” is Tyr. In some cases, a variant 4-1BBL polypeptide of the present disclosure comprises the amino acid sequence set forth in FIG. 2S, where “x” is Trp. In some cases, a variant 4-1BBL polypeptide of the present disclosure comprises the amino acid sequence set forth in FIG. 2S, where “x” is Ser. In some cases, a variant 4-1BBL polypeptide of the present disclosure comprises the amino acid sequence set forth in FIG. 2S, where “x” is Thr. In some cases, a variant 4-1BBL polypeptide of the present disclosure comprises the amino acid sequence set forth in FIG. 2S, where “x” is Cys. In some cases, a variant 4-1BBL polypeptide of the present disclosure comprises the amino acid sequence set forth in FIG. 2S, where “x” is Met. In some cases, a variant 4-1BBL polypeptide of the present disclosure comprises the amino acid sequence set forth in FIG. 2S, where “x” is Asn. In some cases, a variant 4-1BBL polypeptide of the present disclosure comprises the amino acid sequence set forth in FIG. 2S, where “x” is Gln. In some cases, a variant 4-1BBL polypeptide of the present disclosure comprises the amino acid sequence set forth in FIG. 2S, where “x” is Lys. In some cases, a variant 4-1BBL polypeptide of the present disclosure comprises the amino acid sequence set forth in FIG. 2S, where “x” is Arg. In some cases, a variant 4-1BBL polypeptide of the present disclosure comprises the amino acid sequence set forth in FIG. 2S, where “x” is His. In some cases, a variant 4-1BBL polypeptide of the present disclosure comprises the amino acid sequence set forth in FIG. 2S, where “x” is Asp. In some cases, a variant 4-1BBL polypeptide of the present disclosure comprises the amino acid sequence set forth in FIG. 2S, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S108 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 (indicated by an “x”) is an amino acid other than a serine, e.g., where amino acid 108 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2T retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2T, where amino acid 108 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S108. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S28. In some cases, a variant 4-1BBL polypeptide of the present disclosure comprises the amino acid sequence set forth in SEQ ID NO:1, with an amino acid substitution at S28. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S29. In some cases, a variant 4-1BBL polypeptide of the present disclosure comprises the amino acid sequence set forth in SEQ ID NO:2, with an amino acid substitution at S29. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S29. In some cases, a variant 4-1BBL polypeptide of the present disclosure comprises the amino acid sequence set forth in SEQ ID NO:3, with an amino acid substitution at S29.

For example, in some cases, a variant 4-1BBL polypeptide of the present disclosure comprises the amino acid sequence set forth in FIG. 2T, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2T, where “x” is Gly. In some cases, a variant 4-1BBL polypeptide of the present disclosure comprises the amino acid sequence set forth in FIG. 2T, where “x” is Ala. In some cases, a variant 4-1BBL polypeptide of the present disclosure comprises the amino acid sequence set forth in FIG. 2T, where “x” is Val. In some cases, a variant 4-1BBL polypeptide of the present disclosure comprises the amino acid sequence set forth in FIG. 2T, where “x” is Leu. In some cases, a variant 4-1BBL polypeptide of the present disclosure comprises the amino acid sequence set forth in FIG. 2T, where “x” is Ile. In some cases, a variant 4-1BBL polypeptide of the present disclosure comprises the amino acid sequence set forth in FIG. 2T, where “x” is Pro. In some cases, a variant 4-1BBL polypeptide of the present disclosure comprises the amino acid sequence set forth in FIG. 2T, where “x” is Phe. In some cases, a variant 4-1BBL polypeptide of the present disclosure comprises the amino acid sequence set forth in FIG. 2T, where “x” is Tyr. In some cases, a variant 4-1BBL polypeptide of the present disclosure comprises the amino acid sequence set forth in FIG. 2T, where “x” is Trp. In some cases, a variant 4-1BBL polypeptide of the present disclosure comprises the amino acid sequence set forth in FIG. 2T, where “x” is Thr. In some cases, a variant 4-1BBL polypeptide of the present disclosure comprises the amino acid sequence set forth in FIG. 2T, where “x” is Cys. In some cases, a variant 4-1BBL polypeptide of the present disclosure comprises the amino acid sequence set forth in FIG. 2T, where “x” is Met. In some cases, a variant 4-1BBL polypeptide of the present disclosure comprises the amino acid sequence set forth in FIG. 2T, where “x” is Asn. In some cases, a variant 4-1BBL polypeptide of the present disclosure comprises the amino acid sequence set forth in FIG. 2T, where “x” is Gln. In some cases, a variant 4-1BBL polypeptide of the present disclosure comprises the amino acid sequence set forth in FIG. 2T, where “x” is Lys. In some cases, a variant 4-1BBL polypeptide of the present disclosure comprises the amino acid sequence set forth in FIG. 2T, where “x” is Arg. In some cases, a variant 4-1BBL polypeptide of the present disclosure comprises the amino acid sequence set forth in FIG. 2T, where “x” is His. In some cases, a variant 4-1BBL polypeptide of the present disclosure comprises the amino acid sequence set forth in FIG. 2T, where “x” is Asp. In some cases, a variant 4-1BBL polypeptide of the present disclosure comprises the amino acid sequence set forth in FIG. 2T, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### W109 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 (indicated by an “x”) is an amino acid other than a tryptophan, e.g., where amino acid 109 is Gly, Ala, Val, Leu, Ile, Pro, Phe, Tyr, Ser, Thr, Cys, Met, Asn, Gln, Lys, Arg, His, Asp, or Glu. The amino acid numbering in FIG. 2U retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2U, where amino acid 109 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at W109. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at W29. In some cases, a variant 4-1BBL polypeptide of the present disclosure comprises the amino acid sequence set forth in SEQ ID NO: 1, with an amino acid substitution at W29. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at W30. In some cases, a variant 4-1BBL polypeptide of the present disclosure comprises the amino acid sequence set forth in SEQ ID NO:2, with an amino acid substitution at W30. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at W30. In some cases, a variant 4-1BBL polypeptide of the present disclosure comprises the amino acid sequence set forth in SEQ ID NO:3, with an amino acid substitution at W30.

For example, in some cases, a variant 4-1BBL polypeptide of the present disclosure comprises the amino acid sequence set forth in FIG. 2U, where “x” is any amino acid other than tryptophan; for example, “x” can be Gly, Ala, Val, Leu, Ile, Pro, Phe, Tyr, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2U, where “x” is Gly. In some cases, a variant 4-1BBL polypeptide of the present disclosure comprises the amino acid sequence set forth in FIG. 2U, where “x” is Ala. In some cases, a variant 4-1BBL polypeptide of the present disclosure comprises the amino acid sequence set forth in FIG. 2U, where “x” is Val. In some cases, a variant 4-1BBL polypeptide of the present disclosure comprises the amino acid sequence set forth in FIG. 2U, where “x” is Leu. In some cases, a variant 4-1BBL polypeptide of the present disclosure comprises the amino acid sequence set forth in FIG. 2U, where “x” is Ile. In some cases, a variant 4-1BBL polypeptide of the present disclosure comprises the amino acid sequence set forth in FIG. 2U, where “x” is Pro. In some cases, a variant 4-1BBL polypeptide of the present disclosure comprises the amino acid sequence set forth in FIG. 2U, where “x” is Phe. In some cases, a variant 4-1BBL polypeptide of the present disclosure comprises the amino acid sequence set forth in FIG. 2U, where “x” is Tyr. In some cases, a variant 4-1BBL polypeptide of the present disclosure comprises the amino acid sequence set forth in FIG. 2U, where “x” is Ser. In some cases, a variant 4-1BBL polypeptide of the present disclosure comprises the amino acid sequence set forth in FIG. 2U, where “x” is Thr. In some cases, a variant 4-1BBL polypeptide of the present disclosure comprises the amino acid sequence set forth in FIG. 2U, where “x” is Cys. In some cases, a variant 4-1BBL polypeptide of the present disclosure comprises the amino acid sequence set forth in FIG. 2U, where “x” is Met. In some cases, a variant 4-1BBL polypeptide of the present disclosure comprises the amino acid sequence set forth in FIG. 2U, where “x” is Asn. In some cases, a variant 4-1BBL polypeptide of the present disclosure comprises the amino acid sequence set forth in FIG. 2U, where “x” is Gln. In some cases, a variant 4-1BBL polypeptide of the present disclosure comprises the amino acid sequence set forth in FIG. 2U, where “x” is Lys. In some cases, a variant 4-1BBL polypeptide of the present disclosure comprises the amino acid sequence set forth in FIG. 2U, where “x” is Arg. In some cases, a variant 4-1BBL polypeptide of the present disclosure comprises the amino acid sequence set forth in FIG. 2U, where “x” is His. In some cases, a variant 4-1BBL polypeptide of the present disclosure comprises the amino acid sequence set forth in FIG. 2U, where “x” is Asp. In some cases, a variant 4-1BBL polypeptide of the present disclosure comprises the amino acid sequence set forth in FIG. 2U, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Y110 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 (indicated by an “x”) is an amino acid other than a tyrosine, e.g., where amino acid 110 is Gly, Ala, Val, Leu, Ile, Pro, Phe, Trp, Ser, Thr, Cys, Met, Asn, Gln, Lys, Arg, His, Asp, or Glu. The amino acid numbering in FIG. 2V retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2V, where amino acid 110 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Y110. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at Y30. In some cases, a variant 4-1BBL polypeptide of the present disclosure comprises the amino acid sequence set forth in SEQ ID NO: 1, with an amino acid substitution at Y30. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Y31. In some cases, a variant 4-1BBL polypeptide of the present disclosure comprises the amino acid sequence set forth in SEQ ID NO:2, with an amino acid substitution at Y31. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Y31. In some cases, a variant 4-1BBL polypeptide of the present disclosure comprises the amino acid sequence set forth in SEQ ID NO:3, with an amino acid substitution at Y31.

For example, in some cases, a variant 4-1BBL polypeptide of the present disclosure comprises the amino acid sequence set forth in FIG. 2V, where “x” is any amino acid other than tyrosine; for example, “x” can be Gly, Ala, Val, Leu, Ile, Pro, Phe, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2V, where “x” is Gly. In some cases, a variant 4-1BBL polypeptide of the present disclosure comprises the amino acid sequence set forth in FIG. 2V, where “x” is Ala. In some cases, a variant 4-1BBL polypeptide of the present disclosure comprises the amino acid sequence set forth in FIG. 2V, where “x” is Val. In some cases, a variant 4-1BBL polypeptide of the present disclosure comprises the amino acid sequence set forth in FIG. 2V, where “x” is Leu. In some cases, a variant 4-1BBL polypeptide of the present disclosure comprises the amino acid sequence set forth in FIG. 2V, where “x” is Ile. In some cases, a variant 4-1BBL polypeptide of the present disclosure comprises the amino acid sequence set forth in FIG. 2V, where “x” is Pro. In some cases, a variant 4-1BBL polypeptide of the present disclosure comprises the amino acid sequence set forth in FIG. 2V, where “x” is Phe. In some cases, a variant 4-1BBL polypeptide of the present disclosure comprises the amino acid sequence set forth in FIG. 2V, where “x” is Trp. In some cases, a variant 4-1BBL polypeptide of the present disclosure comprises the amino acid sequence set forth in FIG. 2V, where “x” is Ser. In some cases, a variant 4-1BBL polypeptide of the present disclosure comprises the amino acid sequence set forth in FIG. 2V, where “x” is Thr. In some cases, a variant 4-1BBL polypeptide of the present disclosure comprises the amino acid sequence set forth in FIG. 2V, where “x” is Cys. In some cases, a variant 4-1BBL polypeptide of the present disclosure comprises the amino acid sequence set forth in FIG. 2V, where “x” is Met. In some cases, a variant 4-1BBL polypeptide of the present disclosure comprises the amino acid sequence set forth in FIG. 2V, where “x” is Asn. In some cases, a variant 4-1BBL polypeptide of the present disclosure comprises the amino acid sequence set forth in FIG. 2V, where “x” is Gln. In some cases, a variant 4-1BBL polypeptide of the present disclosure comprises the amino acid sequence set forth in FIG. 2V, where “x” is Lys. In some cases, a variant 4-1BBL polypeptide of the present disclosure comprises the amino acid sequence set forth in FIG. 2V, where “x” is Arg. In some cases, a variant 4-1BBL polypeptide of the present disclosure comprises the amino acid sequence set forth in FIG. 2V, where “x” is His. In some cases, a variant 4-1BBL polypeptide of the present disclosure comprises the amino acid sequence set forth in FIG. 2V, where “x” is Asp. In some cases, a variant 4-1BBL polypeptide of the present disclosure comprises the amino acid sequence set forth in FIG. 2V, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S111 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 (indicated by an “x”) is an amino acid other than a serine, e.g., where amino acid 111 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2W retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2W, where amino acid 111 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S111. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S31. In some cases, a variant 4-1BBL polypeptide of the present disclosure comprises the amino acid sequence set forth in SEQ ID NO: 1, with an amino acid substitution at S31. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S32. In some cases, a variant 4-1BBL polypeptide of the present disclosure comprises the amino acid sequence set forth in SEQ ID NO:2, with an amino acid substitution at S32. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S32. In some cases, a variant 4-1BBL polypeptide of the present disclosure comprises the amino acid sequence set forth in SEQ ID NO:3, with an amino acid substitution at S32.

For example, in some cases, a variant 4-1BBL polypeptide of the present disclosure comprises the amino acid sequence set forth in FIG. 2W, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2W, where “x” is Gly. In some cases, a variant 4-1BBL polypeptide of the present disclosure comprises the amino acid sequence set forth in FIG. 2W, where “x” is Ala. In some cases, a variant 4-1BBL polypeptide of the present disclosure comprises the amino acid sequence set forth in FIG. 2W, where “x” is Val. In some cases, a variant 4-1BBL polypeptide of the present disclosure comprises the amino acid sequence set forth in FIG. 2W, where “x” is Leu. In some cases, a variant 4-1BBL polypeptide of the present disclosure comprises the amino acid sequence set forth in FIG. 2W, where “x” is Ile. In some cases, a variant 4-1BBL polypeptide of the present disclosure comprises the amino acid sequence set forth in FIG. 2W, where “x” is Pro. In some cases, a variant 4-1BBL polypeptide of the present disclosure comprises the amino acid sequence set forth in FIG. 2W, where “x” is Phe. In some cases, a variant 4-1BBL polypeptide of the present disclosure comprises the amino acid sequence set forth in FIG. 2W, where “x” is Tyr. In some cases, a variant 4-1BBL polypeptide of the present disclosure comprises the amino acid sequence set forth in FIG. 2W, where “x” is Trp. In some cases, a variant 4-1BBL polypeptide of the present disclosure comprises the amino acid sequence set forth in FIG. 2W, where “x” is Thr. In some cases, a variant 4-1BBL polypeptide of the present disclosure comprises the amino acid sequence set forth in FIG. 2W, where “x” is Cys. In some cases, a variant 4-1BBL polypeptide of the present disclosure comprises the amino acid sequence set forth in FIG. 2W, where “x” is Met. In some cases, a variant 4-1BBL polypeptide of the present disclosure comprises the amino acid sequence set forth in FIG. 2W, where “x” is Asn. In some cases, a variant 4-1BBL polypeptide of the present disclosure comprises the amino acid sequence set forth in FIG. 2W, where “x” is Gln. In some cases, a variant 4-1BBL polypeptide of the present disclosure comprises the amino acid sequence set forth in FIG. 2W, where “x” is Lys. In some cases, a variant 4-1BBL polypeptide of the present disclosure comprises the amino acid sequence set forth in FIG. 2W, where “x” is Arg. In some cases, a variant 4-1BBL polypeptide of the present disclosure comprises the amino acid sequence set forth in FIG. 2W, where “x” is His. In some cases, a variant 4-1BBL polypeptide of the present disclosure comprises the amino acid sequence set forth in FIG. 2W, where “x” is Asp. In some cases, a variant 4-1BBL polypeptide of the present disclosure comprises the amino acid sequence set forth in FIG. 2W, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### D112 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 (indicated by an “x”) is an amino acid other than an aspartic acid, e.g., where amino acid 112 is Gly, Ala, Val, Leu, Ile, Pro, Phe, Tyr, Trp, Ser, Thr, Cys, Met, Asn, Gln, Lys, Arg, His, or Glu. The amino acid numbering in FIG. 2X retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 112 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2X, where amino acid 2X is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at D112. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at D32. In some cases, a variant 4-1BBL polypeptide of the present disclosure comprises the amino acid sequence set forth in SEQ ID NO: 1, with an amino acid substitution at D32. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D33. In some cases, a variant 4-1BBL polypeptide of the present disclosure comprises the amino acid sequence set forth in SEQ ID NO:2, with an amino acid substitution D33. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D33. In some cases, a variant 4-1BBL polypeptide of the present disclosure comprises the amino acid sequence set forth in SEQ ID NO:3, with an amino acid substitution at D33.

For example, in some cases, a variant 4-1BBL polypeptide of the present disclosure comprises the amino acid sequence set forth in FIG. 2X, where “x” is any amino acid other than aspartic acid; for example, “x” can be Gly, Ala, Val, Leu, Ile, Pro, Phe, Tyr, Trp, Ser, Thr, Cys, Met, Asn, Gln, Lys, Arg, His, or Glu. In some cases, a variant 4-1BBL polypeptide of the present disclosure comprises the amino acid sequence set forth in FIG. 2X, where “x” is Gly. In some cases, a variant 4-1BBL polypeptide of the present disclosure comprises the amino acid sequence set forth in FIG. 2X, where “x” is Ala. In some cases, a variant 4-1BBL polypeptide of the present disclosure comprises the amino acid sequence set forth in FIG. 2X, where “x” is Val. In some cases, a variant 4-1BBL polypeptide of the present disclosure comprises the amino acid sequence set forth in FIG. 2X, where “x” is Leu. In some cases, a variant 4-1BBL polypeptide of the present disclosure comprises the amino acid sequence set forth in FIG. 2X, where “x” is Ile. In some cases, a variant 4-1BBL polypeptide of the present disclosure comprises the amino acid sequence set forth in FIG. 2X, where “x” is Pro. In some cases, a variant 4-1BBL polypeptide of the present disclosure comprises the amino acid sequence set forth in FIG. 2X, where “x” is Phe. In some cases, a variant 4-1BBL polypeptide of the present disclosure comprises the amino acid sequence set forth in FIG. 2X, where “x” is Tyr. In some cases, a variant 4-1BBL polypeptide of the present disclosure comprises the amino acid sequence set forth in FIG. 2X, where “x” is Trp. In some cases, a variant 4-1BBL polypeptide of the present disclosure comprises the amino acid sequence set forth in FIG. 2X, where “x” is Ser. In some cases, a variant 4-1BBL polypeptide of the present disclosure comprises the amino acid sequence set forth in FIG. 2X, where “x” is Thr. In some cases, a variant 4-1BBL polypeptide of the present disclosure comprises the amino acid sequence set forth in FIG. 2X, where “x” is Cys. In some cases, a variant 4-1BBL polypeptide of the present disclosure comprises the amino acid sequence set forth in FIG. 2X, where “x” is Met. In some cases, a variant 4-1BBL polypeptide of the present disclosure comprises the amino acid sequence set forth in FIG. 2X, where “x” is Asn. In some cases, a variant 4-1BBL polypeptide of the present disclosure comprises the amino acid sequence set forth in FIG. 2X, where “x” is Gln. In some cases, a variant 4-1BBL polypeptide of the present disclosure comprises the amino acid sequence set forth in FIG. 2X, where “x” is Lys. In some cases, a variant 4-1BBL polypeptide of the present disclosure comprises the amino acid sequence set forth in FIG. 2X, where “x” is Arg. In some cases, a variant 4-1BBL polypeptide of the present disclosure comprises the amino acid sequence set forth in FIG. 2X, where “x” is His. In some cases, a variant 4-1BBL polypeptide of the present disclosure comprises the amino acid sequence set forth in FIG. 2X, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### P113 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 (indicated by an “x”) is an amino acid other than a proline, e.g., where amino acid 113 is Gly, Ala, Val, Leu, Ile, Phe, Tyr, Trp, Ser, Thr, Cys, Met, Asn, Gln, Lys, Arg, His, Asp, or Glu. The amino acid numbering in FIG. 2Y retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 1113 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Y, where amino acid 113 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at P113. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at P33. In some cases, a variant 4-1BBL polypeptide of the present disclosure comprises the amino acid sequence set forth in SEQ ID NO:1, with an amino acid substitution at P33. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P34. In some cases, a variant 4-1BBL polypeptide of the present disclosure comprises the amino acid sequence set forth in SEQ ID NO:2, with an amino acid substitution P34. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P34. In some cases, a variant 4-1BBL polypeptide of the present disclosure comprises the amino acid sequence set forth in SEQ ID NO:3, with an amino acid substitution at P34.

For example, in some cases, a variant 4-1BBL polypeptide of the present disclosure comprises the amino acid sequence set forth in FIG. 2Y, where “x” is any amino acid other than methionine; for example, “x” can be Gly, Ala, Val, Leu, Ile, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2Y, where “x” is Gly. In some cases, a variant 4-1BBL polypeptide of the present disclosure comprises the amino acid sequence set forth in FIG. 2Y, where “x” is Ala. In some cases, a variant 4-1BBL polypeptide of the present disclosure comprises the amino acid sequence set forth in FIG. 2Y, where “x” is Val. In some cases, a variant 4-1BBL polypeptide of the present disclosure comprises the amino acid sequence set forth in FIG. 2Y, where “x” is Leu. In some cases, a variant 4-1BBL polypeptide of the present disclosure comprises the amino acid sequence set forth in FIG. 2Y, where “x” is Ile. In some cases, a variant 4-1BBL polypeptide of the present disclosure comprises the amino acid sequence set forth in FIG. 2Y, where “x” is Phe. In some cases, a variant 4-1BBL polypeptide of the present disclosure comprises the amino acid sequence set forth in FIG. 2Y, where “x” is Tyr. In some cases, a variant 4-1BBL polypeptide of the present disclosure comprises the amino acid sequence set forth in FIG. 2Y, where “x” is Trp. In some cases, a variant 4-1BBL polypeptide of the present disclosure comprises the amino acid sequence set forth in FIG. 2Y, where “x” is Ser. In some cases, a variant 4-1BBL polypeptide of the present disclosure comprises the amino acid sequence set forth in FIG. 2Y, where “x” is Thr. In some cases, a variant 4-1BBL polypeptide of the present disclosure comprises the amino acid sequence set forth in FIG. 2Y, where “x” is Cys. In some cases, a variant 4-1BBL polypeptide of the present disclosure comprises the amino acid sequence set forth in FIG. 2Y, where “x” is Met. In some cases, a variant 4-1BBL polypeptide of the present disclosure comprises the amino acid sequence set forth in FIG. 2Y, where “x” is Asn. In some cases, a variant 4-1BBL polypeptide of the present disclosure comprises the amino acid sequence set forth in FIG. 2Y, where “x” is Gln. In some cases, a variant 4-1BBL polypeptide of the present disclosure comprises the amino acid sequence set forth in FIG. 2Y, where “x” is Lys. In some cases, a variant 4-1BBL polypeptide of the present disclosure comprises the amino acid sequence set forth in FIG. 2Y, where “x” is Arg. In some cases, a variant 4-1BBL polypeptide of the present disclosure comprises the amino acid sequence set forth in FIG. 2Y, where “x” is His. In some cases, a variant 4-1BBL polypeptide of the present disclosure comprises the amino acid sequence set forth in FIG. 2Y, where “x” is Asp. In some cases, a variant 4-1BBL polypeptide of the present disclosure comprises the amino acid sequence set forth in FIG. 2Y, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G114 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 (indicated by an “x”) is an amino acid other than a glycine, e.g., where amino acid 114 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2Z retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2Z, where amino acid 114 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G114. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at G34. In some cases, a variant 4-1BBL polypeptide of the present disclosure comprises the amino acid sequence set forth in SEQ ID NO: 1, with an amino acid substitution at G34. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G35. In some cases, a variant 4-1BBL polypeptide of the present disclosure comprises the amino acid sequence set forth in SEQ ID NO:2, with an amino acid substitution G35. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G35. In some cases, a variant 4-1BBL polypeptide of the present disclosure comprises the amino acid sequence set forth in SEQ ID NO:3, with an amino acid substitution at G35.

For example, in some cases, a variant 4-1BBL polypeptide of the present disclosure comprises the amino acid sequence set forth in FIG. 2Z, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2Z, where “x” is Ala. In some cases, a variant 4-1BBL polypeptide of the present disclosure comprises the amino acid sequence set forth in FIG. 2Z, where “x” is Val. In some cases, a variant 4-1BBL polypeptide of the present disclosure comprises the amino acid sequence set forth in FIG. 2Z, where “x” is Leu. In some cases, a variant 4-1BBL polypeptide of the present disclosure comprises the amino acid sequence set forth in FIG. 2Z, where “x” is Ile. In some cases, a variant 4-1BBL polypeptide of the present disclosure comprises the amino acid sequence set forth in FIG. 2Z, where “x” is Pro. In some cases, a variant 4-1BBL polypeptide of the present disclosure comprises the amino acid sequence set forth in FIG. 2Z, where “x” is Phe. In some cases, a variant 4-1BBL polypeptide of the present disclosure comprises the amino acid sequence set forth in FIG. 2Z, where “x” is Tyr. In some cases, a variant 4-1BBL polypeptide of the present disclosure comprises the amino acid sequence set forth in FIG. 2Z, where “x” is Trp. In some cases, a variant 4-1BBL polypeptide of the present disclosure comprises the amino acid sequence set forth in FIG. 2Z, where “x” is Ser. In some cases, a variant 4-1BBL polypeptide of the present disclosure comprises the amino acid sequence set forth in FIG. 2Z, where “x” is Thr. In some cases, a variant 4-1BBL polypeptide of the present disclosure comprises the amino acid sequence set forth in FIG. 2Z, where “x” is Cys. In some cases, a variant 4-1BBL polypeptide of the present disclosure comprises the amino acid sequence set forth in FIG. 2Z, where “x” is Met. In some cases, a variant 4-1BBL polypeptide of the present disclosure comprises the amino acid sequence set forth in FIG. 2Z, where “x” is Asn. In some cases, a variant 4-1BBL polypeptide of the present disclosure comprises the amino acid sequence set forth in FIG. 2Z, where “x” is Gln. In some cases, a variant 4-1BBL polypeptide of the present disclosure comprises the amino acid sequence set forth in FIG. 2Z, where “x” is Lys. In some cases, a variant 4-1BBL polypeptide of the present disclosure comprises the amino acid sequence set forth in FIG. 2Z, where “x” is Arg. In some cases, a variant 4-1BBL polypeptide of the present disclosure comprises the amino acid sequence set forth in FIG. 2Z, where “x” is His. In some cases, a variant 4-1BBL polypeptide of the present disclosure comprises the amino acid sequence set forth in FIG. 2Z, where “x” is Asp. In some cases, a variant 4-1BBL polypeptide of the present disclosure comprises the amino acid sequence set forth in FIG. 2Z, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L115

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 (indicated by an “x”) is an amino acid other than a leucine, e.g., where amino acid 115 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2AA retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AA, where amino acid 115 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L115. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L35. In some cases, a variant 4-1BBL polypeptide of the present disclosure comprises the amino acid sequence set forth in SEQ ID NO:1, with an amino acid substitution at L35. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L36. In some cases, a variant 4-1BBL polypeptide of the present disclosure comprises the amino acid sequence set forth in SEQ ID NO:2, with an amino acid substitution L36. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L36. In some cases, a variant 4-1BBL polypeptide of the present disclosure comprises the amino acid sequence set forth in SEQ ID NO:3, with an amino acid substitution at L36.

For example, in some cases, a variant 4-1BBL polypeptide of the present disclosure comprises the amino acid sequence set forth in FIG. 2AA, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2AA, where “x” is Gly. In some cases, a variant 4-1BBL polypeptide of the present disclosure comprises the amino acid sequence set forth in FIG. 2AA, where “x” is Ala. In some cases, a variant 4-1BBL polypeptide of the present disclosure comprises the amino acid sequence set forth in FIG. 2AA, where “x” is Val. In some cases, a variant 4-1BBL polypeptide of the present disclosure comprises the amino acid sequence set forth in FIG. 2AA, where “x” is Ile. In some cases, a variant 4-1BBL polypeptide of the present disclosure comprises the amino acid sequence set forth in FIG. 2AA, where “x” is Pro. In some cases, a variant 4-1BBL polypeptide of the present disclosure comprises the amino acid sequence set forth in FIG. 2AA, where “x” is Phe. In some cases, a variant 4-1BBL polypeptide of the present disclosure comprises the amino acid sequence set forth in FIG. 2AA, where “x” is Tyr. In some cases, a variant 4-1BBL polypeptide of the present disclosure comprises the amino acid sequence set forth in FIG. 2AA, where “x” is Trp. In some cases, a variant 4-1BBL polypeptide of the present disclosure comprises the amino acid sequence set forth in FIG. 2AA, where “x” is Ser. In some cases, a variant 4-1BBL polypeptide of the present disclosure comprises the amino acid sequence set forth in FIG. 2AA, where “x” is Thr. In some cases, a variant 4-1BBL polypeptide of the present disclosure comprises the amino acid sequence set forth in FIG. 2AA, where “x” is Cys. In some cases, a variant 4-1BBL polypeptide of the present disclosure comprises the amino acid sequence set forth in FIG. 2AA, where “x” is Met. In some cases, a variant 4-1BBL polypeptide of the present disclosure comprises the amino acid sequence set forth in FIG. 2AA, where “x” is Asn. In some cases, a variant 4-1BBL polypeptide of the present disclosure comprises the amino acid sequence set forth in FIG. 2AA, where “x” is Gln. In some cases, a variant 4-1BBL polypeptide of the present disclosure comprises the amino acid sequence set forth in FIG. 2AA, where “x” is Lys. In some cases, a variant 4-1BBL polypeptide of the present disclosure comprises the amino acid sequence set forth in FIG. 2AA, where “x” is Arg. In some cases, a variant 4-1BBL polypeptide of the present disclosure comprises the amino acid sequence set forth in FIG. 2AA, where “x” is His. In some cases, a variant 4-1BBL polypeptide of the present disclosure comprises the amino acid sequence set forth in FIG. 2AA, where “x” is Asp. In some cases, a variant 4-1BBL polypeptide of the present disclosure comprises the amino acid sequence set forth in FIG. 2AA, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G117 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 (indicated by an “x”) is an amino acid other than a glycine, e.g., where amino acid 117 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2BB retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BB, where amino acid 117 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G117. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at G37. In some cases, a variant 4-1BBL polypeptide of the present disclosure comprises the amino acid sequence set forth in SEQ ID NO: 1, with an amino acid substitution at G37. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G38. In some cases, a variant 4-1BBL polypeptide of the present disclosure comprises the amino acid sequence set forth in SEQ ID NO:2, with an amino acid substitution G38. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G38. In some cases, a variant 4-1BBL polypeptide of the present disclosure comprises the amino acid sequence set forth in SEQ ID NO:3, with an amino acid substitution at G38.

For example, in some cases, a variant 4-1BBL polypeptide of the present disclosure comprises the amino acid sequence set forth in FIG. 2BB, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2BB, where “x” is Ala. In some cases, a variant 4-1BBL polypeptide of the present disclosure comprises the amino acid sequence set forth in FIG. 2BB, where “x” is Val. In some cases, a variant 4-1BBL polypeptide of the present disclosure comprises the amino acid sequence set forth in FIG. 2BB, where “x” is Leu. In some cases, a variant 4-1BBL polypeptide of the present disclosure comprises the amino acid sequence set forth in FIG. 2BB, where “x” is Ile. In some cases, a variant 4-1BBL polypeptide of the present disclosure comprises the amino acid sequence set forth in FIG. 2BB, where “x” is Pro. In some cases, a variant 4-1BBL polypeptide of the present disclosure comprises the amino acid sequence set forth in FIG. 2BB, where “x” is Phe. In some cases, a variant 4-1BBL polypeptide of the present disclosure comprises the amino acid sequence set forth in FIG. 2BB, where “x” is Tyr. In some cases, a variant 4-1BBL polypeptide of the present disclosure comprises the amino acid sequence set forth in FIG. 2BB, where “x” is Trp. In some cases, a variant 4-1BBL polypeptide of the present disclosure comprises the amino acid sequence set forth in FIG. 2BB, where “x” is Ser. In some cases, a variant 4-1BBL polypeptide of the present disclosure comprises the amino acid sequence set forth in FIG. 2BB, where “x” is Thr. In some cases, a variant 4-1BBL polypeptide of the present disclosure comprises the amino acid sequence set forth in FIG. 2BB, where “x” is Cys. In some cases, a variant 4-1BBL polypeptide of the present disclosure comprises the amino acid sequence set forth in FIG. 2BB, where “x” is Met. In some cases, a variant 4-1BBL polypeptide of the present disclosure comprises the amino acid sequence set forth in FIG. 2BB, where “x” is Asn. In some cases, a variant 4-1BBL polypeptide of the present disclosure comprises the amino acid sequence set forth in FIG. 2BB, where “x” is Gln. In some cases, a variant 4-1BBL polypeptide of the present disclosure comprises the amino acid sequence set forth in FIG. 2BB, where “x” is Lys. In some cases, a variant 4-1BBL polypeptide of the present disclosure comprises the amino acid sequence set forth in FIG. 2BB, where “x” is Arg. In some cases, a variant 4-1BBL polypeptide of the present disclosure comprises the amino acid sequence set forth in FIG. 2BB, where “x” is His. In some cases, a variant 4-1BBL polypeptide of the present disclosure comprises the amino acid sequence set forth in FIG. 2BB, where “x” is Asp. In some cases, a variant 4-1BBL polypeptide of the present disclosure comprises the amino acid sequence set forth in FIG. 2BB, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### V118

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 (indicated by an “x”) is an amino acid other than a valine, e.g., where amino acid 118 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2CC retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CC, where amino acid 118 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V118. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at V38. In some cases, a variant 4-1BBL polypeptide of the present disclosure comprises the amino acid sequence set forth in SEQ ID NO:1, with an amino acid substitution at V38. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V39. In some cases, a variant 4-1BBL polypeptide of the present disclosure comprises the amino acid sequence set forth in SEQ ID NO:2, with an amino acid substitution V39. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V39. In some cases, a variant 4-1BBL polypeptide of the present disclosure comprises the amino acid sequence set forth in SEQ ID NO:3, with an amino acid substitution at V39.

For example, in some cases, a variant 4-1BBL polypeptide of the present disclosure comprises the amino acid sequence set forth in FIG. 2CC, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2CC, where “x” is Gly. In some cases, a variant 4-1BBL polypeptide of the present disclosure comprises the amino acid sequence set forth in FIG. 2CC, where “x” is Ala. In some cases, a variant 4-1BBL polypeptide of the present disclosure comprises the amino acid sequence set forth in FIG. 2CC, where “x” is Leu. In some cases, a variant 4-1BBL polypeptide of the present disclosure comprises the amino acid sequence set forth in FIG. 2CC, where “x” is Ile. In some cases, a variant 4-1BBL polypeptide of the present disclosure comprises the amino acid sequence set forth in FIG. 2CC, where “x” is Pro. In some cases, a variant 4-1BBL polypeptide of the present disclosure comprises the amino acid sequence set forth in FIG. 2CC, where “x” is Phe. In some cases, a variant 4-1BBL polypeptide of the present disclosure comprises the amino acid sequence set forth in FIG. 2CC, where “x” is Tyr. In some cases, a variant 4-1BBL polypeptide of the present disclosure comprises the amino acid sequence set forth in FIG. 2CC, where “x” is Trp. In some cases, a variant 4-1BBL polypeptide of the present disclosure comprises the amino acid sequence set forth in FIG. 2CC, where “x” is Ser. In some cases, a variant 4-1BBL polypeptide of the present disclosure comprises the amino acid sequence set forth in FIG. 2CC, where “x” is Thr. In some cases, a variant 4-1BBL polypeptide of the present disclosure comprises the amino acid sequence set forth in FIG. 2CC, where “x” is Cys. In some cases, a variant 4-1BBL polypeptide of the present disclosure comprises the amino acid sequence set forth in FIG. 2CC, where “x” is Met. In some cases, a variant 4-1BBL polypeptide of the present disclosure comprises the amino acid sequence set forth in FIG. 2CC, where “x” is Asn. In some cases, a variant 4-1BBL polypeptide of the present disclosure comprises the amino acid sequence set forth in FIG. 2CC, where “x” is Gln. In some cases, a variant 4-1BBL polypeptide of the present disclosure comprises the amino acid sequence set forth in FIG. 2CC, where “x” is Lys. In some cases, a variant 4-1BBL polypeptide of the present disclosure comprises the amino acid sequence set forth in FIG. 2CC, where “x” is Arg. In some cases, a variant 4-1BBL polypeptide of the present disclosure comprises the amino acid sequence set forth in FIG. 2CC, where “x” is His. In some cases, a variant 4-1BBL polypeptide of the present disclosure comprises the amino acid sequence set forth in FIG. 2CC, where “x” is Asp. In some cases, a variant 4-1BBL polypeptide of the present disclosure comprises the amino acid sequence set forth in FIG. 2CC, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### S119 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 (indicated by an “x”) is an amino acid other than a serine, e.g., where amino acid 119 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2DD retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 119 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S119. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at S39. In some cases, a variant 4-1BBL polypeptide of the present disclosure comprises the amino acid sequence set forth in SEQ ID NO: 1, with an amino acid substitution at S39. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S40. In some cases, a variant 4-1BBL polypeptide of the present disclosure comprises the amino acid sequence set forth in SEQ ID NO:2, with an amino acid substitution S40. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S40. In some cases, a variant 4-1BBL polypeptide of the present disclosure comprises the amino acid sequence set forth in SEQ ID NO:3, with an amino acid substitution at S40.

For example, in some cases, a variant 4-1BBL polypeptide of the present disclosure comprises the amino acid sequence set forth in FIG. 2DD, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2DD, where “x” is Gly. In some cases, a variant 4-1BBL polypeptide of the present disclosure comprises the amino acid sequence set forth in FIG. 2DD, where “x” is Ala. In some cases, a variant 4-1BBL polypeptide of the present disclosure comprises the amino acid sequence set forth in FIG. 2DD, where “x” is Val. In some cases, a variant 4-1BBL polypeptide of the present disclosure comprises the amino acid sequence set forth in FIG. 2DD, where “x” is Leu. In some cases, a variant 4-1BBL polypeptide of the present disclosure comprises the amino acid sequence set forth in FIG. 2DD, where “x” is Ile. In some cases, a variant 4-1BBL polypeptide of the present disclosure comprises the amino acid sequence set forth in FIG. 2DD, where “x” is Pro. In some cases, a variant 4-1BBL polypeptide of the present disclosure comprises the amino acid sequence set forth in FIG. 2DD, where “x” is Phe. In some cases, a variant 4-1BBL polypeptide of the present disclosure comprises the amino acid sequence set forth in FIG. 2DD, where “x” is Tyr. In some cases, a variant 4-1BBL polypeptide of the present disclosure comprises the amino acid sequence set forth in FIG. 2DD, where “x” is Trp. In some cases, a variant 4-1BBL polypeptide of the present disclosure comprises the amino acid sequence set forth in FIG. 2DD, where “x” is Thr. In some cases, a variant 4-1BBL polypeptide of the present disclosure comprises the amino acid sequence set forth in FIG. 2DD, where “x” is Cys. In some cases, a variant 4-1BBL polypeptide of the present disclosure comprises the amino acid sequence set forth in FIG. 2DD, where “x” is Met. In some cases, a variant 4-1BBL polypeptide of the present disclosure comprises the amino acid sequence set forth in FIG. 2DD, where “x” is Asn. In some cases, a variant 4-1BBL polypeptide of the present disclosure comprises the amino acid sequence set forth in FIG. 2DD, where “x” is Gln. In some cases, a variant 4-1BBL polypeptide of the present disclosure comprises the amino acid sequence set forth in FIG. 2DD, where “x” is Lys. In some cases, a variant 4-1BBL polypeptide of the present disclosure comprises the amino acid sequence set forth in FIG. 2DD, where “x” is Arg. In some cases, a variant 4-1BBL polypeptide of the present disclosure comprises the amino acid sequence set forth in FIG. 2DD, where “x” is His. In some cases, a variant 4-1BBL polypeptide of the present disclosure comprises the amino acid sequence set forth in FIG. 2DD, where “x” is Asp. In some cases, a variant 4-1BBL polypeptide of the present disclosure comprises the amino acid sequence set forth in FIG. 2DD, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L120 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 (indicated by an “x”) is an amino acid other than a leucine, e.g., where amino acid 120 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2EE retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EE, where amino acid 120 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L120. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L40. In some cases, a variant 4-1BBL polypeptide of the present disclosure comprises the amino acid sequence set forth in SEQ ID NO: 1, with an amino acid substitution at L40. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L41. In some cases, a variant 4-1BBL polypeptide of the present disclosure comprises the amino acid sequence set forth in SEQ ID NO:2, with an amino acid substitution L41. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L41. In some cases, a variant 4-1BBL polypeptide of the present disclosure comprises the amino acid sequence set forth in SEQ ID NO:3, with an amino acid substitution at L41.

For example, in some cases, a variant 4-1BBL polypeptide of the present disclosure comprises the amino acid sequence set forth in FIG. 2EE, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2EE, where “x” is Gly. In some cases, a variant 4-1BBL polypeptide of the present disclosure comprises the amino acid sequence set forth in FIG. 2EE, where “x” is Ala. In some cases, a variant 4-1BBL polypeptide of the present disclosure comprises the amino acid sequence set forth in FIG. 2EE, where “x” is Val. In some cases, a variant 4-1BBL polypeptide of the present disclosure comprises the amino acid sequence set forth in FIG. 2EE, where “x” is Ile. In some cases, a variant 4-1BBL polypeptide of the present disclosure comprises the amino acid sequence set forth in FIG. 2EE, where “x” is Pro. In some cases, a variant 4-1BBL polypeptide of the present disclosure comprises the amino acid sequence set forth in FIG. 2EE, where “x” is Phe. In some cases, a variant 4-1BBL polypeptide of the present disclosure comprises the amino acid sequence set forth in FIG. 2EE, where “x” is Tyr. In some cases, a variant 4-1BBL polypeptide of the present disclosure comprises the amino acid sequence set forth in FIG. 2EE, where “x” is Trp. In some cases, a variant 4-1BBL polypeptide of the present disclosure comprises the amino acid sequence set forth in FIG. 2EE, where “x” is Ser. In some cases, a variant 4-1BBL polypeptide of the present disclosure comprises the amino acid sequence set forth in FIG. 2EE, where “x” is Thr. In some cases, a variant 4-1BBL polypeptide of the present disclosure comprises the amino acid sequence set forth in FIG. 2EE, where “x” is Cys. In some cases, a variant 4-1BBL polypeptide of the present disclosure comprises the amino acid sequence set forth in FIG. 2EE, where “x” is Met. In some cases, a variant 4-1BBL polypeptide of the present disclosure comprises the amino acid sequence set forth in FIG. 2EE, where “x” is Asn. In some cases, a variant 4-1BBL polypeptide of the present disclosure comprises the amino acid sequence set forth in FIG. 2EE, where “x” is Gln. In some cases, a variant 4-1BBL polypeptide of the present disclosure comprises the amino acid sequence set forth in FIG. 2EE, where “x” is Lys. In some cases, a variant 4-1BBL polypeptide of the present disclosure comprises the amino acid sequence set forth in FIG. 2EE, where “x” is Arg. In some cases, a variant 4-1BBL polypeptide of the present disclosure comprises the amino acid sequence set forth in FIG. 2EE, where “x” is His. In some cases, a variant 4-1BBL polypeptide of the present disclosure comprises the amino acid sequence set forth in FIG. 2EE, where “x” is Asp. In some cases, a variant 4-1BBL polypeptide of the present disclosure comprises the amino acid sequence set forth in FIG. 2EE, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### T121 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 (indicated by an “x”) is an amino acid other than a threonine, e.g., where amino acid 121 is Gly, Ala, Val, Leu, Ile, Pro, Phe, Tyr, Trp, Ser, Cys, Met, Asn, Gln, Lys, Arg, His, Asp, or Glu. The amino acid numbering in FIG. 2FF retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FF, where amino acid 121 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at T121. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at T41. In some cases, a variant 4-1BBL polypeptide of the present disclosure comprises the amino acid sequence set forth in SEQ ID NO:1, with an amino acid substitution at T41. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T42. In some cases, a variant 4-1BBL polypeptide of the present disclosure comprises the amino acid sequence set forth in SEQ ID NO:2, with an amino acid substitution T42. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T42. In some cases, a variant 4-1BBL polypeptide of the present disclosure comprises the amino acid sequence set forth in SEQ ID NO:3, with an amino acid substitution at T42.

For example, in some cases, a variant 4-1BBL polypeptide of the present disclosure comprises the amino acid sequence set forth in FIG. 2FF, where “x” is any amino acid other than threonine; for example, “x” can be Gly, Ala, Val, Leu, Ile, Pro, Phe, Tyr, Trp, Ser, Cys, Met, Asn, Gln, Lys, Arg, His, Asp, or Glu. In some cases, a variant 4-1BBL polypeptide of the present disclosure comprises the amino acid sequence set forth in FIG. 2FF, where “x” is Gly. In some cases, a variant 4-1BBL polypeptide of the present disclosure comprises the amino acid sequence set forth in FIG. 2FF, where “x” is Ala. In some cases, a variant 4-1BBL polypeptide of the present disclosure comprises the amino acid sequence set forth in FIG. 2FF, where “x” is Val. In some cases, a variant 4-1BBL polypeptide of the present disclosure comprises the amino acid sequence set forth in FIG. 2FF, where “x” is Leu. In some cases, a variant 4-1BBL polypeptide of the present disclosure comprises the amino acid sequence set forth in FIG. 2FF, where “x” is Ile. In some cases, a variant 4-1BBL polypeptide of the present disclosure comprises the amino acid sequence set forth in FIG. 2FF, where “x” is Pro. In some cases, a variant 4-1BBL polypeptide of the present disclosure comprises the amino acid sequence set forth in FIG. 2FF, where “x” is Phe. In some cases, a variant 4-1BBL polypeptide of the present disclosure comprises the amino acid sequence set forth in FIG. 2FF, where “x” is Tyr. In some cases, a variant 4-1BBL polypeptide of the present disclosure comprises the amino acid sequence set forth in FIG. 2FF, where “x” is Trp. In some cases, a variant 4-1BBL polypeptide of the present disclosure comprises the amino acid sequence set forth in FIG. 2FF, where “x” is Ser. In some cases, a variant 4-1BBL polypeptide of the present disclosure comprises the amino acid sequence set forth in FIG. 2FF, where “x” is Cys. In some cases, a variant 4-1BBL polypeptide of the present disclosure comprises the amino acid sequence set forth in FIG. 2FF, where “x” is Met. In some cases, a variant 4-1BBL polypeptide of the present disclosure comprises the amino acid sequence set forth in FIG. 2FF, where “x” is Asn. In some cases, a variant 4-1BBL polypeptide of the present disclosure comprises the amino acid sequence set forth in FIG. 2FF, where “x” is Gln. In some cases, a variant 4-1BBL polypeptide of the present disclosure comprises the amino acid sequence set forth in FIG. 2FF, where “x” is Lys. In some cases, a variant 4-1BBL polypeptide of the present disclosure comprises the amino acid sequence set forth in FIG. 2FF, where “x” is Arg. In some cases, a variant 4-1BBL polypeptide of the present disclosure comprises the amino acid sequence set forth in FIG. 2FF, where “x” is His. In some cases, a variant 4-1BBL polypeptide of the present disclosure comprises the amino acid sequence set forth in FIG. 2FF, where “x” is Asp. In some cases, a variant 4-1BBL polypeptide of the present disclosure comprises the amino acid sequence set forth in FIG. 2FF, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G122 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 (indicated by an “x”) is an amino acid other than a glycine, e.g., where amino acid 122 is Ala, Val, Leu, Ile, Pro, Phe, Tyr, Trp, Ser, Thr, Cys, Asn, Gln, Lys, Arg, His, Asp, or Glu. The amino acid numbering in FIG. 2GG retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GG, where amino acid 122 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G122. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at G42. In some cases, a variant 4-1BBL polypeptide of the present disclosure comprises the amino acid sequence set forth in SEQ ID NO: 1, with an amino acid substitution at G42. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G43. In some cases, a variant 4-1BBL polypeptide of the present disclosure comprises the amino acid sequence set forth in SEQ ID NO:2, with an amino acid substitution G43. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G43. In some cases, a variant 4-1BBL polypeptide of the present disclosure comprises the amino acid sequence set forth in SEQ ID NO:3, with an amino acid substitution at G43.

For example, in some cases, a variant 4-1BBL polypeptide of the present disclosure comprises the amino acid sequence set forth in FIG. 2GG, where “x” is any amino acid other than glycine; for example, “x” can be Ala, Val, Leu, Ile, Pro, Phe, Tyr, Trp, Ser, Thr, Cys, Asn, Gln, Lys, Arg, His, Asp, or Glu. In some cases, a variant 4-1BBL polypeptide of the present disclosure comprises the amino acid sequence set forth in FIG. 2GG, where “x” is Ala. In some cases, a variant 4-1BBL polypeptide of the present disclosure comprises the amino acid sequence set forth in FIG. 2GG, where “x” is Val. In some cases, a variant 4-1BBL polypeptide of the present disclosure comprises the amino acid sequence set forth in FIG. 2GG, where “x” is Leu. In some cases, a variant 4-1BBL polypeptide of the present disclosure comprises the amino acid sequence set forth in FIG. 2GG, where “x” is Ile. In some cases, a variant 4-1BBL polypeptide of the present disclosure comprises the amino acid sequence set forth in FIG. 2GG, where “x” is Pro. In some cases, a variant 4-1BBL polypeptide of the present disclosure comprises the amino acid sequence set forth in FIG. 2GG, where “x” is Phe. In some cases, a variant 4-1BBL polypeptide of the present disclosure comprises the amino acid sequence set forth in FIG. 2GG, where “x” is Tyr. In some cases, a variant 4-1BBL polypeptide of the present disclosure comprises the amino acid sequence set forth in FIG. 2GG, where “x” is Trp. In some cases, a variant 4-1BBL polypeptide of the present disclosure comprises the amino acid sequence set forth in FIG. 2GG, where “x” is Ser. In some cases, a variant 4-1BBL polypeptide of the present disclosure comprises the amino acid sequence set forth in FIG. 2GG, where “x” is Thr. In some cases, a variant 4-1BBL polypeptide of the present disclosure comprises the amino acid sequence set forth in FIG. 2GG, where “x” is Cys. In some cases, a variant 4-1BBL polypeptide of the present disclosure comprises the amino acid sequence set forth in FIG. 2GG, where “x” is Met. In some cases, a variant 4-1BBL polypeptide of the present disclosure comprises the amino acid sequence set forth in FIG. 2GG, where “x” is Asn. In some cases, a variant 4-1BBL polypeptide of the present disclosure comprises the amino acid sequence set forth in FIG. 2GG, where “x” is Gln. In some cases, a variant 4-1BBL polypeptide of the present disclosure comprises the amino acid sequence set forth in FIG. 2GG, where “x” is Lys. In some cases, a variant 4-1BBL polypeptide of the present disclosure comprises the amino acid sequence set forth in FIG. 2GG, where “x” is Arg. In some cases, a variant 4-1BBL polypeptide of the present disclosure comprises the amino acid sequence set forth in FIG. 2GG, where “x” is His. In some cases, a variant 4-1BBL polypeptide of the present disclosure comprises the amino acid sequence set forth in FIG. 2GG, where “x” is Asp. In some cases, a variant 4-1BBL polypeptide of the present disclosure comprises the amino acid sequence set forth in FIG. 2GG, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### G123 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 (indicated by an “x”) is an amino acid other than a glycine, e.g., where amino acid 123 is Ala, Val, Leu, Ile, Pro, Phe, Tyr, Trp, Ser, Thr, Cys, Asn, Gln, Lys, Arg, His, Asp, or Glu. The amino acid numbering in FIG. 2HH retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HH, where amino acid 123 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G123. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at G43. In some cases, a variant 4-1BBL polypeptide of the present disclosure comprises the amino acid sequence set forth in SEQ ID NO:1, with an amino acid substitution at G43. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G44. In some cases, a variant 4-1BBL polypeptide of the present disclosure comprises the amino acid sequence set forth in SEQ ID NO:2, with an amino acid substitution G44. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G44. In some cases, a variant 4-1BBL polypeptide of the present disclosure comprises the amino acid sequence set forth in SEQ ID NO:3, with an amino acid substitution at G44.

For example, in some cases, a variant 4-1BBL polypeptide of the present disclosure comprises the amino acid sequence set forth in FIG. 2HH, where “x” is any amino acid other than glycine; for example, “x” can be Ala, Val, Leu, Ile, Pro, Phe, Tyr, Trp, Ser, Thr, Cys, Asn, Gln, Lys, Arg, His, Asp, or Glu. In some cases, a variant 4-1BBL polypeptide of the present disclosure comprises the amino acid sequence set forth in FIG. 2HH, where “x” is Ala. In some cases, a variant 4-1BBL polypeptide of the present disclosure comprises the amino acid sequence set forth in FIG. 2HH, where “x” is Val. In some cases, a variant 4-1BBL polypeptide of the present disclosure comprises the amino acid sequence set forth in FIG. 2HH, where “x” is Leu. In some cases, a variant 4-1BBL polypeptide of the present disclosure comprises the amino acid sequence set forth in FIG. 2HH, where “x” is Ile. In some cases, a variant 4-1BBL polypeptide of the present disclosure comprises the amino acid sequence set forth in FIG. 2HH, where “x” is Pro. In some cases, a variant 4-1BBL polypeptide of the present disclosure comprises the amino acid sequence set forth in FIG. 2HH, where “x” is Phe. In some cases, a variant 4-1BBL polypeptide of the present disclosure comprises the amino acid sequence set forth in FIG. 2HH, where “x” is Tyr. In some cases, a variant 4-1BBL polypeptide of the present disclosure comprises the amino acid sequence set forth in FIG. 2HH, where “x” is Trp. In some cases, a variant 4-1BBL polypeptide of the present disclosure comprises the amino acid sequence set forth in FIG. 2HH, where “x” is Ser. In some cases, a variant 4-1BBL polypeptide of the present disclosure comprises the amino acid sequence set forth in FIG. 2HH, where “x” is Thr. In some cases, a variant 4-1BBL polypeptide of the present disclosure comprises the amino acid sequence set forth in FIG. 2HH, where “x” is Cys. In some cases, a variant 4-1BBL polypeptide of the present disclosure comprises the amino acid sequence set forth in FIG. 2HH, where “x” is Met. In some cases, a variant 4-1BBL polypeptide of the present disclosure comprises the amino acid sequence set forth in FIG. 2HH, where “x” is Asn. In some cases, a variant 4-1BBL polypeptide of the present disclosure comprises the amino acid sequence set forth in FIG. 2HH, where “x” is Gln. In some cases, a variant 4-1BBL polypeptide of the present disclosure comprises the amino acid sequence set forth in FIG. 2HH, where “x” is Lys. In some cases, a variant 4-1BBL polypeptide of the present disclosure comprises the amino acid sequence set forth in FIG. 2HH, where “x” is Arg. In some cases, a variant 4-1BBL polypeptide of the present disclosure comprises the amino acid sequence set forth in FIG. 2HH, where “x” is His. In some cases, a variant 4-1BBL polypeptide of the present disclosure comprises the amino acid sequence set forth in FIG. 2HH, where “x” is Asp. In some cases, a variant 4-1BBL polypeptide of the present disclosure comprises the amino acid sequence set forth in FIG. 2HH, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L124 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 (indicated by an “x”) is an amino acid other than a leucine, e.g., where amino acid 124 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2II retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2II, where amino acid 124 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L124. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L44. In some cases, a variant 4-1BBL polypeptide of the present disclosure comprises the amino acid sequence set forth in SEQ ID NO: 1, with an amino acid substitution at L44. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L45. In some cases, a variant 4-1BBL polypeptide of the present disclosure comprises the amino acid sequence set forth in SEQ ID NO:2, with an amino acid substitution L45. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L45. In some cases, a variant 4-1BBL polypeptide of the present disclosure comprises the amino acid sequence set forth in SEQ ID NO:3, with an amino acid substitution at L45.

For example, in some cases, a variant 4-1BBL polypeptide of the present disclosure comprises the amino acid sequence set forth in FIG. 2II, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2II, where “x” is Gly. In some cases, a variant 4-1BBL polypeptide of the present disclosure comprises the amino acid sequence set forth in FIG. 2II, where “x” is Ala. In some cases, a variant 4-1BBL polypeptide of the present disclosure comprises the amino acid sequence set forth in FIG. 2II, where “x” is Val. In some cases, a variant 4-1BBL polypeptide of the present disclosure comprises the amino acid sequence set forth in FIG. 2II, where “x” is Ile. In some cases, a variant 4-1BBL polypeptide of the present disclosure comprises the amino acid sequence set forth in FIG. 2II, where “x” is Pro. In some cases, a variant 4-1BBL polypeptide of the present disclosure comprises the amino acid sequence set forth in FIG. 2II, where “x” is Phe. In some cases, a variant 4-1BBL polypeptide of the present disclosure comprises the amino acid sequence set forth in FIG. 2II, where “x” is Tyr. In some cases, a variant 4-1BBL polypeptide of the present disclosure comprises the amino acid sequence set forth in FIG. 2II, where “x” is Trp. In some cases, a variant 4-1BBL polypeptide of the present disclosure comprises the amino acid sequence set forth in FIG. 2II, where “x” is Ser. In some cases, a variant 4-1BBL polypeptide of the present disclosure comprises the amino acid sequence set forth in FIG. 2II, where “x” is Thr. In some cases, a variant 4-1BBL polypeptide of the present disclosure comprises the amino acid sequence set forth in FIG. 2II, where “x” is Cys. In some cases, a variant 4-1BBL polypeptide of the present disclosure comprises the amino acid sequence set forth in FIG. 2II, where “x” is Met. In some cases, a variant 4-1BBL polypeptide of the present disclosure comprises the amino acid sequence set forth in FIG. 2II, where “x” is Asn. In some cases, a variant 4-1BBL polypeptide of the present disclosure comprises the amino acid sequence set forth in FIG. 2II, where “x” is Gln. In some cases, a variant 4-1BBL polypeptide of the present disclosure comprises the amino acid sequence set forth in FIG. 2II, where “x” is Lys. In some cases, a variant 4-1BBL polypeptide of the present disclosure comprises the amino acid sequence set forth in FIG. 2II, where “x” is Arg. In some cases, a variant 4-1BBL polypeptide of the present disclosure comprises the amino acid sequence set forth in FIG. 2II, where “x” is His. In some cases, a variant 4-1BBL polypeptide of the present disclosure comprises the amino acid sequence set forth in FIG. 2II, where “x” is Asp. In some cases, a variant 4-1BBL polypeptide of the present disclosure comprises the amino acid sequence set forth in FIG. 2II, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S125 Substitution

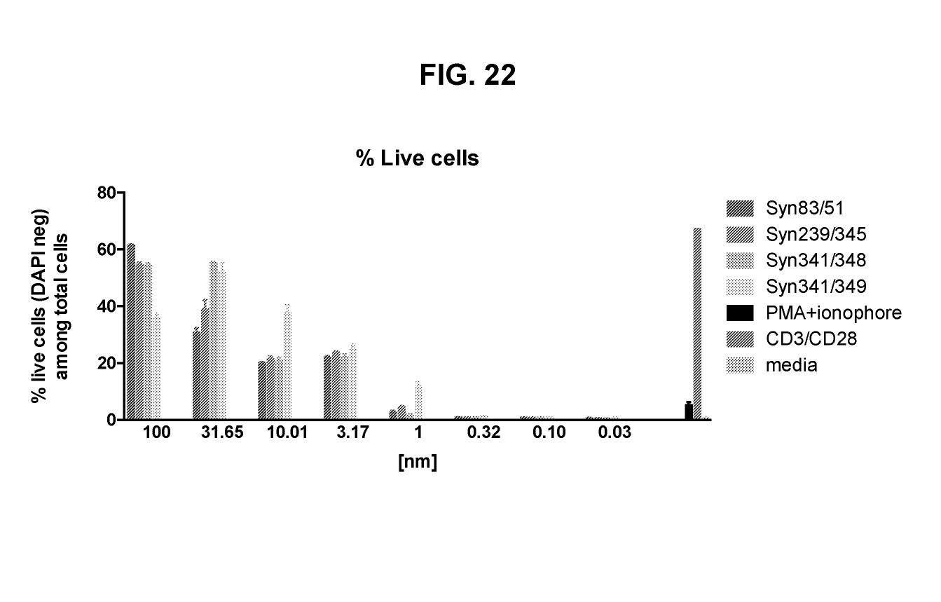

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 (indicated by an “x”) is an amino acid other than a serine, e.g., where amino acid 125 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2JJ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 125 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DD, where amino acid 125 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 22JJ DD, where amino acid 125 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJ, where amino acid 125 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S125. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S45. In some cases, a variant 4-1BBL polypeptide of the present disclosure comprises the amino acid sequence set forth in SEQ ID NO:1, with an amino acid substitution at S45. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S46. In some cases, a variant 4-1BBL polypeptide of the present disclosure comprises the amino acid sequence set forth in SEQ ID NO:2, with an amino acid substitution S46. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S46. In some cases, a variant 4-1BBL polypeptide of the present disclosure comprises the amino acid sequence set forth in SEQ ID NO:3, with an amino acid substitution at S46.

For example, in some cases, a variant 4-1BBL polypeptide of the present disclosure comprises the amino acid sequence set forth in FIG. 2JJ, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2JJ, where “x” is Gly. In some cases, a variant 4-1BBL polypeptide of the present disclosure comprises the amino acid sequence set forth in FIG. 2JJ, where “x” is Ala. In some cases, a variant 4-1BBL polypeptide of the present disclosure comprises the amino acid sequence set forth in FIG. 2JJ, where “x” is Val. In some cases, a variant 4-1BBL polypeptide of the present disclosure comprises the amino acid sequence set forth in FIG. 2JJ, where “x” is Leu. In some cases, a variant 4-1BBL polypeptide of the present disclosure comprises the amino acid sequence set forth in FIG. 2JJ, where “x” is Ile. In some cases, a variant 4-1BBL polypeptide of the present disclosure comprises the amino acid sequence set forth in FIG. 2JJ, where “x” is Pro. In some cases, a variant 4-1BBL polypeptide of the present disclosure comprises the amino acid sequence set forth in FIG. 2JJ, where “x” is Phe. In some cases, a variant 4-1BBL polypeptide of the present disclosure comprises the amino acid sequence set forth in FIG. 2JJ, where “x” is Tyr. In some cases, a variant 4-1BBL polypeptide of the present disclosure comprises the amino acid sequence set forth in FIG. 2JJ, where “x” is Trp. In some cases, a variant 4-1BBL polypeptide of the present disclosure comprises the amino acid sequence set forth in FIG. 2JJ, where “x” is Thr. In some cases, a variant 4-1BBL polypeptide of the present disclosure comprises the amino acid sequence set forth in FIG. 2JJ, where “x” is Cys. In some cases, a variant 4-1BBL polypeptide of the present disclosure comprises the amino acid sequence set forth in FIG. 2JJ, where “x” is Met. In some cases, a variant 4-1BBL polypeptide of the present disclosure comprises the amino acid sequence set forth in FIG. 2JJ, where “x” is Asn. In some cases, a variant 4-1BBL polypeptide of the present disclosure comprises the amino acid sequence set forth in FIG. 2JJ, where “x” is Gln. In some cases, a variant 4-1BBL polypeptide of the present disclosure comprises the amino acid sequence set forth in FIG. 2JJ, where “x” is Lys. In some cases, a variant 4-1BBL polypeptide of the present disclosure comprises the amino acid sequence set forth in FIG. 2JJ, where “x” is Arg. In some cases, a variant 4-1BBL polypeptide of the present disclosure comprises the amino acid sequence set forth in FIG. 2JJ, where “x” is His. In some cases, a variant 4-1BBL polypeptide of the present disclosure comprises the amino acid sequence set forth in FIG. 2JJ, where “x” is Asp. In some cases, a variant 4-1BBL polypeptide of the present disclosure comprises the amino acid sequence set forth in FIG. 2JJ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Y126 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 (indicated by an “x”) is an amino acid other than a tyrosine, e.g., where amino acid 126 is Gly, Ala, Val, Leu, Ile, Pro, Phe, Trp, Ser, Thr, Cys, Met, Asn, Gln, Lys, Arg, His, Asp, or Glu. The amino acid numbering in FIG. 2KK retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 10 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KK, where amino acid 126 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Y126. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at Y46. In some cases, a variant 4-1BBL polypeptide of the present disclosure comprises the amino acid sequence set forth in SEQ ID NO: 1, with an amino acid substitution at Y46. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Y47. In some cases, a variant 4-1BBL polypeptide of the present disclosure comprises the amino acid sequence set forth in SEQ ID NO:2, with an amino acid substitution Y47. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Y47. In some cases, a variant 4-1BBL polypeptide of the present disclosure comprises the amino acid sequence set forth in SEQ ID NO:3, with an amino acid substitution at Y47.

For example, in some cases, a variant 4-1BBL polypeptide of the present disclosure comprises the amino acid sequence set forth in FIG. 2KK, where “x” is any amino acid other than tyrosine; for example, “x” can be Gly, Ala, Val, Leu, Ile, Pro, Phe, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2KK, where “x” is Gly. In some cases, a variant 4-1BBL polypeptide of the present disclosure comprises the amino acid sequence set forth in FIG. 2KK, where “x” is Ala. In some cases, a variant 4-1BBL polypeptide of the present disclosure comprises the amino acid sequence set forth in FIG. 2KK, where “x” is Val. In some cases, a variant 4-1BBL polypeptide of the present disclosure comprises the amino acid sequence set forth in FIG. 2KK, where “x” is Leu. In some cases, a variant 4-1BBL polypeptide of the present disclosure comprises the amino acid sequence set forth in FIG. 2KK, where “x” is Ile. In some cases, a variant 4-1BBL polypeptide of the present disclosure comprises the amino acid sequence set forth in FIG. 2KK, where “x” is Pro. In some cases, a variant 4-1BBL polypeptide of the present disclosure comprises the amino acid sequence set forth in FIG. 2KK, where “x” is Phe. In some cases, a variant 4-1BBL polypeptide of the present disclosure comprises the amino acid sequence set forth in FIG. 2KK, where “x” is Trp. In some cases, a variant 4-1BBL polypeptide of the present disclosure comprises the amino acid sequence set forth in FIG. 2KK, where “x” is Ser. In some cases, a variant 4-1BBL polypeptide of the present disclosure comprises the amino acid sequence set forth in FIG. 2KK, where “x” is Thr. In some cases, a variant 4-1BBL polypeptide of the present disclosure comprises the amino acid sequence set forth in FIG. 2KK, where “x” is Cys. In some cases, a variant 4-1BBL polypeptide of the present disclosure comprises the amino acid sequence set forth in FIG. 2KK, where “x” is Met. In some cases, a variant 4-1BBL polypeptide of the present disclosure comprises the amino acid sequence set forth in FIG. 2KK, where “x” is Asn. In some cases, a variant 4-1BBL polypeptide of the present disclosure comprises the amino acid sequence set forth in FIG. 2KK, where “x” is Gln. In some cases, a variant 4-1BBL polypeptide of the present disclosure comprises the amino acid sequence set forth in FIG. 2KK, where “x” is Lys. In some cases, a variant 4-1BBL polypeptide of the present disclosure comprises the amino acid sequence set forth in FIG. 2KK, where “x” is Arg. In some cases, a variant 4-1BBL polypeptide of the present disclosure comprises the amino acid sequence set forth in FIG. 2KK, where “x” is His. In some cases, a variant 4-1BBL polypeptide of the present disclosure comprises the amino acid sequence set forth in FIG. 2V2KK where “x” is Asp. In some cases, a variant 4-1BBL polypeptide of the present disclosure comprises the amino acid sequence set forth in FIG. 2KK, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### E128 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 (indicated by an “x”) is an amino acid other than a glutamic acid, e.g., where amino acid 128 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2LL retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LL, where amino acid 128 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E128. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at E48. In some cases, a variant 4-1BBL polypeptide of the present disclosure comprises the amino acid sequence set forth in SEQ ID NO:1, with an amino acid substitution at E48. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E49. In some cases, a variant 4-1BBL polypeptide of the present disclosure comprises the amino acid sequence set forth in SEQ ID NO:2, with an amino acid substitution E49. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E49. In some cases, a variant 4-1BBL polypeptide of the present disclosure comprises the amino acid sequence set forth in SEQ ID NO:3, with an amino acid substitution at E49.

For example, in some cases, a variant 4-1BBL polypeptide of the present disclosure comprises the amino acid sequence set forth in FIG. 2LL, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2LL, where “x” is Gly. In some cases, a variant 4-1BBL polypeptide of the present disclosure comprises the amino acid sequence set forth in FIG. 2LL, where “x” is Ala. In some cases, a variant 4-1BBL polypeptide of the present disclosure comprises the amino acid sequence set forth in FIG. 2LL, where “x” is Val. In some cases, a variant 4-1BBL polypeptide of the present disclosure comprises the amino acid sequence set forth in FIG. 2LL, where “x” is Leu. In some cases, a variant 4-1BBL polypeptide of the present disclosure comprises the amino acid sequence set forth in FIG. 2LL, where “x” is Ile. In some cases, a variant 4-1BBL polypeptide of the present disclosure comprises the amino acid sequence set forth in FIG. 2LL, where “x” is Pro. In some cases, a variant 4-1BBL polypeptide of the present disclosure comprises the amino acid sequence set forth in FIG. 2LL, where “x” is Phe. In some cases, a variant 4-1BBL polypeptide of the present disclosure comprises the amino acid sequence set forth in FIG. 2LL, where “x” is Tyr. In some cases, a variant 4-1BBL polypeptide of the present disclosure comprises the amino acid sequence set forth in FIG. 2LL, where “x” is Trp. In some cases, a variant 4-1BBL polypeptide of the present disclosure comprises the amino acid sequence set forth in FIG. 2LL, where “x” is Ser. In some cases, a variant 4-1BBL polypeptide of the present disclosure comprises the amino acid sequence set forth in FIG. 2LL, where “x” is Thr. In some cases, a variant 4-1BBL polypeptide of the present disclosure comprises the amino acid sequence set forth in FIG. 2LL, where “x” is Cys. In some cases, a variant 4-1BBL polypeptide of the present disclosure comprises the amino acid sequence set forth in FIG. 2LL, where “x” is Met. In some cases, a variant 4-1BBL polypeptide of the present disclosure comprises the amino acid sequence set forth in FIG. 2LL, where “x” is Asn. In some cases, a variant 4-1BBL polypeptide of the present disclosure comprises the amino acid sequence set forth in FIG. 2LL, where “x” is Gln. In some cases, a variant 4-1BBL polypeptide of the present disclosure comprises the amino acid sequence set forth in FIG. 2LL, where “x” is Lys. In some cases, a variant 4-1BBL polypeptide of the present disclosure comprises the amino acid sequence set forth in FIG. 2LL, where “x” is Arg. In some cases, a variant 4-1BBL polypeptide of the present disclosure comprises the amino acid sequence set forth in FIG. 2LL, where “x” is His. In some cases, a variant 4-1BBL polypeptide of the present disclosure comprises the amino acid sequence set forth in FIG. 2LL, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### D129 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 (indicated by an “x”) is an amino acid other than an aspartic acid, e.g., where amino acid 129 is Gly, Ala, Val, Leu, Ile, Pro, Phe, Tyr, Trp, Ser, Thr, Cys, Met, Asn, Gln, Lys, Arg, His, or Glu. The amino acid numbering in FIG. 2MM retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 129 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MM, where amino acid 2MM is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at D129. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at D49. In some cases, a variant 4-1BBL polypeptide of the present disclosure comprises the amino acid sequence set forth in SEQ ID NO: 1, with an amino acid substitution at D49. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D50. In some cases, a variant 4-1BBL polypeptide of the present disclosure comprises the amino acid sequence set forth in SEQ ID NO:2, with an amino acid substitution at D50. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D50. In some cases, a variant 4-1BBL polypeptide of the present disclosure comprises the amino acid sequence set forth in SEQ ID NO:3, with an amino acid substitution at D50.

For example, in some cases, a variant 4-1BBL polypeptide of the present disclosure comprises the amino acid sequence set forth in FIG. 2MM, where “x” is any amino acid other than aspartic acid; for example, “x” can be Gly, Ala, Val, Leu, Ile, Pro, Phe, Tyr, Trp, Ser, Thr, Cys, Met, Asn, Gln, Lys, Arg, His, or Glu. In some cases, a variant 4-1BBL polypeptide of the present disclosure comprises the amino acid sequence set forth in FIG. 2MM, where “x” is Gly. In some cases, a variant 4-1BBL polypeptide of the present disclosure comprises the amino acid sequence set forth in FIG. 2MM, where “x” is Ala. In some cases, a variant 4-1BBL polypeptide of the present disclosure comprises the amino acid sequence set forth in FIG. 2MM, where “x” is Val. In some cases, a variant 4-1BBL polypeptide of the present disclosure comprises the amino acid sequence set forth in FIG. 2MM, where “x” is Leu. In some cases, a variant 4-1BBL polypeptide of the present disclosure comprises the amino acid sequence set forth in FIG. 2MM, where “x” is Ile. In some cases, a variant 4-1BBL polypeptide of the present disclosure comprises the amino acid sequence set forth in FIG. 2MM, where “x” is Pro. In some cases, a variant 4-1BBL polypeptide of the present disclosure comprises the amino acid sequence set forth in FIG. 2MM, where “x” is Phe. In some cases, a variant 4-1BBL polypeptide of the present disclosure comprises the amino acid sequence set forth in FIG. 2MM, where “x” is Tyr. In some cases, a variant 4-1BBL polypeptide of the present disclosure comprises the amino acid sequence set forth in FIG. 2MM, where “x” is Trp. In some cases, a variant 4-1BBL polypeptide of the present disclosure comprises the amino acid sequence set forth in FIG. 2MM, where “x” is Ser. In some cases, a variant 4-1BBL polypeptide of the present disclosure comprises the amino acid sequence set forth in FIG. 2MM, where “x” is Thr. In some cases, a variant 4-1BBL polypeptide of the present disclosure comprises the amino acid sequence set forth in FIG. 2MM, where “x” is Cys. In some cases, a variant 4-1BBL polypeptide of the present disclosure comprises the amino acid sequence set forth in FIG. 2MM, where “x” is Met. In some cases, a variant 4-1BBL polypeptide of the present disclosure comprises the amino acid sequence set forth in FIG. 2MM, where “x” is Asn. In some cases, a variant 4-1BBL polypeptide of the present disclosure comprises the amino acid sequence set forth in FIG. 2MM, where “x” is Gln. In some cases, a variant 4-1BBL polypeptide of the present disclosure comprises the amino acid sequence set forth in FIG. 2MM, where “x” is Lys. In some cases, a variant 4-1BBL polypeptide of the present disclosure comprises the amino acid sequence set forth in FIG. 2MM, where “x” is Arg. In some cases, a variant 4-1BBL polypeptide of the present disclosure comprises the amino acid sequence set forth in FIG. 2MM, where “x” is His. In some cases, a variant 4-1BBL polypeptide of the present disclosure comprises the amino acid sequence set forth in FIG. 2MM, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### T130

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 (indicated by an “x”) is an amino acid other than a threonine, e.g., where amino acid 130 is Gly, Ala, Val, Leu, Ile, Pro, Phe, Tyr, Trp, Ser, Cys, Met, Asn, Gln, Lys, Arg, His, Asp, or Glu. The amino acid numbering in FIG. 2NN retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NN, where amino acid 130 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at T130. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at T50. In some cases, a variant 4-1BBL polypeptide of the present disclosure comprises the amino acid sequence set forth in SEQ ID NO: 1, with an amino acid substitution at T50. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T51. In some cases, a variant 4-1BBL polypeptide of the present disclosure comprises the amino acid sequence set forth in SEQ ID NO:2, with an amino acid substitution at T51. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T51. In some cases, a variant 4-1BBL polypeptide of the present disclosure comprises the amino acid sequence set forth in SEQ ID NO:3, with an amino acid substitution at T51.

For example, in some cases, a variant 4-1BBL polypeptide of the present disclosure comprises the amino acid sequence set forth in FIG. 2NN, where “x” is any amino acid other than threonine; for example, “x” can be Gly, Ala, Val, Leu, Ile, Pro, Phe, Tyr, Trp, Ser, Cys, Met, Asn, Gln, Lys, Arg, His, Asp, or Glu. In some cases, a variant 4-1BBL polypeptide of the present disclosure comprises the amino acid sequence set forth in FIG. 2NN, where “x” is Gly. In some cases, a variant 4-1BBL polypeptide of the present disclosure comprises the amino acid sequence set forth in FIG. 2NN, where “x” is Ala. In some cases, a variant 4-1BBL polypeptide of the present disclosure comprises the amino acid sequence set forth in FIG. 2NN, where “x” is Val. In some cases, a variant 4-1BBL polypeptide of the present disclosure comprises the amino acid sequence set forth in FIG. 2NN, where “x” is Leu. In some cases, a variant 4-1BBL polypeptide of the present disclosure comprises the amino acid sequence set forth in FIG. 2NN, where “x” is Ile. In some cases, a variant 4-1BBL polypeptide of the present disclosure comprises the amino acid sequence set forth in FIG. 2NN, where “x” is Pro. In some cases, a variant 4-1BBL polypeptide of the present disclosure comprises the amino acid sequence set forth in FIG. 2NN, where “x” is Phe. In some cases, a variant 4-1BBL polypeptide of the present disclosure comprises the amino acid sequence set forth in FIG. 2NN, where “x” is Tyr. In some cases, a variant 4-1BBL polypeptide of the present disclosure comprises the amino acid sequence set forth in FIG. 2NN, where “x” is Trp. In some cases, a variant 4-1BBL polypeptide of the present disclosure comprises the amino acid sequence set forth in FIG. 2NN, where “x” is Ser. In some cases, a variant 4-1BBL polypeptide of the present disclosure comprises the amino acid sequence set forth in FIG. 2NN, where “x” is Cys. In some cases, a variant 4-1BBL polypeptide of the present disclosure comprises the amino acid sequence set forth in FIG. 2NN, where “x” is Met. In some cases, a variant 4-1BBL polypeptide of the present disclosure comprises the amino acid sequence set forth in FIG. 2NN, where “x” is Asn. In some cases, a variant 4-1BBL polypeptide of the present disclosure comprises the amino acid sequence set forth in FIG. 2NN, where “x” is Gln. In some cases, a variant 4-1BBL polypeptide of the present disclosure comprises the amino acid sequence set forth in FIG. 2NN, where “x” is Lys. In some cases, a variant 4-1BBL polypeptide of the present disclosure comprises the amino acid sequence set forth in FIG. 2NN, where “x” is Arg. In some cases, a variant 4-1BBL polypeptide of the present disclosure comprises the amino acid sequence set forth in FIG. 2NN, where “x” is His. In some cases, a variant 4-1BBL polypeptide of the present disclosure comprises the amino acid sequence set forth in FIG. 2NN, where “x” is Asp. In some cases, a variant 4-1BBL polypeptide of the present disclosure comprises the amino acid sequence set forth in FIG. 2NN, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### K131 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 (indicated by an “x”) is an amino acid other than a lysine, e.g., where amino acid 131 is Gly, Ala, Val, Leu, Ile, Pro, Phe, Tyr, Trp, Ser, Thr, Cys, Met, Asn, Gln, Arg, His, Asp, or Glu. The amino acid numbering in FIG. 2OO retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2E, where amino acid 131 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OO, where amino acid 131 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at K131. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at K51. In some cases, a variant 4-1BBL polypeptide of the present disclosure comprises the amino acid sequence set forth in SEQ ID NO: 1, with an amino acid substitution at K51. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at K52. In some cases, a variant 4-1BBL polypeptide of the present disclosure comprises the amino acid sequence set forth in SEQ ID NO:2, with an amino acid substitution at K52. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at K52. In some cases, a variant 4-1BBL polypeptide of the present disclosure comprises the amino acid sequence set forth in SEQ ID NO:3, with an amino acid substitution at K52.

For example, in some cases, a variant 4-1BBL polypeptide of the present disclosure comprises the amino acid sequence set forth in FIG. 2OO, where “x” is any amino acid other than lysine; for example, “x” can be Gly, Ala, Val, Leu, Ile, Pro, Phe, Tyr, Trp, Ser, Thr, Cys, Met, Asn, Gln, Arg, His, Asp, or Glu. In some cases, a variant 4-1BBL polypeptide of the present disclosure comprises the amino acid sequence set forth in FIG. 2OO, where “x” is Gly. In some cases, a variant 4-1BBL polypeptide of the present disclosure comprises the amino acid sequence set forth in FIG. 2OO, where “x” is Ala. In some cases, a variant 4-1BBL polypeptide of the present disclosure comprises the amino acid sequence set forth in FIG. 2OO, where “x” is Val. In some cases, a variant 4-1BBL polypeptide of the present disclosure comprises the amino acid sequence set forth in FIG. 2OO, where “x” is Leu. In some cases, a variant 4-1BBL polypeptide of the present disclosure comprises the amino acid sequence set forth in FIG. 2OO, where “x” is Ile. In some cases, a variant 4-1BBL polypeptide of the present disclosure comprises the amino acid sequence set forth in FIG. 2OO, where “x” is Pro. In some cases, a variant 4-1BBL polypeptide of the present disclosure comprises the amino acid sequence set forth in FIG. 2OO, where “x” is Phe. In some cases, a variant 4-1BBL polypeptide of the present disclosure comprises the amino acid sequence set forth in FIG. 2OO, where “x” is Tyr. In some cases, a variant 4-1BBL polypeptide of the present disclosure comprises the amino acid sequence set forth in FIG. 2OO, where “x” is Trp. In some cases, a variant 4-1BBL polypeptide of the present disclosure comprises the amino acid sequence set forth in FIG. 2OO, where “x” is Ser. In some cases, a variant 4-1BBL polypeptide of the present disclosure comprises the amino acid sequence set forth in FIG. 2OO, where “x” is Thr. In some cases, a variant 4-1BBL polypeptide of the present disclosure comprises the amino acid sequence set forth in FIG. 2OO, where “x” is Cys. In some cases, a variant 4-1BBL polypeptide of the present disclosure comprises the amino acid sequence set forth in FIG. 2OO, where “x” is Met. In some cases, a variant 4-1BBL polypeptide of the present disclosure comprises the amino acid sequence set forth in FIG. 2OO, where “x” is Asn. In some cases, a variant 4-1BBL polypeptide of the present disclosure comprises the amino acid sequence set forth in FIG. 2OO, where “x” is Gln. In some cases, a variant 4-1BBL polypeptide of the present disclosure comprises the amino acid sequence set forth in FIG. 2OO, where “x” is Arg. In some cases, a variant 4-1BBL polypeptide of the present disclosure comprises the amino acid sequence set forth in FIG. 2OO, where “x” is His. In some cases, a variant 4-1BBL polypeptide of the present disclosure comprises the amino acid sequence set forth in FIG. 2OO, where “x” is Asp. In some cases, a variant 4-1BBL polypeptide of the present disclosure comprises the amino acid sequence set forth in FIG. 2OO, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### E132 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 (indicated by an “x”) is an amino acid other than a glutamic acid, e.g., where amino acid 132 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2PP retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PP, where amino acid 132 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E132. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at E52. In some cases, a variant 4-1BBL polypeptide of the present disclosure comprises the amino acid sequence set forth in SEQ ID NO: 1, with an amino acid substitution at E52. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E53. In some cases, a variant 4-1BBL polypeptide of the present disclosure comprises the amino acid sequence set forth in SEQ ID NO:2, with an amino acid substitution at E53. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E53. In some cases, a variant 4-1BBL polypeptide of the present disclosure comprises the amino acid sequence set forth in SEQ ID NO:3, with an amino acid substitution at E53.

For example, in some cases, a variant 4-1BBL polypeptide of the present disclosure comprises the amino acid sequence set forth in FIG. 2PP, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2PP, where “x” is Gly. In some cases, a variant 4-1BBL polypeptide of the present disclosure comprises the amino acid sequence set forth in FIG. 2PP, where “x” is Ala. In some cases, a variant 4-1BBL polypeptide of the present disclosure comprises the amino acid sequence set forth in FIG. 2L2PP L, where “x” is Val. In some cases, a variant 4-1BBL polypeptide of the present disclosure comprises the amino acid sequence set forth in FIG. 2PP, where “x” is Leu. In some cases, a variant 4-1BBL polypeptide of the present disclosure comprises the amino acid sequence set forth in FIG. 2PP, where “x” is Ile. In some cases, a variant 4-1BBL polypeptide of the present disclosure comprises the amino acid sequence set forth in FIG. 2PP, where “x” is Pro. In some cases, a variant 4-1BBL polypeptide of the present disclosure comprises the amino acid sequence set forth in FIG. 2PP, where “x” is Phe. In some cases, a variant 4-1BBL polypeptide of the present disclosure comprises the amino acid sequence set forth in FIG. 2PP, where “x” is Tyr. In some cases, a variant 4-1BBL polypeptide of the present disclosure comprises the amino acid sequence set forth in FIG. 2PP, where “x” is Trp. In some cases, a variant 4-1BBL polypeptide of the present disclosure comprises the amino acid sequence set forth in FIG. 2PP, where “x” is Ser. In some cases, a variant 4-1BBL polypeptide of the present disclosure comprises the amino acid sequence set forth in FIG. 2PP, where “x” is Thr. In some cases, a variant 4-1BBL polypeptide of the present disclosure comprises the amino acid sequence set forth in FIG. 2PP, where “x” is Cys. In some cases, a variant 4-1BBL polypeptide of the present disclosure comprises the amino acid sequence set forth in FIG. 2PP, where “x” is Met. In some cases, a variant 4-1BBL polypeptide of the present disclosure comprises the amino acid sequence set forth in FIG. 2PP, where “x” is Asn. In some cases, a variant 4-1BBL polypeptide of the present disclosure comprises the amino acid sequence set forth in FIG. 2PP, where “x” is Gln. In some cases, a variant 4-1BBL polypeptide of the present disclosure comprises the amino acid sequence set forth in FIG. 2PP, where “x” is Lys. In some cases, a variant 4-1BBL polypeptide of the present disclosure comprises the amino acid sequence set forth in FIG. 2PP, where “x” is Arg. In some cases, a variant 4-1BBL polypeptide of the present disclosure comprises the amino acid sequence set forth in FIG. 2PP, where “x” is His. In some cases, a variant 4-1BBL polypeptide of the present disclosure comprises the amino acid sequence set forth in FIG. 2PP, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### F144 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 (indicated by an “x”) is an amino acid other than a phenylalanine, e.g., where amino acid 144 is Gly, Ala, Val, Leu, Ile, Pro, Tyr, Trp, Ser, Thr, Cys, Met, Asn, Gln, Lys, Arg, His, Asp, or Glu. The amino acid numbering in FIG. 2QQ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 144 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at F144. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at F64. In some cases, a variant 4-1BBL polypeptide of the present disclosure comprises the amino acid sequence set forth in SEQ ID NO: 1, with an amino acid substitution at F64. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F65. In some cases, a variant 4-1BBL polypeptide of the present disclosure comprises the amino acid sequence set forth in SEQ ID NO:2, with an amino acid substitution at F65. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F65. In some cases, a variant 4-1BBL polypeptide of the present disclosure comprises the amino acid sequence set forth in SEQ ID NO:3, with an amino acid substitution at F65.

For example, in some cases, a variant 4-1BBL polypeptide of the present disclosure comprises the amino acid sequence set forth in FIG. 2QQ, where “x” is any amino acid other than phenylalanine; for example, “x” can be Gly, Ala, Val, Leu, Ile, Pro, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2QQ, where “x” is Gly. In some cases, a variant 4-1BBL polypeptide of the present disclosure comprises the amino acid sequence set forth in FIG. 2QQ, where “x” is Ala. In some cases, a variant 4-1BBL polypeptide of the present disclosure comprises the amino acid sequence set forth in FIG. 2QQ, where “x” is Val. In some cases, a variant 4-1BBL polypeptide of the present disclosure comprises the amino acid sequence set forth in FIG. 2QQ, where “x” is Leu. In some cases, a variant 4-1BBL polypeptide of the present disclosure comprises the amino acid sequence set forth in FIG. 2QQ, where “x” is Ile. In some cases, a variant 4-1BBL polypeptide of the present disclosure comprises the amino acid sequence set forth in FIG. 2QQ, where “x” is Pro. In some cases, a variant 4-1BBL polypeptide of the present disclosure comprises the amino acid sequence set forth in FIG. 2QQ, where “x” is Tyr. In some cases, a variant 4-1BBL polypeptide of the present disclosure comprises the amino acid sequence set forth in FIG. 2QQ, where “x” is Trp. In some cases, a variant 4-1BBL polypeptide of the present disclosure comprises the amino acid sequence set forth in FIG. 2QQ, where “x” is Ser. In some cases, a variant 4-1BBL polypeptide of the present disclosure comprises the amino acid sequence set forth in FIG. 2QQ, where “x” is Thr. In some cases, a variant 4-1BBL polypeptide of the present disclosure comprises the amino acid sequence set forth in FIG. 2QQ, where “x” is Cys. In some cases, a variant 4-1BBL polypeptide of the present disclosure comprises the amino acid sequence set forth in FIG. 2QQ, where “x” is Met. In some cases, a variant 4-1BBL polypeptide of the present disclosure comprises the amino acid sequence set forth in FIG. 2QQ, where “x” is Asn. In some cases, a variant 4-1BBL polypeptide of the present disclosure comprises the amino acid sequence set forth in FIG. 2QQ, where “x” is Gln. In some cases, a variant 4-1BBL polypeptide of the present disclosure comprises the amino acid sequence set forth in FIG. 2QQ, where “x” is Lys. In some cases, a variant 4-1BBL polypeptide of the present disclosure comprises the amino acid sequence set forth in FIG. 2QQ, where “x” is Arg. In some cases, a variant 4-1BBL polypeptide of the present disclosure comprises the amino acid sequence set forth in FIG. 2QQ, where “x” is His. In some cases, a variant 4-1BBL polypeptide of the present disclosure comprises the amino acid sequence set forth in FIG. 2QQ, where “x” is Asp. In some cases, a variant 4-1BBL polypeptide of the present disclosure comprises the amino acid sequence set forth in FIG. 2QQ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### F145 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 (indicated by an “x”) is an amino acid other than a phenylalanine, e.g., where amino acid 145 is Gly, Ala, Val, Leu, Ile, Pro, Tyr, Trp, Ser, Thr, Cys, Met, Asn, Gln, Lys, Arg, His, Asp, or Glu. The amino acid numbering in FIG. 2RR retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RR, where amino acid 145 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQ, where amino acid 145 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at F145. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at F65. In some cases, a variant 4-1BBL polypeptide of the present disclosure comprises the amino acid sequence set forth in SEQ ID NO:1, with an amino acid substitution at F65. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F56. In some cases, a variant 4-1BBL polypeptide of the present disclosure comprises the amino acid sequence set forth in SEQ ID NO:2, with an amino acid substitution at F56. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F56. In some cases, a variant 4-1BBL polypeptide of the present disclosure comprises the amino acid sequence set forth in SEQ ID NO:3, with an amino acid substitution at F56.

For example, in some cases, a variant 4-1BBL polypeptide of the present disclosure comprises the amino acid sequence set forth in FIG. 2RR, where “x” is any amino acid other than phenylalanine; for example, “x” can be Gly, Ala, Val, Leu, Ile, Pro, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2RR, where “x” is Gly. In some cases, a variant 4-1BBL polypeptide of the present disclosure comprises the amino acid sequence set forth in FIG. 2RR, where “x” is Ala. In some cases, a variant 4-1BBL polypeptide of the present disclosure comprises the amino acid sequence set forth in FIG. 2RR, where “x” is Val. In some cases, a variant 4-1BBL polypeptide of the present disclosure comprises the amino acid sequence set forth in FIG. 2RR, where “x” is Leu. In some cases, a variant 4-1BBL polypeptide of the present disclosure comprises the amino acid sequence set forth in FIG. 2RR, where “x” is Ile. In some cases, a variant 4-1BBL polypeptide of the present disclosure comprises the amino acid sequence set forth in FIG. 2RR, where “x” is Pro. In some cases, a variant 4-1BBL polypeptide of the present disclosure comprises the amino acid sequence set forth in FIG. 2RR, where “x” is Tyr. In some cases, a variant 4-1BBL polypeptide of the present disclosure comprises the amino acid sequence set forth in FIG. 2RR, where “x” is Trp. In some cases, a variant 4-1BBL polypeptide of the present disclosure comprises the amino acid sequence set forth in FIG. 2RR, where “x” is Ser. In some cases, a variant 4-1BBL polypeptide of the present disclosure comprises the amino acid sequence set forth in FIG. 2RR, where “x” is Thr. In some cases, a variant 4-1BBL polypeptide of the present disclosure comprises the amino acid sequence set forth in FIG. 2RR, where “x” is Cys. In some cases, a variant 4-1BBL polypeptide of the present disclosure comprises the amino acid sequence set forth in FIG. 2RR, where “x” is Met. In some cases, a variant 4-1BBL polypeptide of the present disclosure comprises the amino acid sequence set forth in FIG. 2RR, where “x” is Asn. In some cases, a variant 4-1BBL polypeptide of the present disclosure comprises the amino acid sequence set forth in FIG. 2RR, where “x” is Gln. In some cases, a variant 4-1BBL polypeptide of the present disclosure comprises the amino acid sequence set forth in FIG. 2RR, where “x” is Lys. In some cases, a variant 4-1BBL polypeptide of the present disclosure comprises the amino acid sequence set forth in FIG. 2RR, where “x” is Arg. In some cases, a variant 4-1BBL polypeptide of the present disclosure comprises the amino acid sequence set forth in FIG. 2RR, where “x” is His. In some cases, a variant 4-1BBL polypeptide of the present disclosure comprises the amino acid sequence set forth in FIG. 2RR, where “x” is Asp. In some cases, a variant 4-1BBL polypeptide of the present disclosure comprises the amino acid sequence set forth in FIG. 2RR, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q146

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 (indicated by an “x”) is an amino acid other than a glutamine, e.g., where amino acid 146 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2SS retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Glu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SS, where amino acid 146 is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q146. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at Q66. In some cases, a variant 4-1BBL polypeptide of the present disclosure comprises the amino acid sequence set forth in SEQ ID NO: 1, with an amino acid substitution at Q66. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q67. In some cases, a variant 4-1BBL polypeptide of the present disclosure comprises the amino acid sequence set forth in SEQ ID NO:2, with an amino acid substitution at Q67. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q67. In some cases, a variant 4-1BBL polypeptide of the present disclosure comprises the amino acid sequence set forth in SEQ ID NO:3, with an amino acid substitution at Q67.

For example, in some cases, a variant 4-1BBL polypeptide of the present disclosure comprises the amino acid sequence set forth in FIG. 2SS, where “x” is any amino acid other than glutamine; for example, “x” can be Gly, Ala, Val, Leu, Ile, Pro, Phe, Tyr, Trp, Ser, Thr, Cys, Met, Asn, Gln, Arg, His, Asp, or Glu. In some cases, a variant 4-1BBL polypeptide of the present disclosure comprises the amino acid sequence set forth in FIG. 2SS, where “x” is Phe. In some cases, a variant 4-1BBL polypeptide of the present disclosure comprises the amino acid sequence set forth in FIG. 2SS, where “x” is Leu. In some cases, a variant 4-1BBL polypeptide of the present disclosure comprises the amino acid sequence set forth in FIG. 2SS, where “x” is Ile. In some cases, a variant 4-1BBL polypeptide of the present disclosure comprises the amino acid sequence set forth in FIG. 2SS, where “x” is Met. In some cases, a variant 4-1BBL polypeptide of the present disclosure comprises the amino acid sequence set forth in FIG. 2SS, where “x” is Val. In some cases, a variant 4-1BBL polypeptide of the present disclosure comprises the amino acid sequence set forth in FIG. 2SS, where “x” is Ser. In some cases, a variant 4-1BBL polypeptide of the present disclosure comprises the amino acid sequence set forth in FIG. 2SS, where “x” is Pro. In some cases, a variant 4-1BBL polypeptide of the present disclosure comprises the amino acid sequence set forth in FIG. 2SS, where “x” is Thr. In some cases, a variant 4-1BBL polypeptide of the present disclosure comprises the amino acid sequence set forth in FIG. 2SS, where “x” is Ala. In some cases, a variant 4-1BBL polypeptide of the present disclosure comprises the amino acid sequence set forth in FIG. 2SS, where “x” is Tyr. In some cases, a variant 4-1BBL polypeptide of the present disclosure comprises the amino acid sequence set forth in FIG. 2SS, where “x” is His. In some cases, a variant 4-1BBL polypeptide of the present disclosure comprises the amino acid sequence set forth in FIG. 2SS, where “x” is Asn. In some cases, a variant 4-1BBL polypeptide of the present disclosure comprises the amino acid sequence set forth in FIG. 2SS, where “x” is Lys. In some cases, a variant 4-1BBL polypeptide of the present disclosure comprises the amino acid sequence set forth in FIG. 2SS, where “x” is Asp. In some cases, a variant 4-1BBL polypeptide of the present disclosure comprises the amino acid sequence set forth in FIG. 2SS, where “x” is Glu. In some cases, a variant 4-1BBL polypeptide of the present disclosure comprises the amino acid sequence set forth in FIG. 2SS, where “x” is Cys. In some cases, a variant 4-1BBL polypeptide of the present disclosure comprises the amino acid sequence set forth in FIG. 2SS, where “x” is Trp. In some cases, a variant 4-1BBL polypeptide of the present disclosure comprises the amino acid sequence set forth in FIG. 2SS, where “x” is Arg. In some cases, a variant 4-1BBL polypeptide of the present disclosure comprises the amino acid sequence set forth in FIG. 2SS where “x” is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L147 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 (indicated by an “x”) is an amino acid other than a leucine, e.g., where amino acid 147 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2TT retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TT, where amino acid 147 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L147. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L67. In some cases, a variant 4-1BBL polypeptide of the present disclosure comprises the amino acid sequence set forth in SEQ ID NO: 1, with an amino acid substitution at L67. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L68. In some cases, a variant 4-1BBL polypeptide of the present disclosure comprises the amino acid sequence set forth in SEQ ID NO:2, with an amino acid substitution at L68. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L68. In some cases, a variant 4-1BBL polypeptide of the present disclosure comprises the amino acid sequence set forth in SEQ ID NO:3, with an amino acid substitution at L68.

For example, in some cases, a variant 4-1BBL polypeptide of the present disclosure comprises the amino acid sequence set forth in FIG. 2TT, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2TT, where “x” is Gly. In some cases, a variant 4-1BBL polypeptide of the present disclosure comprises the amino acid sequence set forth in FIG. 2TT, where “x” is Ala. In some cases, a variant 4-1BBL polypeptide of the present disclosure comprises the amino acid sequence set forth in FIG. 2TT, where “x” is Val. In some cases, a variant 4-1BBL polypeptide of the present disclosure comprises the amino acid sequence set forth in FIG. 2TT, where “x” is Ile. In some cases, a variant 4-1BBL polypeptide of the present disclosure comprises the amino acid sequence set forth in FIG. 2TT, where “x” is Pro. In some cases, a variant 4-1BBL polypeptide of the present disclosure comprises the amino acid sequence set forth in FIG. 2TT, where “x” is Phe. In some cases, a variant 4-1BBL polypeptide of the present disclosure comprises the amino acid sequence set forth in FIG. 2TT, where “x” is Tyr. In some cases, a variant 4-1BBL polypeptide of the present disclosure comprises the amino acid sequence set forth in FIG. 2TT, where “x” is Trp. In some cases, a variant 4-1BBL polypeptide of the present disclosure comprises the amino acid sequence set forth in FIG. 2TT, where “x” is Ser. In some cases, a variant 4-1BBL polypeptide of the present disclosure comprises the amino acid sequence set forth in FIG. 2TT, where “x” is Thr. In some cases, a variant 4-1BBL polypeptide of the present disclosure comprises the amino acid sequence set forth in FIG. 2TT, where “x” is Cys. In some cases, a variant 4-1BBL polypeptide of the present disclosure comprises the amino acid sequence set forth in FIG. 2TT, where “x” is Met. In some cases, a variant 4-1BBL polypeptide of the present disclosure comprises the amino acid sequence set forth in FIG. 2TT, where “x” is Asn. In some cases, a variant 4-1BBL polypeptide of the present disclosure comprises the amino acid sequence set forth in FIG. 2TT, where “x” is Gln. In some cases, a variant 4-1BBL polypeptide of the present disclosure comprises the amino acid sequence set forth in FIG. 2TT, where “x” is Lys. In some cases, a variant 4-1BBL polypeptide of the present disclosure comprises the amino acid sequence set forth in FIG. 2TT, where “x” is Arg. In some cases, a variant 4-1BBL polypeptide of the present disclosure comprises the amino acid sequence set forth in FIG. 2TT, where “x” is His. In some cases, a variant 4-1BBL polypeptide of the present disclosure comprises the amino acid sequence set forth in FIG. 2TT, where “x” is Asp. In some cases, a variant 4-1BBL polypeptide of the present disclosure comprises the amino acid sequence set forth in FIG. 2TT, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### E148 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 (indicated by an “x”) is an amino acid other than a glutamic acid, e.g., where amino acid 148 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2UU retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UU, where amino acid 148 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E148. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at E68. In some cases, a variant 4-1BBL polypeptide of the present disclosure comprises the amino acid sequence set forth in SEQ ID NO:1, with an amino acid substitution at E68. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E69. In some cases, a variant 4-1BBL polypeptide of the present disclosure comprises the amino acid sequence set forth in SEQ ID NO:2, with an amino acid substitution at E69. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E69. In some cases, a variant 4-1BBL polypeptide of the present disclosure comprises the amino acid sequence set forth in SEQ ID NO:3, with an amino acid substitution at E69.

For example, in some cases, a variant 4-1BBL polypeptide of the present disclosure comprises the amino acid sequence set forth in FIG. 2UU, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2UU, where “x” is Gly. In some cases, a variant 4-1BBL polypeptide of the present disclosure comprises the amino acid sequence set forth in FIG. 2UU, where “x” is Ala. In some cases, a variant 4-1BBL polypeptide of the present disclosure comprises the amino acid sequence set forth in FIG. 2UU L, where “x” is Val. In some cases, a variant 4-1BBL polypeptide of the present disclosure comprises the amino acid sequence set forth in FIG. 2UU, where “x” is Leu. In some cases, a variant 4-1BBL polypeptide of the present disclosure comprises the amino acid sequence set forth in FIG. 2UU, where “x” is Ile. In some cases, a variant 4-1BBL polypeptide of the present disclosure comprises the amino acid sequence set forth in FIG. 2UU, where “x” is Pro. In some cases, a variant 4-1BBL polypeptide of the present disclosure comprises the amino acid sequence set forth in FIG. 2UU, where “x” is Phe. In some cases, a variant 4-1BBL polypeptide of the present disclosure comprises the amino acid sequence set forth in FIG. 2UU, where “x” is Tyr. In some cases, a variant 4-1BBL polypeptide of the present disclosure comprises the amino acid sequence set forth in FIG. 2UU, where “x” is Trp. In some cases, a variant 4-1BBL polypeptide of the present disclosure comprises the amino acid sequence set forth in FIG. 2UU, where “x” is Ser. In some cases, a variant 4-1BBL polypeptide of the present disclosure comprises the amino acid sequence set forth in FIG. 2UU, where “x” is Thr. In some cases, a variant 4-1BBL polypeptide of the present disclosure comprises the amino acid sequence set forth in FIG. 2UU, where “x” is Cys. In some cases, a variant 4-1BBL polypeptide of the present disclosure comprises the amino acid sequence set forth in FIG. 2UU, where “x” is Met. In some cases, a variant 4-1BBL polypeptide of the present disclosure comprises the amino acid sequence set forth in FIG. 2UU, where “x” is Asn. In some cases, a variant 4-1BBL polypeptide of the present disclosure comprises the amino acid sequence set forth in FIG. 2UU, where “x” is Gln. In some cases, a variant 4-1BBL polypeptide of the present disclosure comprises the amino acid sequence set forth in FIG. 2UU, where “x” is Lys. In some cases, a variant 4-1BBL polypeptide of the present disclosure comprises the amino acid sequence set forth in FIG. 2UU, where “x” is Arg. In some cases, a variant 4-1BBL polypeptide of the present disclosure comprises the amino acid sequence set forth in FIG. 2UU, where “x” is His. In some cases, a variant 4-1BBL polypeptide of the present disclosure comprises the amino acid sequence set forth in FIG. 2UU, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L149 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 (indicated by an “x”) is an amino acid other than a leucine, e.g., where amino acid 149 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2VV retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VV, where amino acid 149 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L149. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at L69. In some cases, a variant 4-1BBL polypeptide of the present disclosure comprises the amino acid sequence set forth in SEQ ID NO: 1, with an amino acid substitution at L69. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L70. In some cases, a variant 4-1BBL polypeptide of the present disclosure comprises the amino acid sequence set forth in SEQ ID NO:2, with an amino acid substitution at L70. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L70. In some cases, a variant 4-1BBL polypeptide of the present disclosure comprises the amino acid sequence set forth in SEQ ID NO:3, with an amino acid substitution at L70.

For example, in some cases, a variant 4-1BBL polypeptide of the present disclosure comprises the amino acid sequence set forth in FIG. 2VV, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2VV, where “x” is Gly. In some cases, a variant 4-1BBL polypeptide of the present disclosure comprises the amino acid sequence set forth in FIG. 2VV, where “x” is Ala. In some cases, a variant 4-1BBL polypeptide of the present disclosure comprises the amino acid sequence set forth in FIG. 2VV, where “x” is Val. In some cases, a variant 4-1BBL polypeptide of the present disclosure comprises the amino acid sequence set forth in FIG. 2VV, where “x” is Ile. In some cases, a variant 4-1BBL polypeptide of the present disclosure comprises the amino acid sequence set forth in FIG. 2VV, where “x” is Pro. In some cases, a variant 4-1BBL polypeptide of the present disclosure comprises the amino acid sequence set forth in FIG. 2VV, where “x” is Phe. In some cases, a variant 4-1BBL polypeptide of the present disclosure comprises the amino acid sequence set forth in FIG. 2VV, where “x” is Tyr. In some cases, a variant 4-1BBL polypeptide of the present disclosure comprises the amino acid sequence set forth in FIG. 2VV, where “x” is Trp. In some cases, a variant 4-1BBL polypeptide of the present disclosure comprises the amino acid sequence set forth in FIG. 2VV, where “x” is Ser. In some cases, a variant 4-1BBL polypeptide of the present disclosure comprises the amino acid sequence set forth in FIG. 2VV, where “x” is Thr. In some cases, a variant 4-1BBL polypeptide of the present disclosure comprises the amino acid sequence set forth in FIG. 2VV, where “x” is Cys. In some cases, a variant 4-1BBL polypeptide of the present disclosure comprises the amino acid sequence set forth in FIG. 2VV, where “x” is Met. In some cases, a variant 4-1BBL polypeptide of the present disclosure comprises the amino acid sequence set forth in FIG. 2VV, where “x” is Asn. In some cases, a variant 4-1BBL polypeptide of the present disclosure comprises the amino acid sequence set forth in FIG. 2VV, where “x” is Gln. In some cases, a variant 4-1BBL polypeptide of the present disclosure comprises the amino acid sequence set forth in FIG. 2VV, where “x” is Lys. In some cases, a variant 4-1BBL polypeptide of the present disclosure comprises the amino acid sequence set forth in FIG. 2VV, where “x” is Arg. In some cases, a variant 4-1BBL polypeptide of the present disclosure comprises the amino acid sequence set forth in FIG. 2VV, where “x” is His. In some cases, a variant 4-1BBL polypeptide of the present disclosure comprises the amino acid sequence set forth in FIG. 2VV, where “x” is Asp. In some cases, a variant 4-1BBL polypeptide of the present disclosure comprises the amino acid sequence set forth in FIG. 2VV, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R150 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 (indicated by an “x”) is an amino acid other than an arginine, e.g., where amino acid 150 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2WW retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WW, where amino acid 150 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R150. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at R70. In some cases, a variant 4-1BBL polypeptide of the present disclosure comprises the amino acid sequence set forth in SEQ ID NO: 1, with an amino acid substitution at R70. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R71. In some cases, a variant 4-1BBL polypeptide of the present disclosure comprises the amino acid sequence set forth in SEQ ID NO:2, with an amino acid substitution at R71. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R71. In some cases, a variant 4-1BBL polypeptide of the present disclosure comprises the amino acid sequence set forth in SEQ ID NO:3, with an amino acid substitution at R71.

For example, in some cases, a variant 4-1BBL polypeptide of the present disclosure comprises the amino acid sequence set forth in FIG. 2WW, where “x” is any amino acid other than arginine; for example, “x” can be Gly, Ala, Val, Leu, Ile, Pro, Phe, Tyr, Trp, Ser, Thr, Cys, Met, Asn, Gln, Lys, His, Asp, or Glu. In some cases, a variant 4-1BBL polypeptide of the present disclosure comprises the amino acid sequence set forth in FIG. 2WW, where “x” is Gly. In some cases, a variant 4-1BBL polypeptide of the present disclosure comprises the amino acid sequence set forth in FIG. 2WW, where “x” is Ala. In some cases, a variant 4-1BBL polypeptide of the present disclosure comprises the amino acid sequence set forth in FIG. 2WW, where “x” is Val. In some cases, a variant 4-1BBL polypeptide of the present disclosure comprises the amino acid sequence set forth in FIG. 2WW, where “x” is Leu. In some cases, a variant 4-1BBL polypeptide of the present disclosure comprises the amino acid sequence set forth in FIG. 2WW, where “x” is Ile. In some cases, a variant 4-1BBL polypeptide of the present disclosure comprises the amino acid sequence set forth in FIG. 2WW, where “x” is Pro. In some cases, a variant 4-1BBL polypeptide of the present disclosure comprises the amino acid sequence set forth in FIG. 2WW, where “x” is Phe. In some cases, a variant 4-1BBL polypeptide of the present disclosure comprises the amino acid sequence set forth in FIG. 2WW, where “x” is Tyr. In some cases, a variant 4-1BBL polypeptide of the present disclosure comprises the amino acid sequence set forth in FIG. 2WW, where “x” is Trp. In some cases, a variant 4-1BBL polypeptide of the present disclosure comprises the amino acid sequence set forth in FIG. 2WW, where “x” is Ser. In some cases, a variant 4-1BBL polypeptide of the present disclosure comprises the amino acid sequence set forth in FIG. 2WW, where “x” is Thr. In some cases, a variant 4-1BBL polypeptide of the present disclosure comprises the amino acid sequence set forth in FIG. 2WW, where “x” is Cys. In some cases, a variant 4-1BBL polypeptide of the present disclosure comprises the amino acid sequence set forth in FIG. 2WW, where “x” is Met. In some cases, a variant 4-1BBL polypeptide of the present disclosure comprises the amino acid sequence set forth in FIG. 2WW, where “x” is Asn. In some cases, a variant 4-1BBL polypeptide of the present disclosure comprises the amino acid sequence set forth in FIG. 2WW, where “x” is Gln. In some cases, a variant 4-1BBL polypeptide of the present disclosure comprises the amino acid sequence set forth in FIG. 2WW, where “x” is Lys. In some cases, a variant 4-1BBL polypeptide of the present disclosure comprises the amino acid sequence set forth in FIG. 2WW, where “x” is His. In some cases, a variant 4-1BBL polypeptide of the present disclosure comprises the amino acid sequence set forth in FIG. 2WW, where “x” is Asp. In some cases, a variant 4-1BBL polypeptide of the present disclosure comprises the amino acid sequence set forth in FIG. 2WW, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R151 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 (indicated by an “x”) is an amino acid other than an arginine, e.g., where amino acid 150 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2XX retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XX, where amino acid 151 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R151. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at R71. In some cases, a variant 4-1BBL polypeptide of the present disclosure comprises the amino acid sequence set forth in SEQ ID NO:1, with an amino acid substitution at R71. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R72. In some cases, a variant 4-1BBL polypeptide of the present disclosure comprises the amino acid sequence set forth in SEQ ID NO:2, with an amino acid substitution at R72. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R72. In some cases, a variant 4-1BBL polypeptide of the present disclosure comprises the amino acid sequence set forth in SEQ ID NO:3, with an amino acid substitution at R72.

For example, in some cases, a variant 4-1BBL polypeptide of the present disclosure comprises the amino acid sequence set forth in FIG. 2XX, where “x” is any amino acid other than arginine; for example, “x” can be Gly, Ala, Val, Leu, Ile, Pro, Phe, Tyr, Trp, Ser, Thr, Cys, Met, Asn, Gln, Lys, His, Asp, or Glu. In some cases, a variant 4-1BBL polypeptide of the present disclosure comprises the amino acid sequence set forth in FIG. 2XX, where “x” is Gly. In some cases, a variant 4-1BBL polypeptide of the present disclosure comprises the amino acid sequence set forth in FIG. 2XX, where “x” is Ala. In some cases, a variant 4-1BBL polypeptide of the present disclosure comprises the amino acid sequence set forth in FIG. 2XX, where “x” is Val. In some cases, a variant 4-1BBL polypeptide of the present disclosure comprises the amino acid sequence set forth in FIG. 2XX, where “x” is Leu. In some cases, a variant 4-1BBL polypeptide of the present disclosure comprises the amino acid sequence set forth in FIG. 2XX, where “x” is Ile. In some cases, a variant 4-1BBL polypeptide of the present disclosure comprises the amino acid sequence set forth in FIG. 2XX, where “x” is Pro. In some cases, a variant 4-1BBL polypeptide of the present disclosure comprises the amino acid sequence set forth in FIG. 2XX, where “x” is Phe. In some cases, a variant 4-1BBL polypeptide of the present disclosure comprises the amino acid sequence set forth in FIG. 2XX, where “x” is Tyr. In some cases, a variant 4-1BBL polypeptide of the present disclosure comprises the amino acid sequence set forth in FIG. 2XX, where “x” is Trp. In some cases, a variant 4-1BBL polypeptide of the present disclosure comprises the amino acid sequence set forth in FIG. 2XX where “x” is Ser. In some cases, a variant 4-1BBL polypeptide of the present disclosure comprises the amino acid sequence set forth in FIG. 2XX, where “x” is Thr. In some cases, a variant 4-1BBL polypeptide of the present disclosure comprises the amino acid sequence set forth in FIG. 2XX, where “x” is Cys. In some cases, a variant 4-1BBL polypeptide of the present disclosure comprises the amino acid sequence set forth in FIG. 2XX, where “x” is Met. In some cases, a variant 4-1BBL polypeptide of the present disclosure comprises the amino acid sequence set forth in FIG. 2XX, where “x” is Asn. In some cases, a variant 4-1BBL polypeptide of the present disclosure comprises the amino acid sequence set forth in FIG. 2XX, where “x” is Gln. In some cases, a variant 4-1BBL polypeptide of the present disclosure comprises the amino acid sequence set forth in FIG. 2XX, where “x” is Lys. In some cases, a variant 4-1BBL polypeptide of the present disclosure comprises the amino acid sequence set forth in FIG. 2XX, where “x” is His. In some cases, a variant 4-1BBL polypeptide of the present disclosure comprises the amino acid sequence set forth in FIG. 2XX, where “x” is Asp. In some cases, a variant 4-1BBL polypeptide of the present disclosure comprises the amino acid sequence set forth in FIG. 2XX, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V152 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 (indicated by an “x”) is an amino acid other than a valine, e.g., where amino acid 152 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2YY retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YY, where amino acid 152 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V152. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at V72. In some cases, a variant 4-1BBL polypeptide of the present disclosure comprises the amino acid sequence set forth in SEQ ID NO: 1, with an amino acid substitution at V72. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V73. In some cases, a variant 4-1BBL polypeptide of the present disclosure comprises the amino acid sequence set forth in SEQ ID NO:2, with an amino acid substitution at V73. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V73. In some cases, a variant 4-1BBL polypeptide of the present disclosure comprises the amino acid sequence set forth in SEQ ID NO:3, with an amino acid substitution at V73.

For example, in some cases, a variant 4-1BBL polypeptide of the present disclosure comprises the amino acid sequence set forth in FIG. 2YY, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2YY, where “x” is Gly. In some cases, a variant 4-1BBL polypeptide of the present disclosure comprises the amino acid sequence set forth in FIG. 2YY, where “x” is Ala. In some cases, a variant 4-1BBL polypeptide of the present disclosure comprises the amino acid sequence set forth in FIG. 2YY, where “x” is Leu. In some cases, a variant 4-1BBL polypeptide of the present disclosure comprises the amino acid sequence set forth in FIG. 2YY, where “x” is Ile. In some cases, a variant 4-1BBL polypeptide of the present disclosure comprises the amino acid sequence set forth in FIG. 2YY, where “x” is Pro. In some cases, a variant 4-1BBL polypeptide of the present disclosure comprises the amino acid sequence set forth in FIG. 2YY, where “x” is Phe. In some cases, a variant 4-1BBL polypeptide of the present disclosure comprises the amino acid sequence set forth in FIG. 2YY, where “x” is Tyr. In some cases, a variant 4-1BBL polypeptide of the present disclosure comprises the amino acid sequence set forth in FIG. 2YY, where “x” is Trp. In some cases, a variant 4-1BBL polypeptide of the present disclosure comprises the amino acid sequence set forth in FIG. 2YY, where “x” is Ser. In some cases, a variant 4-1BBL polypeptide of the present disclosure comprises the amino acid sequence set forth in FIG. 2YY, where “x” is Thr. In some cases, a variant 4-1BBL polypeptide of the present disclosure comprises the amino acid sequence set forth in FIG. 2YY, where “x” is Cys. In some cases, a variant 4-1BBL polypeptide of the present disclosure comprises the amino acid sequence set forth in FIG. 2YY, where “x” is Met. In some cases, a variant 4-1BBL polypeptide of the present disclosure comprises the amino acid sequence set forth in FIG. 2YY, where “x” is Asn. In some cases, a variant 4-1BBL polypeptide of the present disclosure comprises the amino acid sequence set forth in FIG. 2YY, where “x” is Gln. In some cases, a variant 4-1BBL polypeptide of the present disclosure comprises the amino acid sequence set forth in FIG. 2YY, where “x” is Lys. In some cases, a variant 4-1BBL polypeptide of the present disclosure comprises the amino acid sequence set forth in FIG. 2YY, where “x” is Arg. In some cases, a variant 4-1BBL polypeptide of the present disclosure comprises the amino acid sequence set forth in FIG. 2YY, where “x” is His. In some cases, a variant 4-1BBL polypeptide of the present disclosure comprises the amino acid sequence set forth in FIG. 2YY, where “x” is Asp. In some cases, a variant 4-1BBL polypeptide of the present disclosure comprises the amino acid sequence set forth in FIG. 2YY, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V153 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 153 (indicated by an “x”) is an amino acid other than a valine, e.g., where amino acid 153 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2ZZ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZ, where amino acid 152 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V153. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at V73. In some cases, a variant 4-1BBL polypeptide of the present disclosure comprises the amino acid sequence set forth in SEQ ID NO:1, with an amino acid substitution at V73. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V74. In some cases, a variant 4-1BBL polypeptide of the present disclosure comprises the amino acid sequence set forth in SEQ ID NO:2, with an amino acid substitution at V74. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V74. In some cases, a variant 4-1BBL polypeptide of the present disclosure comprises the amino acid sequence set forth in SEQ ID NO:3, with an amino acid substitution at V74.

For example, in some cases, a variant 4-1BBL polypeptide of the present disclosure comprises the amino acid sequence set forth in FIG. 2ZZ, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2ZZ, where “x” is Gly. In some cases, a variant 4-1BBL polypeptide of the present disclosure comprises the amino acid sequence set forth in FIG. 2ZZ, where “x” is Ala. In some cases, a variant 4-1BBL polypeptide of the present disclosure comprises the amino acid sequence set forth in FIG. 2ZZ, where “x” is Leu. In some cases, a variant 4-1BBL polypeptide of the present disclosure comprises the amino acid sequence set forth in FIG. 2ZZ, where “x” is Ile. In some cases, a variant 4-1BBL polypeptide of the present disclosure comprises the amino acid sequence set forth in FIG. 2ZZ, where “x” is Pro. In some cases, a variant 4-1BBL polypeptide of the present disclosure comprises the amino acid sequence set forth in FIG. 2ZZ, where “x” is Phe. In some cases, a variant 4-1BBL polypeptide of the present disclosure comprises the amino acid sequence set forth in FIG. 2ZZ, where “x” is Tyr. In some cases, a variant 4-1BBL polypeptide of the present disclosure comprises the amino acid sequence set forth in FIG. 2ZZ, where “x” is Trp. In some cases, a variant 4-1BBL polypeptide of the present disclosure comprises the amino acid sequence set forth in FIG. 2ZZ, where “x” is Ser. In some cases, a variant 4-1BBL polypeptide of the present disclosure comprises the amino acid sequence set forth in FIG. 2ZZ, where “x” is Thr. In some cases, a variant 4-1BBL polypeptide of the present disclosure comprises the amino acid sequence set forth in FIG. 2ZZ, where “x” is Cys. In some cases, a variant 4-1BBL polypeptide of the present disclosure comprises the amino acid sequence set forth in FIG. 2ZZ, where “x” is Met. In some cases, a variant 4-1BBL polypeptide of the present disclosure comprises the amino acid sequence set forth in FIG. 2ZZ, where “x” is Asn. In some cases, a variant 4-1BBL polypeptide of the present disclosure comprises the amino acid sequence set forth in FIG. 2ZZ, where “x” is Gln. In some cases, a variant 4-1BBL polypeptide of the present disclosure comprises the amino acid sequence set forth in FIG. 2ZZ, where “x” is Lys. In some cases, a variant 4-1BBL polypeptide of the present disclosure comprises the amino acid sequence set forth in FIG. 2ZZ, where “x” is Arg. In some cases, a variant 4-1BBL polypeptide of the present disclosure comprises the amino acid sequence set forth in FIG. 2ZZ, where “x” is His. In some cases, a variant 4-1BBL polypeptide of the present disclosure comprises the amino acid sequence set forth in FIG. 2ZZ, where “x” is Asp. In some cases, a variant 4-1BBL polypeptide of the present disclosure comprises the amino acid sequence set forth in FIG. 2ZZ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G155 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 (indicated by an “x”) is an amino acid other than a glycine, e.g., where amino acid 155 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2AAA retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino 155 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAA, where amino acid 155 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G155. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at G75. In some cases, a variant 4-1BBL polypeptide of the present disclosure comprises the amino acid sequence set forth in SEQ ID NO:1, with an amino acid substitution at G75. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G76. In some cases, a variant 4-1BBL polypeptide of the present disclosure comprises the amino acid sequence set forth in SEQ ID NO:2, with an amino acid substitution at G76. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G76. In some cases, a variant 4-1BBL polypeptide of the present disclosure comprises the amino acid sequence set forth in SEQ ID NO:3, with an amino acid substitution at G76.

For example, in some cases, a variant 4-1BBL polypeptide of the present disclosure comprises the amino acid sequence set forth in FIG. 2AAA, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2AAA, where “x” is Ala. In some cases, a variant 4-1BBL polypeptide of the present disclosure comprises the amino acid sequence set forth in FIG. 2AAA, where “x” is Val. In some cases, a variant 4-1BBL polypeptide of the present disclosure comprises the amino acid sequence set forth in FIG. 2AAA, where “x” is Leu. In some cases, a variant 4-1BBL polypeptide of the present disclosure comprises the amino acid sequence set forth in FIG. 2AAA, where “x” is Ile. In some cases, a variant 4-1BBL polypeptide of the present disclosure comprises the amino acid sequence set forth in FIG. 2AAA, where “x” is Pro. In some cases, a variant 4-1BBL polypeptide of the present disclosure comprises the amino acid sequence set forth in FIG. 2AAA, where “x” is Phe. In some cases, a variant 4-1BBL polypeptide of the present disclosure comprises the amino acid sequence set forth in FIG. 2AAA, where “x” is Tyr. In some cases, a variant 4-1BBL polypeptide of the present disclosure comprises the amino acid sequence set forth in FIG. 2AAA, where “x” is Trp. In some cases, a variant 4-1BBL polypeptide of the present disclosure comprises the amino acid sequence set forth in FIG. 2AAA, where “x” is Ser. In some cases, a variant 4-1BBL polypeptide of the present disclosure comprises the amino acid sequence set forth in FIG. 2AAA, where “x” is Thr. In some cases, a variant 4-1BBL polypeptide of the present disclosure comprises the amino acid sequence set forth in FIG. 2AAA, where “x” is Cys. In some cases, a variant 4-1BBL polypeptide of the present disclosure comprises the amino acid sequence set forth in FIG. 2AAA, where “x” is Met. In some cases, a variant 4-1BBL polypeptide of the present disclosure comprises the amino acid sequence set forth in FIG. 2AAA, where “x” is Asn. In some cases, a variant 4-1BBL polypeptide of the present disclosure comprises the amino acid sequence set forth in FIG. 2AAA, where “x” is Gln. In some cases, a variant 4-1BBL polypeptide of the present disclosure comprises the amino acid sequence set forth in FIG. 2AAA, where “x” is Lys. In some cases, a variant 4-1BBL polypeptide of the present disclosure comprises the amino acid sequence set forth in FIG. 2AAA, where “x” is Arg. In some cases, a variant 4-1BBL polypeptide of the present disclosure comprises the amino acid sequence set forth in FIG. 2AAA, where “x” is His. In some cases, a variant 4-1BBL polypeptide of the present disclosure comprises the amino acid sequence set forth in FIG. 2AAA, where “x” is Asp. In some cases, a variant 4-1BBL polypeptide of the present disclosure comprises the amino acid sequence set forth in FIG. 2AAA, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### E156 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 (indicated by an “x”) is an amino acid other than a glutamic acid, e.g., where amino acid 156 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2BBB retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBB, where amino acid 156 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E156. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at E76. In some cases, a variant 4-1BBL polypeptide of the present disclosure comprises the amino acid sequence set forth in SEQ ID NO: 1, with an amino acid substitution at E76. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E77. In some cases, a variant 4-1BBL polypeptide of the present disclosure comprises the amino acid sequence set forth in SEQ ID NO:2, with an amino acid substitution at E77. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E77. In some cases, a variant 4-1BBL polypeptide of the present disclosure comprises the amino acid sequence set forth in SEQ ID NO:3, with an amino acid substitution at E77.

For example, in some cases, a variant 4-1BBL polypeptide of the present disclosure comprises the amino acid sequence set forth in FIG. 2BBB, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2BBB, where “x” is Gly. In some cases, a variant 4-1BBL polypeptide of the present disclosure comprises the amino acid sequence set forth in FIG. 2BBB, where “x” is Ala. In some cases, a variant 4-1BBL polypeptide of the present disclosure comprises the amino acid sequence set forth in FIG. 2BBB, where “x” is Val. In some cases, a variant 4-1BBL polypeptide of the present disclosure comprises the amino acid sequence set forth in FIG. 2BBB, where “x” is Leu. In some cases, a variant 4-1BBL polypeptide of the present disclosure comprises the amino acid sequence set forth in FIG. 2BBB, where “x” is Ile. In some cases, a variant 4-1BBL polypeptide of the present disclosure comprises the amino acid sequence set forth in FIG. 2BBB, where “x” is Pro. In some cases, a variant 4-1BBL polypeptide of the present disclosure comprises the amino acid sequence set forth in FIG. 2BBB, where “x” is Phe. In some cases, a variant 4-1BBL polypeptide of the present disclosure comprises the amino acid sequence set forth in FIG. 2BBB, where “x” is Tyr. In some cases, a variant 4-1BBL polypeptide of the present disclosure comprises the amino acid sequence set forth in FIG. 2BBB, where “x” is Trp. In some cases, a variant 4-1BBL polypeptide of the present disclosure comprises the amino acid sequence set forth in FIG. 2BBB, where “x” is Ser. In some cases, a variant 4-1BBL polypeptide of the present disclosure comprises the amino acid sequence set forth in FIG. 2BBB, where “x” is Thr. In some cases, a variant 4-1BBL polypeptide of the present disclosure comprises the amino acid sequence set forth in FIG. 2BBB, where “x” is Cys. In some cases, a variant 4-1BBL polypeptide of the present disclosure comprises the amino acid sequence set forth in FIG. 2BBB, where “x” is Met. In some cases, a variant 4-1BBL polypeptide of the present disclosure comprises the amino acid sequence set forth in FIG. 2BBB, where “x” is Asn. In some cases, a variant 4-1BBL polypeptide of the present disclosure comprises the amino acid sequence set forth in FIG. 2BBB, where “x” is Gln. In some cases, a variant 4-1BBL polypeptide of the present disclosure comprises the amino acid sequence set forth in FIG. 2BBB, where “x” is Lys. In some cases, a variant 4-1BBL polypeptide of the present disclosure comprises the amino acid sequence set forth in FIG. 2BBB, where “x” is Arg. In some cases, a variant 4-1BBL polypeptide of the present disclosure comprises the amino acid sequence set forth in FIG. 2BBB, where “x” is His. In some cases, a variant 4-1BBL polypeptide of the present disclosure comprises the amino acid sequence set forth in FIG. 2BBB, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G157 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 (indicated by an “x”) is an amino acid other than a glycine, e.g., where amino acid 157 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2CCC retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCC, where amino acid 157 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G157. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at G77. In some cases, a variant 4-1BBL polypeptide of the present disclosure comprises the amino acid sequence set forth in SEQ ID NO: 1, with an amino acid substitution at G77. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G78. In some cases, a variant 4-1BBL polypeptide of the present disclosure comprises the amino acid sequence set forth in SEQ ID NO:2, with an amino acid substitution at G78. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G78. In some cases, a variant 4-1BBL polypeptide of the present disclosure comprises the amino acid sequence set forth in SEQ ID NO:3, with an amino acid substitution at G78.

For example, in some cases, a variant 4-1BBL polypeptide of the present disclosure comprises the amino acid sequence set forth in FIG. 2CCC, where “x” is any amino acid other than methionine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2CCC, where “x” is Ala. In some cases, a variant 4-1BBL polypeptide of the present disclosure comprises the amino acid sequence set forth in FIG. 2CCC, where “x” is Val. In some cases, a variant 4-1BBL polypeptide of the present disclosure comprises the amino acid sequence set forth in FIG. 2CCC, where “x” is Leu. In some cases, a variant 4-1BBL polypeptide of the present disclosure comprises the amino acid sequence set forth in FIG. 2CCC, where “x” is Ile. In some cases, a variant 4-1BBL polypeptide of the present disclosure comprises the amino acid sequence set forth in FIG. 2CCC, where “x” is Pro. In some cases, a variant 4-1BBL polypeptide of the present disclosure comprises the amino acid sequence set forth in FIG. 2CCC, where “x” is Phe. In some cases, a variant 4-1BBL polypeptide of the present disclosure comprises the amino acid sequence set forth in FIG. 2CCC, where “x” is Tyr. In some cases, a variant 4-1BBL polypeptide of the present disclosure comprises the amino acid sequence set forth in FIG. 2CCC, where “x” is Trp. In some cases, a variant 4-1BBL polypeptide of the present disclosure comprises the amino acid sequence set forth in FIG. 2CCC, where “x” is Ser. In some cases, a variant 4-1BBL polypeptide of the present disclosure comprises the amino acid sequence set forth in FIG. 2CCC, where “x” is Thr. In some cases, a variant 4-1BBL polypeptide of the present disclosure comprises the amino acid sequence set forth in FIG. 2CCC, where “x” is Cys. In some cases, a variant 4-1BBL polypeptide of the present disclosure comprises the amino acid sequence set forth in FIG. 2CCC, where “x” is Met. In some cases, a variant 4-1BBL polypeptide of the present disclosure comprises the amino acid sequence set forth in FIG. 2CCC, where “x” is Asn. In some cases, a variant 4-1BBL polypeptide of the present disclosure comprises the amino acid sequence set forth in FIG. 2CCC, where “x” is Gln. In some cases, a variant 4-1BBL polypeptide of the present disclosure comprises the amino acid sequence set forth in FIG. 2CCC, where “x” is Lys. In some cases, a variant 4-1BBL polypeptide of the present disclosure comprises the amino acid sequence set forth in FIG. 2CCC, where “x” is Arg. In some cases, a variant 4-1BBL polypeptide of the present disclosure comprises the amino acid sequence set forth in FIG. 2CCC, where “x” is His. In some cases, a variant 4-1BBL polypeptide of the present disclosure comprises the amino acid sequence set forth in FIG. 2CCC, where “x” is Asp. In some cases, a variant 4-1BBL polypeptide of the present disclosure comprises the amino acid sequence set forth in FIG. 2CCC, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S158 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 (indicated by an “x”) is an amino acid other than a serine, e.g., where amino acid 158 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2DDD retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDD, where amino acid 158 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S158. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S78. In some cases, a variant 4-1BBL polypeptide of the present disclosure comprises the amino acid sequence set forth in SEQ ID NO:1, with an amino acid substitution at S78. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S79. In some cases, a variant 4-1BBL polypeptide of the present disclosure comprises the amino acid sequence set forth in SEQ ID NO:2, with an amino acid substitution at S79. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S79. In some cases, a variant 4-1BBL polypeptide of the present disclosure comprises the amino acid sequence set forth in SEQ ID NO:3, with an amino acid substitution at S79.

For example, in some cases, a variant 4-1BBL polypeptide of the present disclosure comprises the amino acid sequence set forth in FIG. 2DDD, where “x” is any amino acid other than methionine; for example, “x” can be Gly, Ala, Val, Leu, Ile, Pro, Phe, Tyr, Trp, Ser, Thr, Cys, Asn, Gln, Lys, Arg, His, Asp, or Glu. In some cases, a variant 4-1BBL polypeptide of the present disclosure comprises the amino acid sequence set forth in FIG. 2DDD, where “x” is Gly. In some cases, a variant 4-1BBL polypeptide of the present disclosure comprises the amino acid sequence set forth in FIG. 2DDD, where “x” is Ala. In some cases, a variant 4-1BBL polypeptide of the present disclosure comprises the amino acid sequence set forth in FIG. 2DDD, where “x” is Val. In some cases, a variant 4-1BBL polypeptide of the present disclosure comprises the amino acid sequence set forth in FIG. 2DDD, where “x” is Leu. In some cases, a variant 4-1BBL polypeptide of the present disclosure comprises the amino acid sequence set forth in FIG. 2DDD, where “x” is Ile. In some cases, a variant 4-1BBL polypeptide of the present disclosure comprises the amino acid sequence set forth in FIG. 2DDD, where “x” is Pro. In some cases, a variant 4-1BBL polypeptide of the present disclosure comprises the amino acid sequence set forth in FIG. 2DDD, where “x” is Phe. In some cases, a variant 4-1BBL polypeptide of the present disclosure comprises the amino acid sequence set forth in FIG. 2DDD, where “x” is Tyr. In some cases, a variant 4-1BBL polypeptide of the present disclosure comprises the amino acid sequence set forth in FIG. 2DDD, where “x” is Trp. In some cases, a variant 4-1BBL polypeptide of the present disclosure comprises the amino acid sequence set forth in FIG. 2DDD, where “x” is Thr. In some cases, a variant 4-1BBL polypeptide of the present disclosure comprises the amino acid sequence set forth in FIG. 2DDD, where “x” is Cys. In some cases, a variant 4-1BBL polypeptide of the present disclosure comprises the amino acid sequence set forth in FIG. 2DDD, where “x” is Met. In some cases, a variant 4-1BBL polypeptide of the present disclosure comprises the amino acid sequence set forth in FIG. 2DDD, where “x” is Asn. In some cases, a variant 4-1BBL polypeptide of the present disclosure comprises the amino acid sequence set forth in FIG. 2DDD, where “x” is Gln. In some cases, a variant 4-1BBL polypeptide of the present disclosure comprises the amino acid sequence set forth in FIG. 2DDD, where “x” is Lys. In some cases, a variant 4-1BBL polypeptide of the present disclosure comprises the amino acid sequence set forth in FIG. 2DDD, where “x” is Arg. In some cases, a variant 4-1BBL polypeptide of the present disclosure comprises the amino acid sequence set forth in FIG. 2DDD, where “x” is His. In some cases, a variant 4-1BBL polypeptide of the present disclosure comprises the amino acid sequence set forth in FIG. 2DDD, where “x” is Asp. In some cases, a variant 4-1BBL polypeptide of the present disclosure comprises the amino acid sequence set forth in FIG. 2DDD, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### D184 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 (indicated by an “x”) is an amino acid other than an aspartic acid, e.g., where amino acid 184 is Gly, Ala, Val, Leu, Ile, Pro, Phe, Tyr, Trp, Ser, Thr, Cys, Met, Asn, Gln, Lys, Arg, His, or Glu. The amino acid numbering in FIG. 2EEE retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 155 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEE, where amino acid 184 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at D184. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at D104. In some cases, a variant 4-1BBL polypeptide of the present disclosure comprises the amino acid sequence set forth in SEQ ID NO:1, with an amino acid substitution at D104. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D105. In some cases, a variant 4-1BBL polypeptide of the present disclosure comprises the amino acid sequence set forth in SEQ ID NO:2, with an amino acid substitution at D105. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D105. In some cases, a variant 4-1BBL polypeptide of the present disclosure comprises the amino acid sequence set forth in SEQ ID NO:3, with an amino acid substitution at D105.

For example, in some cases, a variant 4-1BBL polypeptide of the present disclosure comprises the amino acid sequence set forth in FIG. 2EEE, where “x” is any amino acid other than aspartic acid; for example, “x” can be Gly, Ala, Val, Leu, Ile, Pro, Phe, Tyr, Trp, Ser, Thr, Cys, Met, Asn, Gln, Lys, Arg, His, or Glu. In some cases, a variant 4-1BBL polypeptide of the present disclosure comprises the amino acid sequence set forth in FIG. 2EEE, where “x” is Gly. In some cases, a variant 4-1BBL polypeptide of the present disclosure comprises the amino acid sequence set forth in FIG. 2EEE, where “x” is Ala. In some cases, a variant 4-1BBL polypeptide of the present disclosure comprises the amino acid sequence set forth in FIG. 2EEE, where “x” is Val. In some cases, a variant 4-1BBL polypeptide of the present disclosure comprises the amino acid sequence set forth in FIG. 2EEE, where “x” is Leu. In some cases, a variant 4-1BBL polypeptide of the present disclosure comprises the amino acid sequence set forth in FIG. 2EEE, where “x” is Ile. In some cases, a variant 4-1BBL polypeptide of the present disclosure comprises the amino acid sequence set forth in FIG. 2EEE, where “x” is Pro. In some cases, a variant 4-1BBL polypeptide of the present disclosure comprises the amino acid sequence set forth in FIG. 2EEE, where “x” is Phe. In some cases, a variant 4-1BBL polypeptide of the present disclosure comprises the amino acid sequence set forth in FIG. 2EEE, where “x” is Tyr. In some cases, a variant 4-1BBL polypeptide of the present disclosure comprises the amino acid sequence set forth in FIG. 2EEE, where “x” is Trp. In some cases, a variant 4-1BBL polypeptide of the present disclosure comprises the amino acid sequence set forth in FIG. 2EEE, where “x” is Ser. In some cases, a variant 4-1BBL polypeptide of the present disclosure comprises the amino acid sequence set forth in FIG. 2EEE, where “x” is Thr. In some cases, a variant 4-1BBL polypeptide of the present disclosure comprises the amino acid sequence set forth in FIG. 2EEE, where “x” is Cys. In some cases, a variant 4-1BBL polypeptide of the present disclosure comprises the amino acid sequence set forth in FIG. 2EEE, where “x” is Met. In some cases, a variant 4-1BBL polypeptide of the present disclosure comprises the amino acid sequence set forth in FIG. 2EEE, where “x” is Asn. In some cases, a variant 4-1BBL polypeptide of the present disclosure comprises the amino acid sequence set forth in FIG. 2EEE, where “x” is Gln. In some cases, a variant 4-1BBL polypeptide of the present disclosure comprises the amino acid sequence set forth in FIG. 2EEE, where “x” is Lys. In some cases, a variant 4-1BBL polypeptide of the present disclosure comprises the amino acid sequence set forth in FIG. 2EEE, where “x” is Arg. In some cases, a variant 4-1BBL polypeptide of the present disclosure comprises the amino acid sequence set forth in FIG. 2EEE, where “x” is His. In some cases, a variant 4-1BBL polypeptide of the present disclosure comprises the amino acid sequence set forth in FIG. 2EEE, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L185 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 (indicated by an “x”) is an amino acid other than a leucine, e.g., where amino acid 185 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2FFF retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 184 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFF, where amino acid 185 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L185. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L105. In some cases, a variant 4-1BBL polypeptide of the present disclosure comprises the amino acid sequence set forth in SEQ ID NO:1, with an amino acid substitution at L105. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L106. In some cases, a variant 4-1BBL polypeptide of the present disclosure comprises the amino acid sequence set forth in SEQ ID NO:2, with an amino acid substitution at L106. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L106. In some cases, a variant 4-1BBL polypeptide of the present disclosure comprises the amino acid sequence set forth in SEQ ID NO:3, with an amino acid substitution at L106.

For example, in some cases, a variant 4-1BBL polypeptide of the present disclosure comprises the amino acid sequence set forth in FIG. 2FFF, where “x” is any amino acid other than methionine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2FFF, where “x” is Gly. In some cases, a variant 4-1BBL polypeptide of the present disclosure comprises the amino acid sequence set forth in FIG. 2FFF, where “x” is Ala. In some cases, a variant 4-1BBL polypeptide of the present disclosure comprises the amino acid sequence set forth in FIG. 2FFF, where “x” is Val. In some cases, a variant 4-1BBL polypeptide of the present disclosure comprises the amino acid sequence set forth in FIG. 2FFF, where “x” is Ile. In some cases, a variant 4-1BBL polypeptide of the present disclosure comprises the amino acid sequence set forth in FIG. 2FFF, where “x” is Pro. In some cases, a variant 4-1BBL polypeptide of the present disclosure comprises the amino acid sequence set forth in FIG. 2FFF, where “x” is Phe. In some cases, a variant 4-1BBL polypeptide of the present disclosure comprises the amino acid sequence set forth in FIG. 2FFF, where “x” is Tyr. In some cases, a variant 4-1BBL polypeptide of the present disclosure comprises the amino acid sequence set forth in FIG. 2FFF, where “x” is Trp. In some cases, a variant 4-1BBL polypeptide of the present disclosure comprises the amino acid sequence set forth in FIG. 2FFF, where “x” is Ser. In some cases, a variant 4-1BBL polypeptide of the present disclosure comprises the amino acid sequence set forth in FIG. 2FFF, where “x” is Thr. In some cases, a variant 4-1BBL polypeptide of the present disclosure comprises the amino acid sequence set forth in FIG. 2FFF, where “x” is Cys. In some cases, a variant 4-1BBL polypeptide of the present disclosure comprises the amino acid sequence set forth in FIG. 2FFF, where “x” is Met. In some cases, a variant 4-1BBL polypeptide of the present disclosure comprises the amino acid sequence set forth in FIG. 2FFF, where “x” is Asn. In some cases, a variant 4-1BBL polypeptide of the present disclosure comprises the amino acid sequence set forth in FIG. 2FFF, where “x” is Gln. In some cases, a variant 4-1BBL polypeptide of the present disclosure comprises the amino acid sequence set forth in FIG. 2FFF, where “x” is Lys. In some cases, a variant 4-1BBL polypeptide of the present disclosure comprises the amino acid sequence set forth in FIG. 2FFF, where “x” is Arg. In some cases, a variant 4-1BBL polypeptide of the present disclosure comprises the amino acid sequence set forth in FIG. 2FFF, where “x” is His. In some cases, a variant 4-1BBL polypeptide of the present disclosure comprises the amino acid sequence set forth in FIG. 2FFF, where “x” is Asp. In some cases, a variant 4-1BBL polypeptide of the present disclosure comprises the amino acid sequence set forth in FIG. 2FFF, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### P186 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 (indicated by an “x”) is an amino acid other than a proline, e.g., where amino acid 186 is Gly, Ala, Val, Leu, Ile, Phe, Tyr, Trp, Ser, Thr, Cys, Met, Asn, Gln, Lys, Arg, His, Asp, or Glu. The amino acid numbering in FIG. 2DDD retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGG, where amino acid 186 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at P186. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at P106. In some cases, a variant 4-1BBL polypeptide of the present disclosure comprises the amino acid sequence set forth in SEQ ID NO:1, with an amino acid substitution at P106. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P107. In some cases, a variant 4-1BBL polypeptide of the present disclosure comprises the amino acid sequence set forth in SEQ ID NO:2, with an amino acid substitution at P107. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P107. In some cases, a variant 4-1BBL polypeptide of the present disclosure comprises the amino acid sequence set forth in SEQ ID NO:3, with an amino acid substitution at P107.

For example, in some cases, a variant 4-1BBL polypeptide of the present disclosure comprises the amino acid sequence set forth in FIG. 2GGG, where “x” is any amino acid other than proline; for example, “x” can be Gly, Ala, Val, Leu, Ile, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2GGG, where “x” is Gly. In some cases, a variant 4-1BBL polypeptide of the present disclosure comprises the amino acid sequence set forth in FIG. 2GGG, where “x” is Ala. In some cases, a variant 4-1BBL polypeptide of the present disclosure comprises the amino acid sequence set forth in FIG. 2GGG, where “x” is Val. In some cases, a variant 4-1BBL polypeptide of the present disclosure comprises the amino acid sequence set forth in FIG. 2GGG, where “x” is Leu. In some cases, a variant 4-1BBL polypeptide of the present disclosure comprises the amino acid sequence set forth in FIG. 2GGG, where “x” is Ile. In some cases, a variant 4-1BBL polypeptide of the present disclosure comprises the amino acid sequence set forth in FIG. 2GGG, where “x” is Phe. In some cases, a variant 4-1BBL polypeptide of the present disclosure comprises the amino acid sequence set forth in FIG. 2GGG, where “x” is Tyr. In some cases, a variant 4-1BBL polypeptide of the present disclosure comprises the amino acid sequence set forth in FIG. 2GGG, where “x” is Trp. In some cases, a variant 4-1BBL polypeptide of the present disclosure comprises the amino acid sequence set forth in FIG. 2GGG, where “x” is Ser. In some cases, a variant 4-1BBL polypeptide of the present disclosure comprises the amino acid sequence set forth in FIG. 2GGG, where “x” is Thr. In some cases, a variant 4-1BBL polypeptide of the present disclosure comprises the amino acid sequence set forth in FIG. 2GGG, where “x” is Cys. In some cases, a variant 4-1BBL polypeptide of the present disclosure comprises the amino acid sequence set forth in FIG. 2GGG, where “x” is Met. In some cases, a variant 4-1BBL polypeptide of the present disclosure comprises the amino acid sequence set forth in FIG. 2GGG, where “x” is Asn. In some cases, a variant 4-1BBL polypeptide of the present disclosure comprises the amino acid sequence set forth in FIG. 2GGG, where “x” is Gln. In some cases, a variant 4-1BBL polypeptide of the present disclosure comprises the amino acid sequence set forth in FIG. 2GGG, where “x” is Lys. In some cases, a variant 4-1BBL polypeptide of the present disclosure comprises the amino acid sequence set forth in FIG. 2GGG, where “x” is Arg. In some cases, a variant 4-1BBL polypeptide of the present disclosure comprises the amino acid sequence set forth in FIG. 2GGG, where “x” is His. In some cases, a variant 4-1BBL polypeptide of the present disclosure comprises the amino acid sequence set forth in FIG. 2GGG, where “x” is Asp. In some cases, a variant 4-1BBL polypeptide of the present disclosure comprises the amino acid sequence set forth in FIG. 2GGG, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### P187 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 (indicated by an “x”) is an amino acid other than a proline, e.g., where amino acid 187 is Gly, Ala, Val, Leu, Ile, Phe, Tyr, Trp, Ser, Thr, Cys, Met, Asn, Gln, Lys, Arg, His, Asp, or Glu. The amino acid numbering in FIG. 2DDD retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHH, where amino acid 187 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at P187. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at P107. In some cases, a variant 4-1BBL polypeptide of the present disclosure comprises the amino acid sequence set forth in SEQ ID NO:1, with an amino acid substitution at P107. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P108. In some cases, a variant 4-1BBL polypeptide of the present disclosure comprises the amino acid sequence set forth in SEQ ID NO:2, with an amino acid substitution at P108. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P108. In some cases, a variant 4-1BBL polypeptide of the present disclosure comprises the amino acid sequence set forth in SEQ ID NO:3, with an amino acid substitution at P108.

For example, in some cases, a variant 4-1BBL polypeptide of the present disclosure comprises the amino acid sequence set forth in FIG. 2HHH, where “x” is any amino acid other than proline; for example, “x” can be Gly, Ala, Val, Leu, Ile, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2HHH, where “x” is Gly. In some cases, a variant 4-1BBL polypeptide of the present disclosure comprises the amino acid sequence set forth in FIG. 2HHH, where “x” is Ala. In some cases, a variant 4-1BBL polypeptide of the present disclosure comprises the amino acid sequence set forth in FIG. 2HHH, where “x” is Val. In some cases, a variant 4-1BBL polypeptide of the present disclosure comprises the amino acid sequence set forth in FIG. 2HHH, where “x” is Leu. In some cases, a variant 4-1BBL polypeptide of the present disclosure comprises the amino acid sequence set forth in FIG. 2HHH, where “x” is Ile. In some cases, a variant 4-1BBL polypeptide of the present disclosure comprises the amino acid sequence set forth in FIG. 2HHH, where “x” is Phe. In some cases, a variant 4-1BBL polypeptide of the present disclosure comprises the amino acid sequence set forth in FIG. 2HHH, where “x” is Tyr. In some cases, a variant 4-1BBL polypeptide of the present disclosure comprises the amino acid sequence set forth in FIG. 2HHH, where “x” is Trp. In some cases, a variant 4-1BBL polypeptide of the present disclosure comprises the amino acid sequence set forth in FIG. 2HHH, where “x” is Ser. In some cases, a variant 4-1BBL polypeptide of the present disclosure comprises the amino acid sequence set forth in FIG. 2HHH, where “x” is Thr. In some cases, a variant 4-1BBL polypeptide of the present disclosure comprises the amino acid sequence set forth in FIG. 2HHH, where “x” is Cys. In some cases, a variant 4-1BBL polypeptide of the present disclosure comprises the amino acid sequence set forth in FIG. 2HHH, where “x” is Met. In some cases, a variant 4-1BBL polypeptide of the present disclosure comprises the amino acid sequence set forth in FIG. 2HHH, where “x” is Asn. In some cases, a variant 4-1BBL polypeptide of the present disclosure comprises the amino acid sequence set forth in FIG. 2HHH, where “x” is Gln. In some cases, a variant 4-1BBL polypeptide of the present disclosure comprises the amino acid sequence set forth in FIG. 2HHH, where “x” is Lys. In some cases, a variant 4-1BBL polypeptide of the present disclosure comprises the amino acid sequence set forth in FIG. 2HHH, where “x” is Arg. In some cases, a variant 4-1BBL polypeptide of the present disclosure comprises the amino acid sequence set forth in FIG. 2HHH, where “x” is His. In some cases, a variant 4-1BBL polypeptide of the present disclosure comprises the amino acid sequence set forth in FIG. 2HHH, where “x” is Asp. In some cases, a variant 4-1BBL polypeptide of the present disclosure comprises the amino acid sequence set forth in FIG. 2HHH, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S189 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 (indicated by an “x”) is an amino acid other than a serine, e.g., where amino acid 189 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2III retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2III, where amino acid 189 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S190. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at S109. In some cases, a variant 4-1BBL polypeptide of the present disclosure comprises the amino acid sequence set forth in SEQ ID NO:1, with an amino acid substitution at S109. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S110. In some cases, a variant 4-1BBL polypeptide of the present disclosure comprises the amino acid sequence set forth in SEQ ID NO:2, with an amino acid substitution at S110. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S110. In some cases, a variant 4-1BBL polypeptide of the present disclosure comprises the amino acid sequence set forth in SEQ ID NO:3, with an amino acid substitution at S110.

For example, in some cases, a variant 4-1BBL polypeptide of the present disclosure comprises the amino acid sequence set forth in FIG. 2III, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2III, where “x” is Gly. In some cases, a variant 4-1BBL polypeptide of the present disclosure comprises the amino acid sequence set forth in FIG. 2III, where “x” is Ala. In some cases, a variant 4-1BBL polypeptide of the present disclosure comprises the amino acid sequence set forth in FIG. 2III, where “x” is Val. In some cases, a variant 4-1BBL polypeptide of the present disclosure comprises the amino acid sequence set forth in FIG. 2III, where “x” is Leu. In some cases, a variant 4-1BBL polypeptide of the present disclosure comprises the amino acid sequence set forth in FIG. 2III, where “x” is Ile. In some cases, a variant 4-1BBL polypeptide of the present disclosure comprises the amino acid sequence set forth in FIG. 2III, where “x” is Pro. In some cases, a variant 4-1BBL polypeptide of the present disclosure comprises the amino acid sequence set forth in FIG. 2III, where “x” is Phe. In some cases, a variant 4-1BBL polypeptide of the present disclosure comprises the amino acid sequence set forth in FIG. 2III, where “x” is Tyr. In some cases, a variant 4-1BBL polypeptide of the present disclosure comprises the amino acid sequence set forth in FIG. 2III, where “x” is Trp. In some cases, a variant 4-1BBL polypeptide of the present disclosure comprises the amino acid sequence set forth in FIG. 2III, where “x” is Thr. In some cases, a variant 4-1BBL polypeptide of the present disclosure comprises the amino acid sequence set forth in FIG. 2III, where “x” is Cys. In some cases, a variant 4-1BBL polypeptide of the present disclosure comprises the amino acid sequence set forth in FIG. 2III, where “x” is Met. In some cases, a variant 4-1BBL polypeptide of the present disclosure comprises the amino acid sequence set forth in FIG. 2III, where “x” is Asn. In some cases, a variant 4-1BBL polypeptide of the present disclosure comprises the amino acid sequence set forth in FIG. 2III, where “x” is Gln. In some cases, a variant 4-1BBL polypeptide of the present disclosure comprises the amino acid sequence set forth in FIG. 2III, where “x” is Lys. In some cases, a variant 4-1BBL polypeptide of the present disclosure comprises the amino acid sequence set forth in FIG. 2III, where “x” is Arg. In some cases, a variant 4-1BBL polypeptide of the present disclosure comprises the amino acid sequence set forth in FIG. 2III, where “x” is His. In some cases, a variant 4-1BBL polypeptide of the present disclosure comprises the amino acid sequence set forth in FIG. 2III, where “x” is Asp. In some cases, a variant 4-1BBL polypeptide of the present disclosure comprises the amino acid sequence set forth in FIG. 2III, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S190 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 (indicated by an “x”) is an amino acid other than a serine, e.g., where amino acid 190 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2JJJ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S190. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S110. In some cases, a variant 4-1BBL polypeptide of the present disclosure comprises the amino acid sequence set forth in SEQ ID NO:1, with an amino acid substitution at S110. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S111. In some cases, a variant 4-1BBL polypeptide of the present disclosure comprises the amino acid sequence set forth in SEQ ID NO:2, with an amino acid substitution at S111. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S111. In some cases, a variant 4-1BBL polypeptide of the present disclosure comprises the amino acid sequence set forth in SEQ ID NO:3, with an amino acid substitution at S111.

For example, in some cases, a variant 4-1BBL polypeptide of the present disclosure comprises the amino acid sequence set forth in FIG. 2JJJ, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2JJJ, where “x” is Gly. In some cases, a variant 4-1BBL polypeptide of the present disclosure comprises the amino acid sequence set forth in FIG. 2JJJ, where “x” is Ala. In some cases, a variant 4-1BBL polypeptide of the present disclosure comprises the amino acid sequence set forth in FIG. 2JJJ, where “x” is Val. In some cases, a variant 4-1BBL polypeptide of the present disclosure comprises the amino acid sequence set forth in FIG. 2JJJ, where “x” is Leu. In some cases, a variant 4-1BBL polypeptide of the present disclosure comprises the amino acid sequence set forth in FIG. 2JJJ, where “x” is Ile. In some cases, a variant 4-1BBL polypeptide of the present disclosure comprises the amino acid sequence set forth in FIG. 2JJJ, where “x” is Pro. In some cases, a variant 4-1BBL polypeptide of the present disclosure comprises the amino acid sequence set forth in FIG. 2JJJ, where “x” is Phe. In some cases, a variant 4-1BBL polypeptide of the present disclosure comprises the amino acid sequence set forth in FIG. 2JJJ, where “x” is Tyr. In some cases, a variant 4-1BBL polypeptide of the present disclosure comprises the amino acid sequence set forth in FIG. 2JJJ, where “x” is Trp. In some cases, a variant 4-1BBL polypeptide of the present disclosure comprises the amino acid sequence set forth in FIG. 2JJJ, where “x” is Thr. In some cases, a variant 4-1BBL polypeptide of the present disclosure comprises the amino acid sequence set forth in FIG. 2JJJ, where “x” is Cys. In some cases, a variant 4-1BBL polypeptide of the present disclosure comprises the amino acid sequence set forth in FIG. 2JJJ, where “x” is Met. In some cases, a variant 4-1BBL polypeptide of the present disclosure comprises the amino acid sequence set forth in FIG. 2JJJ, where “x” is Asn. In some cases, a variant 4-1BBL polypeptide of the present disclosure comprises the amino acid sequence set forth in FIG. 2JJJ, where “x” is Gln. In some cases, a variant 4-1BBL polypeptide of the present disclosure comprises the amino acid sequence set forth in FIG. 2JJJ, where “x” is Lys. In some cases, a variant 4-1BBL polypeptide of the present disclosure comprises the amino acid sequence set forth in FIG. 2JJJ, where “x” is Arg. In some cases, a variant 4-1BBL polypeptide of the present disclosure comprises the amino acid sequence set forth in FIG. 2JJJ, where “x” is His. In some cases, a variant 4-1BBL polypeptide of the present disclosure comprises the amino acid sequence set forth in FIG. 2JJJ, where “x” is Asp. In some cases, a variant 4-1BBL polypeptide of the present disclosure comprises the amino acid sequence set forth in FIG. 2JJJ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### E191 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 (indicated by an “x”) is an amino acid other than a glutamic acid, e.g., where amino acid 191 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2KKK retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2KKK, where amino acid 191 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E191. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at E111. In some cases, a variant 4-1BBL polypeptide of the present disclosure comprises the amino acid sequence set forth in SEQ ID NO: 1, with an amino acid substitution at E111. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E112. In some cases, a variant 4-1BBL polypeptide of the present disclosure comprises the amino acid sequence set forth in SEQ ID NO:2, with an amino acid substitution at E112. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E112. In some cases, a variant 4-1BBL polypeptide of the present disclosure comprises the amino acid sequence set forth in SEQ ID NO:3, with an amino acid substitution at E112.

For example, in some cases, a variant 4-1BBL polypeptide of the present disclosure comprises the amino acid sequence set forth in FIG. 2KKK, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2KKK, where “x” is Gly. In some cases, a variant 4-1BBL polypeptide of the present disclosure comprises the amino acid sequence set forth in FIG. 2KKK, where “x” is Ala. In some cases, a variant 4-1BBL polypeptide of the present disclosure comprises the amino acid sequence set forth in FIG. 2KKK, where “x” is Val. In some cases, a variant 4-1BBL polypeptide of the present disclosure comprises the amino acid sequence set forth in FIG. 2KKK, where “x” is Leu. In some cases, a variant 4-1BBL polypeptide of the present disclosure comprises the amino acid sequence set forth in FIG. 2KKK, where “x” is Ile. In some cases, a variant 4-1BBL polypeptide of the present disclosure comprises the amino acid sequence set forth in FIG. 2KKK, where “x” is Pro. In some cases, a variant 4-1BBL polypeptide of the present disclosure comprises the amino acid sequence set forth in FIG. 2KKK, where “x” is Phe. In some cases, a variant 4-1BBL polypeptide of the present disclosure comprises the amino acid sequence set forth in FIG. 2KKK, where “x” is Tyr. In some cases, a variant 4-1BBL polypeptide of the present disclosure comprises the amino acid sequence set forth in FIG. 2KKK, where “x” is Trp. In some cases, a variant 4-1BBL polypeptide of the present disclosure comprises the amino acid sequence set forth in FIG. 2KKK, where “x” is Ser. In some cases, a variant 4-1BBL polypeptide of the present disclosure comprises the amino acid sequence set forth in FIG. 2KKK, where “x” is Thr. In some cases, a variant 4-1BBL polypeptide of the present disclosure comprises the amino acid sequence set forth in FIG. 2KKK, where “x” is Cys. In some cases, a variant 4-1BBL polypeptide of the present disclosure comprises the amino acid sequence set forth in FIG. 2KKK, where “x” is Met. In some cases, a variant 4-1BBL polypeptide of the present disclosure comprises the amino acid sequence set forth in FIG. 2KKK, where “x” is Asn. In some cases, a variant 4-1BBL polypeptide of the present disclosure comprises the amino acid sequence set forth in FIG. 2KKK, where “x” is Gln. In some cases, a variant 4-1BBL polypeptide of the present disclosure comprises the amino acid sequence set forth in FIG. 2KKK, where “x” is Lys. In some cases, a variant 4-1BBL polypeptide of the present disclosure comprises the amino acid sequence set forth in FIG. 2KKK, where “x” is Arg. In some cases, a variant 4-1BBL polypeptide of the present disclosure comprises the amino acid sequence set forth in FIG. 2KKK, where “x” is His. In some cases, a variant 4-1BBL polypeptide of the present disclosure comprises the amino acid sequence set forth in FIG. 2KKK, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R193 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 (indicated by an “x”) is an amino acid other than an arginine, e.g., where amino acid 155 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2LLL retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2LLL, where amino acid 193 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R193. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at R113. In some cases, a variant 4-1BBL polypeptide of the present disclosure comprises the amino acid sequence set forth in SEQ ID NO:1, with an amino acid substitution at R113. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R114. In some cases, a variant 4-1BBL polypeptide of the present disclosure comprises the amino acid sequence set forth in SEQ ID NO:2, with an amino acid substitution at R114. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R114. In some cases, a variant 4-1BBL polypeptide of the present disclosure comprises the amino acid sequence set forth in SEQ ID NO:3, with an amino acid substitution at R114.

For example, in some cases, a variant 4-1BBL polypeptide of the present disclosure comprises the amino acid sequence set forth in FIG. 2LLL, where “x” is any amino acid other than arginine; for example, “x” can be Gly, Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2LLL, where “x” is Gly. In some cases, a variant 4-1BBL polypeptide of the present disclosure comprises the amino acid sequence set forth in FIG. 2LLL, where “x” is Ala. In some cases, a variant 4-1BBL polypeptide of the present disclosure comprises the amino acid sequence set forth in FIG. 2LLL, where “x” is Val. In some cases, a variant 4-1BBL polypeptide of the present disclosure comprises the amino acid sequence set forth in FIG. 2LLL, where “x” is Leu. In some cases, a variant 4-1BBL polypeptide of the present disclosure comprises the amino acid sequence set forth in FIG. 2LLL, where “x” is Ile. In some cases, a variant 4-1BBL polypeptide of the present disclosure comprises the amino acid sequence set forth in FIG. 2LLL, where “x” is Pro. In some cases, a variant 4-1BBL polypeptide of the present disclosure comprises the amino acid sequence set forth in FIG. 2LLL, where “x” is Phe. In some cases, a variant 4-1BBL polypeptide of the present disclosure comprises the amino acid sequence set forth in FIG. 2LLL, where “x” is Tyr. In some cases, a variant 4-1BBL polypeptide of the present disclosure comprises the amino acid sequence set forth in FIG. 2LLL, where “x” is Trp. In some cases, a variant 4-1BBL polypeptide of the present disclosure comprises the amino acid sequence set forth in FIG. 2LLL, where “x” is Ser. In some cases, a variant 4-1BBL polypeptide of the present disclosure comprises the amino acid sequence set forth in FIG. 2LLL, where “x” is Thr. In some cases, a variant 4-1BBL polypeptide of the present disclosure comprises the amino acid sequence set forth in FIG. 2LLL, where “x” is Cys. In some cases, a variant 4-1BBL polypeptide of the present disclosure comprises the amino acid sequence set forth in FIG. 2LLL, where “x” is Met. In some cases, a variant 4-1BBL polypeptide of the present disclosure comprises the amino acid sequence set forth in FIG. 2LLL, where “x” is Asn. In some cases, a variant 4-1BBL polypeptide of the present disclosure comprises the amino acid sequence set forth in FIG. 2LLL, where “x” is Gln. In some cases, a variant 4-1BBL polypeptide of the present disclosure comprises the amino acid sequence set forth in FIG. 2LLL, where “x” is Lys. In some cases, a variant 4-1BBL polypeptide of the present disclosure comprises the amino acid sequence set forth in FIG. 2LLL, where “x” is His. In some cases, a variant 4-1BBL polypeptide of the present disclosure comprises the amino acid sequence set forth in FIG. 2LLL, where “x” is Asp. In some cases, a variant 4-1BBL polypeptide of the present disclosure comprises the amino acid sequence set forth in FIG. 2LLL, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### N194 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 (indicated by an “x”) is an amino acid other than a asparagine, e.g., where amino acid 194 is Gly, Ala, Val, Leu, Ile, Pro, Phe, Tyr, Trp, Ser, Thr, Cys, Met, Gln, Lys, Arg, His, Asp, or Glu. The amino acid numbering in FIG. 2MMM retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2MMM, where amino acid 194 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at N194. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO: 1, with an amino acid substitution at N114. In some cases, a variant 4-1BBL polypeptide of the present disclosure comprises the amino acid sequence set forth in SEQ ID NO: 1, with an amino acid substitution at N114. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at N115. In some cases, a variant 4-1BBL polypeptide of the present disclosure comprises the amino acid sequence set forth in SEQ ID NO:2, with an amino acid substitution at N115. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at N115. In some cases, a variant 4-1BBL polypeptide of the present disclosure comprises the amino acid sequence set forth in SEQ ID NO:3, with an amino acid substitution at N115.

For example, in some cases, a variant 4-1BBL polypeptide of the present disclosure comprises the amino acid sequence set forth in FIG. 2MMM, where “x” is any amino acid other than asparagine; for example, “x” can be Gly, Ala, Val, Leu, Ile, Pro, Phe, Tyr, Trp, Ser, Thr, Cys, Met, Gln, Lys, Arg, His, Asp, or Glu. In some cases, a variant 4-1BBL polypeptide of the present disclosure comprises the amino acid sequence set forth in FIG. 2MMM, where “x” is Gly. In some cases, a variant 4-1BBL polypeptide of the present disclosure comprises the amino acid sequence set forth in FIG. 2MMM, where “x” is Ala. In some cases, a variant 4-1BBL polypeptide of the present disclosure comprises the amino acid sequence set forth in FIG. 2MMM, where “x” is Val. In some cases, a variant 4-1BBL polypeptide of the present disclosure comprises the amino acid sequence set forth in FIG. 2MMM, where “x” is Leu. In some cases, a variant 4-1BBL polypeptide of the present disclosure comprises the amino acid sequence set forth in FIG. 2MMM, where “x” is Ile. In some cases, a variant 4-1BBL polypeptide of the present disclosure comprises the amino acid sequence set forth in FIG. 2MMM, where “x” is Pro. In some cases, a variant 4-1BBL polypeptide of the present disclosure comprises the amino acid sequence set forth in FIG. 2MMM, where “x” is Phe. In some cases, a variant 4-1BBL polypeptide of the present disclosure comprises the amino acid sequence set forth in FIG. 2MMM, where “x” is Tyr. In some cases, a variant 4-1BBL polypeptide of the present disclosure comprises the amino acid sequence set forth in FIG. 2MMM, where “x” is Trp. In some cases, a variant 4-1BBL polypeptide of the present disclosure comprises the amino acid sequence set forth in FIG. 2MMM, where “x” is Ser. In some cases, a variant 4-1BBL polypeptide of the present disclosure comprises the amino acid sequence set forth in FIG. 2MMM, where “x” is Thr. In some cases, a variant 4-1BBL polypeptide of the present disclosure comprises the amino acid sequence set forth in FIG. 2MMM, where “x” is Cys. In some cases, a variant 4-1BBL polypeptide of the present disclosure comprises the amino acid sequence set forth in FIG. 2MMM, where “x” is Met. In some cases, a variant 4-1BBL polypeptide of the present disclosure comprises the amino acid sequence set forth in FIG. 2MMM, where “x” is Gln. In some cases, a variant 4-1BBL polypeptide of the present disclosure comprises the amino acid sequence set forth in FIG. 2MMM, where “x” is Lys. In some cases, a variant 4-1BBL polypeptide of the present disclosure comprises the amino acid sequence set forth in FIG. 2MMM, where “x” is Arg. In some cases, a variant 4-1BBL polypeptide of the present disclosure comprises the amino acid sequence set forth in FIG. 2MMM, where “x” is His. In some cases, a variant 4-1BBL polypeptide of the present disclosure comprises the amino acid sequence set forth in FIG. 2MMM, where “x” is Asp. In some cases, a variant 4-1BBL polypeptide of the present disclosure comprises the amino acid sequence set forth in FIG. 2MMM, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### S195 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 (indicated by an “x”) is an amino acid other than a serine, e.g., where amino acid 195 is Gly, Ala, Val, Leu, Ile, Pro, Phe, Tyr, Trp, Thr, Cys, Met, Asn, Gln, Lys, Arg, His, Asp, or Glu. The amino acid numbering in FIG. 2NNN retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2NNN, where amino acid 195 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at S195. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at S115. In some cases, a variant 4-1BBL polypeptide of the present disclosure comprises the amino acid sequence set forth in SEQ ID NO:1, with an amino acid substitution at S115. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S116. In some cases, a variant 4-1BBL polypeptide of the present disclosure comprises the amino acid sequence set forth in SEQ ID NO:2, with an amino acid substitution at S116. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S116. In some cases, a variant 4-1BBL polypeptide of the present disclosure comprises the amino acid sequence set forth in SEQ ID NO:3, with an amino acid substitution at S116.

For example, in some cases, a variant 4-1BBL polypeptide of the present disclosure comprises the amino acid sequence set forth in FIG. 2NNN, where “x” is any amino acid other than serine; for example, “x” can be Gly, Ala, Val, Leu, Ile, Pro, Phe, Tyr, Trp, Thr, Cys, Met, Asn, Gln, Lys, Arg, His, Asp, or Glu. In some cases, a variant 4-1BBL polypeptide of the present disclosure comprises the amino acid sequence set forth in FIG. 2NNN, where “x” is Gly. In some cases, a variant 4-1BBL polypeptide of the present disclosure comprises the amino acid sequence set forth in FIG. 2NNN, where “x” is Ala. In some cases, a variant 4-1BBL polypeptide of the present disclosure comprises the amino acid sequence set forth in FIG. 2NNN, where “x” is Val. In some cases, a variant 4-1BBL polypeptide of the present disclosure comprises the amino acid sequence set forth in FIG. 2NNN, where “x” is Leu. In some cases, a variant 4-1BBL polypeptide of the present disclosure comprises the amino acid sequence set forth in FIG. 2NNN, where “x” is Ile. In some cases, a variant 4-1BBL polypeptide of the present disclosure comprises the amino acid sequence set forth in FIG. 2NNN, where “x” is Pro. In some cases, a variant 4-1BBL polypeptide of the present disclosure comprises the amino acid sequence set forth in FIG. 2NNN, where “x” is Phe. In some cases, a variant 4-1BBL polypeptide of the present disclosure comprises the amino acid sequence set forth in FIG. 2NNN, where “x” is Tyr. In some cases, a variant 4-1BBL polypeptide of the present disclosure comprises the amino acid sequence set forth in FIG. 2NNN, where “x” is Trp. In some cases, a variant 4-1BBL polypeptide of the present disclosure comprises the amino acid sequence set forth in FIG. 2NNN, where “x” is Thr. In some cases, a variant 4-1BBL polypeptide of the present disclosure comprises the amino acid sequence set forth in FIG. 2NNN, where “x” is Cys. In some cases, a variant 4-1BBL polypeptide of the present disclosure comprises the amino acid sequence set forth in FIG. 2NNN, where “x” is Met. In some cases, a variant 4-1BBL polypeptide of the present disclosure comprises the amino acid sequence set forth in FIG. 2NNN, where “x” is Asn. In some cases, a variant 4-1BBL polypeptide of the present disclosure comprises the amino acid sequence set forth in FIG. 2NNN, where “x” is Gln. In some cases, a variant 4-1BBL polypeptide of the present disclosure comprises the amino acid sequence set forth in FIG. 2NNN, where “x” is Lys. In some cases, a variant 4-1BBL polypeptide of the present disclosure comprises the amino acid sequence set forth in FIG. 2NNN, where “x” is Arg. In some cases, a variant 4-1BBL polypeptide of the present disclosure comprises the amino acid sequence set forth in FIG. 2NNN, where “x” is His. In some cases, a variant 4-1BBL polypeptide of the present disclosure comprises the amino acid sequence set forth in FIG. 2NNN, where “x” is Asp. In some cases, a variant 4-1BBL polypeptide of the present disclosure comprises the amino acid sequence set forth in FIG. 2NNN, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### F197 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 (indicated by an “x”) is an amino acid other than a phenylalanine, e.g., where amino acid 197 is Gly, Ala, Val, Leu, Ile, Pro, Tyr, Trp, Ser, Thr, Cys, Met, Asn, Gln, Lys, Arg, His, Asp, or Glu. The amino acid numbering in FIG. 2OOO retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2OOO, where amino acid 197 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at F197. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at F117. In some cases, a variant 4-1BBL polypeptide of the present disclosure comprises the amino acid sequence set forth in SEQ ID NO: 1, with an amino acid substitution at F117. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F118. In some cases, a variant 4-1BBL polypeptide of the present disclosure comprises the amino acid sequence set forth in SEQ ID NO:2, with an amino acid substitution at F118. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F118. In some cases, a variant 4-1BBL polypeptide of the present disclosure comprises the amino acid sequence set forth in SEQ ID NO:3, with an amino acid substitution at F118.

For example, in some cases, a variant 4-1BBL polypeptide of the present disclosure comprises the amino acid sequence set forth in FIG. 2OOO, where “x” is any amino acid other than phenylalanine; for example, “x” can be Gly, Ala, Val, Leu, Ile, Pro, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2OOO, where “x” is Gly. In some cases, a variant 4-1BBL polypeptide of the present disclosure comprises the amino acid sequence set forth in FIG. 2OOO, where “x” is Ala. In some cases, a variant 4-1BBL polypeptide of the present disclosure comprises the amino acid sequence set forth in FIG. 2OOO, where “x” is Val. In some cases, a variant 4-1BBL polypeptide of the present disclosure comprises the amino acid sequence set forth in FIG. 2OOO, where “x” is Leu. In some cases, a variant 4-1BBL polypeptide of the present disclosure comprises the amino acid sequence set forth in FIG. 2OOO, where “x” is Ile. In some cases, a variant 4-1BBL polypeptide of the present disclosure comprises the amino acid sequence set forth in FIG. 2OOO, where “x” is Pro. In some cases, a variant 4-1BBL polypeptide of the present disclosure comprises the amino acid sequence set forth in FIG. 2OOO, where “x” is Tyr. In some cases, a variant 4-1BBL polypeptide of the present disclosure comprises the amino acid sequence set forth in FIG. 2OOO, where “x” is Trp. In some cases, a variant 4-1BBL polypeptide of the present disclosure comprises the amino acid sequence set forth in FIG. 2OOO, where “x” is Ser. In some cases, a variant 4-1BBL polypeptide of the present disclosure comprises the amino acid sequence set forth in FIG. 2OOO, where “x” is Thr. In some cases, a variant 4-1BBL polypeptide of the present disclosure comprises the amino acid sequence set forth in FIG. 2OOO, where “x” is Cys. In some cases, a variant 4-1BBL polypeptide of the present disclosure comprises the amino acid sequence set forth in FIG. 2OOO, where “x” is Met. In some cases, a variant 4-1BBL polypeptide of the present disclosure comprises the amino acid sequence set forth in FIG. 2OOO, where “x” is Asn. In some cases, a variant 4-1BBL polypeptide of the present disclosure comprises the amino acid sequence set forth in FIG. 2OOO, where “x” is Gln. In some cases, a variant 4-1BBL polypeptide of the present disclosure comprises the amino acid sequence set forth in FIG. 2OOO, where “x” is Lys. In some cases, a variant 4-1BBL polypeptide of the present disclosure comprises the amino acid sequence set forth in FIG. 2OOO, where “x” is Arg. In some cases, a variant 4-1BBL polypeptide of the present disclosure comprises the amino acid sequence set forth in FIG. 2OOO, where “x” is His. In some cases, a variant 4-1BBL polypeptide of the present disclosure comprises the amino acid sequence set forth in FIG. 2OOO, where “x” is Asp. In some cases, a variant 4-1BBL polypeptide of the present disclosure comprises the amino acid sequence set forth in FIG. 2OOO, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q210 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 (indicated by an “x”) is an amino acid other than a glutamine, e.g., where amino acid 210 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2PPP retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2PPP, where amino acid 210 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q210. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at Q130. In some cases, a variant 4-1BBL polypeptide of the present disclosure comprises the amino acid sequence set forth in SEQ ID NO:1, with an amino acid substitution at Q130. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q131. In some cases, a variant 4-1BBL polypeptide of the present disclosure comprises the amino acid sequence set forth in SEQ ID NO:2, with an amino acid substitution at Q131. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q131. In some cases, a variant 4-1BBL polypeptide of the present disclosure comprises the amino acid sequence set forth in SEQ ID NO:3, with an amino acid substitution at Q131.

For example, in some cases, a variant 4-1BBL polypeptide of the present disclosure comprises the amino acid sequence set forth in FIG. 2PPP, where “x” is any amino acid other than methionine; for example, “x” can be Gly, Ala, Val, Leu, Ile, Pro, Phe, Tyr, Trp, Ser, Thr, Cys, Met, Asn, Lys, Arg, His, Asp, or Glu. In some cases, a variant 4-1BBL polypeptide of the present disclosure comprises the amino acid sequence set forth in FIG. 2PPP, where “x” is Gly. In some cases, a variant 4-1BBL polypeptide of the present disclosure comprises the amino acid sequence set forth in FIG. 2PPP, where “x” is Ala. In some cases, a variant 4-1BBL polypeptide of the present disclosure comprises the amino acid sequence set forth in FIG. 2PPP, where “x” is Val. In some cases, a variant 4-1BBL polypeptide of the present disclosure comprises the amino acid sequence set forth in FIG. 2PPP, where “x” is Leu. In some cases, a variant 4-1BBL polypeptide of the present disclosure comprises the amino acid sequence set forth in FIG. 2PPP, where “x” is Ile. In some cases, a variant 4-1BBL polypeptide of the present disclosure comprises the amino acid sequence set forth in FIG. 2PPP, where “x” is Pro. In some cases, a variant 4-1BBL polypeptide of the present disclosure comprises the amino acid sequence set forth in FIG. 2PPP, where “x” is Phe. In some cases, a variant 4-1BBL polypeptide of the present disclosure comprises the amino acid sequence set forth in FIG. 2PPP, where “x” is Tyr. In some cases, a variant 4-1BBL polypeptide of the present disclosure comprises the amino acid sequence set forth in FIG. 2PPP, where “x” is Trp. In some cases, a variant 4-1BBL polypeptide of the present disclosure comprises the amino acid sequence set forth in FIG. 2PPP, where “x” is Ser. In some cases, a variant 4-1BBL polypeptide of the present disclosure comprises the amino acid sequence set forth in FIG. 2PPP, where “x” is Thr. In some cases, a variant 4-1BBL polypeptide of the present disclosure comprises the amino acid sequence set forth in FIG. 2PPP, where “x” is Cys. In some cases, a variant 4-1BBL polypeptide of the present disclosure comprises the amino acid sequence set forth in FIG. 2PPP, where “x” is Met. In some cases, a variant 4-1BBL polypeptide of the present disclosure comprises the amino acid sequence set forth in FIG. 2PPP, where “x” is Asn. In some cases, a variant 4-1BBL polypeptide of the present disclosure comprises the amino acid sequence set forth in FIG. 2PPP, where “x” is Lys. In some cases, a variant 4-1BBL polypeptide of the present disclosure comprises the amino acid sequence set forth in FIG. 2PPP, where “x” is Arg. In some cases, a variant 4-1BBL polypeptide of the present disclosure comprises the amino acid sequence set forth in FIG. 2PPP, where “x” is His. In some cases, a variant 4-1BBL polypeptide of the present disclosure comprises the amino acid sequence set forth in FIG. 2PPP, where “x” is Asp. In some cases, a variant 4-1BBL polypeptide of the present disclosure comprises the amino acid sequence set forth in FIG. 2PPP, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R211 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 (indicated by an “x”) is an amino acid other than an arginine, e.g., where amino acid 211 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2QQQ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R211. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at R131. In some cases, a variant 4-1BBL polypeptide of the present disclosure comprises the amino acid sequence set forth in SEQ ID NO:1, with an amino acid substitution at R131. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R132. In some cases, a variant 4-1BBL polypeptide of the present disclosure comprises the amino acid sequence set forth in SEQ ID NO:2, with an amino acid substitution at R132. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R132. In some cases, a variant 4-1BBL polypeptide of the present disclosure comprises the amino acid sequence set forth in SEQ ID NO:3, with an amino acid substitution at R132.

For example, in some cases, a variant 4-1BBL polypeptide of the present disclosure comprises the amino acid sequence set forth in FIG. 2QQQ, where “x” is any amino acid other than arginine; for example, “x” can be Gly, Ala, Val, Leu, Ile, Pro, Phe, Tyr, Trp, Ser, Thr, Cys, Met, Asn, Gln, Lys, His, Asp, or Glu. In some cases, a variant 4-1BBL polypeptide of the present disclosure comprises the amino acid sequence set forth in FIG. 2QQQ, where “x” is Gly. In some cases, a variant 4-1BBL polypeptide of the present disclosure comprises the amino acid sequence set forth in FIG. 2QQQ, where “x” is Ala. In some cases, a variant 4-1BBL polypeptide of the present disclosure comprises the amino acid sequence set forth in FIG. 2QQQ, where “x” is Val. In some cases, a variant 4-1BBL polypeptide of the present disclosure comprises the amino acid sequence set forth in FIG. 2QQQ, where “x” is Leu. In some cases, a variant 4-1BBL polypeptide of the present disclosure comprises the amino acid sequence set forth in FIG. 2QQQ, where “x” is Ile. In some cases, a variant 4-1BBL polypeptide of the present disclosure comprises the amino acid sequence set forth in FIG. 2QQQ, where “x” is Pro. In some cases, a variant 4-1BBL polypeptide of the present disclosure comprises the amino acid sequence set forth in FIG. 2QQQ, where “x” is Phe. In some cases, a variant 4-1BBL polypeptide of the present disclosure comprises the amino acid sequence set forth in FIG. 2QQQ, where “x” is Tyr. In some cases, a variant 4-1BBL polypeptide of the present disclosure comprises the amino acid sequence set forth in FIG. 2QQQ, where “x” is Trp. In some cases, a variant 4-1BBL polypeptide of the present disclosure comprises the amino acid sequence set forth in FIG. 2QQQ, where “x” is Ser. In some cases, a variant 4-1BBL polypeptide of the present disclosure comprises the amino acid sequence set forth in FIG. 2QQQ, where “x” is Thr. In some cases, a variant 4-1BBL polypeptide of the present disclosure comprises the amino acid sequence set forth in FIG. 2QQQ, where “x” is Cys. In some cases, a variant 4-1BBL polypeptide of the present disclosure comprises the amino acid sequence set forth in FIG. 2QQQ, where “x” is Met. In some cases, a variant 4-1BBL polypeptide of the present disclosure comprises the amino acid sequence set forth in FIG. 2QQQ, where “x” is Asn. In some cases, a variant 4-1BBL polypeptide of the present disclosure comprises the amino acid sequence set forth in FIG. 2QQQ, where “x” is Gln. In some cases, a variant 4-1BBL polypeptide of the present disclosure comprises the amino acid sequence set forth in FIG. 2QQQ, where “x” is Lys. In some cases, a variant 4-1BBL polypeptide of the present disclosure comprises the amino acid sequence set forth in FIG. 2QQQ, where “x” is His. In some cases, a variant 4-1BBL polypeptide of the present disclosure comprises the amino acid sequence set forth in FIG. 2QQQ, where “x” is Asp. In some cases, a variant 4-1BBL polypeptide of the present disclosure comprises the amino acid sequence set forth in FIG. 2QQQ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L212 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 (indicated by an “x”) is an amino acid other than a leucine, e.g., where amino acid 212 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2RRR retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2RRR, where amino acid 212 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L212. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L132. In some cases, a variant 4-1BBL polypeptide of the present disclosure comprises the amino acid sequence set forth in SEQ ID NO:1, with an amino acid substitution at L132. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L133. In some cases, a variant 4-1BBL polypeptide of the present disclosure comprises the amino acid sequence set forth in SEQ ID NO:2, with an amino acid substitution at L133. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L133. In some cases, a variant 4-1BBL polypeptide of the present disclosure comprises the amino acid sequence set forth in SEQ ID NO:3, with an amino acid substitution at L133.

For example, in some cases, a variant 4-1BBL polypeptide of the present disclosure comprises the amino acid sequence set forth in FIG. 2RRR, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2RRR, where “x” is Gly. In some cases, a variant 4-1BBL polypeptide of the present disclosure comprises the amino acid sequence set forth in FIG. 2RRR, where “x” is Ala. In some cases, a variant 4-1BBL polypeptide of the present disclosure comprises the amino acid sequence set forth in FIG. 2RRR, where “x” is Val. In some cases, a variant 4-1BBL polypeptide of the present disclosure comprises the amino acid sequence set forth in FIG. 2RRR, where “x” is Ile. In some cases, a variant 4-1BBL polypeptide of the present disclosure comprises the amino acid sequence set forth in FIG. 2RRR, where “x” is Pro. In some cases, a variant 4-1BBL polypeptide of the present disclosure comprises the amino acid sequence set forth in FIG. 2RRR, where “x” is Phe. In some cases, a variant 4-1BBL polypeptide of the present disclosure comprises the amino acid sequence set forth in FIG. 2RRR, where “x” is Tyr. In some cases, a variant 4-1BBL polypeptide of the present disclosure comprises the amino acid sequence set forth in FIG. 2RRR, where “x” is Trp. In some cases, a variant 4-1BBL polypeptide of the present disclosure comprises the amino acid sequence set forth in FIG. 2RRR, where “x” is Ser. In some cases, a variant 4-1BBL polypeptide of the present disclosure comprises the amino acid sequence set forth in FIG. 2RRR, where “x” is Thr. In some cases, a variant 4-1BBL polypeptide of the present disclosure comprises the amino acid sequence set forth in FIG. 2RRR, where “x” is Cys. In some cases, a variant 4-1BBL polypeptide of the present disclosure comprises the amino acid sequence set forth in FIG. 2RRR, where “x” is Met. In some cases, a variant 4-1BBL polypeptide of the present disclosure comprises the amino acid sequence set forth in FIG. 2RRR, where “x” is Asn. In some cases, a variant 4-1BBL polypeptide of the present disclosure comprises the amino acid sequence set forth in FIG. 2RRR, where “x” is Gln. In some cases, a variant 4-1BBL polypeptide of the present disclosure comprises the amino acid sequence set forth in FIG. 2RRR, where “x” is Lys. In some cases, a variant 4-1BBL polypeptide of the present disclosure comprises the amino acid sequence set forth in FIG. 2RRR, where “x” is Arg. In some cases, a variant 4-1BBL polypeptide of the present disclosure comprises the amino acid sequence set forth in FIG. 2RRR, where “x” is His. In some cases, a variant 4-1BBL polypeptide of the present disclosure comprises the amino acid sequence set forth in FIG. 2RRR, where “x” is Asp. In some cases, a variant 4-1BBL polypeptide of the present disclosure comprises the amino acid sequence set forth in FIG. 2RRR, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G213 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 (indicated by an “x”) is an amino acid other than a glycine, e.g., where amino acid 213 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2SSS retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2SSS, where amino acid 213 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G213. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at G133. In some cases, a variant 4-1BBL polypeptide of the present disclosure comprises the amino acid sequence set forth in SEQ ID NO:1, with an amino acid substitution at G133. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G134. In some cases, a variant 4-1BBL polypeptide of the present disclosure comprises the amino acid sequence set forth in SEQ ID NO:2, with an amino acid substitution at G134. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G134. In some cases, a variant 4-1BBL polypeptide of the present disclosure comprises the amino acid sequence set forth in SEQ ID NO:3, with an amino acid substitution at G134.

For example, in some cases, a variant 4-1BBL polypeptide of the present disclosure comprises the amino acid sequence set forth in FIG. 2SSS, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2SSS, where “x” is Ala. In some cases, a variant 4-1BBL polypeptide of the present disclosure comprises the amino acid sequence set forth in FIG. 2SSS, where “x” is Val. In some cases, a variant 4-1BBL polypeptide of the present disclosure comprises the amino acid sequence set forth in FIG. 2SSS, where “x” is Leu. In some cases, a variant 4-1BBL polypeptide of the present disclosure comprises the amino acid sequence set forth in FIG. 2SSS, where “x” is Ile. In some cases, a variant 4-1BBL polypeptide of the present disclosure comprises the amino acid sequence set forth in FIG. 2SSS, where “x” is Pro. In some cases, a variant 4-1BBL polypeptide of the present disclosure comprises the amino acid sequence set forth in FIG. 2SSS, where “x” is Phe. In some cases, a variant 4-1BBL polypeptide of the present disclosure comprises the amino acid sequence set forth in FIG. 2SSS, where “x” is Tyr. In some cases, a variant 4-1BBL polypeptide of the present disclosure comprises the amino acid sequence set forth in FIG. 2SSS, where “x” is Trp. In some cases, a variant 4-1BBL polypeptide of the present disclosure comprises the amino acid sequence set forth in FIG. 2Q, where “x” is Ser. In some cases, a variant 4-1BBL polypeptide of the present disclosure comprises the amino acid sequence set forth in FIG. 2SSS, where “x” is Thr. In some cases, a variant 4-1BBL polypeptide of the present disclosure comprises the amino acid sequence set forth in FIG. 2SSS, where “x” is Cys. In some cases, a variant 4-1BBL polypeptide of the present disclosure comprises the amino acid sequence set forth in FIG. 2SSS, where “x” is Met. In some cases, a variant 4-1BBL polypeptide of the present disclosure comprises the amino acid sequence set forth in FIG. 2SSS, where “x” is Asn. In some cases, a variant 4-1BBL polypeptide of the present disclosure comprises the amino acid sequence set forth in FIG. 2SSS, where “x” is Gln. In some cases, a variant 4-1BBL polypeptide of the present disclosure comprises the amino acid sequence set forth in FIG. 2SSS, where “x” is Lys. In some cases, a variant 4-1BBL polypeptide of the present disclosure comprises the amino acid sequence set forth in FIG. 2SSS, where “x” is Arg. In some cases, a variant 4-1BBL polypeptide of the present disclosure comprises the amino acid sequence set forth in FIG. 2SSS, where “x” is His. In some cases, a variant 4-1BBL polypeptide of the present disclosure comprises the amino acid sequence set forth in FIG. 2SSS, where “x” is Asp. In some cases, a variant 4-1BBL polypeptide of the present disclosure comprises the amino acid sequence set forth in FIG. 2SSS, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V214 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 (indicated by an “x”) is an amino acid other than a valine, e.g., where amino acid 214 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2TTT retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2TTT, where amino acid 214 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V214. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at V134. In some cases, a variant 4-1BBL polypeptide of the present disclosure comprises the amino acid sequence set forth in SEQ ID NO:1, with an amino acid substitution at V134. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V135. In some cases, a variant 4-1BBL polypeptide of the present disclosure comprises the amino acid sequence set forth in SEQ ID NO:2, with an amino acid substitution at V135. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V135. In some cases, a variant 4-1BBL polypeptide of the present disclosure comprises the amino acid sequence set forth in SEQ ID NO:3, with an amino acid substitution at V135.

For example, in some cases, a variant 4-1BBL polypeptide of the present disclosure comprises the amino acid sequence set forth in FIG. 2TTT, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2TTT, where “x” is Gly. In some cases, a variant 4-1BBL polypeptide of the present disclosure comprises the amino acid sequence set forth in FIG. 2TTT, where “x” is Ala. In some cases, a variant 4-1BBL polypeptide of the present disclosure comprises the amino acid sequence set forth in FIG. 2TTT, where “x” is Leu. In some cases, a variant 4-1BBL polypeptide of the present disclosure comprises the amino acid sequence set forth in FIG. 2TTT, where “x” is Ile. In some cases, a variant 4-1BBL polypeptide of the present disclosure comprises the amino acid sequence set forth in FIG. 2TTT, where “x” is Pro. In some cases, a variant 4-1BBL polypeptide of the present disclosure comprises the amino acid sequence set forth in FIG. 2TTT, where “x” is Phe. In some cases, a variant 4-1BBL polypeptide of the present disclosure comprises the amino acid sequence set forth in FIG. 2TTT, where “x” is Tyr. In some cases, a variant 4-1BBL polypeptide of the present disclosure comprises the amino acid sequence set forth in FIG. 2TTT, where “x” is Trp. In some cases, a variant 4-1BBL polypeptide of the present disclosure comprises the amino acid sequence set forth in FIG. 2TTT, where “x” is Ser. In some cases, a variant 4-1BBL polypeptide of the present disclosure comprises the amino acid sequence set forth in FIG. 2TTT, where “x” is Thr. In some cases, a variant 4-1BBL polypeptide of the present disclosure comprises the amino acid sequence set forth in FIG. 2TTT, where “x” is Cys. In some cases, a variant 4-1BBL polypeptide of the present disclosure comprises the amino acid sequence set forth in FIG. 2TTT, where “x” is Met. In some cases, a variant 4-1BBL polypeptide of the present disclosure comprises the amino acid sequence set forth in FIG. 2TTT, where “x” is Asn. In some cases, a variant 4-1BBL polypeptide of the present disclosure comprises the amino acid sequence set forth in FIG. 2TTT, where “x” is Gln. In some cases, a variant 4-1BBL polypeptide of the present disclosure comprises the amino acid sequence set forth in FIG. 2TTT, where “x” is Lys. In some cases, a variant 4-1BBL polypeptide of the present disclosure comprises the amino acid sequence set forth in FIG. 2TTT, where “x” is Arg. In some cases, a variant 4-1BBL polypeptide of the present disclosure comprises the amino acid sequence set forth in FIG. 2TTT, where “x” is His. In some cases, a variant 4-1BBL polypeptide of the present disclosure comprises the amino acid sequence set forth in FIG. 2TTT, where “x” is Asp. In some cases, a variant 4-1BBL polypeptide of the present disclosure comprises the amino acid sequence set forth in FIG. 2TTT, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### H215 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 (indicated by an “x”) is an amino acid other than a histidine, e.g., where amino acid 215 is Gly, Ala, Val, Leu, Ile, Pro, Phe, Tyr, Trp, Ser, Thr, Cys, Met, Asn, Gln, Lys, Arg, Asp, or Glu. The amino acid numbering in FIG. 2UUU retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2UUU, where amino acid 215 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at H215. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at H135. In some cases, a variant 4-1BBL polypeptide of the present disclosure comprises the amino acid sequence set forth in SEQ ID NO:1, with an amino acid substitution at H135. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H136. In some cases, a variant 4-1BBL polypeptide of the present disclosure comprises the amino acid sequence set forth in SEQ ID NO:2, with an amino acid substitution at H136. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H136. In some cases, a variant 4-1BBL polypeptide of the present disclosure comprises the amino acid sequence set forth in SEQ ID NO:3, with an amino acid substitution at H136.

For example, in some cases, a variant 4-1BBL polypeptide of the present disclosure comprises the amino acid sequence set forth in FIG. 2UUU, where “x” is any amino acid other than histidine; for example, “x” can be Gly, Ala, Val, Leu, Ile, Pro, Phe, Tyr, Trp, Ser, Thr, Cys, Met, Asn, Gln, Lys, Arg, Asp, or Glu. In some cases, a variant 4-1BBL polypeptide of the present disclosure comprises the amino acid sequence set forth in FIG. 2UUU, where “x” is Gly. In some cases, a variant 4-1BBL polypeptide of the present disclosure comprises the amino acid sequence set forth in FIG. 2UUU, where “x” is Ala. In some cases, a variant 4-1BBL polypeptide of the present disclosure comprises the amino acid sequence set forth in FIG. 2UUU, where “x” is Val. In some cases, a variant 4-1BBL polypeptide of the present disclosure comprises the amino acid sequence set forth in FIG. 2UUU, where “x” is Leu. In some cases, a variant 4-1BBL polypeptide of the present disclosure comprises the amino acid sequence set forth in FIG. 2UUU, where “x” is Ile. In some cases, a variant 4-1BBL polypeptide of the present disclosure comprises the amino acid sequence set forth in FIG. 2UUU, where “x” is Pro. In some cases, a variant 4-1BBL polypeptide of the present disclosure comprises the amino acid sequence set forth in FIG. 2UUU, where “x” is Phe. In some cases, a variant 4-1BBL polypeptide of the present disclosure comprises the amino acid sequence set forth in FIG. 2UUU, where “x” is Tyr. In some cases, a variant 4-1BBL polypeptide of the present disclosure comprises the amino acid sequence set forth in FIG. 2UUU, where “x” is Trp. In some cases, a variant 4-1BBL polypeptide of the present disclosure comprises the amino acid sequence set forth in FIG. 2UUU, where “x” is Ser. In some cases, a variant 4-1BBL polypeptide of the present disclosure comprises the amino acid sequence set forth in FIG. 2UUU, where “x” is Thr. In some cases, a variant 4-1BBL polypeptide of the present disclosure comprises the amino acid sequence set forth in FIG. 2UUU, where “x” is Cys. In some cases, a variant 4-1BBL polypeptide of the present disclosure comprises the amino acid sequence set forth in FIG. 2UUU, where “x” is Met. In some cases, a variant 4-1BBL polypeptide of the present disclosure comprises the amino acid sequence set forth in FIG. 2UUU, where “x” is Asn. In some cases, a variant 4-1BBL polypeptide of the present disclosure comprises the amino acid sequence set forth in FIG. 2UUU, where “x” is Gln. In some cases, a variant 4-1BBL polypeptide of the present disclosure comprises the amino acid sequence set forth in FIG. 2UUU, where “x” is Lys. In some cases, a variant 4-1BBL polypeptide of the present disclosure comprises the amino acid sequence set forth in FIG. 2UUU, where “x” is Asp. In some cases, a variant 4-1BBL polypeptide of the present disclosure comprises the amino acid sequence set forth in FIG. 2UUU, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L216 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 (indicated by an “x”) is an amino acid other than a leucine, e.g., where amino acid 216 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2VVV retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2VVV, where amino acid 216 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L216. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L136. In some cases, a variant 4-1BBL polypeptide of the present disclosure comprises the amino acid sequence set forth in SEQ ID NO:1, with an amino acid substitution at L136. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L137. In some cases, a variant 4-1BBL polypeptide of the present disclosure comprises the amino acid sequence set forth in SEQ ID NO:2, with an amino acid substitution at L137. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L137. In some cases, a variant 4-1BBL polypeptide of the present disclosure comprises the amino acid sequence set forth in SEQ ID NO:3, with an amino acid substitution at L137.

For example, in some cases, a variant 4-1BBL polypeptide of the present disclosure comprises the amino acid sequence set forth in FIG. 2VVV, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2VVV, where “x” is Gly. In some cases, a variant 4-1BBL polypeptide of the present disclosure comprises the amino acid sequence set forth in FIG. 2VVV, where “x” is Ala. In some cases, a variant 4-1BBL polypeptide of the present disclosure comprises the amino acid sequence set forth in FIG. 2VVV, where “x” is Val. In some cases, a variant 4-1BBL polypeptide of the present disclosure comprises the amino acid sequence set forth in FIG. 2VVV, where “x” is Ile. In some cases, a variant 4-1BBL polypeptide of the present disclosure comprises the amino acid sequence set forth in FIG. 2VVV, where “x” is Pro. In some cases, a variant 4-1BBL polypeptide of the present disclosure comprises the amino acid sequence set forth in FIG. 2VVV, where “x” is Phe. In some cases, a variant 4-1BBL polypeptide of the present disclosure comprises the amino acid sequence set forth in FIG. 2VVV, where “x” is Tyr. In some cases, a variant 4-1BBL polypeptide of the present disclosure comprises the amino acid sequence set forth in FIG. 2VVV, where “x” is Trp. In some cases, a variant 4-1BBL polypeptide of the present disclosure comprises the amino acid sequence set forth in FIG. 2VVV, where “x” is Ser. In some cases, a variant 4-1BBL polypeptide of the present disclosure comprises the amino acid sequence set forth in FIG. 2VVV, where “x” is Thr. In some cases, a variant 4-1BBL polypeptide of the present disclosure comprises the amino acid sequence set forth in FIG. 2VVV, where “x” is Cys. In some cases, a variant 4-1BBL polypeptide of the present disclosure comprises the amino acid sequence set forth in FIG. 2VVV, where “x” is Met. In some cases, a variant 4-1BBL polypeptide of the present disclosure comprises the amino acid sequence set forth in FIG. 2VVV, where “x” is Asn. In some cases, a variant 4-1BBL polypeptide of the present disclosure comprises the amino acid sequence set forth in FIG. 2VVV, where “x” is Gln. In some cases, a variant 4-1BBL polypeptide of the present disclosure comprises the amino acid sequence set forth in FIG. 2VVV, where “x” is Lys. In some cases, a variant 4-1BBL polypeptide of the present disclosure comprises the amino acid sequence set forth in FIG. 2VVV, where “x” is Arg. In some cases, a variant 4-1BBL polypeptide of the present disclosure comprises the amino acid sequence set forth in FIG. 2VVV, where “x” is His. In some cases, a variant 4-1BBL polypeptide of the present disclosure comprises the amino acid sequence set forth in FIG. 2VVV, where “x” is Asp. In some cases, a variant 4-1BBL polypeptide of the present disclosure comprises the amino acid sequence set forth in FIG. 2VVV, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### H217 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 (indicated by an “x”) is an amino acid other than a histidine, e.g., where amino acid 217 is Gly, Ala, Val, Leu, Ile, Pro, Phe, Tyr, Trp, Ser, Thr, Cys, Met, Asn, Gln, Lys, Arg, Asp, or Glu. The amino acid numbering in FIG. 2WWW retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2WWW, where amino acid 217 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at H217. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at H137. In some cases, a variant 4-1BBL polypeptide of the present disclosure comprises the amino acid sequence set forth in SEQ ID NO:1, with an amino acid substitution at H137. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H138. In some cases, a variant 4-1BBL polypeptide of the present disclosure comprises the amino acid sequence set forth in SEQ ID NO:2, with an amino acid substitution at H138. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H138. In some cases, a variant 4-1BBL polypeptide of the present disclosure comprises the amino acid sequence set forth in SEQ ID NO:3, with an amino acid substitution at H138.

For example, in some cases, a variant 4-1BBL polypeptide of the present disclosure comprises the amino acid sequence set forth in FIG. 2WWW, where “x” is any amino acid other than histidine; for example, “x” can be Gly, Ala, Val, Leu, Ile, Pro, Phe, Tyr, Trp, Ser, Thr, Cys, Met, Asn, Gln, Lys, Arg, Asp, or Glu. In some cases, a variant 4-1BBL polypeptide of the present disclosure comprises the amino acid sequence set forth in FIG. 2WWW, where “x” is Gly. In some cases, a variant 4-1BBL polypeptide of the present disclosure comprises the amino acid sequence set forth in FIG. 2WWW, where “x” is Ala. In some cases, a variant 4-1BBL polypeptide of the present disclosure comprises the amino acid sequence set forth in FIG. 2WWW, where “x” is Val. In some cases, a variant 4-1BBL polypeptide of the present disclosure comprises the amino acid sequence set forth in FIG. 2WWW, where “x” is Leu. In some cases, a variant 4-1BBL polypeptide of the present disclosure comprises the amino acid sequence set forth in FIG. 2WWW, where “x” is Ile. In some cases, a variant 4-1BBL polypeptide of the present disclosure comprises the amino acid sequence set forth in FIG. 2WWW, where “x” is Pro. In some cases, a variant 4-1BBL polypeptide of the present disclosure comprises the amino acid sequence set forth in FIG. 2WWW, where “x” is Phe. In some cases, a variant 4-1BBL polypeptide of the present disclosure comprises the amino acid sequence set forth in FIG. 2WWW, where “x” is Tyr. In some cases, a variant 4-1BBL polypeptide of the present disclosure comprises the amino acid sequence set forth in FIG. 2WWW, where “x” is Trp. In some cases, a variant 4-1BBL polypeptide of the present disclosure comprises the amino acid sequence set forth in FIG. 2WWW, where “x” is Ser. In some cases, a variant 4-1BBL polypeptide of the present disclosure comprises the amino acid sequence set forth in FIG. 2WWW, where “x” is Thr. In some cases, a variant 4-1BBL polypeptide of the present disclosure comprises the amino acid sequence set forth in FIG. 2WWW, where “x” is Cys. In some cases, a variant 4-1BBL polypeptide of the present disclosure comprises the amino acid sequence set forth in FIG. 2WWW, where “x” is Met. In some cases, a variant 4-1BBL polypeptide of the present disclosure comprises the amino acid sequence set forth in FIG. 2BB2WWW BB, where “x” is Asn. In some cases, a variant 4-1BBL polypeptide of the present disclosure comprises the amino acid sequence set forth in FIG. 2WWW, where “x” is Gln. In some cases, a variant 4-1BBL polypeptide of the present disclosure comprises the amino acid sequence set forth in FIG. 2WWW, where “x” is Lys. In some cases, a variant 4-1BBL polypeptide of the present disclosure comprises the amino acid sequence set forth in FIG. 2WWW, where “x” is Asp. In some cases, a variant 4-1BBL polypeptide of the present disclosure comprises the amino acid sequence set forth in FIG. 2WWW, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### T218 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 (indicated by an “x”) is an amino acid other than a threonine, e.g., where amino acid 218 is Gly, Ala, Val, Leu, Ile, Pro, Phe, Tyr, Trp, Ser, Cys, Met, Asn, Gln, Lys, Arg, His, Asp, or Glu. The amino acid numbering in FIG. 2XXX retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2XXX, where amino acid 218 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at T218. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at T138. In some cases, a variant 4-1BBL polypeptide of the present disclosure comprises the amino acid sequence set forth in SEQ ID NO:1, with an amino acid substitution at T138. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T139. In some cases, a variant 4-1BBL polypeptide of the present disclosure comprises the amino acid sequence set forth in SEQ ID NO:2, with an amino acid substitution at T139. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T139. In some cases, a variant 4-1BBL polypeptide of the present disclosure comprises the amino acid sequence set forth in SEQ ID NO:3, with an amino acid substitution at T139.

For example, in some cases, a variant 4-1BBL polypeptide of the present disclosure comprises the amino acid sequence set forth in FIG. 2XXX, where “x” is any amino acid other than threonine; for example, “x” can be Gly, Ala, Val, Leu, Ile, Pro, Phe, Tyr, Trp, Ser, Cys, Met, Asn, Gln, Lys, Arg, His, Asp, or Glu. In some cases, a variant 4-1BBL polypeptide of the present disclosure comprises the amino acid sequence set forth in FIG. 2XXX, where “x” is Gly. In some cases, a variant 4-1BBL polypeptide of the present disclosure comprises the amino acid sequence set forth in FIG. 2XXX, where “x” is Ala. In some cases, a variant 4-1BBL polypeptide of the present disclosure comprises the amino acid sequence set forth in FIG. 2XXX, where “x” is Val. In some cases, a variant 4-1BBL polypeptide of the present disclosure comprises the amino acid sequence set forth in FIG. 2XXX, where “x” is Leu. In some cases, a variant 4-1BBL polypeptide of the present disclosure comprises the amino acid sequence set forth in FIG. 2XXX, where “x” is Ile. In some cases, a variant 4-1BBL polypeptide of the present disclosure comprises the amino acid sequence set forth in FIG. 2XXX, where “x” is Pro. In some cases, a variant 4-1BBL polypeptide of the present disclosure comprises the amino acid sequence set forth in FIG. 2HHHH, where “x” is Phe. In some cases, a variant 4-1BBL polypeptide of the present disclosure comprises the amino acid sequence set forth in FIG. 2XXX, where “x” is Tyr. In some cases, a variant 4-1BBL polypeptide of the present disclosure comprises the amino acid sequence set forth in FIG. 2XXX, where “x” is Trp. In some cases, a variant 4-1BBL polypeptide of the present disclosure comprises the amino acid sequence set forth in FIG. 2XXX, where “x” is Ser. In some cases, a variant 4-1BBL polypeptide of the present disclosure comprises the amino acid sequence set forth in FIG. 2XXX, where “x” is Cys. In some cases, a variant 4-1BBL polypeptide of the present disclosure comprises the amino acid sequence set forth in FIG. 2XXX, where “x” is Met. In some cases, a variant 4-1BBL polypeptide of the present disclosure comprises the amino acid sequence set forth in FIG. 2XXX, where “x” is Asn. In some cases, a variant 4-1BBL polypeptide of the present disclosure comprises the amino acid sequence set forth in FIG. 2XXX, where “x” is Gln. In some cases, a variant 4-1BBL polypeptide of the present disclosure comprises the amino acid sequence set forth in FIG. 2XXX, where “x” is Lys. In some cases, a variant 4-1BBL polypeptide of the present disclosure comprises the amino acid sequence set forth in FIG. 2XXX, where “x” is Arg. In some cases, a variant 4-1BBL polypeptide of the present disclosure comprises the amino acid sequence set forth in FIG. 2XXX, where “x” is His. In some cases, a variant 4-1BBL polypeptide of the present disclosure comprises the amino acid sequence set forth in FIG. 2XXX, where “x” is Asp. In some cases, a variant 4-1BBL polypeptide of the present disclosure comprises the amino acid sequence set forth in FIG. 2XXX, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### E219 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 (indicated by an “x”) is an amino acid other than a glutamic acid, e.g., where amino acid 219 is Gly, Ala, Val, Leu, Ile, Pro, Phe, Tyr, Trp, Ser, Thr, Cys, Met, Asn, Gln, Lys, Arg, His, or Asp. The amino acid numbering in FIG. 2YYY retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2YYY, where amino acid 219 is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at E219. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at E139. In some cases, a variant 4-1BBL polypeptide of the present disclosure comprises the amino acid sequence set forth in SEQ ID NO:1, with an amino acid substitution at E139. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E140. In some cases, a variant 4-1BBL polypeptide of the present disclosure comprises the amino acid sequence set forth in SEQ ID NO:2, with an amino acid substitution at E140. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E140. In some cases, a variant 4-1BBL polypeptide of the present disclosure comprises the amino acid sequence set forth in SEQ ID NO:3, with an amino acid substitution at E140.

For example, in some cases, a variant 4-1BBL polypeptide of the present disclosure comprises the amino acid sequence set forth in FIG. 2YYY, where “x” is any amino acid other than glutamic acid; for example, “x” can be Gly, Ala, Val, Leu, Ile, Pro, Phe, Tyr, Trp, Ser, Thr, Cys, Met, Asn, Gln, Lys, Arg, His, or Asp. In some cases, a variant 4-1BBL polypeptide of the present disclosure comprises the amino acid sequence set forth in FIG. 2YYY, where “x” is Gly. In some cases, a variant 4-1BBL polypeptide of the present disclosure comprises the amino acid sequence set forth in FIG. 2YYY, where “x” is Ala. In some cases, a variant 4-1BBL polypeptide of the present disclosure comprises the amino acid sequence set forth in FIG. 2YYY, where “x” is Val. In some cases, a variant 4-1BBL polypeptide of the present disclosure comprises the amino acid sequence set forth in FIG. 2YYY, where “x” is Leu. In some cases, a variant 4-1BBL polypeptide of the present disclosure comprises the amino acid sequence set forth in FIG. 2YYY, where “x” is Ile. In some cases, a variant 4-1BBL polypeptide of the present disclosure comprises the amino acid sequence set forth in FIG. 2YYY, where “x” is Pro. In some cases, a variant 4-1BBL polypeptide of the present disclosure comprises the amino acid sequence set forth in FIG. 2YYY, where “x” is Phe. In some cases, a variant 4-1BBL polypeptide of the present disclosure comprises the amino acid sequence set forth in FIG. 2YYY, where “x” is Tyr. In some cases, a variant 4-1BBL polypeptide of the present disclosure comprises the amino acid sequence set forth in FIG. 2YYY, where “x” is Trp. In some cases, a variant 4-1BBL polypeptide of the present disclosure comprises the amino acid sequence set forth in FIG. 2YYY, where “x” is Ser. In some cases, a variant 4-1BBL polypeptide of the present disclosure comprises the amino acid sequence set forth in FIG. 2YYY, where “x” is Thr. In some cases, a variant 4-1BBL polypeptide of the present disclosure comprises the amino acid sequence set forth in FIG. 2YYY, where “x” is Cys. In some cases, a variant 4-1BBL polypeptide of the present disclosure comprises the amino acid sequence set forth in FIG. 2YYY, where “x” is Met. In some cases, a variant 4-1BBL polypeptide of the present disclosure comprises the amino acid sequence set forth in FIG. 2UU, where “x” is Asn. In some cases, a variant 4-1BBL polypeptide of the present disclosure comprises the amino acid sequence set forth in FIG. 2YYY, where “x” is Gln. In some cases, a variant 4-1BBL polypeptide of the present disclosure comprises the amino acid sequence set forth in FIG. 2YYY, where “x” is Lys. In some cases, a variant 4-1BBL polypeptide of the present disclosure comprises the amino acid sequence set forth in FIG. 2YYY, where “x” is Arg. In some cases, a variant 4-1BBL polypeptide of the present disclosure comprises the amino acid sequence set forth in FIG. 2YYY, where “x” is His. In some cases, a variant 4-1BBL polypeptide of the present disclosure comprises the amino acid sequence set forth in FIG. 2YYY, where “x” is Asp. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R221 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 (indicated by an “x”) is an amino acid other than an arginine, e.g., where amino acid 221 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2ZZZ retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R221. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at R141. In some cases, a variant 4-1BBL polypeptide of the present disclosure comprises the amino acid sequence set forth in SEQ ID NO:1, with an amino acid substitution at R141. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R142. In some cases, a variant 4-1BBL polypeptide of the present disclosure comprises the amino acid sequence set forth in SEQ ID NO:2, with an amino acid substitution at R142. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R142. In some cases, a variant 4-1BBL polypeptide of the present disclosure comprises the amino acid sequence set forth in SEQ ID NO:3, with an amino acid substitution at R142.

For example, in some cases, a variant 4-1BBL polypeptide of the present disclosure comprises the amino acid sequence set forth in FIG. 2ZZZ, where “x” is any amino acid other than arginine; for example, “x” can be Gly, Ala, Val, Leu, Ile, Pro, Phe, Tyr, Trp, Ser, Thr, Cys, Met, Asn, Gln, Lys, His, Asp, or Glu. In some cases, a variant 4-1BBL polypeptide of the present disclosure comprises the amino acid sequence set forth in FIG. 2ZZZ, where “x” is Gly. In some cases, a variant 4-1BBL polypeptide of the present disclosure comprises the amino acid sequence set forth in FIG. 2ZZZ, where “x” is Ala. In some cases, a variant 4-1BBL polypeptide of the present disclosure comprises the amino acid sequence set forth in FIG. 2ZZZ, where “x” is Val. In some cases, a variant 4-1BBL polypeptide of the present disclosure comprises the amino acid sequence set forth in FIG. 2ZZZ, where “x” is Leu. In some cases, a variant 4-1BBL polypeptide of the present disclosure comprises the amino acid sequence set forth in FIG. 2ZZZ, where “x” is Ile. In some cases, a variant 4-1BBL polypeptide of the present disclosure comprises the amino acid sequence set forth in FIG. 2ZZZ, where “x” is Pro. In some cases, a variant 4-1BBL polypeptide of the present disclosure comprises the amino acid sequence set forth in FIG. 2ZZZ, where “x” is Phe. In some cases, a variant 4-1BBL polypeptide of the present disclosure comprises the amino acid sequence set forth in FIG. 2ZZZ, where “x” is Tyr. In some cases, a variant 4-1BBL polypeptide of the present disclosure comprises the amino acid sequence set forth in FIG. 2ZZZ, where “x” is Trp. In some cases, a variant 4-1BBL polypeptide of the present disclosure comprises the amino acid sequence set forth in FIG. 2ZZZ where “x” is Ser. In some cases, a variant 4-1BBL polypeptide of the present disclosure comprises the amino acid sequence set forth in FIG. 2ZZZ, where “x” is Thr. In some cases, a variant 4-1BBL polypeptide of the present disclosure comprises the amino acid sequence set forth in FIG. 2ZZZ, where “x” is Cys. In some cases, a variant 4-1BBL polypeptide of the present disclosure comprises the amino acid sequence set forth in FIG. 2ZZZ, where “x” is Met. In some cases, a variant 4-1BBL polypeptide of the present disclosure comprises the amino acid sequence set forth in FIG. 2ZZZ, where “x” is Asn. In some cases, a variant 4-1BBL polypeptide of the present disclosure comprises the amino acid sequence set forth in FIG. 2ZZZ, where “x” is Gln. In some cases, a variant 4-1BBL polypeptide of the present disclosure comprises the amino acid sequence set forth in FIG. 2ZZZ, where “x” is Lys. In some cases, a variant 4-1BBL polypeptide of the present disclosure comprises the amino acid sequence set forth in FIG. 2ZZZ, where “x” is His. In some cases, a variant 4-1BBL polypeptide of the present disclosure comprises the amino acid sequence set forth in FIG. 2ZZZ, where “x” is Asp. In some cases, a variant 4-1BBL polypeptide of the present disclosure comprises the amino acid sequence set forth in FIG. 2ZZZ, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### R223 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 (indicated by an “x”) is an amino acid other than an arginine, e.g., where amino acid 223 is Gly, Ala, Val, Leu, Ile, Pro, Phe, Tyr, Trp, Ser, Thr, Cys, Met, Asn, Gln, Lys, His, Asp, or Glu. The amino acid numbering in FIG. 2AAAA retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at R223. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at R143. In some cases, a variant 4-1BBL polypeptide of the present disclosure comprises the amino acid sequence set forth in SEQ ID NO:1, with an amino acid substitution at R143. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R144. In some cases, a variant 4-1BBL polypeptide of the present disclosure comprises the amino acid sequence set forth in SEQ ID NO:2, with an amino acid substitution at R144. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R144. In some cases, a variant 4-1BBL polypeptide of the present disclosure comprises the amino acid sequence set forth in SEQ ID NO:3, with an amino acid substitution at R144.

For example, in some cases, a variant 4-1BBL polypeptide of the present disclosure comprises the amino acid sequence set forth in FIG. 2AAAA, where “x” is any amino acid other than arginine; for example, “x” can be Gly, Ala, Val, Leu, Ile, Pro, Phe, Tyr, Trp, Ser, Thr, Cys, Met, Asn, Gln, Lys, His, Asp, or Glu. In some cases, a variant 4-1BBL polypeptide of the present disclosure comprises the amino acid sequence set forth in FIG. 2AAAA, where “x” is Gly. In some cases, a variant 4-1BBL polypeptide of the present disclosure comprises the amino acid sequence set forth in FIG. 2AAAA, where “x” is Ala. In some cases, a variant 4-1BBL polypeptide of the present disclosure comprises the amino acid sequence set forth in FIG. 2AAAA, where “x” is Val. In some cases, a variant 4-1BBL polypeptide of the present disclosure comprises the amino acid sequence set forth in FIG. 2AAAA, where “x” is Leu. In some cases, a variant 4-1BBL polypeptide of the present disclosure comprises the amino acid sequence set forth in FIG. 2AAAA, where “x” is Ile. In some cases, a variant 4-1BBL polypeptide of the present disclosure comprises the amino acid sequence set forth in FIG. 2AAAA, where “x” is Pro. In some cases, a variant 4-1BBL polypeptide of the present disclosure comprises the amino acid sequence set forth in FIG. 2AAAA, where “x” is Phe. In some cases, a variant 4-1BBL polypeptide of the present disclosure comprises the amino acid sequence set forth in FIG. 2AAAA, where “x” is Tyr. In some cases, a variant 4-1BBL polypeptide of the present disclosure comprises the amino acid sequence set forth in FIG. 2AAAA, where “x” is Trp. In some cases, a variant 4-1BBL polypeptide of the present disclosure comprises the amino acid sequence set forth in FIG. 2AAAA where “x” is Ser. In some cases, a variant 4-1BBL polypeptide of the present disclosure comprises the amino acid sequence set forth in FIG. 2AAAA, where “x” is Thr. In some cases, a variant 4-1BBL polypeptide of the present disclosure comprises the amino acid sequence set forth in FIG. 2AAAA, where “x” is Cys. In some cases, a variant 4-1BBL polypeptide of the present disclosure comprises the amino acid sequence set forth in FIG. 2AAAA, where “x” is Met. In some cases, a variant 4-1BBL polypeptide of the present disclosure comprises the amino acid sequence set forth in FIG. 2AAAA, where “x” is Asn. In some cases, a variant 4-1BBL polypeptide of the present disclosure comprises the amino acid sequence set forth in FIG. 2AAAA, where “x” is Gln. In some cases, a variant 4-1BBL polypeptide of the present disclosure comprises the amino acid sequence set forth in FIG. 2AAAA, where “x” is Lys. In some cases, a variant 4-1BBL polypeptide of the present disclosure comprises the amino acid sequence set forth in FIG. 2AAAA, where “x” is His. In some cases, a variant 4-1BBL polypeptide of the present disclosure comprises the amino acid sequence set forth in FIG. 2AAAA, where “x” is Asp. In some cases, a variant 4-1BBL polypeptide of the present disclosure comprises the amino acid sequence set forth in FIG. 2AAAA, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### H224 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 (indicated by an “x”) is an amino acid other than a histidine, e.g., where amino acid 224 is Gly, Ala, Val, Leu, Ile, Pro, Phe, Tyr, Trp, Ser, Thr, Cys, Met, Asn, Gln, Lys, Arg, Asp, or Glu. The amino acid numbering in FIG. 2BBBB retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at H224. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at H144. In some cases, a variant 4-1BBL polypeptide of the present disclosure comprises the amino acid sequence set forth in SEQ ID NO:1, with an amino acid substitution at H144. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H145. In some cases, a variant 4-1BBL polypeptide of the present disclosure comprises the amino acid sequence set forth in SEQ ID NO:2, with an amino acid substitution at H145. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H145. In some cases, a variant 4-1BBL polypeptide of the present disclosure comprises the amino acid sequence set forth in SEQ ID NO:3, with an amino acid substitution at H145.

For example, in some cases, a variant 4-1BBL polypeptide of the present disclosure comprises the amino acid sequence set forth in FIG. 2BBBB, where “x” is any amino acid other than histidine; for example, “x” can be Gly, Ala, Val, Leu, Ile, Pro, Phe, Tyr, Trp, Ser, Thr, Cys, Met, Asn, Gln, Lys, Arg, Asp, or Glu. In some cases, a variant 4-1BBL polypeptide of the present disclosure comprises the amino acid sequence set forth in FIG. 2BBBB, where “x” is Gly. In some cases, a variant 4-1BBL polypeptide of the present disclosure comprises the amino acid sequence set forth in FIG. 2BBBB, where “x” is Ala. In some cases, a variant 4-1BBL polypeptide of the present disclosure comprises the amino acid sequence set forth in FIG. 2BBBB, where “x” is Val. In some cases, a variant 4-1BBL polypeptide of the present disclosure comprises the amino acid sequence set forth in FIG. 2BBBB, where “x” is Leu. In some cases, a variant 4-1BBL polypeptide of the present disclosure comprises the amino acid sequence set forth in FIG. 2BBBB, where “x” is Ile. In some cases, a variant 4-1BBL polypeptide of the present disclosure comprises the amino acid sequence set forth in FIG. 2BBBB, where “x” is Pro. In some cases, a variant 4-1BBL polypeptide of the present disclosure comprises the amino acid sequence set forth in FIG. 2BBBB, where “x” is Phe. In some cases, a variant 4-1BBL polypeptide of the present disclosure comprises the amino acid sequence set forth in FIG. 2BBBB, where “x” is Tyr. In some cases, a variant 4-1BBL polypeptide of the present disclosure comprises the amino acid sequence set forth in FIG. 2BBBB, where “x” is Trp. In some cases, a variant 4-1BBL polypeptide of the present disclosure comprises the amino acid sequence set forth in FIG. 2BBBB, where “x” is Ser. In some cases, a variant 4-1BBL polypeptide of the present disclosure comprises the amino acid sequence set forth in FIG. 2BBBB, where “x” is Thr. In some cases, a variant 4-1BBL polypeptide of the present disclosure comprises the amino acid sequence set forth in FIG. 2BBBB, where “x” is Cys. In some cases, a variant 4-1BBL polypeptide of the present disclosure comprises the amino acid sequence set forth in FIG. 2BBBB, where “x” is Met. In some cases, a variant 4-1BBL polypeptide of the present disclosure comprises the amino acid sequence set forth in FIG. 2BBBB, where “x” is Asn. In some cases, a variant 4-1BBL polypeptide of the present disclosure comprises the amino acid sequence set forth in FIG. 2BBBB, where “x” is Gln. In some cases, a variant 4-1BBL polypeptide of the present disclosure comprises the amino acid sequence set forth in FIG. 2BBBB, where “x” is Lys. In some cases, a variant 4-1BBL polypeptide of the present disclosure comprises the amino acid sequence set forth in FIG. 2BBBB, where “x” is Asp. In some cases, a variant 4-1BBL polypeptide of the present disclosure comprises the amino acid sequence set forth in FIG. 2BBBB, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### W226 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 (indicated by an “x”) is an amino acid other than a tryptophan, e.g., where amino acid 226 is Gly, Ala, Val, Leu, Ile, Pro, Phe, Tyr, Ser, Thr, Cys, Met, Asn, Gln, Lys, Arg, His, Asp, or Glu. The amino acid numbering in FIG. 2CCCC retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at W226. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at W146. In some cases, a variant 4-1BBL polypeptide of the present disclosure comprises the amino acid sequence set forth in SEQ ID NO:1, with an amino acid substitution at W146. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at W147. In some cases, a variant 4-1BBL polypeptide of the present disclosure comprises the amino acid sequence set forth in SEQ ID NO:2, with an amino acid substitution at W147. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at W147. In some cases, a variant 4-1BBL polypeptide of the present disclosure comprises the amino acid sequence set forth in SEQ ID NO:3, with an amino acid substitution at W147.

For example, in some cases, a variant 4-1BBL polypeptide of the present disclosure comprises the amino acid sequence set forth in FIG. 2CCCC, where “x” is any amino acid other than tryptophan; for example, “x” can be Gly, Ala, Val, Leu, Ile, Pro, Phe, Tyr, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2CCCC, where “x” is Gly. In some cases, a variant 4-1BBL polypeptide of the present disclosure comprises the amino acid sequence set forth in FIG. 2CCCC, where “x” is Ala. In some cases, a variant 4-1BBL polypeptide of the present disclosure comprises the amino acid sequence set forth in FIG. 2CCCC, where “x” is Val. In some cases, a variant 4-1BBL polypeptide of the present disclosure comprises the amino acid sequence set forth in FIG. 2CCCC, where “x” is Leu. In some cases, a variant 4-1BBL polypeptide of the present disclosure comprises the amino acid sequence set forth in FIG. 2CCCC, where “x” is Ile. In some cases, a variant 4-1BBL polypeptide of the present disclosure comprises the amino acid sequence set forth in FIG. 2CCCC, where “x” is Pro. In some cases, a variant 4-1BBL polypeptide of the present disclosure comprises the amino acid sequence set forth in FIG. 2CCCC, where “x” is Phe. In some cases, a variant 4-1BBL polypeptide of the present disclosure comprises the amino acid sequence set forth in FIG. 2CCCC, where “x” is Tyr. In some cases, a variant 4-1BBL polypeptide of the present disclosure comprises the amino acid sequence set forth in FIG. 2CCCC, where “x” is Ser. In some cases, a variant 4-1BBL polypeptide of the present disclosure comprises the amino acid sequence set forth in FIG. 2CCCC, where “x” is Thr. In some cases, a variant 4-1BBL polypeptide of the present disclosure comprises the amino acid sequence set forth in FIG. 2CCCC, where “x” is Cys. In some cases, a variant 4-1BBL polypeptide of the present disclosure comprises the amino acid sequence set forth in FIG. 2CCCC, where “x” is Met. In some cases, a variant 4-1BBL polypeptide of the present disclosure comprises the amino acid sequence set forth in FIG. 2CCCC, where “x” is Asn. In some cases, a variant 4-1BBL polypeptide of the present disclosure comprises the amino acid sequence set forth in FIG. 2CCCC, where “x” is Gln. In some cases, a variant 4-1BBL polypeptide of the present disclosure comprises the amino acid sequence set forth in FIG. 2CCCC, where “x” is Lys. In some cases, a variant 4-1BBL polypeptide of the present disclosure comprises the amino acid sequence set forth in FIG. 2CCCC, where “x” is Arg. In some cases, a variant 4-1BBL polypeptide of the present disclosure comprises the amino acid sequence set forth in FIG. 2CCCC, where “x” is His. In some cases, a variant 4-1BBL polypeptide of the present disclosure comprises the amino acid sequence set forth in FIG. 2CCCC, where “x” is Asp. In some cases, a variant 4-1BBL polypeptide of the present disclosure comprises the amino acid sequence set forth in FIG. 2CCCC, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### L228 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 (indicated by an “x”) is an amino acid other than a leucine, e.g., where amino acid 228 is Gly, Ala, Val, Ile, Pro, Phe, Tyr, Trp, Ser, Thr, Cys, Met, Asn, Gln, Lys, Arg, His, Asp, or Glu. The amino acid numbering in FIG. 2DDDD retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at L228. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at L148. In some cases, a variant 4-1BBL polypeptide of the present disclosure comprises the amino acid sequence set forth in SEQ ID NO:1, with an amino acid substitution at L148. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L149. In some cases, a variant 4-1BBL polypeptide of the present disclosure comprises the amino acid sequence set forth in SEQ ID NO:2, with an amino acid substitution at L149. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L149. In some cases, a variant 4-1BBL polypeptide of the present disclosure comprises the amino acid sequence set forth in SEQ ID NO:3, with an amino acid substitution at L149.

For example, in some cases, a variant 4-1BBL polypeptide of the present disclosure comprises the amino acid sequence set forth in FIG. 2DDDD, where “x” is any amino acid other than leucine; for example, “x” can be Gly, Ala, Val, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2DDDD, where “x” is Gly. In some cases, a variant 4-1BBL polypeptide of the present disclosure comprises the amino acid sequence set forth in FIG. 2DDDD, where “x” is Ala. In some cases, a variant 4-1BBL polypeptide of the present disclosure comprises the amino acid sequence set forth in FIG. 2DDDD, where “x” is Val. In some cases, a variant 4-1BBL polypeptide of the present disclosure comprises the amino acid sequence set forth in FIG. 2DDDD, where “x” is Ile. In some cases, a variant 4-1BBL polypeptide of the present disclosure comprises the amino acid sequence set forth in FIG. 2DDDD, where “x” is Pro. In some cases, a variant 4-1BBL polypeptide of the present disclosure comprises the amino acid sequence set forth in FIG. 2DDDD, where “x” is Phe. In some cases, a variant 4-1BBL polypeptide of the present disclosure comprises the amino acid sequence set forth in FIG. 2DDDD, where “x” is Tyr. In some cases, a variant 4-1BBL polypeptide of the present disclosure comprises the amino acid sequence set forth in FIG. 2DDDD, where “x” is Trp. In some cases, a variant 4-1BBL polypeptide of the present disclosure comprises the amino acid sequence set forth in FIG. 2DDDD, where “x” is Ser. In some cases, a variant 4-1BBL polypeptide of the present disclosure comprises the amino acid sequence set forth in FIG. 2DDDD, where “x” is Thr. In some cases, a variant 4-1BBL polypeptide of the present disclosure comprises the amino acid sequence set forth in FIG. 2DDDD, where “x” is Cys. In some cases, a variant 4-1BBL polypeptide of the present disclosure comprises the amino acid sequence set forth in FIG. 2DDDD, where “x” is Met. In some cases, a variant 4-1BBL polypeptide of the present disclosure comprises the amino acid sequence set forth in FIG. 2DDDD, where “x” is Asn. In some cases, a variant 4-1BBL polypeptide of the present disclosure comprises the amino acid sequence set forth in FIG. 2DDDD, where “x” is Gln. In some cases, a variant 4-1BBL polypeptide of the present disclosure comprises the amino acid sequence set forth in FIG. 2DDDD, where “x” is Lys. In some cases, a variant 4-1BBL polypeptide of the present disclosure comprises the amino acid sequence set forth in FIG. 2TT, where “x” is Arg. In some cases, a variant 4-1BBL polypeptide of the present disclosure comprises the amino acid sequence set forth in FIG. 2DDDD, where “x” is His. In some cases, a variant 4-1BBL polypeptide of the present disclosure comprises the amino acid sequence set forth in FIG. 2DDDD, where “x” is Asp. In some cases, a variant 4-1BBL polypeptide of the present disclosure comprises the amino acid sequence set forth in FIG. 2DDDD, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### T229 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 (indicated by an “x”) is an amino acid other than a threonine, e.g., where amino acid 229 is Gly, Ala, Val, Leu, Ile, Pro, Phe, Tyr, Trp, Ser, Cys, Met, Asn, Gln, Lys, Arg, His, Asp, or Glu. The amino acid numbering in FIG. 2EEEE retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at T229. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at T149. In some cases, a variant 4-1BBL polypeptide of the present disclosure comprises the amino acid sequence set forth in SEQ ID NO:1, with an amino acid substitution at T149. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T150. In some cases, a variant 4-1BBL polypeptide of the present disclosure comprises the amino acid sequence set forth in SEQ ID NO:2, with an amino acid substitution at T150. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T150. In some cases, a variant 4-1BBL polypeptide of the present disclosure comprises the amino acid sequence set forth in SEQ ID NO:3, with an amino acid substitution at T150.

For example, in some cases, a variant 4-1BBL polypeptide of the present disclosure comprises the amino acid sequence set forth in FIG. 2EEEE, where “x” is any amino acid other than threonine; for example, “x” can be Gly, Ala, Val, Leu, Ile, Pro, Phe, Tyr, Trp, Ser, Cys, Met, Asn, Gln, Lys, Arg, His, Asp, or Glu. In some cases, a variant 4-1BBL polypeptide of the present disclosure comprises the amino acid sequence set forth in FIG. 2EEEE, where “x” is Gly. In some cases, a variant 4-1BBL polypeptide of the present disclosure comprises the amino acid sequence set forth in FIG. 2EEEE, where “x” is Ala. In some cases, a variant 4-1BBL polypeptide of the present disclosure comprises the amino acid sequence set forth in FIG. 2EEEE, where “x” is Val. In some cases, a variant 4-1BBL polypeptide of the present disclosure comprises the amino acid sequence set forth in FIG. 2EEEE, where “x” is Leu. In some cases, a variant 4-1BBL polypeptide of the present disclosure comprises the amino acid sequence set forth in FIG. 2EEEE, where “x” is Ile. In some cases, a variant 4-1BBL polypeptide of the present disclosure comprises the amino acid sequence set forth in FIG. 2EEEE, where “x” is Pro. In some cases, a variant 4-1BBL polypeptide of the present disclosure comprises the amino acid sequence set forth in FIG. 2EEEE, where “x” is Phe. In some cases, a variant 4-1BBL polypeptide of the present disclosure comprises the amino acid sequence set forth in FIG. 2EEEE, where “x” is Tyr. In some cases, a variant 4-1BBL polypeptide of the present disclosure comprises the amino acid sequence set forth in FIG. 2EEEE, where “x” is Trp. In some cases, a variant 4-1BBL polypeptide of the present disclosure comprises the amino acid sequence set forth in FIG. 2EEEE, where “x” is Ser. In some cases, a variant 4-1BBL polypeptide of the present disclosure comprises the amino acid sequence set forth in FIG. 2EEEE, where “x” is Cys. In some cases, a variant 4-1BBL polypeptide of the present disclosure comprises the amino acid sequence set forth in FIG. 2EEEE, where “x” is Met. In some cases, a variant 4-1BBL polypeptide of the present disclosure comprises the amino acid sequence set forth in FIG. 2EEEE, where “x” is Asn. In some cases, a variant 4-1BBL polypeptide of the present disclosure comprises the amino acid sequence set forth in FIG. 2EEEE, where “x” is Gln. In some cases, a variant 4-1BBL polypeptide of the present disclosure comprises the amino acid sequence set forth in FIG. 2EEEE, where “x” is Lys. In some cases, a variant 4-1BBL polypeptide of the present disclosure comprises the amino acid sequence set forth in FIG. 2EEEE, where “x” is Arg. In some cases, a variant 4-1BBL polypeptide of the present disclosure comprises the amino acid sequence set forth in FIG. 2EEEE, where “x” is His. In some cases, a variant 4-1BBL polypeptide of the present disclosure comprises the amino acid sequence set forth in FIG. 2EEEE, where “x” is Asp. In some cases, a variant 4-1BBL polypeptide of the present disclosure comprises the amino acid sequence set forth in FIG. 2EEEE, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Q230 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 (indicated by an “x”) is an amino acid other than a glutamine, e.g., where amino acid 230 is Gly, Ala, Val, Leu, Ile, Pro, Phe, Tyr, Trp, Ser, Thr, Cys, Met, Asn, Lys, Arg, His, Asp, or Glu. The amino acid numbering in FIG. 2FFFF retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Glu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at Q230. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at Q150. In some cases, a variant 4-1BBL polypeptide of the present disclosure comprises the amino acid sequence set forth in SEQ ID NO:1, with an amino acid substitution at Q150. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q151. In some cases, a variant 4-1BBL polypeptide of the present disclosure comprises the amino acid sequence set forth in SEQ ID NO:2, with an amino acid substitution at Q151. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q151. In some cases, a variant 4-1BBL polypeptide of the present disclosure comprises the amino acid sequence set forth in SEQ ID NO:3, with an amino acid substitution at Q151.

For example, in some cases, a variant 4-1BBL polypeptide of the present disclosure comprises the amino acid sequence set forth in FIG. 2FFFF, where “x” is any amino acid other than glutamine; for example, “x” can be Gly, Ala, Val, Leu, Ile, Pro, Phe, Tyr, Trp, Ser, Thr, Cys, Met, Asn, Gln, Arg, His, Asp, or Glu. In some cases, a variant 4-1BBL polypeptide of the present disclosure comprises the amino acid sequence set forth in FIG. 2FFFF, where “x” is Phe. In some cases, a variant 4-1BBL polypeptide of the present disclosure comprises the amino acid sequence set forth in FIG. 2FFFF, where “x” is Leu. In some cases, a variant 4-1BBL polypeptide of the present disclosure comprises the amino acid sequence set forth in FIG. 2FFFF, where “x” is Ile. In some cases, a variant 4-1BBL polypeptide of the present disclosure comprises the amino acid sequence set forth in FIG. 2FFFF, where “x” is Met. In some cases, a variant 4-1BBL polypeptide of the present disclosure comprises the amino acid sequence set forth in FIG. 2FFFF, where “x” is Val. In some cases, a variant 4-1BBL polypeptide of the present disclosure comprises the amino acid sequence set forth in FIG. 2FFFF, where “x” is Ser. In some cases, a variant 4-1BBL polypeptide of the present disclosure comprises the amino acid sequence set forth in FIG. 2FFFF, where “x” is Pro. In some cases, a variant 4-1BBL polypeptide of the present disclosure comprises the amino acid sequence set forth in FIG. 2FFFF, where “x” is Thr. In some cases, a variant 4-1BBL polypeptide of the present disclosure comprises the amino acid sequence set forth in FIG. 2FFFF, where “x” is Ala. In some cases, a variant 4-1BBL polypeptide of the present disclosure comprises the amino acid sequence set forth in FIG. 2FFFF, where “x” is Tyr. In some cases, a variant 4-1BBL polypeptide of the present disclosure comprises the amino acid sequence set forth in FIG. 2FFFF, where “x” is His. In some cases, a variant 4-1BBL polypeptide of the present disclosure comprises the amino acid sequence set forth in FIG. 2FFFF, where “x” is Asn. In some cases, a variant 4-1BBL polypeptide of the present disclosure comprises the amino acid sequence set forth in FIG. 2FFFF, where “x” is Lys. In some cases, a variant 4-1BBL polypeptide of the present disclosure comprises the amino acid sequence set forth in FIG. 2FFFF, where “x” is Asp. In some cases, a variant 4-1BBL polypeptide of the present disclosure comprises the amino acid sequence set forth in FIG. 2FFFF, where “x” is Glu. In some cases, a variant 4-1BBL polypeptide of the present disclosure comprises the amino acid sequence set forth in FIG. 2FFFF, where “x” is Cys. In some cases, a variant 4-1BBL polypeptide of the present disclosure comprises the amino acid sequence set forth in FIG. 2SS, where “x” is Trp. In some cases, a variant 4-1BBL polypeptide of the present disclosure comprises the amino acid sequence set forth in FIG. 2FFFF, where “x” is Arg. In some cases, a variant 4-1BBL polypeptide of the present disclosure comprises the amino acid sequence set forth in FIG. 2FFFF where “x” is Gly. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### G231 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 (indicated by an “x”) is an amino acid other than a glycine, e.g., where amino acid 231 is Ala, Val, Leu, Ile, Pro, Phe, Tyr, Trp, Ser, Thr, Cys, Met, Asn, Gln, Lys, Arg, His, Asp, or Glu. The amino acid numbering in FIG. 2GGGG retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at G231. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at G151. In some cases, a variant 4-1BBL polypeptide of the present disclosure comprises the amino acid sequence set forth in SEQ ID NO:1, with an amino acid substitution at G151. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G152. In some cases, a variant 4-1BBL polypeptide of the present disclosure comprises the amino acid sequence set forth in SEQ ID NO:2, with an amino acid substitution at G152. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G152. In some cases, a variant 4-1BBL polypeptide of the present disclosure comprises the amino acid sequence set forth in SEQ ID NO:3, with an amino acid substitution at G152.

For example, in some cases, a variant 4-1BBL polypeptide of the present disclosure comprises the amino acid sequence set forth in FIG. 2GGGG, where “x” is any amino acid other than glycine; for example, “x” can be Ala, Val, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2GGGG, where “x” is Ala. In some cases, a variant 4-1BBL polypeptide of the present disclosure comprises the amino acid sequence set forth in FIG. 2GGGG, where “x” is Val. In some cases, a variant 4-1BBL polypeptide of the present disclosure comprises the amino acid sequence set forth in FIG. 2GGGG, where “x” is Leu. In some cases, a variant 4-1BBL polypeptide of the present disclosure comprises the amino acid sequence set forth in FIG. 2GGGG, where “x” is Ile. In some cases, a variant 4-1BBL polypeptide of the present disclosure comprises the amino acid sequence set forth in FIG. 2GGGG, where “x” is Pro. In some cases, a variant 4-1BBL polypeptide of the present disclosure comprises the amino acid sequence set forth in FIG. 2GGGG, where “x” is Phe. In some cases, a variant 4-1BBL polypeptide of the present disclosure comprises the amino acid sequence set forth in FIG. 2GGGG, where “x” is Tyr. In some cases, a variant 4-1BBL polypeptide of the present disclosure comprises the amino acid sequence set forth in FIG. 2GGGG, where “x” is Trp. In some cases, a variant 4-1BBL polypeptide of the present disclosure comprises the amino acid sequence set forth in FIG. 2GGGG, where “x” is Ser. In some cases, a variant 4-1BBL polypeptide of the present disclosure comprises the amino acid sequence set forth in FIG. 2GGGG, where “x” is Thr. In some cases, a variant 4-1BBL polypeptide of the present disclosure comprises the amino acid sequence set forth in FIG. 2GGGG, where “x” is Cys. In some cases, a variant 4-1BBL polypeptide of the present disclosure comprises the amino acid sequence set forth in FIG. 2GGGG, where “x” is Met. In some cases, a variant 4-1BBL polypeptide of the present disclosure comprises the amino acid sequence set forth in FIG. 2GGGG, where “x” is Asn. In some cases, a variant 4-1BBL polypeptide of the present disclosure comprises the amino acid sequence set forth in FIG. 2GGGG, where “x” is Gln. In some cases, a variant 4-1BBL polypeptide of the present disclosure comprises the amino acid sequence set forth in FIG. 2GGGG, where “x” is Lys. In some cases, a variant 4-1BBL polypeptide of the present disclosure comprises the amino acid sequence set forth in FIG. 2GGGG, where “x” is Arg. In some cases, a variant 4-1BBL polypeptide of the present disclosure comprises the amino acid sequence set forth in FIG. 2GGGG, where “x” is His. In some cases, a variant 4-1BBL polypeptide of the present disclosure comprises the amino acid sequence set forth in FIG. 2GGGG, where “x” is Asp. In some cases, a variant 4-1BBL polypeptide of the present disclosure comprises the amino acid sequence set forth in FIG. 2GGGG, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 M, or from about 75 μM to about 100 μM.

### T233 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 (indicated by an “x”) is an amino acid other than a threonine, e.g., where amino acid 233 is Gly, Ala, Val, Leu, Ile, Pro, Phe, Tyr, Trp, Ser, Cys, Met, Asn, Gln, Lys, Arg, His, Asp, or Glu. The amino acid numbering in FIG. 2HHHH retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Val. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at T233. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at T153. In some cases, a variant 4-1BBL polypeptide of the present disclosure comprises the amino acid sequence set forth in SEQ ID NO:1, with an amino acid substitution at T153. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T154. In some cases, a variant 4-1BBL polypeptide of the present disclosure comprises the amino acid sequence set forth in SEQ ID NO:2, with an amino acid substitution at T154. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T154. In some cases, a variant 4-1BBL polypeptide of the present disclosure comprises the amino acid sequence set forth in SEQ ID NO:3, with an amino acid substitution at T154.

For example, in some cases, a variant 4-1BBL polypeptide of the present disclosure comprises the amino acid sequence set forth in FIG. 2HHHH, where “x” is any amino acid other than threonine; for example, “x” can be Gly, Ala, Val, Leu, Ile, Pro, Phe, Tyr, Trp, Ser, Cys, Met, Asn, Gln, Lys, Arg, His, Asp, or Glu. In some cases, a variant 4-1BBL polypeptide of the present disclosure comprises the amino acid sequence set forth in FIG. 2HHHH, where “x” is Gly. In some cases, a variant 4-1BBL polypeptide of the present disclosure comprises the amino acid sequence set forth in FIG. 2HHHH, where “x” is Ala. In some cases, a variant 4-1BBL polypeptide of the present disclosure comprises the amino acid sequence set forth in FIG. 2HHHH, where “x” is Val. In some cases, a variant 4-1BBL polypeptide of the present disclosure comprises the amino acid sequence set forth in FIG. 2HHHH, where “x” is Leu. In some cases, a variant 4-1BBL polypeptide of the present disclosure comprises the amino acid sequence set forth in FIG. 2HHHH, where “x” is Ile. In some cases, a variant 4-1BBL polypeptide of the present disclosure comprises the amino acid sequence set forth in FIG. 2HHHH, where “x” is Pro. In some cases, a variant 4-1BBL polypeptide of the present disclosure comprises the amino acid sequence set forth in FIG. 2HHHH, where “x” is Phe. In some cases, a variant 4-1BBL polypeptide of the present disclosure comprises the amino acid sequence set forth in FIG. 2HHHH, where “x” is Tyr. In some cases, a variant 4-1BBL polypeptide of the present disclosure comprises the amino acid sequence set forth in FIG. 2HHHH, where “x” is Trp. In some cases, a variant 4-1BBL polypeptide of the present disclosure comprises the amino acid sequence set forth in FIG. 2HHHH, where “x” is Ser. In some cases, a variant 4-1BBL polypeptide of the present disclosure comprises the amino acid sequence set forth in FIG. 2HHHH, where “x” is Cys. In some cases, a variant 4-1BBL polypeptide of the present disclosure comprises the amino acid sequence set forth in FIG. 2HHHH, where “x” is Met. In some cases, a variant 4-1BBL polypeptide of the present disclosure comprises the amino acid sequence set forth in FIG. 2HHHH, where “x” is Asn. In some cases, a variant 4-1BBL polypeptide of the present disclosure comprises the amino acid sequence set forth in FIG. 2HHHH, where “x” is Gln. In some cases, a variant 4-1BBL polypeptide of the present disclosure comprises the amino acid sequence set forth in FIG. 2HHHH, where “x” is Lys. In some cases, a variant 4-1BBL polypeptide of the present disclosure comprises the amino acid sequence set forth in FIG. 2HHHH, where “x” is Arg. In some cases, a variant 4-1BBL polypeptide of the present disclosure comprises the amino acid sequence set forth in FIG. 2HHHH, where “x” is His. In some cases, a variant 4-1BBL polypeptide of the present disclosure comprises the amino acid sequence set forth in FIG. 2HHHH, where “x” is Asp. In some cases, a variant 4-1BBL polypeptide of the present disclosure comprises the amino acid sequence set forth in FIG. 2HHHH, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### V234 Substitution

In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 (indicated by an “x”) is an amino acid other than a valine, e.g., where amino acid 234 is Gly, Ala, Leu, Ile, Pro, Phe, Tyr, Trp, Ser, Thr, Cys, Met, Asn, Gln, Lys, Arg, His, Asp, or Glu. The amino acid numbering in FIG. 2IIII retains the numbering of FIG. 2A. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Gly. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Ala. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Leu. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Ile. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Pro. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Phe. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Tyr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Trp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Ser. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Thr. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Cys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Met. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Asn. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Gln. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Lys. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Arg. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is His. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Asp. In some cases, a variant 4-1BBL polypeptide of the present disclosure comprises an amino acid sequence having at least 90%, at least 95%, at least 98%, or at least 99%, amino acid sequence identity to the amino acid sequence depicted in FIG. 2IIII, where amino acid 234 is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide of the present disclosure comprises the amino acid sequence set forth in FIG. 2A, with an amino acid substitution at V234. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:1, with an amino acid substitution at V154. In some cases, a variant 4-1BBL polypeptide of the present disclosure comprises the amino acid sequence set forth in SEQ ID NO:1, with an amino acid substitution at V154. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V155. In some cases, a variant 4-1BBL polypeptide of the present disclosure comprises the amino acid sequence set forth in SEQ ID NO:2, with an amino acid substitution at V155. In some cases, a variant 4-1BBL polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V155. In some cases, a variant 4-1BBL polypeptide of the present disclosure comprises the amino acid sequence set forth in SEQ ID NO:3, with an amino acid substitution at V155.

For example, in some cases, a variant 4-1BBL polypeptide of the present disclosure comprises the amino acid sequence set forth in FIG. 2IIII, where “x” is any amino acid other than valine; for example, “x” can be Gly, Ala, Leu, Ile, Pro, Phe, Tyr, Trp, Ser, Thr, Cys, Met, Asn, Gln, Lys, Arg, His, Asp, or Glu. In some cases, a variant 4-1BBL polypeptide of the present disclosure comprises the amino acid sequence set forth in FIG. 2IIII, where “x” is Gly. In some cases, a variant 4-1BBL polypeptide of the present disclosure comprises the amino acid sequence set forth in FIG. 2IIII, where “x” is Ala. In some cases, a variant 4-1BBL polypeptide of the present disclosure comprises the amino acid sequence set forth in FIG. 2IIII, where “x” is Leu. In some cases, a variant 4-1BBL polypeptide of the present disclosure comprises the amino acid sequence set forth in FIG. 2IIII, where “x” is Ile. In some cases, a variant 4-1BBL polypeptide of the present disclosure comprises the amino acid sequence set forth in FIG. 2IIII, where “x” is Pro. In some cases, a variant 4-1BBL polypeptide of the present disclosure comprises the amino acid sequence set forth in FIG. 2IIII, where “x” is Phe. In some cases, a variant 4-1BBL polypeptide of the present disclosure comprises the amino acid sequence set forth in FIG. 2IIII, where “x” is Tyr. In some cases, a variant 4-1BBL polypeptide of the present disclosure comprises the amino acid sequence set forth in FIG. 2IIII, where “x” is Trp. In some cases, a variant 4-1BBL polypeptide of the present disclosure comprises the amino acid sequence set forth in FIG. 2IIII, where “x” is Ser. In some cases, a variant 4-1BBL polypeptide of the present disclosure comprises the amino acid sequence set forth in FIG. 2IIII, where “x” is Thr. In some cases, a variant 4-1BBL polypeptide of the present disclosure comprises the amino acid sequence set forth in FIG. 2IIII, where “x” is Cys. In some cases, a variant 4-1BBL polypeptide of the present disclosure comprises the amino acid sequence set forth in FIG. 2IIII, where “x” is Met. In some cases, a variant 4-1BBL polypeptide of the present disclosure comprises the amino acid sequence set forth in FIG. 2IIII, where “x” is Asn. In some cases, a variant 4-1BBL polypeptide of the present disclosure comprises the amino acid sequence set forth in FIG. 2ZZ, where “x” is Gln. In some cases, a variant 4-1BBL polypeptide of the present disclosure comprises the amino acid sequence set forth in FIG. 2IIII, where “x” is Lys. In some cases, a variant 4-1BBL polypeptide of the present disclosure comprises the amino acid sequence set forth in FIG. 2IIII, where “x” is Arg. In some cases, a variant 4-1BBL polypeptide of the present disclosure comprises the amino acid sequence set forth in FIG. 2IIII, where “x” is His. In some cases, a variant 4-1BBL polypeptide of the present disclosure comprises the amino acid sequence set forth in FIG. 2IIII, where “x” is Asp. In some cases, a variant 4-1BBL polypeptide of the present disclosure comprises the amino acid sequence set forth in FIG. 2IIII, where “x” is Glu. In some cases, the variant 4-1BBL polypeptide has a binding affinity to CD137 that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Fusion Polypeptides

The present disclosure provides 4-1BBL fusion polypeptides. A fusion polypeptide of the present disclosure comprises: a) a variant 4-1BBL polypeptide of the present disclosure; and b) a heterologous fusion partner. In some cases, the heterologous fusion partner is fused to the N-terminus of the variant 4-1BBL polypeptide. In some cases, the heterologous fusion partner is fused to the C-terminus of the variant 4-1BBL polypeptide. In some cases, a 4-1BBL fusion polypeptide of the present disclosure comprises a first heterologous fusion partner fused to the N-terminus of the variant 4-1BBL polypeptide, and a second heterologous fusion partner fused to the C-terminus of the variant 4-1BBL polypeptide.

The total length of a 4-1BBL fusion polypeptide of the present disclosure can range from 215 amino acids to 2000 amino acids. For example, a 4-1BBL fusion polypeptide of the present disclosure can range from 215 amino acids to 225 amino acids, from 225 amino acids to 250 amino acids, from 250 amino acids to 275 amino acids, from 275 amino acids to 300 amino acids, from 300 amino acids to 350 amino acids, from 350 amino acids, from 350 amino acids to 400 amino acids, from 400 amino acids, from 400 amino acids to 450 amino acids, from 450 amino acids to 500 amino acids, from 500 amino acids to 600 amino acids, from 600 amino acids to 700 amino acids, from 700 amino acids to 800 amino acids, from 800 amino acids to 900 amino acids, from 900 amino acids to 1000 amino acids, from 1000 amino acids to 1250 amino acids, from 1250 amino acids to 1500 amino acids, from 1500 amino acids to 1750 amino acids, or from 1750 amino acids to 2000 amino acids.

Suitable fusion partners include, but are not limited to, a transmembrane polypeptide; an antibody Fc polypeptide; an antigen-binding region of an antibody; a cytokine; an immunomodulatory polypeptide (e.g., a 4-1BBL immunomodulatory polypeptide; an immunomodulatory polypeptide other than 4-BBL); an intracellular signaling polypeptide; and the like.

### T-Cell Modulatory Multimeric Polypeptides

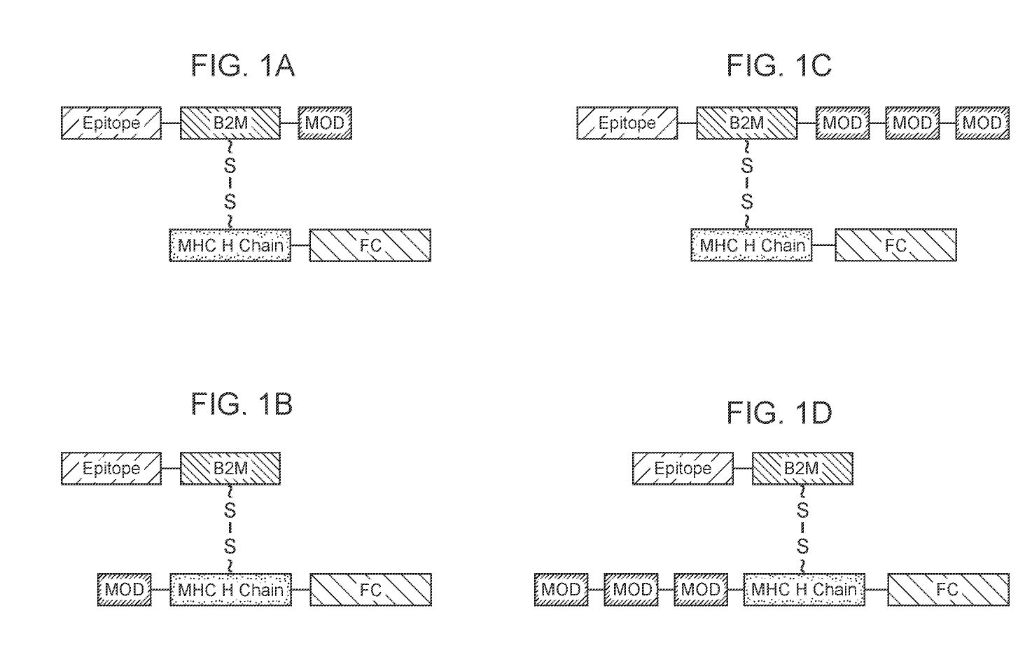

The present disclosure provides multimeric (e.g., heterodimeric, heterotrimeric) polypeptides. The multimeric polypeptides are T cell modulatory polypeptides, and are also referred to herein as “T-cell modulatory multimeric polypeptides,” or “synTac” (for “immunological synapse for T cell activation”). FIG. 1A-1D provide schematic depictions of examples of T-cell modulatory multimeric polypeptides of the present disclosure. A T-cell modulatory multimeric polypeptide of the present disclosure is also referred to as a “synTac polypeptide” or a “multimeric polypeptide.”

A synTac polypeptide of the present disclosure comprises a variant immunomodulatory polypeptide of the present disclosure. As noted above, a variant 4-1BBL polypeptide present in a multimeric polypeptide of the present disclosure exhibits reduced binding affinity to 4-1BB (CD137), compared to the binding affinity of wild-type 4-1BBL to 4-1BB. A multimeric polypeptide of the present disclosure that comprises a variant 4-1BBL polypeptide of the present disclosure also exhibits reduced binding affinity to 4-1BB, compared to a control multimeric polypeptide comprising a wild-type 4-1BBL (e.g., a 4-1BBL polypeptide comprising the amino acid sequence depicted in FIG. 2A or comprising the amino acid sequence set forth in SEQ ID NO:1).

In some cases, a synTac polypeptide of the present disclosure exhibits reduced binding affinity to 4-1BB (e.g., a 4-1BB polypeptide comprising the amino acid sequence depicted in FIG. 3), compared to the binding affinity of a 4-1BBL polypeptide comprising the amino acid sequence depicted in FIG. 2A for 4-1BB. For example, in some cases, a synTac polypeptide of the present disclosure binds 4-1BB with a binding affinity that is less than the binding affinity of a control synTac polypeptide comprising a 4-1BBL polypeptide comprising the amino acid sequence depicted in FIG. 2A for a 4-1BB polypeptide comprising the amino acid sequence depicted in FIG. 3. For example, in some cases, a synTac polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a control synTac polypeptide comprising a 4-1BBL polypeptide comprising the amino acid sequence depicted in FIG. 2A for 4-1BB (e.g., a 4-1BB polypeptide comprising the amino acid sequence depicted in FIG. 3).

In some cases, a synTac polypeptide of the present disclosure exhibits reduced binding affinity to 4-1BB (e.g., a 4-1BB polypeptide comprising the amino acid sequence depicted in FIG. 3), compared to the binding affinity of a control synTac polypeptide comprising a 4-1BBL polypeptide comprising the amino acid sequence depicted in SEQ ID NO:1 for 4-1BB. For example, in some cases, a synTac polypeptide of the present disclosure binds CD28 with a binding affinity that is less than the binding affinity of a control synTac polypeptide comprises a 4-1BBL polypeptide comprising the amino acid sequence depicted in SEQ ID NO:1 for a 4-1BB polypeptide comprising the amino acid sequence depicted in FIG. 3. For example, in some cases, a synTac polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a control synTac polypeptide comprising a 4-1BBL polypeptide comprising the amino acid sequence depicted in SEQ ID NO:1 for 4-1BB (e.g., a 4-1BB polypeptide comprising the amino acid sequence depicted in FIG. 3).

In some cases, a synTac polypeptide of the present disclosure has a binding affinity to 4-1BB that is from 100 nm to about 100 μM. In some cases, a synTac polypeptide of the present disclosure has a binding affinity to 4-1BB that is from about 100 nM to 500 nM. For example, in some cases, a synTac polypeptide of the present disclosure has a binding affinity for 4-1BB (e.g., a 4-1BB polypeptide comprising the amino acid sequence depicted in FIG. 3) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, or from about 450 nM to about 500 nM. In some cases, a synTac polypeptide of the present disclosure has a binding affinity for 4-1BB (e.g., a 4-1BB polypeptide comprising the amino acid sequence depicted in FIG. 3) that is from about 500 nM to 1 μM. For example, in some cases, a synTac polypeptide of the present disclosure has a binding affinity for 4-1BB (e.g., a 4-1BB polypeptide comprising the amino acid sequence depicted in FIG. 3) that is from about 500 nM to about 600 nM, from about 600 nM to about 700 nM, from about 700 nM to about 800 nM, from about 800 nM to about 900 nM, or from about 900 nM to about 1 μM. In some cases, a synTac polypeptide of the present disclosure has a binding affinity for 4-1BB (e.g., a 4-1BB polypeptide comprising the amino acid sequence depicted in FIG. 3) that is from about 1 μM to 10 μM. For example, in some cases, a synTac polypeptide of the present disclosure has a binding affinity for 4-1BB (e.g., a 4-1BB polypeptide comprising the amino acid sequence depicted in FIG. 3) that is from about 1 μM to 2 μM, from about 2 μM to about 3 μM, from about 3 μM to about 4 μM, from about 4 μM to about 5 μM, from about 5 μM to about 6 μM, from about 6 μM to about 7 μM, from about 7 μM to about 8 μM, from about 8 μM to about 9 μM, or from about 9 μM to about 10 μM. In some cases, a synTac polypeptide of the present disclosure has a binding affinity for 4-1BB (e.g., a 4-1BB polypeptide comprising the amino acid sequence depicted in FIG. 3) that is from about 10 μM to 100 μM. For example, in some cases, a synTac polypeptide of the present disclosure has a binding affinity for 4-1BB (e.g., a 4-1BB polypeptide comprising the amino acid sequence depicted in FIG. 3) that is from about 10 μM to about 20 μM, from about 20 μM to about 30 μM, from about 30 μM to about 40 μM, from about 40 μM to about 50 μM, from about 50 μM to about 60 μM, from about 60 μM to about 70 μM, from about 70 μM to about 80 μM, from about 80 μM to about 90 μM, or from about 90 μM to about 100 μM.

In some cases, a synTac polypeptide of the present disclosure (comprising a variant 4-1BBL polypeptide of the present disclosure) exhibits increased production in a mammalian host cell, compared to the production in the same mammalian host cell of a control synTac polypeptide comprising a wild-type 4-1BBL polypeptide (e.g., a 4-1BBL polypeptide comprising the amino acid sequence depicted in FIG. 2A or as set forth in SEQ ID NO:1). For example, in some cases, a synTac polypeptide of the present disclosure, when expressed in a mammalian host cell, is produced in an amount that is from 25% higher to about 50% higher, from about 50% higher to about 75% higher, from about 75% higher to about 2-fold higher, from about 2-fold higher to about 5-fold higher, from about 5-fold higher to about 10-fold higher, from about 10-fold higher to about 20-fold higher, from about 20-fold higher to about 30-fold higher, from about 30-fold higher to about 40-fold higher, from about 40-fold higher to about 50-fold higher, from about 50-fold higher to about 75-fold higher, from about 75-fold higher to about 100-fold higher, or more than 100-fold higher, than the amount of a control synTac polypeptide comprising a wild-type 4-1BBL polypeptide (e.g., a 4-1BBL polypeptide comprising the amino acid sequence depicted in FIG. 2A or as set forth in SEQ ID NO:1) produced in the same mammalian host cell.

In some cases, a synTac polypeptide of the present disclosure (comprising a variant 4-1BBL polypeptide of the present disclosure) is produced in a mammalian host cell in an amount of from about 50 mg/L to about 75 mg/L, from about 75 mg/L to about 100 mg/L, from about 100 mg/L to about 150 mg/L, from about 150 mg/L to about 200 mg/L, from about 200 mg/L to about 250 mg/L, from about 250 mg/L to about 500 mg/L, or more than 500 mg/L. In some cases, a synTac polypeptide of the present disclosure (comprising a variant 4-1BBL polypeptide of the present disclosure) is produced in a mammalian host cell in an amount of from about 10 mg/L to about 15 mg/L, from about 15 mg/L to about 20 mg/L, from about 20 mg/L to about 25 mg/L, from about 25 mg/L to about 30 mg/L, from about 35 mg/L to about 40 mg/L, from about 40 mg/L to about 45 mg/L, or from about 45 mg/L to about 50 mg/L.

A variant 4-1BBL polypeptide present in a synTac polypeptide of the present disclosure can have a single amino acid substitution relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has from 2 to 10 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 2 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 3 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 4 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 5 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 6 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 7 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 8 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 9 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). In some cases, a variant 4-1BBL polypeptide present in a synTac polypeptide of the present disclosure has 10 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3).

In some cases, a variant 4-1BBL polypeptide present in a multimeric polypeptide of the present disclosure has from 11 to 50 amino acid substitutions relative to a wild-type 4-1BBL polypeptide (e.g., a 4-1BBL polypeptide comprising the amino acid sequence depicted in FIG. 2A or as set forth in one of SEQ ID NOs:1-3). For example, in some cases, a variant 4-1BBL polypeptide present in a multimeric polypeptide of the present disclosure has from 11 to 15, from 15 to 20, from 20 to 25, from 25 to 30, from 30 to 35, from 35 to 40, from 40 to 45, or from 45 to 50, amino acid substitutions relative to a wild-type 4-1BBL polypeptide (e.g., a 4-1BBL polypeptide comprising the amino acid sequence depicted in FIG. 2A or as set forth in one of SEQ ID NOs:1-3).

A variant 4-1BBL polypeptide present in a synTac polypeptide of the present disclosure can have a length of from 200 amino acids to 254 amino acids. A variant 4-1BBL polypeptide present in a synTac polypeptide of the present disclosure can have a length of from 150 amino acids to 200 amino acids. For example, in some cases, a variant 4-1BBL polypeptide present in a synTac polypeptide of the present disclosure has a length of from 150 amino acids to 160 amino acids, from 160 amino acids o 170 amino acids, from 170 amino acids to 180 amino acids, from 180 amino acids to 190 amino acids, or from 190 amino acids to 200 amino acids. In some cases, a variant 4-1BBL polypeptide present in a synTac polypeptide of the present disclosure has a length of from 160 amino acids to 175 amino acids. In some cases, a variant 4-1BBL polypeptide present in a synTac polypeptide of the present disclosure has a length of 162 amino acids. In some cases, a variant 4-1BBL polypeptide of the present disclosure has a length of 174 amino acids.

In some cases, a multimeric polypeptide of the present disclosure comprises a first polypeptide and a second polypeptide, where the first polypeptide comprises, in order from amino terminus (N-terminus) to carboxyl terminus (C-terminus): a) an epitope (e.g., a T-cell epitope); b) a first major histocompatibility complex (MHC) polypeptide and c) an immunomodulatory polypeptide (e.g., a variant 4-1BBL polypeptide of the present disclosure); and where the second polypeptide comprises, in order from N-terminus to C-terminus: a) a second MHC polypeptide; and b) an immunoglobulin (Ig) Fc polypeptide. In other cases, a multimeric polypeptide of the present disclosure comprises a first polypeptide and a second polypeptide, where the first polypeptide comprises, in order from N-terminus to C-terminus: a) an epitope (e.g., a T-cell epitope); and b) a first MHC polypeptide; and where the second polypeptide comprises, in order from N-terminus to C-terminus: a) an immunomodulatory polypeptide (e.g., a variant 4-1BBL polypeptide of the present disclosure); b) a second MHC polypeptide; and c) an Ig Fc polypeptide. In some instances, the first and the second MHC polypeptides are Class I MHC polypeptides; e.g., in some cases, the first MHC polypeptide is an MHC Class I β2-microglobulin (B2M or β2M) polypeptide, and the second MHC polypeptide is an MHC Class I heavy chain (H chain); or the first MHC polypeptide is an MHC Class I H chain, and the second MHC polypeptide is an MHC Class I β2M polypeptide). In other cases, the first and the second MHC polypeptides are Class II MHC polypeptides; e.g., in some cases, the first MHC polypeptide is an MHC Class II α-chain polypeptide, and the second MHC polypeptide is an MHC Class II β-chain polypeptide. In other cases, the first polypeptide is an MHC Class II β-chain polypeptide, and the second MHC polypeptide is an MHC Class II α-chain polypeptide. In some cases, the multimeric polypeptide includes two or more immunomodulatory polypeptides, where at least one of the immunomodulatory polypeptides is a variant 4-1BBL immunomodulatory polypeptide of the present disclosure. Where a multimeric polypeptide of the present disclosure includes two or more immunomodulatory polypeptides, in some cases, the two or more immunomodulatory polypeptides are present in the same polypeptide chain, and may be in tandem. Where a multimeric polypeptide of the present disclosure includes two or more immunomodulatory polypeptides, in some cases, the two or more immunomodulatory polypeptides are present in separate polypeptides. In some cases, a multimeric polypeptide of the present disclosure is a heterodimer. In some cases, a multimeric polypeptide of the present disclosure is a trimeric polypeptide.

In some cases, a multimeric polypeptide of the present disclosure comprises: a) a first polypeptide comprising, in order from N-terminus to C-terminus: i) an epitope; and ii) a first MHC polypeptide; and b) a second polypeptide comprising, in order from N-terminus to C-terminus: i) a second MHC polypeptide; and ii) an Ig Fc polypeptide; and iii) an immunomodulatory domain (e.g., a variant 4-1BBL polypeptide of the present disclosure). In some cases, a multimeric polypeptide of the present disclosure comprises: a) a first polypeptide comprising, in order from N-terminus to C-terminus: i) an epitope; and ii) a first MHC polypeptide; and b) a second polypeptide comprising, in order from N-terminus to C-terminus: i) a second MHC polypeptide; and ii) an immunomodulatory domain (e.g., a variant 4-1BBL polypeptide of the present disclosure). In some cases, a multimeric polypeptide of the present disclosure comprises: a) a first polypeptide comprising, in order from N-terminus to C-terminus: i) an epitope; and ii) a first MHC polypeptide; and b) a second polypeptide comprising, in order from N-terminus to C-terminus: i) an immunomodulatory domain (e.g., a variant 4-1BBL polypeptide of the present disclosure); and ii) a second MHC polypeptide. In some cases, a multimeric polypeptide of the present disclosure comprises: a) a first polypeptide comprising, in order from N-terminus to C-terminus: i) an epitope; ii) a first MHC polypeptide; and iii) an immunomodulatory domain (e.g., a variant 4-1BBL polypeptide of the present disclosure); and b) a second polypeptide comprising, in order from N-terminus to C-terminus: i) a second MHC polypeptide. In some cases, where a multimeric polypeptide of the present disclosure comprises a non-Ig scaffold, the non-Ig scaffold is an XTEN peptide, a transferrin polypeptide, an Fc receptor polypeptide, an elastin-like polypeptide, a silk-like polypeptide, or a silk-elastin-like polypeptide.

In some cases, a multimeric polypeptide of the present disclosure is monovalent. In some cases, a multimeric polypeptide of the present disclosure is multivalent. In some cases, a multivalent multimeric polypeptide of the present disclosure comprises an immunoglobulin Fc polypeptide on one of the first or the second polypeptide. For example, depending on the Fc polypeptide present in a multimeric polypeptide of the present disclosure, the multimeric polypeptide can be a homodimer, where two molecules of the multimeric polypeptide are present in the homodimer, where the two molecules of the multimeric polypeptide can be disulfide linked to one another, e.g., via the Fc polypeptide present in the two molecules. As another example, a multimeric polypeptide of the present disclosure can comprise three, four, or five molecules of the multimeric polypeptide, where the molecules of the multimeric polypeptide can be disulfide linked to one another, e.g., via the Fc polypeptide present in the molecules.

In some cases, a multimeric polypeptide of the present disclosure comprises: a) a first polypeptide comprising, in order from N-terminus to C-terminus: i) an epitope; ii) a β2M polypeptide; and iii) a variant 4-1BBL polypeptide of the present disclosure; and b) a second polypeptide comprising, in order from N-terminus to C-terminus: i) a Class I MHC heavy chain; and ii) an Fc polypeptide. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a variant 4-1BBL polypeptide of the present disclosure; ii) a Class I MHC heavy chain; and iii) an Fc polypeptide. In some cases, a multimeric polypeptide of the present disclosure comprises: a) a first polypeptide comprising, in order from N-terminus to C-terminus: i) an epitope; ii) a β2M polypeptide; iii) a first variant 4-1BBL polypeptide of the present disclosure; iv) a second variant 4-1BBL polypeptide of the present disclosure; and v) a third variant 4-1BBL polypeptide of the present disclosure; and b) a second polypeptide comprising, in order from N-terminus to C-terminus: i) a Class I MHC heavy chain; and ii) an Fc polypeptide. In some cases, the first, second, and third variant 4-1BBL polypeptides have the same amino acid sequence. In some cases, the first, second, and third variant 4-1BBL polypeptides differ from one another in amino acid sequence. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a first variant 4-1BBL polypeptide of the present disclosure; ii) a second variant 4-1BBL polypeptide of the present disclosure; and iii) a third variant 4-1BBL polypeptide of the present disclosure; iv) a Class I MHC heavy chain; and v) an Fc polypeptide. In some cases, the first, second, and third variant 4-1BBL polypeptides have the same amino acid sequence. In some cases, the first, second, and third variant 4-1BBL polypeptides differ from one another in amino acid sequence.

### Linkers

A multimeric polypeptide of the present disclosure can include linker peptides interposed between, e.g., an epitope and an MHC polypeptide; between an MHC polypeptide and an immunomodulatory polypeptide; between an MHC polypeptide and an Ig Fc polypeptide; between a first variant 4-1BBL polypeptide and a second variant 4-1BBL polypeptide; or a between a second variant 4-1BBL polypeptide and a third variant 4-1BBL polypeptide.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length.

Exemplary linkers include glycine polymers (G)ₙ, glycine-serine polymers (including, for example, (GS)ₙ, (GSGGS)ₙ (SEQ ID NO:123) and (GGGS)ₙ (SEQ ID NO:124), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:125), GGSGG (SEQ ID NO: 126), GSGSG (SEQ ID NO:127), GSGGG (SEQ ID NO:128), GGGSG (SEQ ID NO:129), GSSSG (SEQ ID NO:130), and the like. Exemplary linkers can include, e.g., Gly(Ser₄)n, (SEQ ID NO:131) where n is 1, 2, 3, 4, 5, 6, 7, 8, 9, or 10. In some cases, a linker comprises the amino acid sequence (GSSSS)n (SEQ ID NO:131), where n is 4. In some cases, a linker comprises the amino acid sequence (GSSSS)n (SEQ ID NO:131) where n is 5. Exemplary linkers can include, e.g., (Gly)ₙSer, where n is 1, 2, 3, 4, 5, 6, 7, 8, 9, or 10. In some cases, a linker comprises the amino acid sequence (GGGGS)n (SEQ ID NO:132), where n is 4. In some cases, a linker comprises the amino acid sequence (GGGGS)n (SEQ ID NO:132), where n is 5.

In some cases, a linker polypeptide, present in a first polypeptide of a multimeric polypeptide of the present disclosure, includes a cysteine residue that can form a disulfide bond with a cysteine residue present in a second polypeptide of a multimeric polypeptide of the present disclosure. In some cases, for example, a suitable linker comprises the amino acid sequence GCGASGGGGSGGGGS (SEQ ID NO:133).

### Epitopes

An epitope present in a multimeric polypeptide of the present disclosure can have a length of from about 4 amino acids to about 25 amino acids, e.g., the epitope can have a length of from 4 amino acids (aa) to 10 aa, from 10 aa to 15 aa, from 15 aa to 20 aa, or from 20 aa to 25 aa. For example, an epitope present in a multimeric polypeptide of the present disclosure can have a length of 4 amino acids (aa), 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, an epitope present in a multimeric polypeptide of the present disclosure has a length of from 5 amino acids to 10 amino acids, e.g., 5 aa, 6 aa, 7 aa, 8 aa, 9 aa, or 10 aa.

An epitope present in a multimeric polypeptide of the present disclosure is specifically bound by a T-cell, i.e., the epitope is specifically bound by an epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

Suitable epitopes include, but are not limited to, epitopes present in a cancer-associated antigen. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); and vascular endothelial growth factor receptor-2 (VEGF-R2). See, e.g., Vigneron et al. (2013) Cancer Immunity 13:15; and Vigneron (2015) BioMed Res. Int'l Article ID 948501.

### MHC Polypeptides

As noted above, a multimeric polypeptide of the present disclosure includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain) and MHC Class II polypeptides (e.g., MHC Class II a polypeptide and MHC Class II β polypeptide).

As noted above, in some embodiments of a multimeric polypeptide of the present disclosure, the first and the second MHC polypeptides are Class I MHC polypeptides; e.g., in some cases, the first MHC polypeptide is an MHC Class I β2-microglobulin (β2M) polypeptide, and the second MHC polypeptide is an MHC Class I heavy chain (H chain). In other cases, the first and the second MHC polypeptides are Class II MHC polypeptides; e.g., in some cases, the first MHC polypeptide is an MHC Class II α-chain polypeptide, and the second MHC polypeptide is an MHC Class II β-chain polypeptide. In other cases, the first polypeptide is an MHC Class II β-chain polypeptide, and the second MHC polypeptide is an MHC Class II α-chain polypeptide.

In some cases, an MHC polypeptide of a multimeric polypeptide of the present disclosure is a human MHC polypeptide, where human MHC polypeptides are also referred to as “human leukocyte antigen” (“HLA”) polypeptides. In some cases, an MHC polypeptide of a multimeric polypeptide of the present disclosure is a Class I HLA polypeptide, e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides. In some cases, an MHC polypeptide of a multimeric polypeptide of the present disclosure is a Class II HLA polypeptide, e.g., a Class II HLA α chain or a Class II HLA β chain. MHC Class II polypeptides include MCH Class II DP α and β polypeptides, DM a and β polypeptides, DOA a and β polypeptides, DOB α and β polypeptides, DQ α and β polypeptides, and DR α and β polypeptides.

As an example, an MHC Class I heavy chain polypeptide of a multimeric polypeptide of the present disclosure can comprise an amino acid sequence having at least 75%, at least 80%, at least 85%, at least 90%, at least 95%, at least 98%, at least 99%, or 100%, amino acid sequence identity to amino acids 25-365 of the amino acid sequence of the human HLA-A heavy chain polypeptide depicted in FIG. 5A.

As an example, an MHC Class I heavy chain polypeptide of a multimeric polypeptide of the present disclosure can comprise an amino acid sequence having at least 75%, at least 80%, at least 85%, at least 90%, at least 95%, at least 98%, at least 99%, or 100%, amino acid sequence identity to amino acids 25-365 of the amino acid sequence of the following human HLA-A heavy chain amino acid sequence:

(SEQ ID NO: 134)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP.

As another example, an MHC Class I heavy chain polypeptide of a multimeric polypeptide of the present disclosure can comprise an amino acid sequence having at least 75%, at least 80%, at least 85%, at least 90%, at least 95%, at least 98%, at least 99%, or 100%, amino acid sequence identity to amino acids 25-362 of the amino acid sequence of the human HLA-B heavy chain polypeptide depicted in FIG. 5B.

As another example, an MHC Class I heavy chain polypeptide of a multimeric polypeptide of the present disclosure can comprise an amino acid sequence having at least 75%, at least 80%, at least 85%, at least 90%, at least 95%, at least 98%, at least 99%, or 100%, amino acid sequence identity to amino acids 25-362 of the amino acid sequence of the human HLA-C heavy chain polypeptide depicted in FIG. 5C.

As another example, an MHC Class I heavy chain polypeptide of a multimeric polypeptide of the present disclosure can comprise an amino acid sequence having at least 75%, at least 80%, at least 85%, at least 90%, at least 95%, at least 98%, at least 99%, or 100%, amino acid sequence identity to the following amino acid sequence:

(SEQ ID NO: 135)
GPHSLRYFVTAVSRPGLGEPRFIAVGYVDDTQFVRFDSDADNPRFEPRAP
WMEQEGPEYWEEQTQRAKSDEQWFRVSLRTAQRYYNQSKGGSHTFQRMFG
CDVGSDWRLLRGYQQFAYDGRDYIALNEDLKTWTAADTAALITRRKWEQA
GDAEYYRAYLEGECVEWLRRYLELGNETLLRTDSPKAHVTYHPRSQVDVT
LRCWALGFYPADITLTWQLNGEDLTQDMELVETRPAGDGTFQKWAAVVVP
LGKEQNYTCHVHHKGLPEPLTLRW.

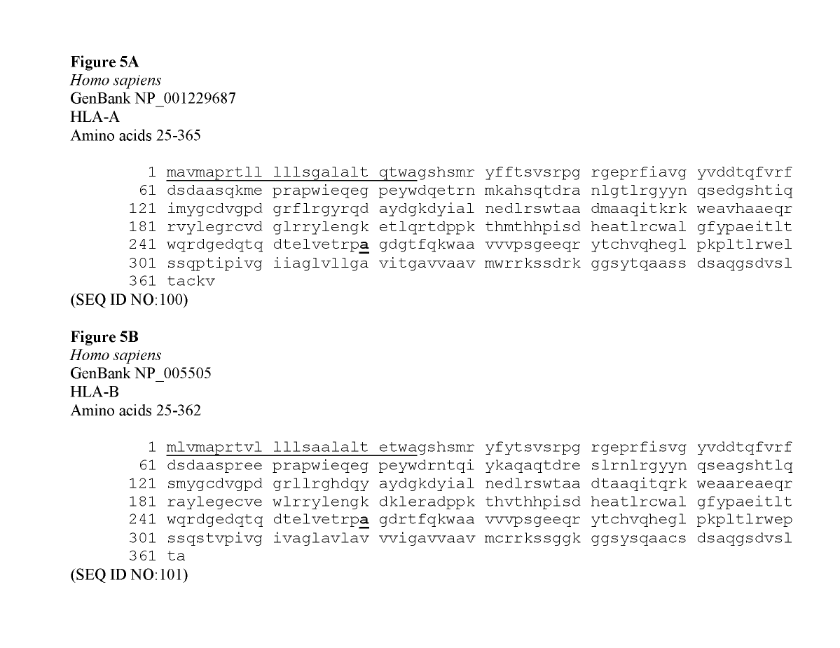

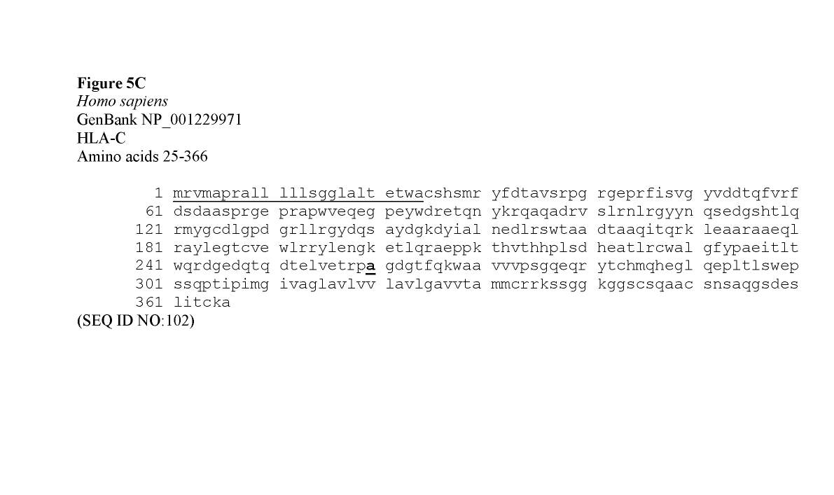

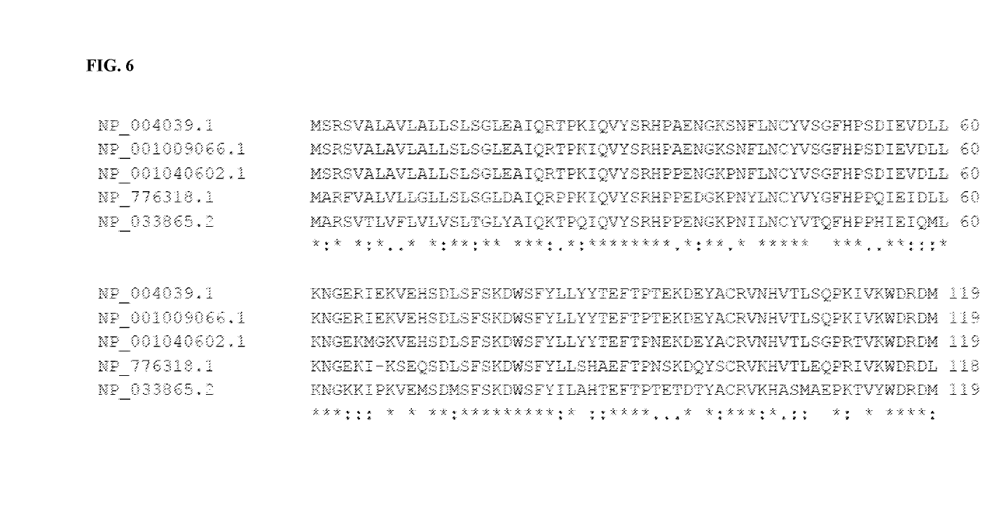

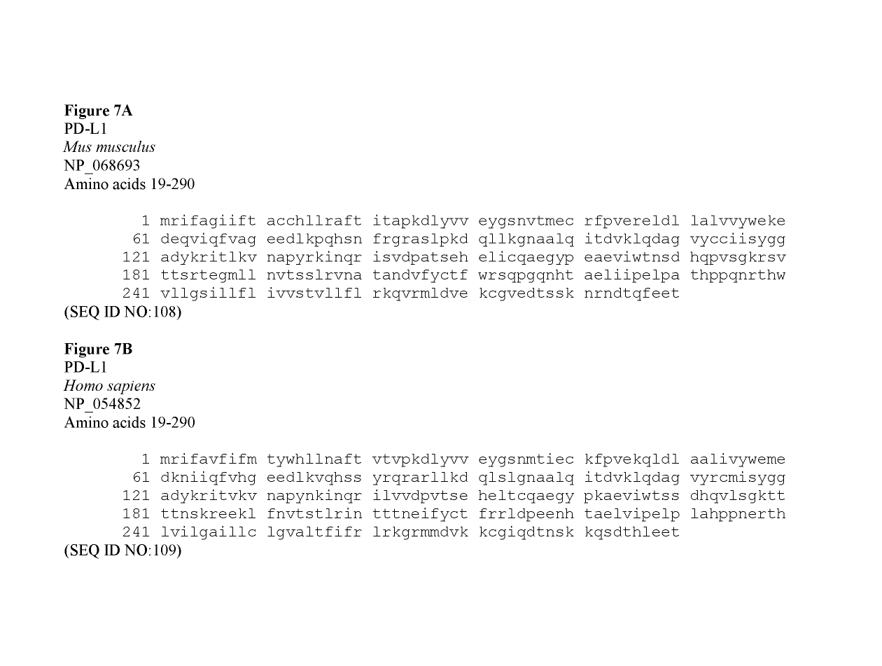

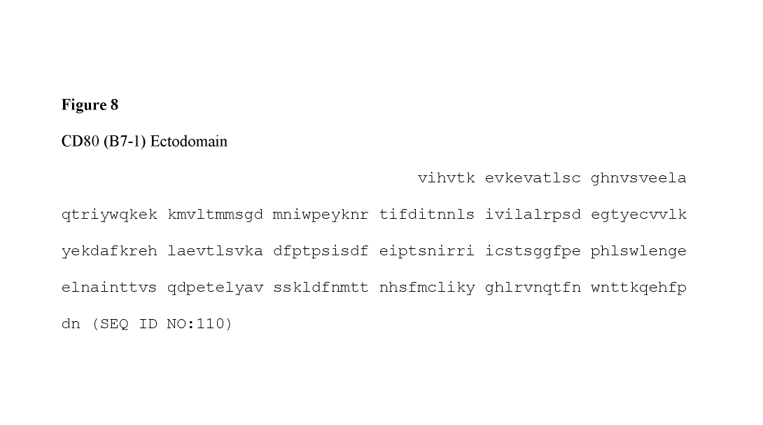

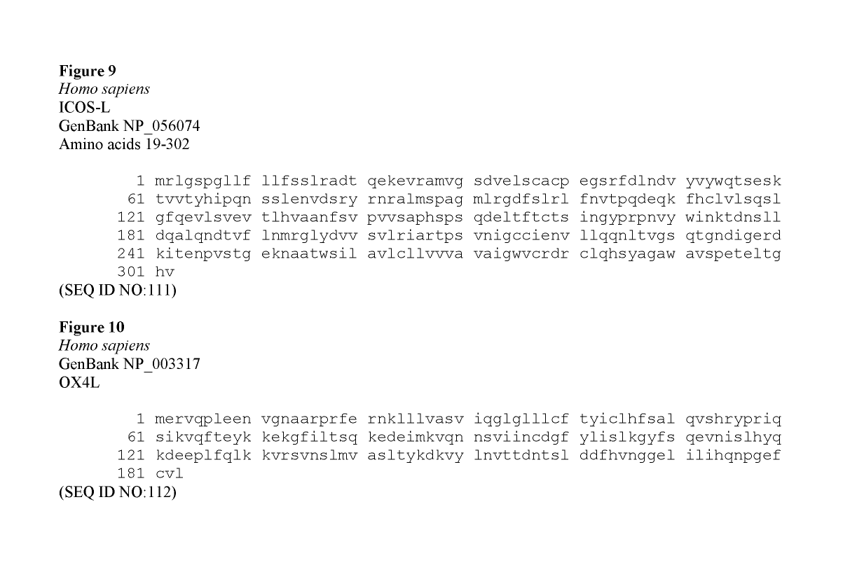

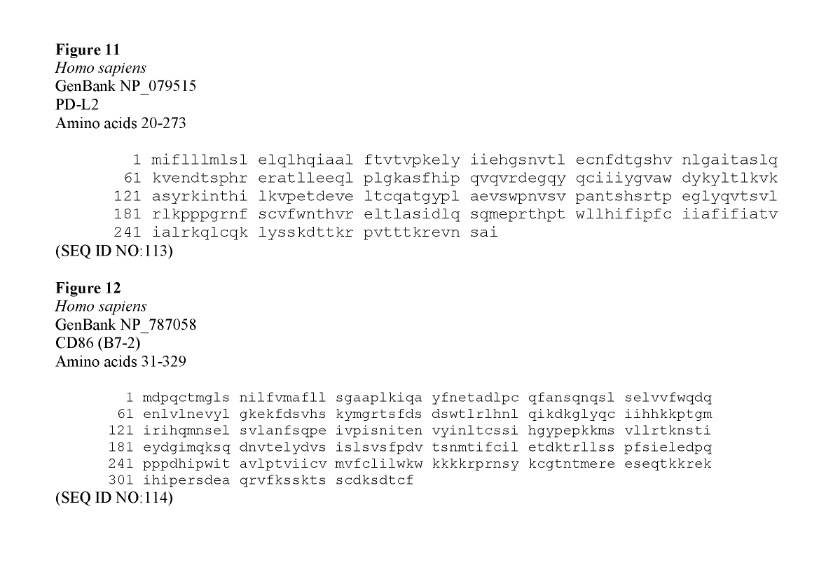

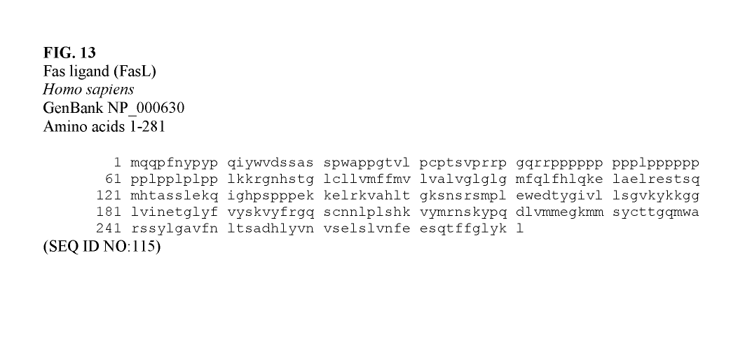

A β2-microglobulin (β2M) polypeptide of a multimeric polypeptide of the present disclosure can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to a β2M amino acid sequence depicted in FIG. 6. In some instances, a β2M polypeptide comprises an amino acid sequence having at least 75%, at least 80%, at least 85%, at least 90%, at least 95%, at least 98%, at least 99%, or 100%, amino acid sequence identity to amino acids 21 to 119 of a β2M amino acid sequence depicted in FIG. 6.

In some cases, an MHC polypeptide comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues, when present in an MHC polypeptide of a first polypeptide of a multimeric polypeptide of the present disclosure, can form a disulfide bond with a cysteine residue present in a second polypeptide chain of a multimeric polypeptide of the present disclosure.

In some cases, a first MHC polypeptide in a first polypeptide of a multimeric polypeptide of the present disclosure, and/or the second MHC polypeptide in the second polypeptide of a multimeric polypeptide of the present disclosure, includes an amino acid substitution to substitute an amino acid with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of following pairs of residues in an HLA β2-microglobulin and an HLA Class I heavy chain is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in the amino acid sequence depicted in FIG. 5A, which includes a signal peptide, Gly120 is Gly144; Gln96 is Gln120; etc. In some cases, the β2M polypeptide comprises an R12C substitution, and the HLA Class I heavy chain comprises an A236C substitution; in such cases, a disulfide bond forms between Cys-12 of the β2M polypeptide and Cys-236 of the HLA Class I heavy chain. For example, in some cases, residue 236 of the mature HLA-A amino acid sequence (i.e., residue 260 of the amino acid sequence depicted in FIG. 5A) is substituted with a Cys. In some cases, residue 236 of the mature HLA-B amino acid sequence (i.e., residue 260 of the amino acid sequence depicted in FIG. 5B) is substituted with a Cys. In some cases, residue 236 of the mature HLA-C amino acid sequence (i.e., residue 260 of the amino acid sequence depicted in FIG. 5C) is substituted with a Cys. In some cases, residue 32 (corresponding to Arg-12 of mature β2M) of an amino acid sequence depicted in FIG. 6 is substituted with a Cys.

In some cases, a β2M polypeptide comprises the amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM. In some cases, a β2M polypeptide comprises the amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM.

In some cases, an HLA Class I heavy chain polypeptide comprises the amino acid sequence:

(SEQ ID NO: 136)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPAGDGTFQKWAAVVVP
SGQEQRYTCHVQHEGLPKPLTLRWEP.

In some cases, an HLA Class I heavy chain polypeptide comprises the amino acid sequence:

(SEQ ID NO: 137)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWEP.

In some cases, the β2M polypeptide comprises the following amino acid sequence:

IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:138); and the HLA Class I heavy chain polypeptide of a multimeric polypeptide of the present disclosure comprises the following amino acid sequence:

(SEQ ID NO: 139)
GSHSMRYFFTSVSRPGRGEPRFIAVGYVDDTQFVRFDSDAASQRMEPRAP
WIEQEGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGEDQTQDTELVETRPCGDGTFQKWAAVVVP
SGQEQRYTCHVQHEGLPKPLTLRWEP,

where the Cys residues that are underlined and in bold form a disulfide bond with one another in the multimeric polypeptide.

### Immunomodulatory Polypeptides

A multimeric polypeptide of the present disclosure comprises a variant immunomodulatory polypeptide, as described above. Thus, a multimeric polypeptide of the present disclosure comprises a variant 4-1BBL polypeptide of the present disclosure.

In some cases, the variant 4-1BBL polypeptide present in a multimeric polypeptide of the present disclosure exhibits reduced binding affinity to 4-1BB, compared to the binding affinity of a 4-1BBL polypeptide comprising the amino acid sequence depicted in FIG. 2A for 4-1BB. For example, in some cases, the variant 4-1BBL polypeptide present in a multimeric polypeptide of the present disclosure binds 4-1BB with a binding affinity that is less than the binding affinity of a 4-1BBL polypeptide comprising the amino acid sequence depicted in FIG. 2A for a 4-1BB polypeptide comprising the amino acid sequence depicted in FIG. 3. For example, in some cases, the variant 4-1BBL polypeptide present in a multimeric polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence depicted in FIG. 2A for 4-1BB (e.g., a 4-1BB polypeptide comprising the amino acid sequence depicted in FIG. 3).

In some cases, the variant 4-1BBL polypeptide present in a multimeric polypeptide of the present disclosure exhibits reduced binding affinity to 4-1BB, compared to the binding affinity of a 4-1BBL polypeptide comprising the amino acid sequence depicted in SEQ ID NO:1 for 4-1BB. For example, in some cases, the variant 4-1BBL polypeptide present in a multimeric polypeptide of the present disclosure binds 4-1BB with a binding affinity that is less than the binding affinity of a 4-1BBL polypeptide comprising the amino acid sequence depicted in SEQ ID NO:1 for a 4-1BB polypeptide comprising the amino acid sequence depicted in one of FIG. 3A-3C. For example, in some cases, the variant 4-1BBL polypeptide present in a multimeric polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence depicted in SEQ ID NO:1 for 4-1BB (e.g., a 4-1BB polypeptide comprising the amino acid sequence depicted in FIG. 3).

In some cases, a variant 4-1BBL polypeptide present in a multimeric polypeptide of the present disclosure has a binding affinity to 4-1BB that is from 100 nM to 100 μM. As another example, in some cases, a variant 4-1BBL polypeptide present in a multimeric polypeptide of the present disclosure has a binding affinity for 4-1BB (e.g., a 4-1BB polypeptide comprising the amino acid sequence depicted in FIG. 3) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide present in a multimeric polypeptide of the present disclosure exhibits increased production in a mammalian host cell, compared to the production in the same mammalian host cell of a control multimeric polypeptide comprising a wild-type 4-1BBL polypeptide (e.g., a 4-1BBL polypeptide comprising the amino acid sequence depicted in FIG. 2A or as set forth in SEQ ID NO:1). For example, in some cases, a variant 4-1BBL polypeptide present in a multimeric polypeptide of the present disclosure, when expressed in a mammalian host cell, is produced in an amount that is from 25% higher to about 50% higher, from about 50% higher to about 75% higher, from about 75% higher to about 2-fold higher, from about 2-fold higher to about 5-fold higher, from about 5-fold higher to about 10-fold higher, from about 10-fold higher to about 20-fold higher, from about 20-fold higher to about 30-fold higher, from about 30-fold higher to about 40-fold higher, from about 40-fold higher to about 50-fold higher, from about 50-fold higher to about 75-fold higher, from about 75-fold higher to about 100-fold higher, or more than 100-fold higher, than the amount of a control multimeric polypeptide comprising a wild-type 4-1BBL polypeptide (e.g., a 4-1BBL polypeptide comprising the amino acid sequence depicted in FIG. 2A or as set forth in SEQ ID NO: 1) produced in the same mammalian host cell.

In some cases, a variant 4-1BBL polypeptide present in a multimeric polypeptide of the present disclosure is produced in a mammalian host cell in an amount of from about 50 mg/L to about 75 mg/L, from about 75 mg/L to about 100 mg/L, from about 100 mg/L to about 150 mg/L, from about 150 mg/L to about 200 mg/L, from about 200 mg/L to about 250 mg/L, from about 250 mg/L to about 500 mg/L, or more than 500 mg/L. In some cases, a variant 4-1BBL polypeptide present in a multimeric polypeptide of the present disclosure is produced in a mammalian host cell in an amount of from about 10 mg/L to about 15 mg/L, from about 15 mg/L to about 20 mg/L, from about 20 mg/L to about 25 mg/L, from about 25 mg/L to about 30 mg/L, from about 35 mg/L to about 40 mg/L, from about 40 mg/L to about 45 mg/L, or from about 45 mg/L to about 50 mg/L.

A variant 4-1BBL polypeptide present in a multimeric polypeptide of the present disclosure can have a single amino acid substitution relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has from 2 to 10 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 2 amino acid substitutions relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide present in a multimeric polypeptide of the present disclosure has 3 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 4 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 5 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 6 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide of the present disclosure has 7 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 8 amino acid substitutions relative to a wild-type 4-1BBL polypeptide (e.g., a 4-1BBL polypeptide comprising the amino acid sequence depicted in FIG. 2A or as set forth in SEQ ID NO:1). In some cases, a variant 4-1BBL polypeptide present in a multimeric polypeptide of the present disclosure has 9 amino acid substitutions relative to a wild-type 4-1BBL polypeptide (e.g., a 4-1BBL polypeptide comprising the amino acid sequence depicted in FIG. 2A or as set forth in SEQ ID NO: 1). In some cases, a variant 4-1BBL polypeptide present in a multimeric polypeptide of the present disclosure has 10 amino acid substitutions relative to a wild-type 4-1BBL polypeptide (e.g., a 4-1BBL polypeptide comprising the amino acid sequence depicted in FIG. 2A or as set forth in SEQ ID NO:1).

In some cases, a variant 4-1BBL polypeptide present in a multimeric polypeptide of the present disclosure has from 11 to 50 amino acid substitutions relative to a wild-type 4-1BBL polypeptide (e.g., a 4-1BBL polypeptide comprising the amino acid sequence depicted in FIG. 2A or as set forth in SEQ ID NO:1). For example, in some cases, a variant 4-1BBL polypeptide present in a multimeric polypeptide of the present disclosure has from 11 to 15, from 15 to 20, from 20 to 25, from 25 to 30, from 30 to 35, from 35 to 40, from 40 to 45, or from 45 to 50, amino acid substitutions relative to a wild-type 4-1BBL polypeptide (e.g., a 4-1BBL polypeptide comprising the amino acid sequence depicted in FIG. 2A or as set forth in SEQ ID NO:1).

Suitable variant 4-1BBL polypeptides that can be included in a multimeric polypeptide of the present disclosure include those described above.

4-1BBL with K127 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at K48. In some cases, a variant 4-1BBL polypeptide present in a multimeric polypeptide of the present disclosure comprises the amino acid sequence set forth in SEQ ID NO:2, with an amino acid substitution at K48. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at K48. In some cases, a variant 4-1BBL polypeptide present in a multimeric polypeptide of the present disclosure comprises the amino acid sequence set forth in SEQ ID NO:3, with an amino acid substitution at K48.

### K127+M91 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at M91, where amino acid 91 is Gly, Ala, Val, Leu, Ile, Pro, Phe, Tyr, Trp, Ser, Thr, Cys, Asn, Gln, Lys, Arg, His, Asp, or Glu. In some cases, amino acid 127 is Ala; and amino acid 91 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 11 is other than methionine, e.g., where amino acid 11 is Gly, Ala, Val, Leu, Ile, Pro, Phe, Tyr, Trp, Ser, Thr, Cys, Asn, Gln, Lys, Arg, His, Asp, or Glu. In some cases, amino acid 47 is Ala; and amino acid 11 is Ala.

### K127+F92 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at F92, where amino acid 92 is Gly, Ala, Val, Leu, Ile, Pro, Tyr, Trp, Ser, Thr, Cys, Met, Asn, Gln, Lys, Arg, His, Asp, or Glu. In some cases, amino acid 127 is Ala; and amino acid 92 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 12 is other than phenylalanine, e.g., where amino acid 12 is Gly, Ala, Val, Leu, Ile, Pro, Tyr, Trp, Ser, Thr, Cys, Met, Asn, Gln, Lys, Arg, His, Asp, or Glu. In some cases, amino acid 47 is Ala; and amino acid 12 is Ala.

### K127+Q94 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at Q94, where amino acid 94 is Gly, Ala, Val, Leu, Ile, Pro, Phe, Tyr, Trp, Ser, Thr, Cys, Met, Asn, Lys, Arg, His, Asp, or Glu. In some cases, amino acid 127 is Ala; and amino acid 94 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 14 is other than glutamine, e.g., where amino acid 14 is Gly, Ala, Val, Leu, Ile, Pro, Phe, Tyr, Trp, Ser, Thr, Cys, Met, Asn, Lys, Arg, His, Asp, or Glu. In some cases, amino acid 47 is Ala; and amino acid 14 is Ala.

### K127+L95 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at L95, where amino acid 95 is Gly, Ala, Val, Ile, Pro, Phe, Tyr, Trp, Ser, Thr, Cys, Met, Asn, Gln, Lys, Arg, His, Asp, or Glu. In some cases, amino acid 127 is Ala; and amino acid 95 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 15 is other than leucine, e.g., where amino acid 15 is Gly, Ala, Val, Ile, Pro, Phe, Tyr, Trp, Ser, Thr, Cys, Met, Asn, Gln, Lys, Arg, His, Asp, or Glu. In some cases, amino acid 47 is Ala; and amino acid 15 is Ala.

### K127+V96 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at V96, where amino acid 96 is Gly, Ala, Leu, Ile, Pro, Phe, Tyr, Trp, Ser, Thr, Cys, Met, Asn, Gln, Lys, Arg, His, Asp, or Glu. In some cases, amino acid 127 is Ala; and amino acid 96 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 16 is other than a valine, e.g., where amino acid 16 is Gly, Ala, Leu, Ile, Pro, Phe, Tyr, Trp, Ser, Thr, Cys, Met, Asn, Gln, Lys, Arg, His, Asp, or Glu. In some cases, amino acid 47 is Ala; and amino acid 16 is Ala.

### K127+Q98 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at Q98, where amino acid 98 is Gly, Ala, Val, Leu, Ile, Pro, Phe, Tyr, Trp, Ser, Thr, Cys, Met, Asn, Lys, Arg, His, Asp, or Glu. In some cases, amino acid 127 is Ala; and amino acid 98 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 18 is other than glutamine, e.g., where amino acid 18 is Gly, Ala, Val, Leu, Ile, Pro, Phe, Tyr, Trp, Ser, Thr, Cys, Met, Asn, Lys, Arg, His, Asp, or Glu. In some cases, amino acid 47 is Ala; and amino acid 18 is Ala.

### K127+N99 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at N99, where amino acid 99 is Gly, Ala, Val, Leu, Ile, Pro, Phe, Tyr, Trp, Ser, Thr, Cys, Met, Gln, Lys, Arg, His, Asp, or Glu. In some cases, amino acid 127 is Ala; and amino acid 99 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 19 is other than an asparagine, e.g., where amino acid 19 is Gly, Ala, Val, Leu, Ile, Pro, Phe, Tyr, Trp, Ser, Thr, Cys, Met, Gln, Lys, Arg, His, Asp, or Glu. In some cases, amino acid 47 is Ala; and amino acid 19 is Ala.

### K127+V100 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at V100, where amino acid 100 is Gly, Ala, Leu, Ile, Pro, Phe, Tyr, Trp, Ser, Thr, Cys, Met, Asn, Gln, Lys, Arg, His, Asp, or Glu. In some cases, amino acid 127 is Ala; and amino acid 100 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 20 is other than a valine, e.g., where amino acid 20 is Gly, Ala, Leu, Ile, Pro, Phe, Tyr, Trp, Ser, Thr, Cys, Met, Asn, Gln, Lys, Arg, His, Asp, or Glu. In some cases, amino acid 47 is Ala; and amino acid 20 is Ala.

### K127+L101 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at L101, where amino acid 101 is Gly, Ala, Val, Ile, Pro, Phe, Tyr, Trp, Ser, Thr, Cys, Met, Asn, Gln, Lys, Arg, His, Asp, or Glu. In some cases, amino acid 127 is Ala; and amino acid 101 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 21 is other than leucine, e.g., where amino acid 21 is Gly, Ala, Val, Ile, Pro, Phe, Tyr, Trp, Ser, Thr, Cys, Met, Asn, Gln, Lys, Arg, His, Asp, or Glu. In some cases, amino acid 47 is Ala; and amino acid 21 is Ala.

### K127+L102 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at L102, where amino acid 102 is Gly, Ala, Val, Ile, Pro, Phe, Tyr, Trp, Ser, Thr, Cys, Met, Asn, Gln, Lys, Arg, His, Asp, or Glu. In some cases, amino acid 127 is Ala; and amino acid 102 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 22 is other than leucine, e.g., where amino acid 22 is Gly, Ala, Val, Ile, Pro, Phe, Tyr, Trp, Ser, Thr, Cys, Met, Asn, Gln, Lys, Arg, His, Asp, or Glu. In some cases, amino acid 47 is Ala; and amino acid 22 is Ala.

### K127+I103 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at I103, where amino acid 103 is Gly, Ala, Val, Leu, Pro, Phe, Tyr, Trp, Ser, Thr, Cys, Met, Asn, Gln, Lys, Arg, His, Asp, or Glu. In some cases, amino acid 127 is Ala; and amino acid 103 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 23 is other than isoleucine, e.g., where amino acid 23 is Gly, Ala, Val, Leu, Pro, Phe, Tyr, Trp, Ser, Thr, Cys, Met, Asn, Gln, Lys, Arg, His, Asp, or Glu. In some cases, amino acid 47 is Ala; and amino acid 23 is Ala.

### K127+D104 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at D104, where amino acid 104 is Gly, Ala, Val, Leu, Ile, Pro, Phe, Tyr, Trp, Ser, Thr, Cys, Met, Asn, Gln, Lys, Arg, His, or Glu. In some cases, amino acid 127 is Ala; and amino acid 104 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 24 is other than aspartic acid, e.g., where amino acid 24 is Gly, Ala, Val, Leu, Ile, Pro, Phe, Tyr, Trp, Ser, Thr, Cys, Met, Asn, Gln, Lys, Arg, His, or Glu. In some cases, amino acid 47 is Ala; and amino acid 24 is Ala.

### K127+G105 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at G105, where amino acid 105 is Ala, Val, Leu, Ile, Pro, Phe, Tyr, Trp, Ser, Thr, Cys, Met, Asn, Gln, Lys, Arg, His, Asp, or Glu. In some cases, amino acid 127 is Ala; and amino acid 105 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 25 is other than glycine, e.g., where amino acid 25 is Ala, Val, Leu, Ile, Pro, Phe, Tyr, Trp, Ser, Thr, Cys, Met, Asn, Gln, Lys, Arg, His, Asp, or Glu. In some cases, amino acid 47 is Ala; and amino acid 25 is Ala.

### K127+P106 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at P106, where amino acid 106 is Gly, Ala, Val, Leu, Ile, Phe, Tyr, Trp, Ser, Thr, Cys, Met, Asn, Gln, Lys, Arg, His, Asp, or Glu. In some cases, amino acid 127 is Ala; and amino acid 106 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 26 is other than proline, e.g., where amino acid 26 is Gly, Ala, Val, Leu, Ile, Phe, Tyr, Trp, Ser, Thr, Cys, Met, Asn, Gln, Lys, Arg, His, Asp, or Glu. In some cases, amino acid 47 is Ala; and amino acid 26 is Ala.

### K127+L107 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at L107, where amino acid 107 is Gly, Ala, Val, Ile, Pro, Phe, Tyr, Trp, Ser, Thr, Cys, Met, Asn, Gln, Lys, Arg, His, Asp, or Glu. In some cases, amino acid 127 is Ala; and amino acid 107 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 27 is other than leucine, e.g., where amino acid 27 is Gly, Ala, Val, Ile, Pro, Phe, Tyr, Trp, Ser, Thr, Cys, Met, Asn, Gln, Lys, Arg, His, Asp, or Glu. In some cases, amino acid 47 is Ala; and amino acid 27 is Ala.

### K127+S108 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at S108, where amino acid 108 is Gly, Ala, Val, Leu, Ile, Pro, Phe, Tyr, Trp, Thr, Cys, Met, Asn, Gln, Lys, Arg, His, Asp, or Glu. In some cases, amino acid 127 is Ala; and amino acid 108 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 28 is other than serine, e.g., where amino acid 28 is Gly, Ala, Val, Leu, Ile, Pro, Phe, Tyr, Trp, Thr, Cys, Met, Asn, Gln, Lys, Arg, His, Asp, or Glu. In some cases, amino acid 47 is Ala; and amino acid 28 is Ala.

### K127+W109 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at W109, where amino acid 109 is Gly, Ala, Val, Leu, Ile, Pro, Phe, Tyr, Ser, Thr, Cys, Met, Asn, Gln, Lys, Arg, His, Asp, or Glu. In some cases, amino acid 127 is Ala; and amino acid 109 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 29 is other than tryptophan, e.g., where amino acid 29 is Gly, Ala, Val, Leu, Ile, Pro, Phe, Tyr, Ser, Thr, Cys, Met, Asn, Gln, Lys, Arg, His, Asp, or Glu. In some cases, amino acid 47 is Ala; and amino acid 29 is Ala.

### K127+Y110 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at Y110, where amino acid 110 is Gly, Ala, Val, Leu, Ile, Pro, Phe, Trp, Ser, Thr, Cys, Met, Asn, Gln, Lys, Arg, His, Asp, or Glu. In some cases, amino acid 127 is Ala; and amino acid 110 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 30 is other than tyrosine, e.g., where amino acid 30 is Gly, Ala, Val, Leu, Ile, Pro, Phe, Trp, Ser, Thr, Cys, Met, Asn, Gln, Lys, Arg, His, Asp, or Glu. In some cases, amino acid 47 is Ala; and amino acid 30 is Ala.

### K127+S111 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at S111, where amino acid 111 is Gly, Ala, Val, Leu, Ile, Pro, Phe, Tyr, Trp, Thr, Cys, Met, Asn, Gln, Lys, Arg, His, Asp, or Glu. In some cases, amino acid 127 is Ala; and amino acid 111 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 31 is other than serine, e.g., where amino acid 31 is Gly, Ala, Val, Leu, Ile, Pro, Phe, Tyr, Trp, Thr, Cys, Met, Asn, Gln, Lys, Arg, His, Asp, or Glu. In some cases, amino acid 47 is Ala; and amino acid 31 is Ala.

### K127+D112 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at D112, where amino acid 112 is Gly, Ala, Val, Leu, Ile, Pro, Phe, Tyr, Trp, Ser, Thr, Cys, Met, Asn, Gln, Lys, Arg, His, or Glu. In some cases, amino acid 127 is Ala; and amino acid 112 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 32 is other than aspartic acid, e.g., where amino acid 32 is Gly, Ala, Val, Leu, Ile, Pro, Phe, Tyr, Trp, Ser, Thr, Cys, Met, Asn, Gln, Lys, Arg, His, or Glu. In some cases, amino acid 47 is Ala; and amino acid 32 is Ala.

### K127+P113 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at P113, where amino acid 113 is Gly, Ala, Val, Leu, Ile, Phe, Tyr, Trp, Ser, Thr, Cys, Met, Asn, Gln, Lys, Arg, His, Asp, or Glu. In some cases, amino acid 127 is Ala; and amino acid 113 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 33 is other than proline, e.g., where amino acid 33 is Gly, Ala, Val, Leu, Ile, Phe, Tyr, Trp, Ser, Thr, Cys, Met, Asn, Gln, Lys, Arg, His, Asp, or Glu. In some cases, amino acid 47 is Ala; and amino acid 33 is Ala.

### K127+G114 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at G114, where amino acid 114 is Ala, Val, Leu, Ile, Pro, Phe, Tyr, Trp, Ser, Thr, Cys, Met, Asn, Gln, Lys, Arg, His, Asp, or Glu. In some cases, amino acid 127 is Ala; and amino acid 114 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 34 is other than glycine, e.g., where amino acid 34 is Ala, Val, Leu, Ile, Pro, Phe, Tyr, Trp, Ser, Thr, Cys, Met, Asn, Gln, Lys, Arg, His, Asp, or Glu. In some cases, amino acid 47 is Ala; and amino acid 34 is Ala.

### K127+L115 Substitutions

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2B, where: i) amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; and ii) an amino acid substitution at L115, where amino acid 115 is Gly, Ala, Val, Ile, Pro, Phe, Tyr, Trp, Ser, Thr, Cys, Met, Asn, Gln, Lys, Arg, His, Asp, or Glu. In some cases, amino acid 127 is Ala; and amino acid 115 is Ala. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where: i) amino acid 47 is an amino acid other than a lysine, e.g., where amino acid 47 is Gly, Ala, Val, Leu, Ile, Pro, Phe, Tyr, Trp, Ser, Thr, Cys, Met, Asn, Gln, Arg, His, Asp, or Glu; and ii) amino acid 35 is other than leucine, e.g., where amino acid 35 is Gly, Ala, Val, Ile, Pro, Phe, Tyr, Trp, Ser, Thr, Cys, Met, Asn, Gln, Lys, Arg, His, Asp, or Glu. In some cases, amino acid 47 is Ala; and amino acid 35 is Ala.

4-1BBL with Q227 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2D where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 147 is other than glutamine, e.g., where amino acid 147 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q148. In some cases, a variant 4-1BBL polypeptide present in a multimeric polypeptide of the present disclosure comprises the amino acid sequence set forth in SEQ ID NO:2, with an amino acid substitution at Q148. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q148. In some cases, a variant 4-1BBL polypeptide present in a multimeric polypeptide of the present disclosure comprises the amino acid sequence set forth in SEQ ID NO:3, with an amino acid substitution at Q148.

4-1BBL with M91 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2E, where amino acid 91 (indicated by an “x”) is an amino acid other than a methionine, e.g., where amino acid 91 is Gly, Ala, Val, Leu, Ile, Pro, Phe, Tyr, Trp, Ser, Thr, Cys, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 11 is other than a methionine, e.g., where amino acid 11 is Gly, Ala, Val, Leu, Ile, Pro, Phe, Tyr, Trp, Ser, Thr, Cys, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at M12. In some cases, a variant 4-1BBL polypeptide present in a multimeric polypeptide of the present disclosure comprises the amino acid sequence set forth in SEQ ID NO:2, with an amino acid substitution at M12. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at M12. In some cases, a variant 4-1BBL polypeptide present in a multimeric polypeptide of the present disclosure comprises the amino acid sequence set forth in SEQ ID NO:3, with an amino acid substitution at M12.

4-1BBL with F92 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2F, where amino acid 92 (indicated by an “x”) is an amino acid other than a phenylalanine, e.g., where amino acid 92 is Gly, Ala, Val, Leu, Ile, Pro, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 12 is other than a phenylalanine, e.g., where amino acid 12 is Gly, Ala, Val, Leu, Ile, Pro, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F13. In some cases, a variant 4-1BBL polypeptide present in a multimeric polypeptide of the present disclosure comprises the amino acid sequence set forth in SEQ ID NO:2, with an amino acid substitution at F13. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F13. In some cases, a variant 4-1BBL polypeptide present in a multimeric polypeptide of the present disclosure comprises the amino acid sequence set forth in SEQ ID NO:3, with an amino acid substitution at F13.

4-1BBL with Q94 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2G, where amino acid 94 (indicated by an “x”) is an amino acid other than a glutamine, e.g., where amino acid 94 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 14 is other than a glutamine, e.g., where amino acid 14 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q15. In some cases, a variant 4-1BBL polypeptide present in a multimeric polypeptide of the present disclosure comprises the amino acid sequence set forth in SEQ ID NO:2, with an amino acid substitution at Q15. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q15. In some cases, a variant 4-1BBL polypeptide present in a multimeric polypeptide of the present disclosure comprises the amino acid sequence set forth in SEQ ID NO:3, with an amino acid substitution at Q15.

4-1BBL with L95 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2H, where amino acid 95 (indicated by an “x”) is an amino acid other than a leucine, e.g., where amino acid 95 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 15 is other than a leucine, e.g., where amino acid 15 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L16. In some cases, a variant 4-1BBL polypeptide present in a multimeric polypeptide of the present disclosure comprises the amino acid sequence set forth in SEQ ID NO:2, with an amino acid substitution at L16. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L16. In some cases, a variant 4-1BBL polypeptide present in a multimeric polypeptide of the present disclosure comprises the amino acid sequence set forth in SEQ ID NO:3, with an amino acid substitution at L16.

4-1BBL with V96 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2I, where amino acid 96 (indicated by an “x”) is an amino acid other than a valine, e.g., where amino acid 96 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 16 is other than a valine, e.g., where amino acid 16 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V17. In some cases, a variant 4-1BBL polypeptide present in a multimeric polypeptide of the present disclosure comprises the amino acid sequence set forth in SEQ ID NO:2, with an amino acid substitution at V17. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V17. In some cases, a variant 4-1BBL polypeptide present in a multimeric polypeptide of the present disclosure comprises the amino acid sequence set forth in SEQ ID NO:3, with an amino acid substitution at V17.

4-1BBL with Q98 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2J, where amino acid 98 (indicated by an “x”) is an amino acid other than a glutamine, e.g., where amino acid 98 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 18 is other than a glutamine, e.g., where amino acid 18 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q19. In some cases, a variant 4-1BBL polypeptide present in a multimeric polypeptide of the present disclosure comprises the amino acid sequence set forth in SEQ ID NO:2, with an amino acid substitution at Q19. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q19. In some cases, a variant 4-1BBL polypeptide present in a multimeric polypeptide of the present disclosure comprises the amino acid sequence set forth in SEQ ID NO:3, with an amino acid substitution at Q19.

4-1BBL with N99 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2K, where amino acid 99 (indicated by an “x”) is an amino acid other than an asparagine, e.g., where amino acid 99 is Gly, Ala, Val, Leu, Ile, Pro, Phe, Tyr, Trp, Ser, Thr, Cys, Met, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 19 is other than an asparagine, e.g., where amino acid 19 is Gly, Ala, Val, Leu, Ile, Pro, Phe, Tyr, Trp, Ser, Thr, Cys, Met, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at N20. In some cases, a variant 4-1BBL polypeptide present in a multimeric polypeptide of the present disclosure comprises the amino acid sequence set forth in SEQ ID NO:2, with an amino acid substitution at N20. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at N20. In some cases, a variant 4-1BBL polypeptide present in a multimeric polypeptide of the present disclosure comprises the amino acid sequence set forth in SEQ ID NO:3, with an amino acid substitution at N20.

4-1BBL with V100 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2L, where amino acid 100 (indicated by an “x”) is an amino acid other than a valine, e.g., where amino acid 100 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 20 is other than a valine, e.g., where amino acid 20 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V21. In some cases, a variant 4-1BBL polypeptide present in a multimeric polypeptide of the present disclosure comprises the amino acid sequence set forth in SEQ ID NO:2, with an amino acid substitution at V21. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V21. In some cases, a variant 4-1BBL polypeptide present in a multimeric polypeptide of the present disclosure comprises the amino acid sequence set forth in SEQ ID NO:3, with an amino acid substitution at V21.

4-1BBL with L101 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2M, where amino acid 101 (indicated by an “x”) is an amino acid other than a leucine, e.g., where amino acid 101 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 21 is other than a leucine, e.g., where amino acid 21 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L22. In some cases, a variant 4-1BBL polypeptide present in a multimeric polypeptide of the present disclosure comprises the amino acid sequence set forth in SEQ ID NO:2, with an amino acid substitution at L22. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L22. In some cases, a variant 4-1BBL polypeptide present in a multimeric polypeptide of the present disclosure comprises the amino acid sequence set forth in SEQ ID NO:3, with an amino acid substitution at L22.

4-1BBL with L102 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2N, where amino acid 102 (indicated by an “x”) is an amino acid other than a leucine, e.g., where amino acid 102 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 22 is other than a leucine, e.g., where amino acid 22 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L23. In some cases, a variant 4-1BBL polypeptide present in a multimeric polypeptide of the present disclosure comprises the amino acid sequence set forth in SEQ ID NO:2, with an amino acid substitution at L23. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L23. In some cases, a variant 4-1BBL polypeptide present in a multimeric polypeptide of the present disclosure comprises the amino acid sequence set forth in SEQ ID NO:3, with an amino acid substitution at L23.

4-1BBL with I103 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2O, where amino acid 103 (indicated by an “x”) is an amino acid other than an isoleucine, e.g., where amino acid 103 is Gly, Ala, Val, Leu, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 23 is other than an isoleucine, e.g., where amino acid 23 is Gly, Ala, Val, Leu, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at 124. In some cases, a variant 4-1BBL polypeptide present in a multimeric polypeptide of the present disclosure comprises the amino acid sequence set forth in SEQ ID NO:2, with an amino acid substitution at 124. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at 124. In some cases, a variant 4-1BBL polypeptide present in a multimeric polypeptide of the present disclosure comprises the amino acid sequence set forth in SEQ ID NO:3, with an amino acid substitution at 124.

4-1BBL with D104 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2P, where amino acid 104 (indicated by an “x”) is an amino acid other than an aspartic acid, e.g., where amino acid 104 is Gly, Ala, Val, Leu, Ile, Pro, Phe, Tyr, Trp, Ser, Thr, Cys, Met, Asn, Gln, Lys, Arg, His, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 24 is other than an aspartic acid, e.g., where amino acid 24 is Gly, Ala, Val, Leu, Ile, Pro, Phe, Tyr, Trp, Ser, Thr, Cys, Met, Asn, Gln, Lys, Arg, His, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D25. In some cases, a variant 4-1BBL polypeptide present in a multimeric polypeptide of the present disclosure comprises the amino acid sequence set forth in SEQ ID NO:2, with an amino acid substitution at D25. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D25. In some cases, a variant 4-1BBL polypeptide present in a multimeric polypeptide of the present disclosure comprises the amino acid sequence set forth in SEQ ID NO:3, with an amino acid substitution at D25.

4-1BBL with G105 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2Q, where amino acid 105 (indicated by an “x”) is an amino acid other than a glycine, e.g., where amino acid 105 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 25 is other than a glycine, e.g., where amino acid 25 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G26. In some cases, a variant 4-1BBL polypeptide present in a multimeric polypeptide of the present disclosure comprises the amino acid sequence set forth in SEQ ID NO:2, with an amino acid substitution at G26. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G26. In some cases, a variant 4-1BBL polypeptide present in a multimeric polypeptide of the present disclosure comprises the amino acid sequence set forth in SEQ ID NO:3, with an amino acid substitution at G26.

4-1BBL with P106 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2R, where amino acid 106 (indicated by an “x”) is an amino acid other than a proline, e.g., where amino acid 106 is Gly, Ala, Val, Leu, Ile, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 26 is other than a proline, e.g., where amino acid 26 is Gly, Ala, Val, Leu, Ile, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P27. In some cases, a variant 4-1BBL polypeptide present in a multimeric polypeptide of the present disclosure comprises the amino acid sequence set forth in SEQ ID NO:2, with an amino acid substitution at P27. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P27. In some cases, a variant 4-1BBL polypeptide present in a multimeric polypeptide of the present disclosure comprises the amino acid sequence set forth in SEQ ID NO:3, with an amino acid substitution at P27.

4-1BBL with L107 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2S, where amino acid 107 (indicated by an “x”) is an amino acid other than a leucine, e.g., where amino acid 107 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 27 is other than a leucine, e.g., where amino acid 27 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L28. In some cases, a variant 4-1BBL polypeptide present in a multimeric polypeptide of the present disclosure comprises the amino acid sequence set forth in SEQ ID NO:2, with an amino acid substitution at L28. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L28. In some cases, a variant 4-1BBL polypeptide present in a multimeric polypeptide of the present disclosure comprises the amino acid sequence set forth in SEQ ID NO:3, with an amino acid substitution at L28.

4-1BBL with S108 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2T, where amino acid 108 (indicated by an “x”) is an amino acid other than a serine, e.g., where amino acid 108 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 28 is other than a serine, e.g., where amino acid 28 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S29. In some cases, a variant 4-1BBL polypeptide present in a multimeric polypeptide of the present disclosure comprises the amino acid sequence set forth in SEQ ID NO:2, with an amino acid substitution at S29. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S29. In some cases, a variant 4-1BBL polypeptide present in a multimeric polypeptide of the present disclosure comprises the amino acid sequence set forth in SEQ ID NO:3, with an amino acid substitution at S29.

4-1BBL with W109 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2U, where amino acid 109 (indicated by an “x”) is an amino acid other than a tryptophan, e.g., where amino acid 109 is Gly, Ala, Val, Leu, Ile, Pro, Phe, Tyr, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 29 is other than a tryptophan, e.g., where amino acid 29 is Gly, Ala, Val, Leu, Ile, Pro, Phe, Tyr, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at W30. In some cases, a variant 4-1BBL polypeptide present in a multimeric polypeptide of the present disclosure comprises the amino acid sequence set forth in SEQ ID NO:2, with an amino acid substitution at W30. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at W30. In some cases, a variant 4-1BBL polypeptide present in a multimeric polypeptide of the present disclosure comprises the amino acid sequence set forth in SEQ ID NO:3, with an amino acid substitution at W30.

4-1BBL with Y110 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2V, where amino acid 110 (indicated by an “x”) is an amino acid other than a tyrosine, e.g., where amino acid 110 is Gly, Ala, Val, Leu, Ile, Pro, Phe, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 30 is other than a tyrosine, e.g., where amino acid 30 is Gly, Ala, Val, Leu, Ile, Pro, Phe, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Y31. In some cases, a variant 4-1BBL polypeptide present in a multimeric polypeptide of the present disclosure comprises the amino acid sequence set forth in SEQ ID NO:2, with an amino acid substitution at Y31. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Y31. In some cases, a variant 4-1BBL polypeptide present in a multimeric polypeptide of the present disclosure comprises the amino acid sequence set forth in SEQ ID NO:3, with an amino acid substitution at Y31.

4-1BBL with S111 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2W, where amino acid 111 (indicated by an “x”) is an amino acid other than a serine, e.g., where amino acid 111 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 31 is other than a serine, e.g., where amino acid 31 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S32. In some cases, a variant 4-1BBL polypeptide present in a multimeric polypeptide of the present disclosure comprises the amino acid sequence set forth in SEQ ID NO:2, with an amino acid substitution at S32. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S32. In some cases, a variant 4-1BBL polypeptide present in a multimeric polypeptide of the present disclosure comprises the amino acid sequence set forth in SEQ ID NO:3, with an amino acid substitution at S32.

4-1BBL with D112 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2X, where amino acid 112 (indicated by an “x”) is an amino acid other than an aspartic acid, e.g., where amino acid 112 is Gly, Ala, Val, Leu, Ile, Pro, Phe, Tyr, Trp, Ser, Thr, Cys, Met, Asn, Gln, Lys, Arg, His, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 32 is other than an aspartic acid, e.g., where amino acid 32 is Gly, Ala, Val, Leu, Ile, Pro, Phe, Tyr, Trp, Ser, Thr, Cys, Met, Asn, Gln, Lys, Arg, His, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D33. In some cases, a variant 4-1BBL polypeptide present in a multimeric polypeptide of the present disclosure comprises the amino acid sequence set forth in SEQ ID NO:2, with an amino acid substitution at D33. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D33. In some cases, a variant 4-1BBL polypeptide present in a multimeric polypeptide of the present disclosure comprises the amino acid sequence set forth in SEQ ID NO:3, with an amino acid substitution at D33.

4-1BBL with P113 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2Y, where amino acid 113 (indicated by an “x”) is an amino acid other than a proline, e.g., where amino acid 113 is Gly, Ala, Val, Leu, Ile, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 33 is other than a proline, e.g., where amino acid 33 is Gly, Ala, Val, Leu, Ile, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P34. In some cases, a variant 4-1BBL polypeptide present in a multimeric polypeptide of the present disclosure comprises the amino acid sequence set forth in SEQ ID NO:2, with an amino acid substitution at P34. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P34. In some cases, a variant 4-1BBL polypeptide present in a multimeric polypeptide of the present disclosure comprises the amino acid sequence set forth in SEQ ID NO:3, with an amino acid substitution at P34.

4-1BBL with G114 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2Z, where amino acid 114 (indicated by an “x”) is an amino acid other than a glycine, e.g., where amino acid 114 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 34 is other than a glycine, e.g., where amino acid 34 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G35. In some cases, a variant 4-1BBL polypeptide present in a multimeric polypeptide of the present disclosure comprises the amino acid sequence set forth in SEQ ID NO:2, with an amino acid substitution at G35. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G35. In some cases, a variant 4-1BBL polypeptide present in a multimeric polypeptide of the present disclosure comprises the amino acid sequence set forth in SEQ ID NO:3, with an amino acid substitution at G35.

4-1BBL with L115 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2AA, where amino acid 115 (indicated by an “x”) is an amino acid other than a leucine, e.g., where amino acid 115 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 35 is other than a leucine, e.g., where amino acid 35 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L36. In some cases, a variant 4-1BBL polypeptide present in a multimeric polypeptide of the present disclosure comprises the amino acid sequence set forth in SEQ ID NO:2, with an amino acid substitution at L36. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L36. In some cases, a variant 4-1BBL polypeptide present in a multimeric polypeptide of the present disclosure comprises the amino acid sequence set forth in SEQ ID NO:3, with an amino acid substitution at L36.

4-1BBL with G117 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2BB, where amino acid 117 (indicated by an “x”) is an amino acid other than a glycine, e.g., where amino acid 117 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 37 is other than a glycine, e.g., where amino acid 37 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G38. In some cases, a variant 4-1BBL polypeptide present in a multimeric polypeptide of the present disclosure comprises the amino acid sequence set forth in SEQ ID NO:2, with an amino acid substitution at G38. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G38. In some cases, a variant 4-1BBL polypeptide present in a multimeric polypeptide of the present disclosure comprises the amino acid sequence set forth in SEQ ID NO:3, with an amino acid substitution at G38.

4-1BBL with V118 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2CC, where amino acid 118 (indicated by an “x”) is an amino acid other than a valine, e.g., where amino acid 118 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 38 is other than a valine, e.g., where amino acid 38 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V39. In some cases, a variant 4-1BBL polypeptide present in a multimeric polypeptide of the present disclosure comprises the amino acid sequence set forth in SEQ ID NO:2, with an amino acid substitution at V39. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V39. In some cases, a variant 4-1BBL polypeptide present in a multimeric polypeptide of the present disclosure comprises the amino acid sequence set forth in SEQ ID NO:3, with an amino acid substitution at V39.

4-1BBL with S119 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2DD, where amino acid 119 (indicated by an “x”) is an amino acid other than a serine, e.g., where amino acid 119 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 39 is other than a serine, e.g., where amino acid 39 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S40. In some cases, a variant 4-1BBL polypeptide present in a multimeric polypeptide of the present disclosure comprises the amino acid sequence set forth in SEQ ID NO:2, with an amino acid substitution at S40. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S40. In some cases, a variant 4-1BBL polypeptide present in a multimeric polypeptide of the present disclosure comprises the amino acid sequence set forth in SEQ ID NO:3, with an amino acid substitution at S40.

4-1BBL with L120 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2EE, where amino acid 120 (indicated by an “x”) is an amino acid other than a leucine, e.g., where amino acid 120 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 40 is other than a leucine, e.g., where amino acid 40 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L41. In some cases, a variant 4-1BBL polypeptide present in a multimeric polypeptide of the present disclosure comprises the amino acid sequence set forth in SEQ ID NO:2, with an amino acid substitution at L41. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L41. In some cases, a variant 4-1BBL polypeptide present in a multimeric polypeptide of the present disclosure comprises the amino acid sequence set forth in SEQ ID NO:3, with an amino acid substitution at L41.

4-1BBL with T121 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2FF, where amino acid 121 (indicated by an “x”) is an amino acid other than a threonine, e.g., where amino acid 121 is Gly, Ala, Val, Leu, Ile, Pro, Phe, Tyr, Trp, Ser, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 41 is other than a threonine, e.g., where amino acid 41 is Gly, Ala, Val, Leu, Ile, Pro, Phe, Tyr, Trp, Ser, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T42. In some cases, a variant 4-1BBL polypeptide present in a multimeric polypeptide of the present disclosure comprises the amino acid sequence set forth in SEQ ID NO:2, with an amino acid substitution at T42. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T42. In some cases, a variant 4-1BBL polypeptide present in a multimeric polypeptide of the present disclosure comprises the amino acid sequence set forth in SEQ ID NO:3, with an amino acid substitution at T42.

4-1BBL with G122 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2GG, where amino acid 122 (indicated by an “x”) is an amino acid other than a glycine, e.g., where amino acid 122 is Ala, Val, Leu, Ile, Pro, Phe, Tyr, Trp, Ser, Thr, Cys, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 42 is other than a glycine, e.g., where amino acid 42 is Ala, Val, Leu, Ile, Pro, Phe, Tyr, Trp, Ser, Thr, Cys, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G43. In some cases, a variant 4-1BBL polypeptide present in a multimeric polypeptide of the present disclosure comprises the amino acid sequence set forth in SEQ ID NO:2, with an amino acid substitution at G43. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G43. In some cases, a variant 4-1BBL polypeptide present in a multimeric polypeptide of the present disclosure comprises the amino acid sequence set forth in SEQ ID NO:3, with an amino acid substitution at G43.

4-1BBL with G123 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2HH, where amino acid 123 (indicated by an “x”) is an amino acid other than a glycine, e.g., where amino acid 123 is Ala, Val, Leu, Ile, Pro, Phe, Tyr, Trp, Ser, Thr, Cys, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 43 is other than a glycine, e.g., where amino acid 43 is Ala, Val, Leu, Ile, Pro, Phe, Tyr, Trp, Ser, Thr, Cys, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G44. In some cases, a variant 4-1BBL polypeptide present in a multimeric polypeptide of the present disclosure comprises the amino acid sequence set forth in SEQ ID NO:2, with an amino acid substitution at G44. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G44. In some cases, a variant 4-1BBL polypeptide present in a multimeric polypeptide of the present disclosure comprises the amino acid sequence set forth in SEQ ID NO:3, with an amino acid substitution at G44.

4-1BBL with L124 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2II, where amino acid 124 (indicated by an “x”) is an amino acid other than a leucine, e.g., where amino acid 124 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 44 is other than a leucine, e.g., where amino acid 44 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L45. In some cases, a variant 4-1BBL polypeptide present in a multimeric polypeptide of the present disclosure comprises the amino acid sequence set forth in SEQ ID NO:2, with an amino acid substitution at L45. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L45. In some cases, a variant 4-1BBL polypeptide present in a multimeric polypeptide of the present disclosure comprises the amino acid sequence set forth in SEQ ID NO:3, with an amino acid substitution at L45.

4-1BBL with S125 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2JJ, where amino acid 125 (indicated by an “x”) is an amino acid other than a serine, e.g., where amino acid 125 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 45 is other than a serine, e.g., where amino acid 45 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S46. In some cases, a variant 4-1BBL polypeptide present in a multimeric polypeptide of the present disclosure comprises the amino acid sequence set forth in SEQ ID NO:2, with an amino acid substitution at S46. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S46. In some cases, a variant 4-1BBL polypeptide present in a multimeric polypeptide of the present disclosure comprises the amino acid sequence set forth in SEQ ID NO:3, with an amino acid substitution at S46.

4-1BBL with Y126 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2KK, where amino acid 126 (indicated by an “x”) is an amino acid other than a tyrosine, e.g., where amino acid 126 is Gly, Ala, Val, Leu, Ile, Pro, Phe, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 46 is other than a tyrosine, e.g., where amino acid 46 is Gly, Ala, Val, Leu, Ile, Pro, Phe, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Y47. In some cases, a variant 4-1BBL polypeptide present in a multimeric polypeptide of the present disclosure comprises the amino acid sequence set forth in SEQ ID NO:2, with an amino acid substitution at Y47. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Y47. In some cases, a variant 4-1BBL polypeptide present in a multimeric polypeptide of the present disclosure comprises the amino acid sequence set forth in SEQ ID NO:3, with an amino acid substitution at Y47.

4-1BBL with E128 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2LL, where amino acid 128 (indicated by an “x”) is an amino acid other than a glutamic acid, e.g., where amino acid 128 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 48 is other than a glutamic acid, e.g., where amino acid 48 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E49. In some cases, a variant 4-1BBL polypeptide present in a multimeric polypeptide of the present disclosure comprises the amino acid sequence set forth in SEQ ID NO:2, with an amino acid substitution at E49. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E49. In some cases, a variant 4-1BBL polypeptide present in a multimeric polypeptide of the present disclosure comprises the amino acid sequence set forth in SEQ ID NO:3, with an amino acid substitution at E49.

4-1BBL with D129 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2MM, where amino acid 129 (indicated by an “x”) is an amino acid other than an aspartic acid, e.g., where amino acid 129 is Gly, Ala, Val, Leu, Ile, Pro, Phe, Tyr, Trp, Ser, Thr, Cys, Met, Asn, Gln, Lys, Arg, His, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 49 is other than an aspartic acid, e.g., where amino acid 49 is Gly, Ala, Val, Leu, Ile, Pro, Phe, Tyr, Trp, Ser, Thr, Cys, Met, Asn, Gln, Lys, Arg, His, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D50. In some cases, a variant 4-1BBL polypeptide present in a multimeric polypeptide of the present disclosure comprises the amino acid sequence set forth in SEQ ID NO:2, with an amino acid substitution at D50. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D50. In some cases, a variant 4-1BBL polypeptide present in a multimeric polypeptide of the present disclosure comprises the amino acid sequence set forth in SEQ ID NO:3, with an amino acid substitution at D50.

4-1BBL with T130 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2NN, where amino acid 130 (indicated by an “x”) is an amino acid other than a threonine, e.g., where amino acid 130 is Gly, Ala, Val, Leu, Ile, Pro, Phe, Tyr, Trp, Ser, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 50 is other than a threonine, e.g., where amino acid 50 is Gly, Ala, Val, Leu, Ile, Pro, Phe, Tyr, Trp, Ser, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T51. In some cases, a variant 4-1BBL polypeptide present in a multimeric polypeptide of the present disclosure comprises the amino acid sequence set forth in SEQ ID NO:2, with an amino acid substitution at T51. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T51. In some cases, a variant 4-1BBL polypeptide present in a multimeric polypeptide of the present disclosure comprises the amino acid sequence set forth in SEQ ID NO:3, with an amino acid substitution at T51.

4-1BBL with K131 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2OO, where amino acid 131 (indicated by an “x”) is an amino acid other than a lysine, e.g., where amino acid 131 is Gly, Ala, Val, Leu, Ile, Pro, Phe, Tyr, Trp, Ser, Thr, Cys, Met, Asn, Gln, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 51 is other than a lysine, e.g., where amino acid 51 is Gly, Ala, Val, Leu, Ile, Pro, Phe, Tyr, Trp, Ser, Thr, Cys, Met, Asn, Gln, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at K52. In some cases, a variant 4-1BBL polypeptide present in a multimeric polypeptide of the present disclosure comprises the amino acid sequence set forth in SEQ ID NO:2, with an amino acid substitution at K52. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at K52. In some cases, a variant 4-1BBL polypeptide present in a multimeric polypeptide of the present disclosure comprises the amino acid sequence set forth in SEQ ID NO:3, with an amino acid substitution at K52.

4-1BBL with E132 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2PP, where amino acid 132 (indicated by an “x”) is an amino acid other than a glutamic acid, e.g., where amino acid 132 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 52 is other than a glutamic acid, e.g., where amino acid 52 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E53. In some cases, a variant 4-1BBL polypeptide present in a multimeric polypeptide of the present disclosure comprises the amino acid sequence set forth in SEQ ID NO:2, with an amino acid substitution at E53. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E53. In some cases, a variant 4-1BBL polypeptide present in a multimeric polypeptide of the present disclosure comprises the amino acid sequence set forth in SEQ ID NO:3, with an amino acid substitution at E53.

4-1BBL with F144 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2QQ, where amino acid 144 (indicated by an “x”) is an amino acid other than a phenylalanine, e.g., where amino acid 144 is Gly, Ala, Val, Leu, Ile, Pro, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 64 is other than a phenylalanine, e.g., where amino acid 64 is Gly, Ala, Val, Leu, Ile, Pro, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F65. In some cases, a variant 4-1BBL polypeptide present in a multimeric polypeptide of the present disclosure comprises the amino acid sequence set forth in SEQ ID NO:2, with an amino acid substitution at F65. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F65. In some cases, a variant 4-1BBL polypeptide present in a multimeric polypeptide of the present disclosure comprises the amino acid sequence set forth in SEQ ID NO:3, with an amino acid substitution at F65.

4-1BBL with F145 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2RR, where amino acid 145 (indicated by an “x”) is an amino acid other than a phenylalanine, e.g., where amino acid 145 is Gly, Ala, Val, Leu, Ile, Pro, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 65 is other than a phenylalanine, e.g., where amino acid 65 is Gly, Ala, Val, Leu, Ile, Pro, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F66. In some cases, a variant 4-1BBL polypeptide present in a multimeric polypeptide of the present disclosure comprises the amino acid sequence set forth in SEQ ID NO:2, with an amino acid substitution at F66. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F66. In some cases, a variant 4-1BBL polypeptide present in a multimeric polypeptide of the present disclosure comprises the amino acid sequence set forth in SEQ ID NO:3, with an amino acid substitution at F66.

4-1BBL with Q146 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2SS, where amino acid 146 (indicated by an “x”) is an amino acid other than a glutamine, e.g., where amino acid 146 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 66 is other than a glutamine, e.g., where amino acid 66 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q67. In some cases, a variant 4-1BBL polypeptide present in a multimeric polypeptide of the present disclosure comprises the amino acid sequence set forth in SEQ ID NO:2, with an amino acid substitution at Q67. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q67. In some cases, a variant 4-1BBL polypeptide present in a multimeric polypeptide of the present disclosure comprises the amino acid sequence set forth in SEQ ID NO:3, with an amino acid substitution at Q67.

4-1BBL with L147 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2TT, where amino acid 147 (indicated by an “x”) is an amino acid other than a leucine, e.g., where amino acid 147 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 67 is other than a leucine, e.g., where amino acid 67 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L68. In some cases, a variant 4-1BBL polypeptide present in a multimeric polypeptide of the present disclosure comprises the amino acid sequence set forth in SEQ ID NO:2, with an amino acid substitution at L68. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L68. In some cases, a variant 4-1BBL polypeptide present in a multimeric polypeptide of the present disclosure comprises the amino acid sequence set forth in SEQ ID NO:3, with an amino acid substitution at L68.

4-1BBL with E148 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2UU, where amino acid 148 (indicated by an “x”) is an amino acid other than a glutamic acid, e.g., where amino acid 148 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 68 is other than a glutamic acid, e.g., where amino acid 68 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E69. In some cases, a variant 4-1BBL polypeptide present in a multimeric polypeptide of the present disclosure comprises the amino acid sequence set forth in SEQ ID NO:2, with an amino acid substitution at E69. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E69. In some cases, a variant 4-1BBL polypeptide present in a multimeric polypeptide of the present disclosure comprises the amino acid sequence set forth in SEQ ID NO:3, with an amino acid substitution at E69.

4-1BBL with L149 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2VV, where amino acid 149 (indicated by an “x”) is an amino acid other than a leucine, e.g., where amino acid 149 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 69 is other than a leucine, e.g., where amino acid 69 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L70. In some cases, a variant 4-1BBL polypeptide present in a multimeric polypeptide of the present disclosure comprises the amino acid sequence set forth in SEQ ID NO:2, with an amino acid substitution at L70. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L70. In some cases, a variant 4-1BBL polypeptide present in a multimeric polypeptide of the present disclosure comprises the amino acid sequence set forth in SEQ ID NO:3, with an amino acid substitution at L70.

4-1BBL with R150 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2WW, where amino acid 150 (indicated by an “x”) is an amino acid other than an arginine, e.g., where amino acid 150 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 70 is other than an arginine, e.g., where amino acid 70 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R71. In some cases, a variant 4-1BBL polypeptide present in a multimeric polypeptide of the present disclosure comprises the amino acid sequence set forth in SEQ ID NO:2, with an amino acid substitution at R71. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R71. In some cases, a variant 4-1BBL polypeptide present in a multimeric polypeptide of the present disclosure comprises the amino acid sequence set forth in SEQ ID NO:3, with an amino acid substitution at R71.

4-1BBL with R151 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2XX, where amino acid 151 (indicated by an “x”) is an amino acid other than an arginine, e.g., where amino acid 151 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 71 is is other than an arginine, e.g., where amino acid 71 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R72. In some cases, a variant 4-1BBL polypeptide present in a multimeric polypeptide of the present disclosure comprises the amino acid sequence set forth in SEQ ID NO:2, with an amino acid substitution at R72. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R72. In some cases, a variant 4-1BBL polypeptide present in a multimeric polypeptide of the present disclosure comprises the amino acid sequence set forth in SEQ ID NO:3, with an amino acid substitution at R72.

4-1BBL with V152 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2YY, where amino acid 152 (indicated by an “x”) is an amino acid other than a valine, e.g., where amino acid 152 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 72 is other than a valine, e.g., where amino acid 72 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V73. In some cases, a variant 4-1BBL polypeptide present in a multimeric polypeptide of the present disclosure comprises the amino acid sequence set forth in SEQ ID NO:2, with an amino acid substitution at V73. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V73. In some cases, a variant 4-1BBL polypeptide present in a multimeric polypeptide of the present disclosure comprises the amino acid sequence set forth in SEQ ID NO:3, with an amino acid substitution at V73.

4-1BBL with V153 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2ZZ, where amino acid 153 (indicated by an “x”) is an amino acid other than a valine, e.g., where amino acid 153 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 73 is other than a valine, e.g., where amino acid 73 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V74. In some cases, a variant 4-1BBL polypeptide present in a multimeric polypeptide of the present disclosure comprises the amino acid sequence set forth in SEQ ID NO:2, with an amino acid substitution at V74. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V74. In some cases, a variant 4-1BBL polypeptide present in a multimeric polypeptide of the present disclosure comprises the amino acid sequence set forth in SEQ ID NO:3, with an amino acid substitution at V74.

4-1BBL with G155 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2AAA, where amino acid 155 (indicated by an “x”) is an amino acid other than a glycine, e.g., where amino acid 155 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 75 is other than a glycine, e.g., where amino acid 75 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G76. In some cases, a variant 4-1BBL polypeptide present in a multimeric polypeptide of the present disclosure comprises the amino acid sequence set forth in SEQ ID NO:2, with an amino acid substitution at G76. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G76. In some cases, a variant 4-1BBL polypeptide present in a multimeric polypeptide of the present disclosure comprises the amino acid sequence set forth in SEQ ID NO:3, with an amino acid substitution at G76.

4-1BBL with E156 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2BBB, where amino acid 156 (indicated by an “x”) is an amino acid other than a glutamic acid, e.g., where amino acid 156 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 76 is other than a glutamic acid, e.g., where amino acid 76 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E77. In some cases, a variant 4-1BBL polypeptide present in a multimeric polypeptide of the present disclosure comprises the amino acid sequence set forth in SEQ ID NO:2, with an amino acid substitution at E77. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E77. In some cases, a variant 4-1BBL polypeptide present in a multimeric polypeptide of the present disclosure comprises the amino acid sequence set forth in SEQ ID NO:3, with an amino acid substitution at E77.

4-1BBL with G157 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2CCC, where amino acid 157 (indicated by an “x”) is an amino acid other than a glycine, e.g., where amino acid 157 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 77 is other than a glycine, e.g., where amino acid 77 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G78. In some cases, a variant 4-1BBL polypeptide present in a multimeric polypeptide of the present disclosure comprises the amino acid sequence set forth in SEQ ID NO:2, with an amino acid substitution at G78. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G78. In some cases, a variant 4-1BBL polypeptide present in a multimeric polypeptide of the present disclosure comprises the amino acid sequence set forth in SEQ ID NO:3, with an amino acid substitution at G78.

4-1BBL with S158 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2DDD, where amino acid 158 (indicated by an “x”) is an amino acid other than a serine, e.g., where amino acid 158 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 78 is other than a serine, e.g., where amino acid 78 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S79. In some cases, a variant 4-1BBL polypeptide present in a multimeric polypeptide of the present disclosure comprises the amino acid sequence set forth in SEQ ID NO:2, with an amino acid substitution at S79. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S79. In some cases, a variant 4-1BBL polypeptide present in a multimeric polypeptide of the present disclosure comprises the amino acid sequence set forth in SEQ ID NO:3, with an amino acid substitution at S79.

4-1BBL with D184 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2EEE, where amino acid 184 (indicated by an “x”) is an amino acid other than an aspartic acid, e.g., where amino acid 184 is Gly, Ala, Val, Leu, Ile, Pro, Phe, Tyr, Trp, Ser, Thr, Cys, Met, Asn, Gln, Lys, Arg, His, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 104 is other than an aspartic acid, e.g., where amino acid 104 is Gly, Ala, Val, Leu, Ile, Pro, Phe, Tyr, Trp, Ser, Thr, Cys, Met, Asn, Gln, Lys, Arg, His, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at D105. In some cases, a variant 4-1BBL polypeptide present in a multimeric polypeptide of the present disclosure comprises the amino acid sequence set forth in SEQ ID NO:2, with an amino acid substitution at D105. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at D105. In some cases, a variant 4-1BBL polypeptide present in a multimeric polypeptide of the present disclosure comprises the amino acid sequence set forth in SEQ ID NO:3, with an amino acid substitution at D105.

4-1BBL with L185 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2FFF, where amino acid 185 (indicated by an “x”) is an amino acid other than a leucine, e.g., where amino acid 185 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 105 is other than a leucine, e.g., where amino acid 105 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L106. In some cases, a variant 4-1BBL polypeptide present in a multimeric polypeptide of the present disclosure comprises the amino acid sequence set forth in SEQ ID NO:2, with an amino acid substitution at L106. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L106. In some cases, a variant 4-1BBL polypeptide present in a multimeric polypeptide of the present disclosure comprises the amino acid sequence set forth in SEQ ID NO:3, with an amino acid substitution at L106.

4-1BBL with P186 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2GGG, where amino acid 186 (indicated by an “x”) is an amino acid other than a proline, e.g., where amino acid 186 is Gly, Ala, Val, Leu, Ile, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 106 is other than a proline, e.g., where amino acid 106 is Gly, Ala, Val, Leu, Ile, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P107. In some cases, a variant 4-1BBL polypeptide present in a multimeric polypeptide of the present disclosure comprises the amino acid sequence set forth in SEQ ID NO:2, with an amino acid substitution at P107. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P107. In some cases, a variant 4-1BBL polypeptide present in a multimeric polypeptide of the present disclosure comprises the amino acid sequence set forth in SEQ ID NO:3, with an amino acid substitution at P107.

4-1BBL with P187 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2HHH, where amino acid 187 (indicated by an “x”) is an amino acid other than a proline, e.g., where amino acid 187 is Gly, Ala, Val, Leu, Ile, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 107 is other than a proline, e.g., where amino acid 107 is Gly, Ala, Val, Leu, Ile, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at P108. In some cases, a variant 4-1BBL polypeptide present in a multimeric polypeptide of the present disclosure comprises the amino acid sequence set forth in SEQ ID NO:2, with an amino acid substitution at P108. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at P108. In some cases, a variant 4-1BBL polypeptide present in a multimeric polypeptide of the present disclosure comprises the amino acid sequence set forth in SEQ ID NO:3, with an amino acid substitution at P108.

4-1BBL with S189 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2III, where amino acid 189 (indicated by an “x”) is an amino acid other than a serine, e.g., where amino acid 189 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 109 is other than a serine, e.g., where amino acid 109 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S110. In some cases, a variant 4-1BBL polypeptide present in a multimeric polypeptide of the present disclosure comprises the amino acid sequence set forth in SEQ ID NO:2, with an amino acid substitution at S110. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S110. In some cases, a variant 4-1BBL polypeptide present in a multimeric polypeptide of the present disclosure comprises the amino acid sequence set forth in SEQ ID NO:3, with an amino acid substitution at S110.

4-1BBL with S190 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2JJJ, where amino acid 190 (indicated by an “x”) is an amino acid other than a serine, e.g., where amino acid 190 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 110 is other than a serine, e.g., where amino acid 110 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S111. In some cases, a variant 4-1BBL polypeptide present in a multimeric polypeptide of the present disclosure comprises the amino acid sequence set forth in SEQ ID NO:2, with an amino acid substitution at S111. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S111. In some cases, a variant 4-1BBL polypeptide present in a multimeric polypeptide of the present disclosure comprises the amino acid sequence set forth in SEQ ID NO:3, with an amino acid substitution at S111.

4-1BBL with E191 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2KKK, where amino acid 191 (indicated by an “x”) is an amino acid other than a glutamic acid, e.g., where amino acid 191 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 111 is other than a glutamic acid, e.g., where amino acid 111 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E112. In some cases, a variant 4-1BBL polypeptide present in a multimeric polypeptide of the present disclosure comprises the amino acid sequence set forth in SEQ ID NO:2, with an amino acid substitution at E112. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E112. In some cases, a variant 4-1BBL polypeptide present in a multimeric polypeptide of the present disclosure comprises the amino acid sequence set forth in SEQ ID NO:3, with an amino acid substitution at E112.

4-1BBL with R193 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2LLL, where amino acid 193 (indicated by an “x”) is an amino acid other than an arginine, e.g., where amino acid 193 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 113 is other than arginine, e.g., where amino acid 113 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R114. In some cases, a variant 4-1BBL polypeptide present in a multimeric polypeptide of the present disclosure comprises the amino acid sequence set forth in SEQ ID NO:2, with an amino acid substitution at R114. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R114. In some cases, a variant 4-1BBL polypeptide present in a multimeric polypeptide of the present disclosure comprises the amino acid sequence set forth in SEQ ID NO:3, with an amino acid substitution at R114.

4-1BBL with N194 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2MMM, where amino acid 194 (indicated by an “x”) is an amino acid other than a asparagine, e.g., where amino acid 194 is Gly, Ala, Val, Leu, Ile, Pro, Phe, Tyr, Trp, Ser, Thr, Cys, Met, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 114 is other than a asparagine, e.g., where amino acid 114 is Gly, Ala, Val, Leu, Ile, Pro, Phe, Tyr, Trp, Ser, Thr, Cys, Met, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at N115. In some cases, a variant 4-1BBL polypeptide present in a multimeric polypeptide of the present disclosure comprises the amino acid sequence set forth in SEQ ID NO:2, with an amino acid substitution at N115. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at N115. In some cases, a variant 4-1BBL polypeptide present in a multimeric polypeptide of the present disclosure comprises the amino acid sequence set forth in SEQ ID NO:3, with an amino acid substitution at N115.

4-1BBL with S195 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2NNN, where amino acid 195 (indicated by an “x”) is an amino acid other than a serine, e.g., where amino acid 195 is Gly, Ala, Val, Leu, Ile, Pro, Phe, Tyr, Trp, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 115 is other than a serine, e.g., where amino acid 115 is Gly, Ala, Val, Leu, Ile, Pro, Phe, Tyr, Trp, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at S116. In some cases, a variant 4-1BBL polypeptide present in a multimeric polypeptide of the present disclosure comprises the amino acid sequence set forth in SEQ ID NO:2, with an amino acid substitution at S116. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at S116. In some cases, a variant 4-1BBL polypeptide present in a multimeric polypeptide of the present disclosure comprises the amino acid sequence set forth in SEQ ID NO:3, with an amino acid substitution at S116.

4-1BBL with F197 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2OOO, where amino acid 197 (indicated by an “x”) is an amino acid other than a phenylalanine, e.g., where amino acid 197 is Gly, Ala, Val, Leu, Ile, Pro, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO: 1, where amino acid 117 is other than a phenylalanine, e.g., where amino acid 117 is Gly, Ala, Val, Leu, Ile, Pro, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at F118. In some cases, a variant 4-1BBL polypeptide present in a multimeric polypeptide of the present disclosure comprises the amino acid sequence set forth in SEQ ID NO:2, with an amino acid substitution at F118. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at F118. In some cases, a variant 4-1BBL polypeptide present in a multimeric polypeptide of the present disclosure comprises the amino acid sequence set forth in SEQ ID NO:3, with an amino acid substitution at F118.

4-1BBL with Q210 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2PPP, where amino acid 210 (indicated by an “x”) is an amino acid other than a glutamine, e.g., where amino acid 210 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 130 is other than a glutamine, e.g., where amino acid 130 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q131. In some cases, a variant 4-1BBL polypeptide present in a multimeric polypeptide of the present disclosure comprises the amino acid sequence set forth in SEQ ID NO:2, with an amino acid substitution at Q131. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q131. In some cases, a variant 4-1BBL polypeptide present in a multimeric polypeptide of the present disclosure comprises the amino acid sequence set forth in SEQ ID NO:3, with an amino acid substitution at Q131.

4-1BBL with R211 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2QQQ, where amino acid 211 (indicated by an “x”) is an amino acid other than an arginine, e.g., where amino acid 211 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 131 is other than an arginine, e.g., where amino acid 131 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R132. In some cases, a variant 4-1BBL polypeptide present in a multimeric polypeptide of the present disclosure comprises the amino acid sequence set forth in SEQ ID NO:2, with an amino acid substitution at R132. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R132. In some cases, a variant 4-1BBL polypeptide present in a multimeric polypeptide of the present disclosure comprises the amino acid sequence set forth in SEQ ID NO:3, with an amino acid substitution at R132.

4-1BBL with L212 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2RRR, where amino acid 212 (indicated by an “x”) is an amino acid other than a leucine, e.g., where amino acid 212 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 132 is other than a leucine, e.g., where amino acid 132 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L133. In some cases, a variant 4-1BBL polypeptide present in a multimeric polypeptide of the present disclosure comprises the amino acid sequence set forth in SEQ ID NO:2, with an amino acid substitution at L133. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L133. In some cases, a variant 4-1BBL polypeptide present in a multimeric polypeptide of the present disclosure comprises the amino acid sequence set forth in SEQ ID NO:3, with an amino acid substitution at L133.

4-1BBL with G213 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2SSS, where amino acid 213 (indicated by an “x”) is an amino acid other than a glycine, e.g., where amino acid 213 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 133 is other than a glycine, e.g., where amino acid 133 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G134. In some cases, a variant 4-1BBL polypeptide present in a multimeric polypeptide of the present disclosure comprises the amino acid sequence set forth in SEQ ID NO:2, with an amino acid substitution at G134. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G134. In some cases, a variant 4-1BBL polypeptide present in a multimeric polypeptide of the present disclosure comprises the amino acid sequence set forth in SEQ ID NO:3, with an amino acid substitution at G134.

4-1BBL with V214 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2TTT, where amino acid 214 (indicated by an “x”) is an amino acid other than a valine, e.g., where amino acid 214 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 134 is other than a valine, e.g., where amino acid 134 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V135. In some cases, a variant 4-1BBL polypeptide present in a multimeric polypeptide of the present disclosure comprises the amino acid sequence set forth in SEQ ID NO:2, with an amino acid substitution at V135. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V135. In some cases, a variant 4-1BBL polypeptide present in a multimeric polypeptide of the present disclosure comprises the amino acid sequence set forth in SEQ ID NO:3, with an amino acid substitution at V135.

4-1BBL with H215 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2UUU, where amino acid 215 (indicated by an “x”) is an amino acid other than a histidine, e.g., where amino acid 215 is Gly, Ala, Val, Leu, Ile, Pro, Phe, Tyr, Trp, Ser, Thr, Cys, Met, Asn, Gln, Lys, Arg, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 135 is other than a histidine, e.g., where amino acid 135 is Gly, Ala, Val, Leu, Ile, Pro, Phe, Tyr, Trp, Ser, Thr, Cys, Met, Asn, Gln, Lys, Arg, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H136. In some cases, a variant 4-1BBL polypeptide present in a multimeric polypeptide of the present disclosure comprises the amino acid sequence set forth in SEQ ID NO:2, with an amino acid substitution at H136. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H136. In some cases, a variant 4-1BBL polypeptide present in a multimeric polypeptide of the present disclosure comprises the amino acid sequence set forth in SEQ ID NO:3, with an amino acid substitution at H136.

4-1BBL with L216 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2VVV, where amino acid 216 (indicated by an “x”) is an amino acid other than a leucine, e.g., where amino acid 216 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 136 is other than a leucine, e.g., where amino acid 136 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L137. In some cases, a variant 4-1BBL polypeptide present in a multimeric polypeptide of the present disclosure comprises the amino acid sequence set forth in SEQ ID NO:2, with an amino acid substitution at L137. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L137. In some cases, a variant 4-1BBL polypeptide present in a multimeric polypeptide of the present disclosure comprises the amino acid sequence set forth in SEQ ID NO:3, with an amino acid substitution at L137.

4-1BBL with H217 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2WWW, where amino acid 217 (indicated by an “x”) is an amino acid other than a histidine, e.g., where amino acid 217 is Gly, Ala, Val, Leu, Ile, Pro, Phe, Tyr, Trp, Ser, Thr, Cys, Met, Asn, Gln, Lys, Arg, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 137 is other than a histidine, e.g., where amino acid 137 is Gly, Ala, Val, Leu, Ile, Pro, Phe, Tyr, Trp, Ser, Thr, Cys, Met, Asn, Gln, Lys, Arg, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H138. In some cases, a variant 4-1BBL polypeptide present in a multimeric polypeptide of the present disclosure comprises the amino acid sequence set forth in SEQ ID NO:2, with an amino acid substitution at H138. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H138. In some cases, a variant 4-1BBL polypeptide present in a multimeric polypeptide of the present disclosure comprises the amino acid sequence set forth in SEQ ID NO:3, with an amino acid substitution at H138.

4-1BBL with T218 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2XXX, where amino acid 218 (indicated by an “x”) is an amino acid other than a threonine, e.g., where amino acid 218 is Gly, Ala, Val, Leu, Ile, Pro, Phe, Tyr, Trp, Ser, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 138 is other than a threonine, e.g., where amino acid 138 is Gly, Ala, Val, Leu, Ile, Pro, Phe, Tyr, Trp, Ser, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T139. In some cases, a variant 4-1BBL polypeptide present in a multimeric polypeptide of the present disclosure comprises the amino acid sequence set forth in SEQ ID NO:2, with an amino acid substitution at T139. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T139. In some cases, a variant 4-1BBL polypeptide present in a multimeric polypeptide of the present disclosure comprises the amino acid sequence set forth in SEQ ID NO:3, with an amino acid substitution at T139.

4-1BBL with E219 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2YYY, where amino acid 219 (indicated by an “x”) is an amino acid other than a glutamic acid, e.g., where amino acid 219 is Gly, Ala, Val, Leu, Ile, Pro, Phe, Tyr, Trp, Ser, Thr, Cys, Met, Asn, Gln, Lys, Arg, His, or Asp. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 139 is other than a glutamic acid, e.g., where amino acid 139 is Gly, Ala, Val, Leu, Ile, Pro, Phe, Tyr, Trp, Ser, Thr, Cys, Met, Asn, Gln, Lys, Arg, His, or Asp.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at E140. In some cases, a variant 4-1BBL polypeptide present in a multimeric polypeptide of the present disclosure comprises the amino acid sequence set forth in SEQ ID NO:2, with an amino acid substitution at E140. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at E140. In some cases, a variant 4-1BBL polypeptide present in a multimeric polypeptide of the present disclosure comprises the amino acid sequence set forth in SEQ ID NO:3, with an amino acid substitution at E140.

4-1BBL with R221 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2ZZZ, where amino acid 221 (indicated by an “x”) is an amino acid other than an arginine, e.g., where amino acid 221 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 141 is other than an arginine, e.g., where amino acid 141 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R142. In some cases, a variant 4-1BBL polypeptide present in a multimeric polypeptide of the present disclosure comprises the amino acid sequence set forth in SEQ ID NO:2, with an amino acid substitution at R142. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R142. In some cases, a variant 4-1BBL polypeptide present in a multimeric polypeptide of the present disclosure comprises the amino acid sequence set forth in SEQ ID NO:3, with an amino acid substitution at R142.

4-1BBL with R223 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2AAAA, where amino acid 223 (indicated by an “x”) is an amino acid other than an arginine, e.g., where amino acid 223 is Gly, Ala, Val, Leu, Ile, Pro, Phe, Tyr, Trp, Ser, Thr, Cys, Met, Asn, Gln, Lys, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 143 is other than an arginine, e.g., where amino acid 143 is Gly, Ala, Val, Leu, Ile, Pro, Phe, Tyr, Trp, Ser, Thr, Cys, Met, Asn, Gln, Lys, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at R144. In some cases, a variant 4-1BBL polypeptide present in a multimeric polypeptide of the present disclosure comprises the amino acid sequence set forth in SEQ ID NO:2, with an amino acid substitution at R144. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at R144. In some cases, a variant 4-1BBL polypeptide present in a multimeric polypeptide of the present disclosure comprises the amino acid sequence set forth in SEQ ID NO:3, with an amino acid substitution at R144.

4-1BBL with H224 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2BBBB, where amino acid 224 (indicated by an “x”) is an amino acid other than a histidine, e.g., where amino acid 224 is Gly, Ala, Val, Leu, Ile, Pro, Phe, Tyr, Trp, Ser, Thr, Cys, Met, Asn, Gln, Lys, Arg, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 144 is other than a histidine, e.g., where amino acid 144 is Gly, Ala, Val, Leu, Ile, Pro, Phe, Tyr, Trp, Ser, Thr, Cys, Met, Asn, Gln, Lys, Arg, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at H145. In some cases, a variant 4-1BBL polypeptide present in a multimeric polypeptide of the present disclosure comprises the amino acid sequence set forth in SEQ ID NO:2, with an amino acid substitution at H145. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at H145. In some cases, a variant 4-1BBL polypeptide present in a multimeric polypeptide of the present disclosure comprises the amino acid sequence set forth in SEQ ID NO:3, with an amino acid substitution at H145.

4-1BBL with W226 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2CCCC, where amino acid 226 (indicated by an “x”) is an amino acid other than a tryptophan, e.g., where amino acid 226 is Gly, Ala, Val, Leu, Ile, Pro, Phe, Tyr, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 146 is other than a tryptophan, e.g., where amino acid 146 is Gly, Ala, Val, Leu, Ile, Pro, Phe, Tyr, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at W147. In some cases, a variant 4-1BBL polypeptide present in a multimeric polypeptide of the present disclosure comprises the amino acid sequence set forth in SEQ ID NO:2, with an amino acid substitution at W147. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at W147. In some cases, a variant 4-1BBL polypeptide present in a multimeric polypeptide of the present disclosure comprises the amino acid sequence set forth in SEQ ID NO:3, with an amino acid substitution at W147.

4-1BBL with L228 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2DDDD, where amino acid 228 (indicated by an “x”) is an amino acid other than a leucine, e.g., where amino acid 228 is Gly, Ala, Val, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 148 is other than a leucine, e.g., where amino acid 148 is Gly, Ala, Val, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at L149. In some cases, a variant 4-1BBL polypeptide present in a multimeric polypeptide of the present disclosure comprises the amino acid sequence set forth in SEQ ID NO:2, with an amino acid substitution at L149. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at L149. In some cases, a variant 4-1BBL polypeptide present in a multimeric polypeptide of the present disclosure comprises the amino acid sequence set forth in SEQ ID NO:3, with an amino acid substitution at L149.

4-1BBL with T229 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2EEEE, where amino acid 229 (indicated by an “x”) is an amino acid other than a threonine, e.g., where amino acid 229 is Gly, Ala, Val, Leu, Ile, Pro, Phe, Tyr, Trp, Ser, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 149 is other than a threonine, e.g., where amino acid 149 is Gly, Ala, Val, Leu, Ile, Pro, Phe, Tyr, Trp, Ser, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T150. In some cases, a variant 4-1BBL polypeptide present in a multimeric polypeptide of the present disclosure comprises the amino acid sequence set forth in SEQ ID NO:2, with an amino acid substitution at T150. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T150. In some cases, a variant 4-1BBL polypeptide present in a multimeric polypeptide of the present disclosure comprises the amino acid sequence set forth in SEQ ID NO:3, with an amino acid substitution at T150.

4-1BBL with Q230 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2FFFF, where amino acid 230 (indicated by an “x”) is an amino acid other than a glutamine, e.g., where amino acid 230 is Gly, Ala, Val, Leu, Ile, Pro, Phe, Tyr, Trp, Ser, Thr, Cys, Met, Asn, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 150 is other than a glutamine, e.g., where amino acid 150 is Gly, Ala, Val, Leu, Ile, Pro, Phe, Tyr, Trp, Ser, Thr, Cys, Met, Asn, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at Q151. In some cases, a variant 4-1BBL polypeptide present in a multimeric polypeptide of the present disclosure comprises the amino acid sequence set forth in SEQ ID NO:2, with an amino acid substitution at Q151. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at Q151. In some cases, a variant 4-1BBL polypeptide present in a multimeric polypeptide of the present disclosure comprises the amino acid sequence set forth in SEQ ID NO:3, with an amino acid substitution at Q151.

4-1BBL with G231 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2GGGG, where amino acid 231 (indicated by an “x”) is an amino acid other than a glycine, e.g., where amino acid 231 is Ala, Val, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 151 is other than a glycine, e.g., where amino acid 151 is Ala, Val, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at G152. In some cases, a variant 4-1BBL polypeptide present in a multimeric polypeptide of the present disclosure comprises the amino acid sequence set forth in SEQ ID NO:2, with an amino acid substitution at G152. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at G152. In some cases, a variant 4-1BBL polypeptide present in a multimeric polypeptide of the present disclosure comprises the amino acid sequence set forth in SEQ ID NO:3, with an amino acid substitution at G152.

4-1BBL with T233 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2HHHH, where amino acid 233 (indicated by an “x”) is an amino acid other than a threonine, e.g., where amino acid 233 is Gly, Ala, Val, Leu, Ile, Pro, Phe, Tyr, Trp, Ser, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 153 is other than a threonine, e.g., where amino acid 153 is Gly, Ala, Val, Leu, Ile, Pro, Phe, Tyr, Trp, Ser, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at T154. In some cases, a variant 4-1BBL polypeptide present in a multimeric polypeptide of the present disclosure comprises the amino acid sequence set forth in SEQ ID NO:2, with an amino acid substitution at T154. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at T154. In some cases, a variant 4-1BBL polypeptide present in a multimeric polypeptide of the present disclosure comprises the amino acid sequence set forth in SEQ ID NO:3, with an amino acid substitution at T154.

4-1BBL with V234 Substitution

In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence depicted in FIG. 2IIII, where amino acid 234 (indicated by an “x”) is an amino acid other than a valine, e.g., where amino acid 234 is Gly, Ala, Leu, Ile, Pro, Phe, Tyr, Trp, Ser, Thr, Cys, Met, Asn, Gln, Lys, Arg, His, Asp, or Glu. In some cases, the variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:1, where amino acid 154 is other than a valine, e.g., where amino acid 154 is Gly, Ala, Leu, Ile, Pro, Phe, Tyr, Trp, Ser, Thr, Cys, Met, Asn, Gln, Lys, Arg, His, Asp, or Glu.

In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:2, with an amino acid substitution at V155. In some cases, a variant 4-1BBL polypeptide present in a multimeric polypeptide of the present disclosure comprises the amino acid sequence set forth in SEQ ID NO:2, with an amino acid substitution at V155. In some cases, a variant 4-1BBL polypeptide present in a multimeric polypeptide of the present disclosure comprises an amino acid sequence having at least 90%, at least 95%, at least 98%, or at least 99%, amino acid sequence identity to the amino acid sequence set forth in SEQ ID NO:3, with an amino acid substitution at V155. In some cases, a variant 4-1BBL polypeptide present in a multimeric polypeptide of the present disclosure comprises the amino acid sequence set forth in SEQ ID NO:3, with an amino acid substitution at V155.

### Additional Immunomodulatory Polypeptides

An immunomodulatory polypeptide of a multimeric polypeptide of the present disclosure can be an activating immunomodulatory polypeptide or an inhibitory immunomodulatory polypeptide. In some cases, a multimeric polypeptide of the present disclosure includes a single immunomodulatory polypeptide. In some cases, a multimeric polypeptide of the present disclosure includes two immunomodulatory polypeptides. In some cases, the two immunomodulatory polypeptides are in tandem in a polypeptide chain. In some cases, the two immunomodulatory polypeptides are in separate polypeptide chains. In some cases, the two immunomodulatory polypeptides are in separate polypeptide chains and are disulfide linked to one another.

An immunomodulatory polypeptide of a multimeric polypeptide of the present disclosure is in some cases a T-cell modulatory polypeptide. In some cases, the T-cell modulatory polypeptide is a stimulatory (activating) T-cell modulatory polypeptide. In some cases, the T-cell modulatory polypeptide is an inhibitory T-cell modulatory polypeptide. A T-cell modulatory polypeptide can be an antibody, a peptide ligand, a T-cell co-stimulatory polypeptide, a cytokine, or a toxin.

In some cases, an immunomodulatory polypeptide of a multimeric polypeptide of the present disclosure is an antibody-based or non-antibody-based recognition moiety that specifically binds a co-stimulatory polypeptide that is expressed on the surface of an epitope-specific T cell. Antibody-based recognition moieties include, e.g., antibodies; fragments of antibodies that retain specific binding to antigen, including, but not limited to, Fab, Fv, single-chain Fv (scFv), and Fd fragments; chimeric antibodies; humanized antibodies; single-chain antibodies (scAb), single domain antibodies (dAb); single domain heavy chain antibodies; single domain light chain antibodies; and the like. Suitable non-antibody-based recognition moieties include, e.g., affibodies; engineered Kunitz domains; monobodies (adnectins); anticalins; aptamers; designed ankyrin repeat domains (DARPins); a binding site of a cysteine-rich polypeptide (e.g., cysteine-rich knottin peptides); avimers; afflins; and the like. An antibody-based or non-antibody-based recognition moiety specifically binds co-stimulatory polypeptide that is expressed on the surface of an epitope-specific T cell, where such co-stimulatory polypeptides include, but are not limited to, CTLA4, PD1, ICOS, OX40, CD20, and 4-1BB. Co-stimulatory polypeptides that are expressed on the surface of an epitope-specific T cell are known in the art.

### Multiple Immunomodulatory Domains

As noted above, in some cases, a multimeric polypeptide of the present disclosure comprises two or more immunomodulatory polypeptides, where at least one of the two or more immunomodulatory polypeptide is a variant 4-1BBL polypeptide of the present disclosure.

In some cases, a multimeric polypeptide of the present disclosure comprises two or more copies of a variant 4-1BBL polypeptide of the present disclosure. In some cases, the two or more variant 4-1BBL polypeptides are on the same polypeptide chain of a multimeric polypeptide of the present disclosure. In some cases, the two or more variant 4-1BBL polypeptides are on separate polypeptide chains of a multimeric polypeptide of the present disclosure.

In some cases, a multimeric polypeptide of the present disclosure comprises a first immunomodulatory polypeptide, and at least a second immunomodulatory polypeptide, where the first immunomodulatory polypeptide is a variant 4-1BBL polypeptide of the present disclosure, and the second immunomodulatory polypeptide is not a 4-1BBL polypeptide. For example, in some cases, the second immunomodulatory polypeptide is a member of the tumor necrosis factor (TNF) superfamily; e.g., a FasL polypeptide, a CD80 polypeptide, a CD40 polypeptide, an OX40L polypeptide, a CD30L polypeptide, a CD70 polypeptide, etc. In some cases, the second immunomodulatory polypeptide of a multimeric polypeptide of the present disclosure is a T-cell co-stimulatory polypeptide and is a member of the immunoglobulin (Ig) superfamily; e.g., a CD7 polypeptide, a CD86 polypeptide, an ICAM polypeptide, etc. In some cases, the second immunomodulatory polypeptide is CD80, OX40L, ICOS-L, ICAM, PD-L1, FasL, and PD-L2. Suitable immunomodulatory polypeptides of a multimeric polypeptide of the present disclosure include, e.g., CD7, CD30L, CD40, CD70, CD83, HLA-G, MICA, MICB, HVEM, lymphotoxin beta receptor, 3/TR6, ILT3, ILT4, or HVEM.

Further T cell modulatory domains (MODs) that can be included in a multimeric polypeptide of the present disclosure include naturally occurring or synthetic human gene products (protein), affinity reagents (e.g., an antibody, antibody fragment, single chain Fvs, aptamers, nanobody) targeting a human gene product, including, but not limited to all secreted proteins arising from classical and non-classical (e.g., FGF2, IL1, S100A4) secretion mechanisms, and ecto-domains of all cell surface proteins anchored by naturally occurring genetically encoded protein segments (single or multiple membrane spans) or post-translational modifications such as GPI linkages). Any naturally occurring or synthetic affinity reagent (e.g., antibody, antibody fragment, single chain Fvs, aptamer, nanobody, lectin, etc) targeting a cell surface glycan or other post-translational modification (e.g., sulfation). Examples include, but are not limited to, members of the TNF/TNFR family (OX40L, ICOSL, FASL, LTA, LTB TRAIL, CD153, TNFSF9, RANKL, TWEAK, TNFSF13, TNFSF13b, TNFSF14, TNFSF15, TNFSF18, CD40LG, CD70) or affinity reagents directed at the TNF/TNFR family members; members of the Immunoglobulin superfamily (VISTA, PD1, PD-L1, PD-L2, B71, B72, CTLA4, CD28, TIM3, CD4, CD8, CD19, T cell receptor chains, ICOS, ICOS ligand, HHLA2, butyrophilins, BTLA, B7-H3, B7-H4, CD3, CD79a, CD79b, IgSF CAMS (including CD2, CD58, CD48, CD150, CD229, CD244, ICAM-1), Leukocyte immunoglobulin like receptors (LILR), killer cell immunoglobulin like receptors (KIR)), lectin superfamily members, selectins, cytokines/chemokine and cytokine/chemokine receptors, growth factors and growth factor receptors), adhesion molecules (integrins, fibronectins, cadherins), or ecto-domains of multi-span integral membrane protein, or affinity reagents directed at the Immunoglobulin superfamily and listed gene products. In addition, active homologs/orthologs of these gene products, including but not limited to, viral sequences (e.g., CMV, EBV), bacterial sequences, fungal sequences, eukaryotic pathogens (e.g., Schistosoma, Plasmodium, Babesia, Eimeria, Theileria, Toxoplasma, Entamoeba, Leishmania, and Trypanosoma), and mammalian-derived coding regions. In addition. a MOD may comprise a small molecules drug targeting a human gene product.

### Scaffold Polypeptides

A T-cell modulatory multimeric polypeptide of the present disclosure comprises an Fc polypeptide, or another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly), where X iany amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life (e.g., the serum half-life) of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

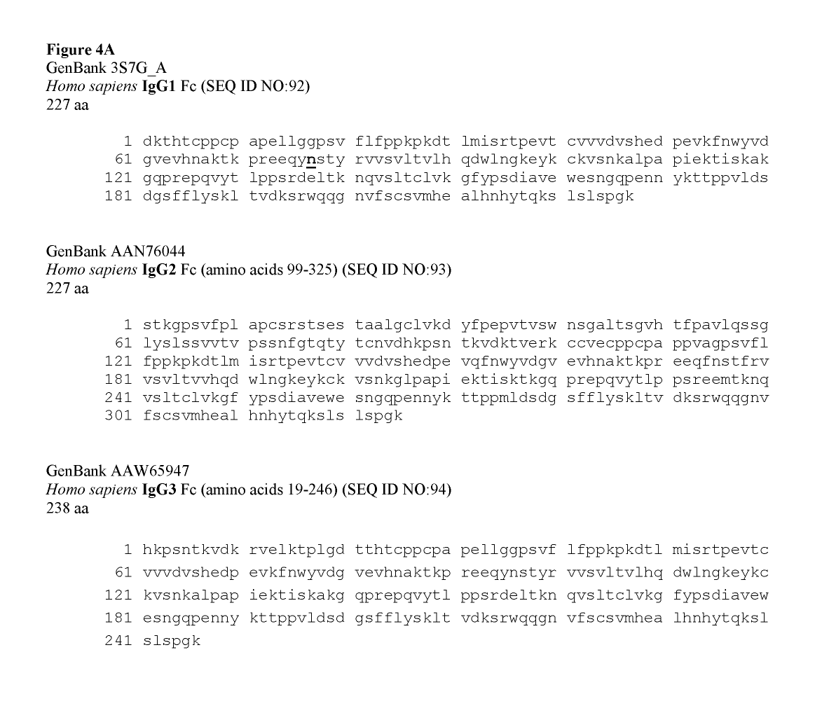

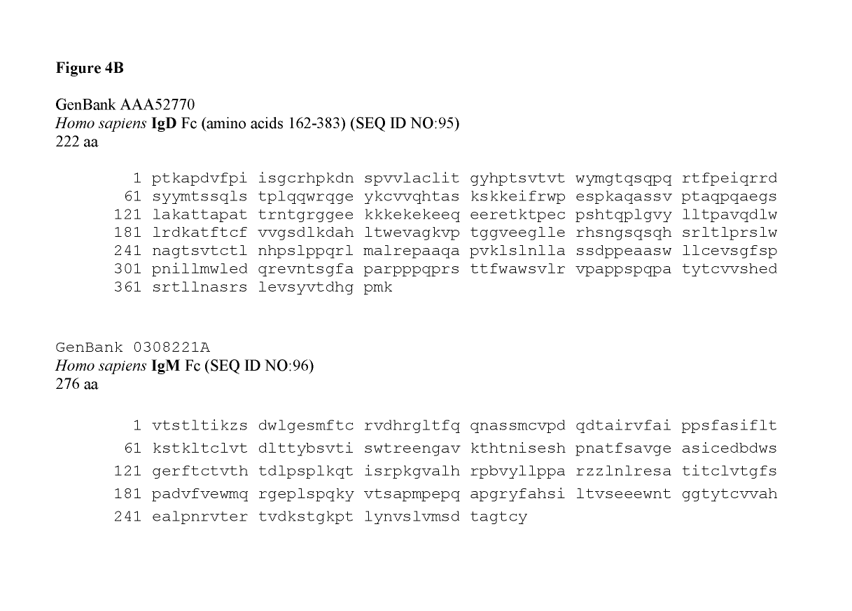

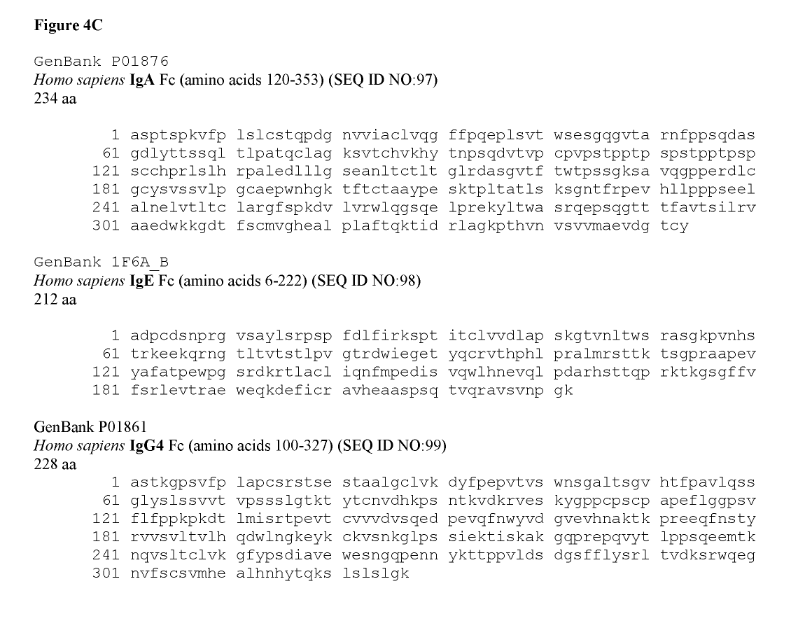

In some cases, the first and/or the second polypeptide chain of a multimeric polypeptide of the present disclosure comprises an Fc polypeptide. The Fc polypeptide of a multimeric polypeptide of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIGS. 4A-C. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 4A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 4A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 4A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 4A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 4A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 4A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 4B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 4B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 4C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 4C.

### Additional Polypeptides

A polypeptide chain of a multimeric polypeptide of the present disclosure can include one or more polypeptides in addition to those described above. Suitable additional polypeptides include epitope tags and affinity domains. The one or more additional polypeptide can be included at the N-terminus of a polypeptide chain of a multimeric polypeptide of the present disclosure, at the C-terminus of a polypeptide chain of a multimeric polypeptide of the present disclosure, or internally within a polypeptide chain of a multimeric polypeptide of the present disclosure.

### Epitope Tag

Suitable epitope tags include, but are not limited to, hemagglutinin (HA; e.g., YPYDVPDYA (SEQ ID NO:140); FLAG (e.g., DYKDDDDK (SEQ ID NO:141); c-myc (e.g., EQKLISEEDL; SEQ ID NO: 142), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO: 143), HisX6 (HHHHHH) (SEQ ID NO: 144), C-myc (EQKLISEEDL) (SEQ ID NO: 145), Flag (DYKDDDDK) (SEQ ID NO: 146), StrepTag (WSHPQFEK) (SEQ ID NO: 147), hemagglutinin, e.g., HA Tag (YPYDVPDYA) (SEQ ID NO: 148), glutathione-S-transferase (GST), thioredoxin, cellulose binding domain, RYIRS (SEQ ID NO:149), Phe-His-His-Thr (SEQ ID NO:150), chitin binding domain, S-peptide, T7 peptide, SH2 domain, C-end RNA tag, WEAAAREACCRECCARA (SEQ ID NO:151), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Exemplary Multimeric Polypeptides

Exemplary multimeric polypeptides of the present disclosure are described below.

### K127

In some cases, a multimeric polypeptide of the present disclosure comprises: a) a first polypeptide comprising, in order from N-terminus to C-terminus: i) an epitope; ii) a β2M polypeptide; and iii) a variant 4-1BBL polypeptide comprising an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; or a variant 4-1BBL polypeptide of the present disclosure comprising an amino acid sequence having at least 90%, at least 95%, at least 98%, or at least 99%, amino acid sequence identity to the amino acid sequence set forth in SEQ ID NO:1, with an amino acid substitution at K47, e.g., where amino acid 47 is Gly, Ala, Val, Leu, Ile, Pro, Phe, Tyr, Trp, Ser, Thr, Cys, Met, Asn, Gln, Arg, His, Asp, or Glu; and b) a second polypeptide comprising, in order from N-terminus to C-terminus: i) a Class I MHC heavy chain; and ii) an Fc polypeptide. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a variant 4-1BBL polypeptide comprising an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; or a variant 4-1BBL polypeptide of the present disclosure comprising an amino acid sequence having at least 90%, at least 95%, at least 98%, or at least 99%, amino acid sequence identity to the amino acid sequence set forth in SEQ ID NO:1, with an amino acid substitution at K47, e.g., where amino acid 47 is Gly, Ala, Val, Leu, Ile, Pro, Phe, Tyr, Trp, Ser, Thr, Cys, Met, Asn, Gln, Arg, His, Asp, or Glu; ii) a Class I MHC heavy chain; and iii) an Fc polypeptide. In some cases, a multimeric polypeptide of the present disclosure comprises: a) a first polypeptide comprising, in order from N-terminus to C-terminus: i) an epitope; ii) a β2M polypeptide; iii) a first variant 4-1BBL polypeptide of the present disclosure; iv) a second variant 4-1BBL polypeptide of the present disclosure; and v) a third variant 4-1BBL polypeptide of the present disclosure; and b) a second polypeptide comprising, in order from N-terminus to C-terminus: i) a Class I MHC heavy chain; and ii) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; e.g., each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO: 1, with an amino acid substitution at K47, e.g., where amino acid 47 is Gly, Ala, Val, Leu, Ile, Pro, Phe, Tyr, Trp, Ser, Thr, Cys, Met, Asn, Gln, Arg, His, Asp, or Glu. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a first variant 4-1BBL polypeptide of the present disclosure; ii) a second variant 4-1BBL polypeptide of the present disclosure; and iii) a third variant 4-1BBL polypeptide of the present disclosure; iv) a Class I MHC heavy chain; and v) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; e.g., each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO: 1, with an amino acid substitution at K47, e.g., where amino acid 47 is Gly, Ala, Val, Leu, Ile, Pro, Phe, Tyr, Trp, Ser, Thr, Cys, Met, Asn, Gln, Arg, His, Asp, or Glu. In some cases, the variant 4-1BBL polypeptide comprises a K127 substitution and an M91 substitution (based on the numbering depicted in FIG. 2A). In some cases, the variant 4-1BBL polypeptide comprises a K127 substitution and an F92 substitution (based on the numbering depicted in FIG. 2A). In some cases, the variant 4-1BBL polypeptide comprises a K127 substitution and a Q94 substitution (based on the numbering depicted in FIG. 2A). In some cases, the variant 4-1BBL polypeptide comprises a K127 substitution and an L95 substitution (based on the numbering depicted in FIG. 2A). In some cases, the variant 4-1BBL polypeptide comprises a K127 substitution and a V96 substitution (based on the numbering depicted in FIG. 2A). In some cases, the variant 4-1BBL polypeptide comprises a K127 substitution and a Q98 substitution (based on the numbering depicted in FIG. 2A). In some cases, the variant 4-1BBL polypeptide comprises a K127 substitution and an N99 substitution (based on the numbering depicted in FIG. 2A). In some cases, the variant 4-1BBL polypeptide comprises a K127 substitution and a V100 substitution (based on the numbering depicted in FIG. 2A). In some cases, the variant 4-1BBL polypeptide comprises a K127 substitution and an L101 substitution (based on the numbering depicted in FIG. 2A). In some cases, the variant 4-1BBL polypeptide comprises a K127 substitution and an L102 substitution (based on the numbering depicted in FIG. 2A). In some cases, the variant 4-1BBL polypeptide comprises a K127 substitution and an I103 substitution (based on the numbering depicted in FIG. 2A). In some cases, the variant 4-1BBL polypeptide comprises a K127 substitution and a D104 substitution (based on the numbering depicted in FIG. 2A). In some cases, the variant 4-1BBL polypeptide comprises a K127 substitution and a G105 substitution (based on the numbering depicted in FIG. 2A). In some cases, the variant 4-1BBL polypeptide comprises a K127 substitution and a P106 substitution (based on the numbering depicted in FIG. 2A). In some cases, the variant 4-1BBL polypeptide comprises a K127 substitution and an L107 substitution (based on the numbering depicted in FIG. 2A). In some cases, the variant 4-1BBL polypeptide comprises a K127 substitution and an S108 substitution (based on the numbering depicted in FIG. 2A). In some cases, the variant 4-1BBL polypeptide comprises a K127 substitution and a W109 substitution (based on the numbering depicted in FIG. 2A). In some cases, the variant 4-1BBL polypeptide comprises a K127 substitution and a Y110 substitution (based on the numbering depicted in FIG. 2A). In some cases, the variant 4-1BBL polypeptide comprises a K127 substitution and an S111 substitution (based on the numbering depicted in FIG. 2A). In some cases, the variant 4-1BBL polypeptide comprises a K127 substitution and a D112 substitution (based on the numbering depicted in FIG. 2A). In some cases, the variant 4-1BBL polypeptide comprises a K127 substitution and a P113 substitution (based on the numbering depicted in FIG. 2A). In some cases, the variant 4-1BBL polypeptide comprises a K127 substitution and a G114 substitution (based on the numbering depicted in FIG. 2A). In some cases, the variant 4-1BBL polypeptide comprises a K127 substitution and an L115 substitution (based on the numbering depicted in FIG. 2A).

In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a first variant 4-1BBL polypeptide of the present disclosure; ii) a linker; iii) a second variant 4-1BBL polypeptide of the present disclosure; iv) a linker; v) a third variant 4-1BBL polypeptide of the present disclosure; vi) a Class I MHC heavy chain; and vii) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2B, where amino acid 127 (indicated by an “x”) is an amino acid other than a lysine, e.g., where amino acid 127 is Gly, Ala, Val, Leu, Ile, Pro, Phe, Tyr, Trp, Ser, Thr, Cys, Met, Asn, Gln, Arg, His, Asp, or Glu; e.g., each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO:1, with an amino acid substitution at K47, e.g., where amino acid 47 is Gly, Ala, Val, Leu, Ile, Pro, Phe, Tyr, Trp, Ser, Thr, Cys, Met, Asn, Gln, Arg, His, Asp, or Glu. In some cases, the linker comprises a (GSSSS)n (SEQ ID NO:131) sequence, where n is 1, 2, 3, 4, or 5. In some cases, n is 4. In some cases, n is 5. In some cases, the variant 4-1BBL polypeptide comprises a K127 substitution and an M91 substitution (based on the numbering depicted in FIG. 2A). In some cases, the variant 4-1BBL polypeptide comprises a K127 substitution and an F92 substitution (based on the numbering depicted in FIG. 2A). In some cases, the variant 4-1BBL polypeptide comprises a K127 substitution and a Q94 substitution (based on the numbering depicted in FIG. 2A). In some cases, the variant 4-1BBL polypeptide comprises a K127 substitution and an L95 substitution (based on the numbering depicted in FIG. 2A). In some cases, the variant 4-1BBL polypeptide comprises a K127 substitution and a V96 substitution (based on the numbering depicted in FIG. 2A). In some cases, the variant 4-1BBL polypeptide comprises a K127 substitution and a Q98 substitution (based on the numbering depicted in FIG. 2A). In some cases, the variant 4-1BBL polypeptide comprises a K127 substitution and an N99 substitution (based on the numbering depicted in FIG. 2A). In some cases, the variant 4-1BBL polypeptide comprises a K127 substitution and a V100 substitution (based on the numbering depicted in FIG. 2A). In some cases, the variant 4-1BBL polypeptide comprises a K127 substitution and an L101 substitution (based on the numbering depicted in FIG. 2A). In some cases, the variant 4-1BBL polypeptide comprises a K127 substitution and an L102 substitution (based on the numbering depicted in FIG. 2A). In some cases, the variant 4-1BBL polypeptide comprises a K127 substitution and an I103 substitution (based on the numbering depicted in FIG. 2A). In some cases, the variant 4-1BBL polypeptide comprises a K127 substitution and a D104 substitution (based on the numbering depicted in FIG. 2A). In some cases, the variant 4-1BBL polypeptide comprises a K127 substitution and a G105 substitution (based on the numbering depicted in FIG. 2A). In some cases, the variant 4-1BBL polypeptide comprises a K127 substitution and a P106 substitution (based on the numbering depicted in FIG. 2A). In some cases, the variant 4-1BBL polypeptide comprises a K127 substitution and an L107 substitution (based on the numbering depicted in FIG. 2A). In some cases, the variant 4-1BBL polypeptide comprises a K127 substitution and an S108 substitution (based on the numbering depicted in FIG. 2A). In some cases, the variant 4-1BBL polypeptide comprises a K127 substitution and a W109 substitution (based on the numbering depicted in FIG. 2A). In some cases, the variant 4-1BBL polypeptide comprises a K127 substitution and a Y110 substitution (based on the numbering depicted in FIG. 2A). In some cases, the variant 4-1BBL polypeptide comprises a K127 substitution and an S111 substitution (based on the numbering depicted in FIG. 2A). In some cases, the variant 4-1BBL polypeptide comprises a K127 substitution and a D112 substitution (based on the numbering depicted in FIG. 2A). In some cases, the variant 4-1BBL polypeptide comprises a K127 substitution and a P113 substitution (based on the numbering depicted in FIG. 2A). In some cases, the variant 4-1BBL polypeptide comprises a K127 substitution and a G114 substitution (based on the numbering depicted in FIG. 2A). In some cases, the variant 4-1BBL polypeptide comprises a K127 substitution and an L115 substitution (based on the numbering depicted in FIG. 2A).

### Q227

In some cases, a multimeric polypeptide of the present disclosure comprises: a) a first polypeptide comprising, in order from N-terminus to C-terminus: i) an epitope; ii) a β2M polypeptide; and iii) a variant 4-1BBL polypeptide comprising an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu; or a variant 4-1BBL polypeptide of the present disclosure comprising an amino acid sequence having at least 90%, at least 95%, at least 98%, or at least 99%, amino acid sequence identity to the amino acid sequence set forth in SEQ ID NO:1, with an amino acid substitution at Q147, e.g., where amino acid 147 is Gly, Ala, Val, Leu, Ile, Pro, Phe, Tyr, Trp, Ser, Thr, Cys, Met, Asn, Lys, Arg, His, Asp, or Glu; and b) a second polypeptide comprising, in order from N-terminus to C-terminus: i) a Class I MHC heavy chain; and ii) an Fc polypeptide. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a variant 4-1BBL polypeptide comprising an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu; or a variant 4-1BBL polypeptide of the present disclosure comprising an amino acid sequence having at least 90%, at least 95%, at least 98%, or at least 99%, amino acid sequence identity to the amino acid sequence set forth in SEQ ID NO:1, with an amino acid substitution at Q147, e.g., where amino acid 147 is Gly, Ala, Val, Leu, Ile, Pro, Phe, Tyr, Trp, Ser, Thr, Cys, Met, Asn, Lys, Arg, His, Asp, or Glu; ii) a Class I MHC heavy chain; and iii) an Fc polypeptide. In some cases, a multimeric polypeptide of the present disclosure comprises: a) a first polypeptide comprising, in order from N-terminus to C-terminus: i) an epitope; ii) a β2M polypeptide; iii) a first variant 4-1BBL polypeptide of the present disclosure; iv) a second variant 4-1BBL polypeptide of the present disclosure; and v) a third variant 4-1BBL polypeptide of the present disclosure; and b) a second polypeptide comprising, in order from N-terminus to C-terminus: i) a Class I MHC heavy chain; and ii) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu; or each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO:1, with an amino acid substitution at Q147, e.g., where amino acid 147 is Gly, Ala, Val, Leu, Ile, Pro, Phe, Tyr, Trp, Ser, Thr, Cys, Met, Asn, Lys, Arg, His, Asp, or Glu. In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a first variant 4-1BBL polypeptide of the present disclosure; ii) a second variant 4-1BBL polypeptide of the present disclosure; and iii) a third variant 4-1BBL polypeptide of the present disclosure; iv) a Class I MHC heavy chain; and v) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu; or each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO: 1, with an amino acid substitution at Q147, e.g., where amino acid 147 is Gly, Ala, Val, Leu, Ile, Pro, Phe, Tyr, Trp, Ser, Thr, Cys, Met, Asn, Lys, Arg, His, Asp, or Glu.

In some cases, a multimeric polypeptide of the present disclosure comprises: a) a first polypeptide comprising, in order from N-terminus to C-terminus: i) an epitope; and ii) a β2M polypeptide; and b) a second polypeptide comprising, in order from N-terminus to C-terminus: i) a first variant 4-1BBL polypeptide of the present disclosure; ii) a linker; iii) a second variant 4-1BBL polypeptide of the present disclosure; iv) a linker; v) a third variant 4-1BBL polypeptide of the present disclosure; vi) a Class I MHC heavy chain; and vii) an Fc polypeptide. In some cases, each of the first, second, and third variant 4-1BBL polypeptides comprises an amino acid sequence having at least 90%, at least 95%, at least 98%, or at least 99%, amino acid sequence identity to the amino acid sequence depicted in FIG. 2D, where amino acid 227 (indicated by an “x”) is an amino acid other than a glutamine, e.g., where amino acid 227 is Gly, Ala, Val, Leu, Ile, Pro, Phe, Tyr, Trp, Ser, Thr, Cys, Met, Asn, Lys, Arg, His, Asp, or Glu; or each of the first, second, and third variant 4-1BBL polypeptides comprises an amino acid sequence having at least 90%, at least 95%, at least 98%, or at least 99%, amino acid sequence identity to the amino acid sequence set forth in SEQ ID NO:1, with an amino acid substitution at Q147, e.g., where amino acid 147 is Gly, Ala, Val, Leu, Ile, Pro, Phe, Tyr, Trp, Ser, Thr, Cys, Met, Asn, Lys, Arg, His, Asp, or Glu. In some cases, the linker comprises a (GSSSS)n (SEQ ID NO:131) sequence, where n is 1, 2, 3, 4, or 5. In some cases, n is 4. In some cases, n is 5.

### Nucleic Acids

The present disclosure provides a nucleic acid comprising a nucleotide sequence encoding a variant 4-1BBL polypeptide of the present disclosure. The present disclosure provides a nucleic acid comprising a nucleotide sequence encoding a 4-1BBL fusion polypeptide of the present disclosure.

The present disclosure provides nucleic acids comprising nucleotide sequences encoding a multimeric polypeptide of the present disclosure. In some cases, the individual polypeptide chains of a multimeric polypeptide of the present disclosure are encoded in separate nucleic acids. In some cases, all polypeptide chains of a multimeric polypeptide of the present disclosure are encoded in a single nucleic acid. In some cases, a first nucleic acid comprises a nucleotide sequence encoding a first polypeptide of a multimeric polypeptide of the present disclosure; and a second nucleic acid comprises a nucleotide sequence encoding a second polypeptide of a multimeric polypeptide of the present disclosure. In some cases, single nucleic acid comprises a nucleotide sequence encoding a first polypeptide of a multimeric polypeptide of the present disclosure and a second polypeptide of a multimeric polypeptide of the present disclosure.

### Separate Nucleic Acids Encoding Individual Polypeptide Chains of a Multimeric Polypeptide

The present disclosure provides nucleic acids comprising nucleotide sequences encoding a multimeric polypeptide of the present disclosure. As noted above, in some cases, the individual polypeptide chains of a multimeric polypeptide of the present disclosure are encoded in separate nucleic acids. In some cases, nucleotide sequences encoding the separate polypeptide chains of a multimeric polypeptide of the present disclosure are operably linked to transcriptional control elements, e.g., promoters, such as promoters that are functional in a eukaryotic cell, where the promoter can be a constitutive promoter or an inducible promoter.

The present disclosure provides a first nucleic acid and a second nucleic acid, where the first nucleic acid comprises a nucleotide sequence encoding a first polypeptide of a multimeric polypeptide of the present disclosure, where the first polypeptide comprises, in order from N-terminus to C-terminus: a) an epitope (e.g., a T-cell epitope); b) a first MHC polypeptide; and c) an immunomodulatory polypeptide (e.g., a variant 4-1BBL polypeptide of the present disclosure); and where the second nucleic acid comprises a nucleotide sequence encoding a second polypeptide of a multimeric polypeptide of the present disclosure, where the second polypeptide comprises, in order from N-terminus to C-terminus: a) a second MHC polypeptide; and b) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a multimeric polypeptide of the present disclosure, where the first polypeptide comprises, in order from N-terminus to C-terminus: a) an epitope (e.g., a T-cell epitope); and b) a first MHC polypeptide; and where the second nucleic acid comprises a nucleotide sequence encoding a second polypeptide of a multimeric polypeptide of the present disclosure, where the second polypeptide comprises, in order from N-terminus to C-terminus: a) an immunomodulatory polypeptide (e.g., a variant 4-1BBL polypeptide of the present disclosure); b) a second MHC polypeptide; and c) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Multimeric Polypeptide

The present disclosure provides a nucleic acid comprising nucleotide sequences encoding at least the first polypeptide and the second polypeptide of a multimeric polypeptide of the present disclosure. In some cases, where a multimeric polypeptide of the present disclosure includes a first, second, and third polypeptide, the nucleic acid includes a nucleotide sequence encoding the first, second, and third polypeptides. In some cases, the nucleotide sequences encoding the first polypeptide and the second polypeptide of a multimeric polypeptide of the present disclosure includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a multimeric polypeptide of the present disclosure includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a multimeric polypeptide of the present disclosure includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a multimeric polypeptide of the present disclosure; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a multimeric polypeptide of the present disclosure; and a second nucleic acid (e.g., a recombinant expression vector, an mRNA, a viral RNA, etc.) comprises a nucleotide sequence encoding a second polypeptide chain of a multimeric polypeptide of the present disclosure. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus: a) an epitope (e.g., a T-cell epitope); b) a first MHC polypeptide; c) an immunomodulatory polypeptide (e.g., a variant 4-1BBL polypeptide of the present disclosure); d) a proteolytically cleavable linker; e) a second MHC polypeptide; and f) an immunoglobulin (Ig) Fc polypeptide. The present disclosure provides a nucleic acid comprising a nucleotide sequence encoding a recombinant polypeptide, where the recombinant polypeptide comprises, in order from N-terminus to C-terminus: a) a first leader peptide; b) the epitope; c) the first MHC polypeptide; d) the immunomodulatory polypeptide (e.g., a variant 4-1BBL polypeptide of the present disclosure); e) the proteolytically cleavable linker; f) a second leader peptide; g) the second MHC polypeptide; and h) the Ig Fc polypeptide. The present disclosure provides a nucleic acid comprising a nucleotide sequence encoding a recombinant polypeptide, where the recombinant polypeptide comprises, in order from N-terminus to C-terminus: a) an epitope; b) a first MHC polypeptide; c) a proteolytically cleavable linker; d) an immunomodulatory polypeptide (e.g., a variant 4-1BBL polypeptide of the present disclosure); e) a second MHC polypeptide; and f) an Ig Fc polypeptide. In some cases, the first leader peptide and the second leader peptide is a β2-M leader peptide. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to the amino acid sequence set forth in SEQ ID NO: 103. In some cases, the β2-microglobulin polypeptide comprises an amino acid sequence having at least 85% amino acid sequence identity to the amino acid sequence set forth in SEQ ID NO:138. In some cases, the MHC class I heavy chain polypeptide is an HLA-A, HLA-B, HLA-C, HLA-E, HLA-F, HLA-G, HLA-K, or HLA-L heavy chain. In some cases, the MHC class I heavy chain polypeptide comprises an amino acid sequence having at least 85% amino acid sequence identity to the amino acid sequence set forth in SEQ ID NO: 100. In some cases, the MHC class I heavy chain polypeptide comprises an amino acid sequence having at least 85% amino acid sequence identity to the amino acid sequence set forth in SEQ ID NO:101. In some cases, the MHC class I heavy chain polypeptide comprises an amino acid sequence having at least 85% amino acid sequence identity to the amino acid sequence set forth in SEQ ID NO: 102. In some cases, the MHC class I heavy chain polypeptide comprises an amino acid sequence having at least 85% amino acid sequence identity to the amino acid sequence set forth in SEQ ID NO: 134. In some cases, the MHC class I heavy chain polypeptide comprises an amino acid sequence having at least 85% amino acid sequence identity to the amino acid sequence set forth in SEQ ID NO:135. In some cases, the MHC class I heavy chain polypeptide comprises an amino acid sequence having at least 85% amino acid sequence identity to the amino acid sequence set forth in SEQ ID NO:136. In some cases, the MHC class I heavy chain polypeptide comprises an amino acid sequence having at least 85% amino acid sequence identity to the amino acid sequence set forth in SEQ ID NO:137. In some cases, the MHC class I heavy chain polypeptide comprises an amino acid sequence having at least 85% amino acid sequence identity to the amino acid sequence set forth in SEQ ID NO:139. In some cases, the first MHC polypeptide is an MHC Class II alpha chain polypeptide; and wherein the second MHC polypeptide is an MHC class II beta chain polypeptide.

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIGS. 4A-4C.

Suitable immunomodulatory polypeptides are described above.

Suitable proteolytically cleavable linkers are described above. In some cases, the proteolytically cleavable linker comprises an amino acid sequence selected from: a) LEVLFQGP (SEQ ID NO:116); b) ENLYTQS (SEQ ID NO:117); c) DDDDK (SEQ ID NO:152); d) LVPR (SEQ ID NO: 118); and e) GSGATNFSLLKQAGDVEENPGP (SEQ ID NO: 119).

In some cases, the proteolytically cleavable linker comprises an amino acid sequence selected from: a) LEVLFQGP (SEQ ID NO:116); b) ENLYTQS (SEQ ID NO:117); c) a furin cleavage site; d) LVPR (SEQ ID NO: 118); e) GSGATNFSLLKQAGDVEENPGP (SEQ ID NO: 119); f) GSGEGRGSLLTCGDVEENPGP (SEQ ID NO: 120); g) GSGQCTNYALLKLAGDVESNPGP (SEQ ID NO:121); and h) GSGVKQTLNFDLLKLAGDVESNPGP (SEQ ID NO:122).

In some cases, a linker between the epitope and the first MHC polypeptide comprises a first Cys residue, and the second MHC polypeptide comprises an amino acid substitution to provide a second Cys residue, such that the first and the second Cys residues provide for a disulfide linkage between the linker and the second MHC polypeptide. In some cases, first MHC polypeptide comprises an amino acid substitution to provide a first Cys residue, and the second MHC polypeptide comprises an amino acid substitution to provide a second Cys residue, such that the first Cys residue and the second Cys residue provide for a disulfide linkage between the first MHC polypeptide and the second MHC polypeptide.

### Recombinant Expression Vectors

The present disclosure provides recombinant expression vectors comprising nucleic acids of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding a DNA-targeting RNA and/or a site-directed modifying polypeptide is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), Chinese hamster ovary (CHO) cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2-M.

### Methods of Producing a Multimeric Polypeptide

The present disclosure provides methods of producing a multimeric polypeptide of the present disclosure. The methods generally involve culturing, in a culture medium, a host cell that is genetically modified with a recombinant expression vector comprising a nucleotide sequence encoding the multimeric polypeptide; and isolating the multimeric polypeptide from the genetically modified host cell and/or the culture medium. A host cell that is genetically modified with a recombinant expression vector comprising a nucleotide sequence encoding the multimeric polypeptide is also referred to as an “expression host.” As noted above, in some cases, the individual polypeptide chains of a multimeric polypeptide of the present disclosure are encoded in separate recombinant expression vectors. In some cases, all polypeptide chains of a multimeric polypeptide of the present disclosure are encoded in a single recombinant expression vector.

Isolation of the multimeric polypeptide from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography, gel electrophoresis, affinity chromatography, or other purification technique. Alternatively, where the multimeric polypeptide is secreted from the expression host cell into the culture medium, the multimeric polypeptide can be purified from the culture medium using HPLC, exclusion chromatography, gel electrophoresis, affinity chromatography, or other purification technique. In some cases, the compositions which are used will comprise at least 80% by weight of the desired product, at least about 85% by weight, at least about 95% by weight, or at least about 99.5% by weight, in relation to contaminants related to the method of preparation of the product and its purification. The percentages can be based upon total protein.

In some cases, e.g., where the multimeric polypeptide comprises an affinity tag, the multimeric polypeptide can be purified using an immobilized binding partner of the affinity tag.

### Compositions

The present disclosure provides compositions, including pharmaceutical compositions, comprising a variant 4-1BBL polypeptide of the present disclosure. The present disclosure provides compositions, including pharmaceutical compositions, comprising a multimeric polypeptide of the present disclosure. The present disclosure provides compositions, including pharmaceutical compositions, comprising a nucleic acid or a recombinant expression vector of the present disclosure.

### Compositions Comprising a Multimeric Polypeptide

A composition of the present disclosure can comprise, in addition to a multimeric polypeptide of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a multimeric polypeptide of the present disclosure, and a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Where a multimeric polypeptide of the present disclosure is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a multimeric polypeptide of the present disclosure in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

The present disclosure provides compositions, including pharmaceutical compositions, comprising a variant 4-1BBL polypeptide of the present disclosure. A composition can comprise: a) a variant 4-1BBL polypeptide of the present disclosure; and b) an excipient, as described above for the multimeric polypeptides. In some cases, the excipient is a pharmaceutically acceptable excipient.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) a subject nucleic acid or recombinant expression vector; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some embodiments, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

### Methods of Modulating T Cell Activity

The present disclosure provides a method of selectively modulating the activity of an epitope-specific T cell, the method comprising contacting the T cell with a multimeric polypeptide of the present disclosure, where contacting the T cell with a multimeric polypeptide of the present disclosure selectively modulates the activity of the epitope-specific T cell. In some cases, the contacting occurs in vitro. In some cases, the contacting occurs in vivo. In some cases, the contacting occurs ex vivo.

In some cases, e.g., where the target T cell is a CD8⁺ T cell, the multimeric polypeptide comprises Class I MHC polypeptides (e.g., β2-microglobulin and Class I MHC heavy chain). In some cases, e.g., where the target T cell is a CD4⁺ T cell, the multimeric polypeptide comprises Class II MHC polypeptides (e.g., Class II MHC α chain; Class II MHC β chain).

Where a multimeric polypeptide of the present disclosure includes an immunomodulatory polypeptide that is an activating polypeptide, contacting the T cell with the multimeric polypeptide activates the epitope-specific T cell. In some instances, the epitope-specific T cell is a T cell that is specific for an epitope present on a cancer cell, and contacting the epitope-specific T cell with the multimeric polypeptide increases cytotoxic activity of the T cell toward the cancer cell. In some instances, the epitope-specific T cell is a T cell that is specific for an epitope present on a cancer cell, and contacting the epitope-specific T cell with the multimeric polypeptide increases the number of the epitope-specific T cells.

In some instances, the epitope-specific T cell is a T cell that is specific for an epitope present on a virus-infected cell, and contacting the epitope-specific T cell with the multimeric polypeptide increases cytotoxic activity of the T cell toward the virus-infected cell. In some instances, the epitope-specific T cell is a T cell that is specific for an epitope present on a virus-infected cell, and contacting the epitope-specific T cell with the multimeric polypeptide increases the number of the epitope-specific T cells.

Where a multimeric polypeptide of the present disclosure includes an immunomodulatory polypeptide that is an inhibiting polypeptide, contacting the T cell with the multimeric inhibits the epitope-specific T cell. In some instances, the epitope-specific T cell is a self-reactive T cell that is specific for an epitope present in a self antigen, and the contacting reduces the number of the self-reactive T cells.

### Treatment Methods

The present invention provides a method of selectively modulating the activity of an epitope-specific T cell in an individual, the method comprising administering to the individual an amount of the multimeric polypeptide of the present disclosure, or one or more nucleic acids encoding the multimeric polypeptide, effective to selectively modulate the activity of an epitope-specific T cell in an individual. In some cases, a treatment method of the present disclosure comprises administering to an individual in need thereof one or more recombinant expression vectors comprising nucleotide sequences encoding a multimeric polypeptide of the present disclosure. In some cases, a treatment method of the present disclosure comprises administering to an individual in need thereof one or more mRNA molecules comprising nucleotide sequences encoding a multimeric polypeptide of the present disclosure. In some cases, a treatment method of the present disclosure comprises administering to an individual in need thereof a multimeric polypeptide of the present disclosure.

The present disclosure provides a method of selectively modulating the activity of an epitope-specific T cell in an individual, the method comprising administering to the individual an effective amount of a multimeric polypeptide of the present disclosure, or one or more nucleic acids (e.g., expression vectors; mRNA; etc.) comprising nucleotide sequences encoding the multimeric polypeptide, where the multimeric polypeptide selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a multimeric polypeptide of the present disclosure.

In some cases, the immunomodulatory polypeptide is an activating polypeptide, and the multimeric polypeptide activates the epitope-specific T cell. In some cases, the epitope is a cancer-associated epitope, and the multimeric polypeptide increases the activity of a T cell specific for the cancer-associate epitope.

The present disclosure provides a method of treating cancer in an individual, the method comprising administering to the individual an effective amount of a multimeric polypeptide of the present disclosure, or one or more nucleic acids (e.g., expression vectors; mRNA; etc.) comprising nucleotide sequences encoding the multimeric polypeptide, where the multimeric polypeptide comprises a T-cell epitope that is a cancer epitope, and where the multimeric polypeptide comprises a stimulatory immunomodulatory polypeptide. In some cases, an “effective amount” of a multimeric polypeptide is an amount that, when administered in one or more doses to an individual in need thereof, reduces the number of cancer cells in the individual. For example, in some cases, an “effective amount” of a multimeric polypeptide of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the multimeric polypeptide, or in the absence of administration with the multimeric polypeptide. In some cases, an “effective amount” of a multimeric polypeptide of the present disclosure is an amount that, when administered in one or more doses to an individual in need thereof, reduces the number of cancer cells in the individual to undetectable levels. In some cases, an “effective amount” of a multimeric polypeptide of the present disclosure is an amount that, when administered in one or more doses to an individual in need thereof, reduces the tumor mass (e.g., tumor volume) in the individual. For example, in some cases, an “effective amount” of a multimeric polypeptide of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the multimeric polypeptide, or in the absence of administration with the multimeric polypeptide. In some cases, an “effective amount” of a multimeric polypeptide of the present disclosure is an amount that, when administered in one or more doses to an individual in need thereof, increases survival time of the individual. For example, in some cases, an “effective amount” of a multimeric polypeptide of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the multimeric polypeptide.

In some instances, the epitope-specific T cell is a T cell that is specific for an epitope present on a virus-infected cell, and contacting the epitope-specific T cell with the multimeric polypeptide increases cytotoxic activity of the T cell toward the virus-infected cell. In some instances, the epitope-specific T cell is a T cell that is specific for an epitope present on a virus-infected cell, and contacting the epitope-specific T cell with the multimeric polypeptide increases the number of the epitope-specific T cells.

Thus, the present disclosure provides a method of treating a virus infection in an individual, the method comprising administering to the individual an effective amount of a multimeric polypeptide of the present disclosure, or one or more nucleic acids comprising nucleotide sequences encoding the multimeric polypeptide, where the multimeric polypeptide comprises a T-cell epitope that is a viral epitope, and where the multimeric polypeptide comprises a stimulatory immunomodulatory polypeptide. In some cases, an “effective amount” of a multimeric polypeptide is an amount that, when administered in one or more doses to an individual in need thereof, reduces the number of virus-infected cells in the individual. For example, in some cases, an “effective amount” of a multimeric polypeptide of the present disclosure is an amount that, when administered in one or more doses to an individual in need thereof, reduces the number of virus-infected cells in the individual by at least 10%, at least 15%, at least 20%, at least 25%, at least 30%, at least 40%, at least 50%, at least 60%, at least 70%, at least 80%, at least 90%, or at least 95%, compared to the number of virus-infected cells in the individual before administration of the multimeric polypeptide, or in the absence of administration with the multimeric polypeptide. In some cases, an “effective amount” of a multimeric polypeptide of the present disclosure is an amount that, when administered in one or more doses to an individual in need thereof, reduces the number of virus-infected cells in the individual to undetectable levels.

Thus, the present disclosure provides a method of treating an infection in an individual, the method comprising administering to the individual an effective amount of a multimeric polypeptide of the present disclosure, or one or more nucleic acids comprising nucleotide sequences encoding the multimeric polypeptide, where the multimeric polypeptide comprises a T-cell epitope that is a pathogen-associated epitope, and where the multimeric polypeptide comprises a stimulatory immunomodulatory polypeptide. In some cases, an “effective amount” of a multimeric polypeptide is an amount that, when administered in one or more doses to an individual in need thereof, reduces the number of pathogens in the individual. For example, in some cases, an “effective amount” of a multimeric polypeptide of the present disclosure is an amount that, when administered in one or more doses to an individual in need thereof, reduces the number of pathogens in the individual by at least 10%, at least 15%, at least 20%, at least 25%, at least 30%, at least 40%, at least 50%, at least 60%, at least 70%, at least 80%, at least 90%, or at least 95%, compared to the number of pathogens in the individual before administration of the multimeric polypeptide, or in the absence of administration with the multimeric polypeptide. In some cases, an “effective amount” of a multimeric polypeptide of the present disclosure is an amount that, when administered in one or more doses to an individual in need thereof, reduces the number of pathogens in the individual to undetectable levels. Pathogens include viruses, bacteria, protozoans, and the like.

In some cases, the immunomodulatory polypeptide is an inhibitory polypeptide, and the multimeric polypeptide inhibits activity of the epitope-specific T cell. In some cases, the epitope is a self-epitope, and the multimeric polypeptide selectively inhibits the activity of a T cell specific for the self-epitope.

The present disclosure provides a method of treating an autoimmune disorder in an individual, the method comprising administering to the individual an effective amount of a multimeric polypeptide of the present disclosure, or one or more nucleic acids comprising nucleotide sequences encoding the multimeric polypeptide, where the multimeric polypeptide comprises a T-cell epitope that is a self epitope, and where the multimeric polypeptide comprises an inhibitory immunomodulatory polypeptide. In some cases, an “effective amount” of a multimeric polypeptide is an amount that, when administered in one or more doses to an individual in need thereof, reduces the number self-reactive T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the multimeric polypeptide, or in the absence of administration with the multimeric polypeptide. In some cases, an “effective amount” of a multimeric polypeptide is an amount that, when administered in one or more doses to an individual in need thereof, reduces production of Th2 cytokines in the individual. In some cases, an “effective amount” of a multimeric polypeptide is an amount that, when administered in one or more doses to an individual in need thereof, ameliorates one or more symptoms associated with an autoimmune disease in the individual.

As noted above, in some cases, in carrying out a subject treatment method, a multimeric polypeptide of the present disclosure is administered to an individual in need thereof, as the polypeptide per se. In other instances, in carrying out a subject treatment method, one or more nucleic acids comprising nucleotide sequences encoding a multimeric polypeptide of the present disclosure is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a multimeric polypeptide of the present disclosure; and b) a pharmaceutically acceptable excipient. In some cases, a suitable formulation comprises: a) a nucleic acid comprising a nucleotide sequence encoding a multimeric polypeptide of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a multimeric polypeptide of the present disclosure; b) a second nucleic acid comprising a nucleotide sequence encoding the second polypeptide of a multimeric polypeptide of the present disclosure; and c) a pharmaceutically acceptable excipient. In some cases, a suitable formulation comprises: a) a recombinant expression vector comprising a nucleotide sequence encoding a multimeric polypeptide of the present disclosure; and b) a pharmaceutically acceptable excipient. In some cases, a suitable formulation comprises: a) a first recombinant expression vector comprising a nucleotide sequence encoding the first polypeptide of a multimeric polypeptide of the present disclosure; b) a second recombinant expression vector comprising a nucleotide sequence encoding the second polypeptide of a multimeric polypeptide of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A multimeric polypeptide of the present disclosure may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute.

In some cases, a suitable dose of a multimeric polypeptide of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a multimeric polypeptide of the present disclosure is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific multimeric polypeptide, the severity of the symptoms and the susceptibility of the subject to side effects. Preferred dosages for a given compound are readily determinable by those of skill in the art by a variety of means.

In some embodiments, multiple doses of a multimeric polypeptide of the present disclosure, a nucleic acid of the present disclosure, or a recombinant expression vector of the present disclosure are administered. The frequency of administration of a multimeric polypeptide of the present disclosure, a nucleic acid of the present disclosure, or a recombinant expression vector of the present disclosure can vary depending on any of a variety of factors, e.g., severity of the symptoms, etc. For example, in some embodiments, a multimeric polypeptide of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a multimeric polypeptide of the present disclosure, a nucleic acid of the present disclosure, or a recombinant expression vector of the present disclosure, e.g., the period of time over which a multimeric polypeptide of the present disclosure, a nucleic acid of the present disclosure, or a recombinant expression vector of the present disclosure is administered, can vary, depending on any of a variety of factors, e.g., patient response, etc. For example, a multimeric polypeptide of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a multimeric polypeptide of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the multimeric polypeptide and/or the desired effect. A multimeric polypeptide of the present disclosure, or a nucleic acid or recombinant expression vector of the present disclosure, can be administered in a single dose or in multiple doses.

In some embodiments, a multimeric polypeptide of the present disclosure, a nucleic acid of the present disclosure, or a recombinant expression vector of the present disclosure is administered intravenously. In some embodiments, a multimeric polypeptide of the present disclosure, a nucleic acid of the present disclosure, or a recombinant expression vector of the present disclosure is administered intramuscularly. In some embodiments, a multimeric polypeptide of the present disclosure, a nucleic acid of the present disclosure, or a recombinant expression vector of the present disclosure is administered locally. In some embodiments, a multimeric polypeptide of the present disclosure, a nucleic acid of the present disclosure, or a recombinant expression vector of the present disclosure is administered intratumorally. In some embodiments, a multimeric polypeptide of the present disclosure, a nucleic acid of the present disclosure, or a recombinant expression vector of the present disclosure is administered peritumorally. In some embodiments, a multimeric polypeptide of the present disclosure, a nucleic acid of the present disclosure, or a recombinant expression vector of the present disclosure is administered intracranially. In some embodiments, a multimeric polypeptide of the present disclosure, a nucleic acid of the present disclosure, or a recombinant expression vector of the present disclosure is administered subcutaneously.

In some embodiments, a multimeric polypeptide of the present disclosure is administered intravenously. In some embodiments, a multimeric polypeptide of the present disclosure is administered intramuscularly. In some embodiments, a multimeric polypeptide of the present disclosure is administered locally. In some embodiments, a multimeric polypeptide of the present disclosure is administered intratumorally. In some embodiments, a multimeric polypeptide of the present disclosure is administered peritumorally. In some embodiments, a multimeric polypeptide of the present disclosure is administered intracranially. In some embodiments, a multimeric polypeptide is administered subcutaneously.

A multimeric polypeptide of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, peritumoral, and intravenous routes, i.e., any route of administration other than through the alimentary canal. Parenteral administration can be carried to effect systemic or local delivery of a multimeric polypeptide of the present disclosure, a nucleic acid of the present disclosure, or a recombinant expression vector of the present disclosure. Where systemic delivery is desired, administration typically involves invasive or systemically absorbed topical or mucosal administration of pharmaceutical preparations.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment with a method of the present disclosure include individuals who have an infection (e.g., an infection with a pathogen such as a bacterium, a virus, a protozoan, etc.), including individuals who have been diagnosed as having an infection, and individuals who have been treated for an infection but who failed to respond to the treatment. Subjects suitable for treatment with a method of the present disclosure include individuals who have bacterial infection, including individuals who have been diagnosed as having a bacterial infection, and individuals who have been treated for a bacterial infection but who failed to respond to the treatment. Subjects suitable for treatment with a method of the present disclosure include individuals who have a viral infection, including individuals who have been diagnosed as having a viral infection, and individuals who have been treated for a viral infection but who failed to respond to the treatment. Subjects suitable for treatment with a method of the present disclosure include individuals who have an autoimmune disease, including individuals who have been diagnosed as having an autoimmune disease, and individuals who have been treated for a autoimmune disease but who failed to respond to the treatment.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generation and Characterization of synTac Polypeptides with Variant 4-1BBL

synTac polypeptides were synthesized and characterized. The following synTac polypeptides were tested for activity on ovalbumin (OVA)-specific T cells:

1) Syn83/51. The light chain of Syn83/51 comprises: a) an OVA T-cell epitope; b) amino acids 50-254 of a wild-type 4-1BBL polypeptide; and c) β2M; and the heavy chain of Syn83/51 comprises: a) MHC heavy chain; and b) Ig Fc.

2) Syn239/345. The light chain of Syn239/345 comprises: a) an OVA T-cell epitope; b) a trimer of amino acids 80-254 of wild-type 4-1BBL; and c) β2M; and the heavy chain of Syn239/345 comprises: a) MHC heavy chain; and b) IgG2a Fc.

3) Syn341/348. The light chain of Syn341/348 comprises: a) an OVA T-cell epitope; b) a trimer of wild-type 4-1BBL; and c) β2M; and the heavy chain of Syn239/345 comprises: a) MHC heavy chain; and b) IgG2a Fc. In Syn341/348 the first unit of the 4-1BBL trimer comprises amino acids 50-254 of wild-type 4-1BBL; the second and third units of the 4-1BBL trimer comprise amino acids 80 to 254 of wild-type 4-1BBL.

4) Syn341/349. The light chain of Syn341/349 comprises: an OVA T-cell epitope; b) a trimer of amino acids 80-254 of 4-1BBL comprising a K127A substitution in each unit of the trimer, with a linker GlySerSerSerSer between the first and second units and between the second and third units of the trimer; and c) β2M; and the heavy chain of Syn239/345 comprises: a) MHC heavy chain; and b) IgG2a Fc.

The resulting synTac heterodimers were cultured in vitro with ovalbumin-specific T cells for 3 days or 5 days, at concentrations of 0, 1, 3.17, 10.01, 31.65, and 100 nM synTac. Controls included: a) medium alone; b) phorbol 12-myristate 13-acetate (PMA) and the ionophore A23187; and c) an anti-CD3 antibody and an anti-CD28 antibody.

After 3 days, and after 5 days, the concentration of IFN-γ, IL-2, IL-6, TNF, IL-10, IL-17A, and IL-4 in the culture medium was determined. In addition, the viability of the OVA-specific T cells, and the proliferation of the OVA-specific T cells, was determined.

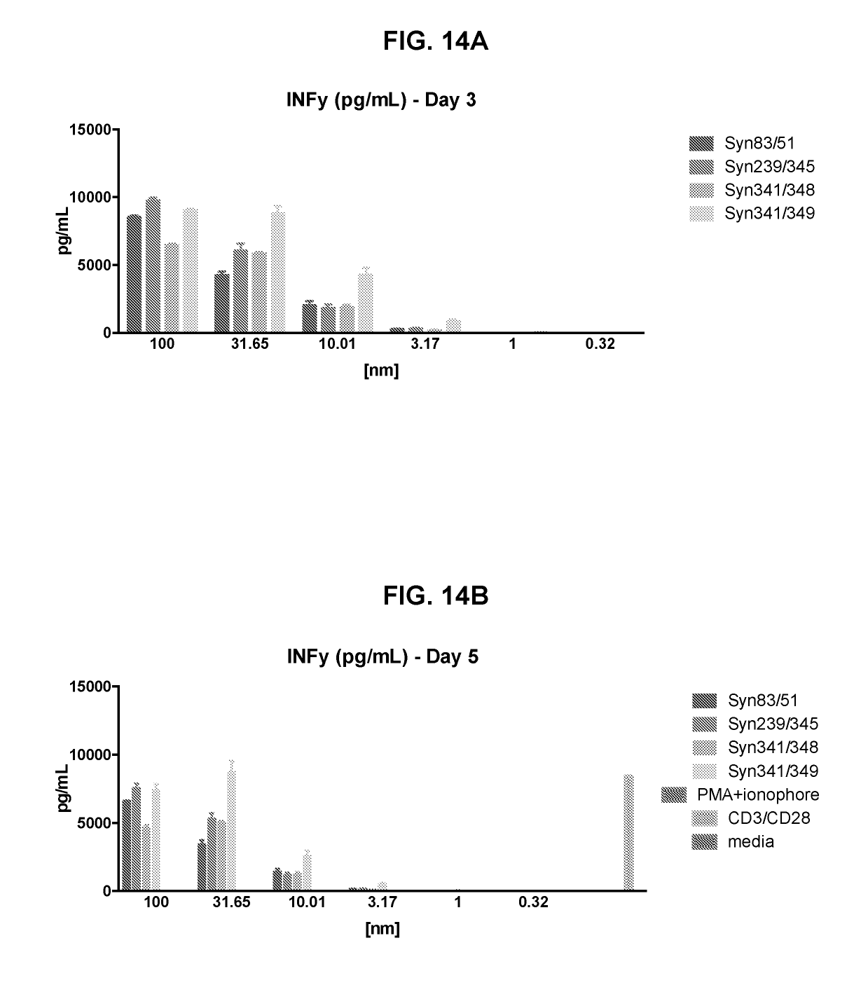

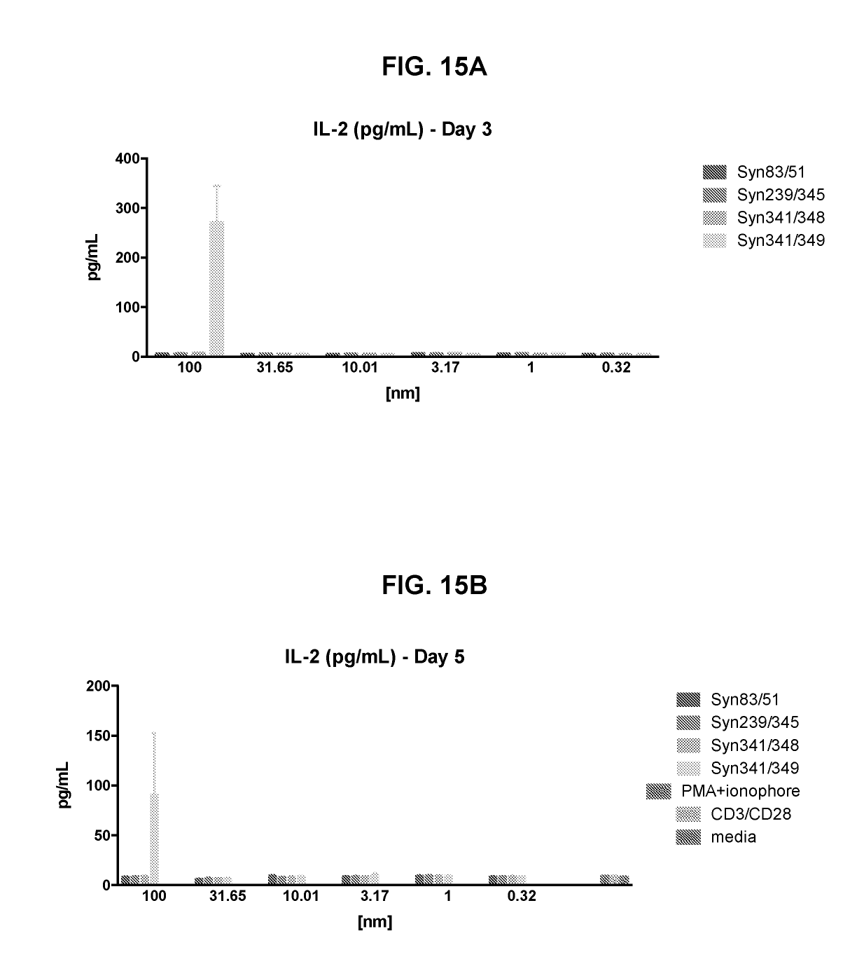

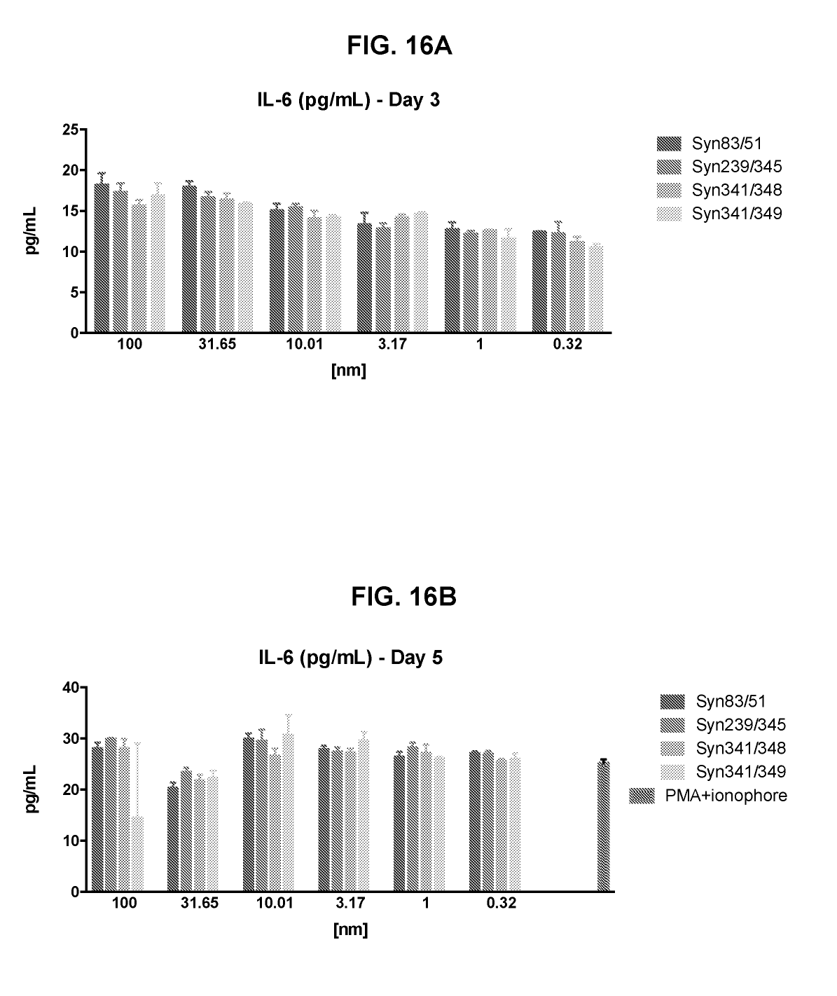

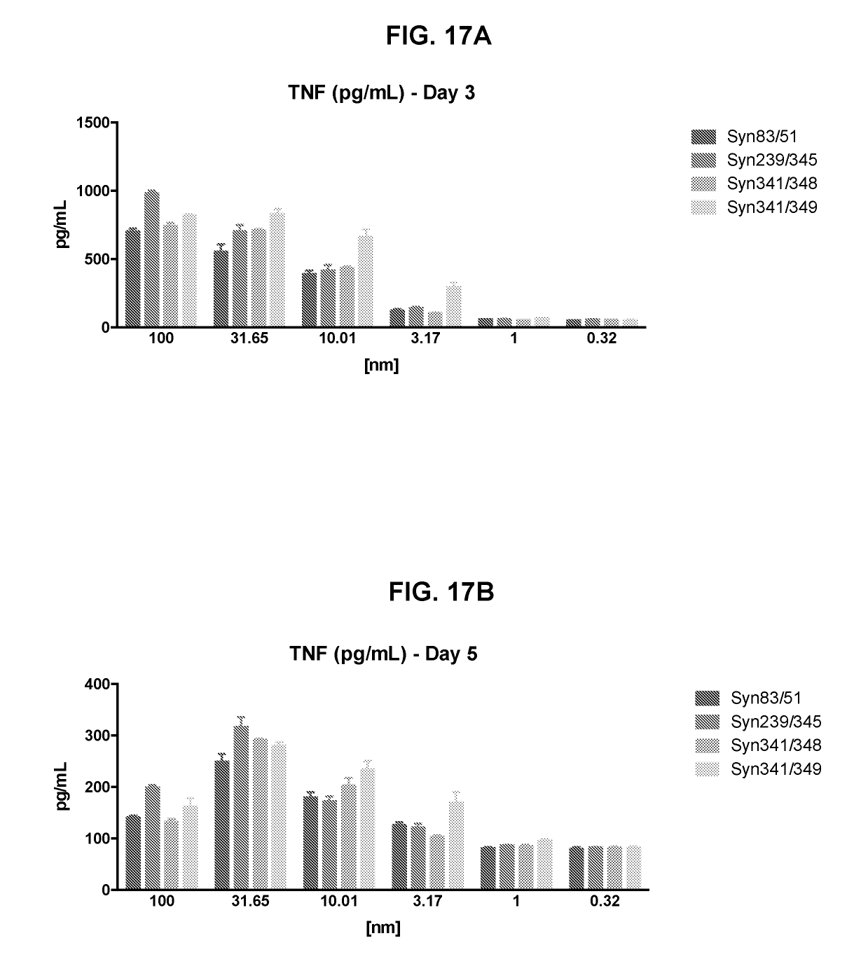

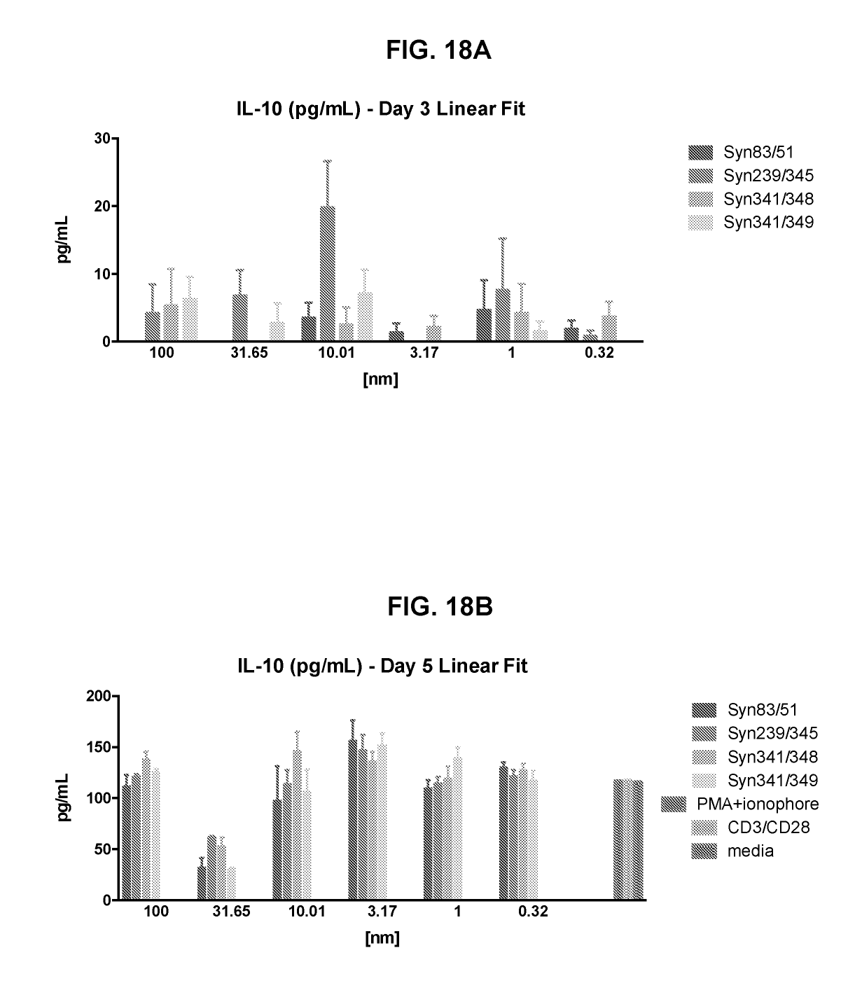

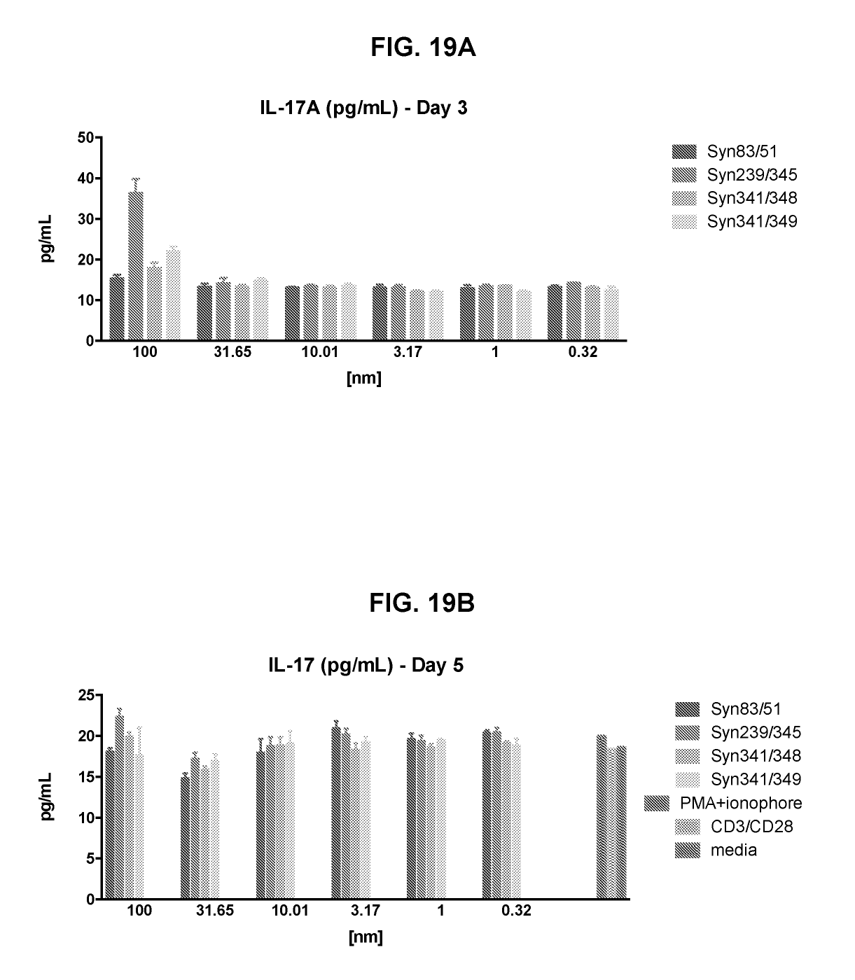

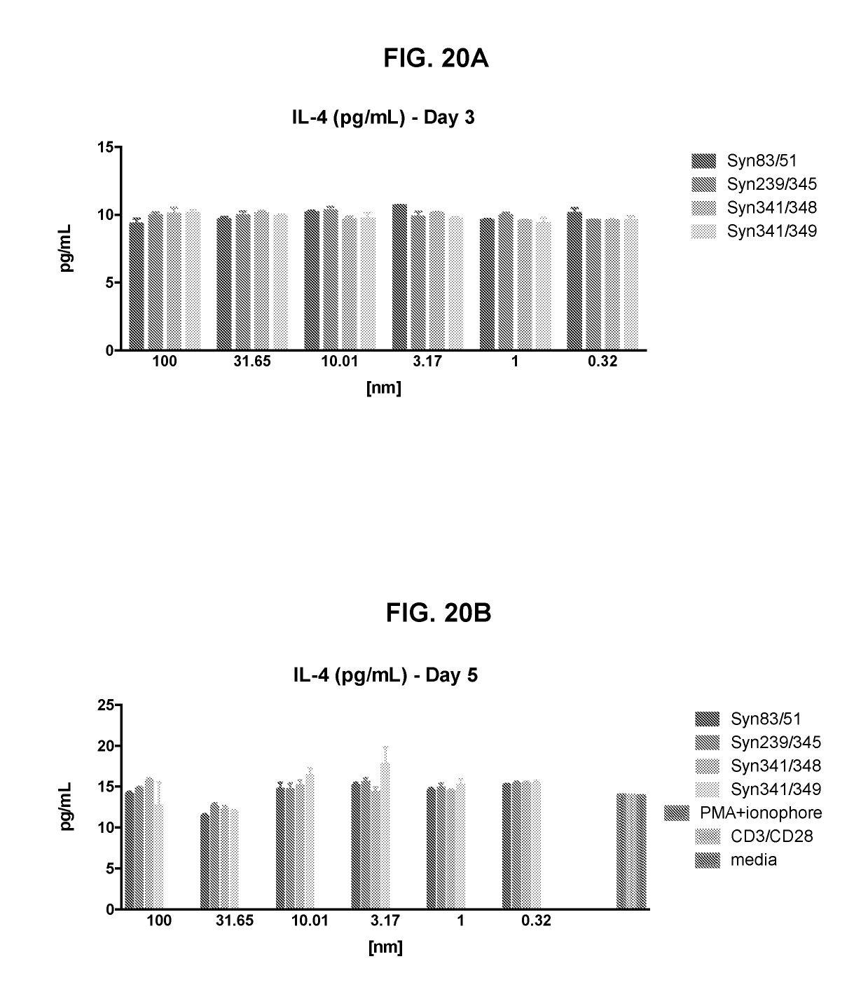

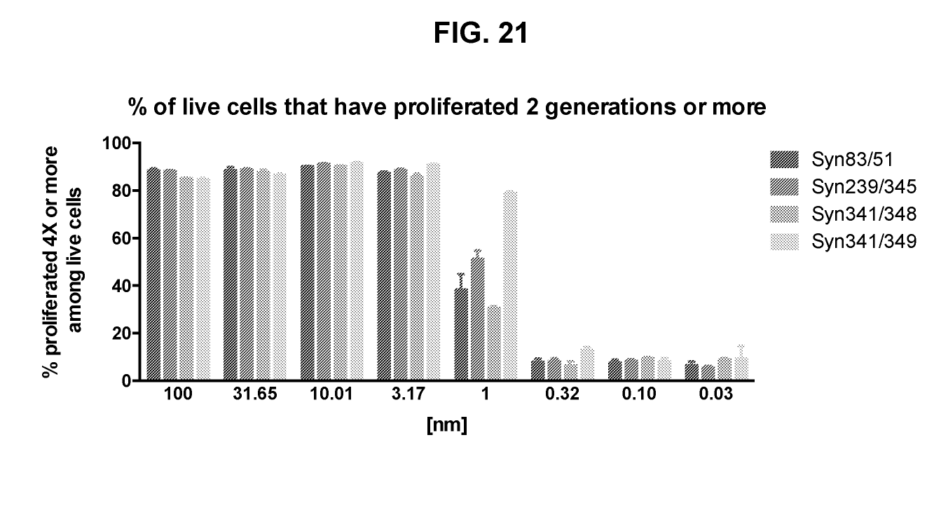

The data are depicted in FIGS. 14-22.

As shown in FIG. 14 through FIG. 22, Syn 341/349 induces production of IL-2 (a cellular fitness cytokine); induces production of cytotoxic cytokines TNFα and IFN-γ; and also induces proliferation and enhances viability of epitope-specific T cells.

### Example 2: Production of synTacs in CHO Cells

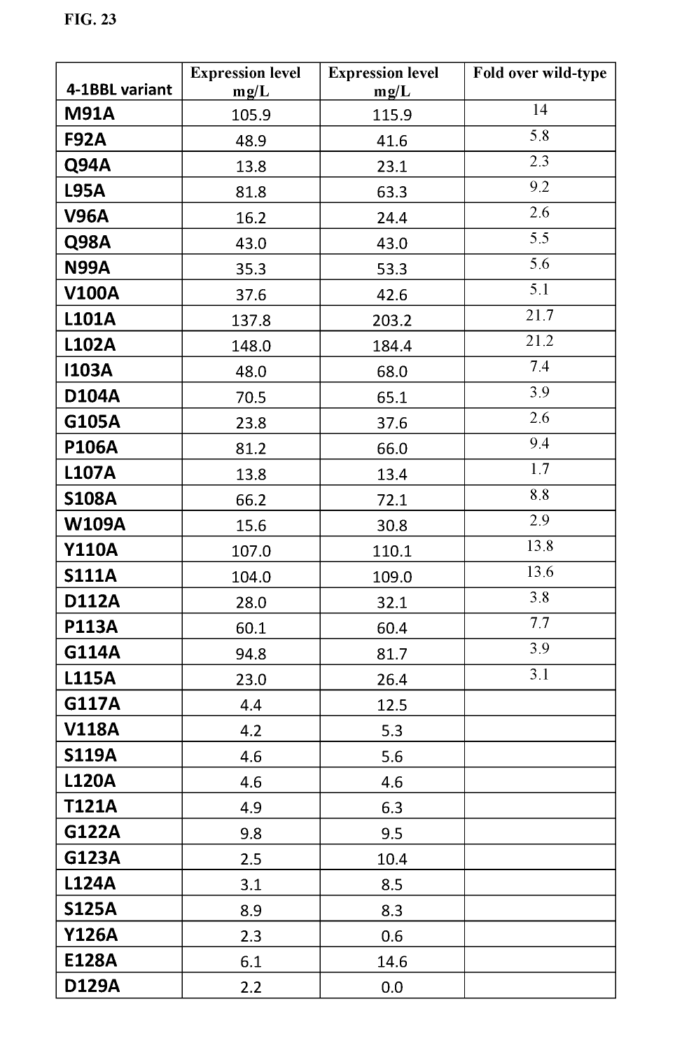

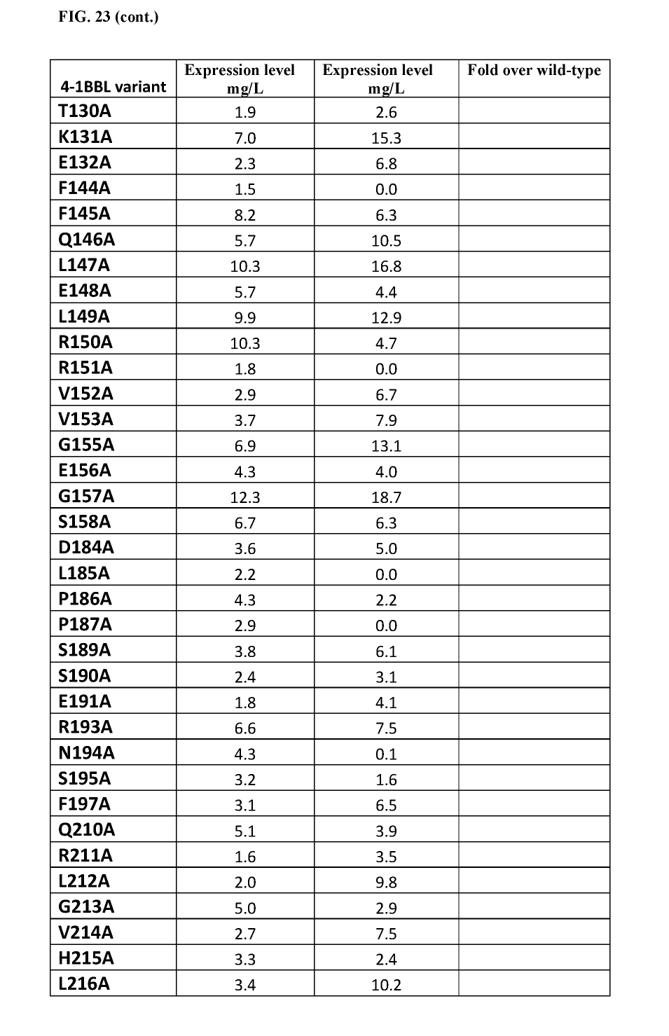

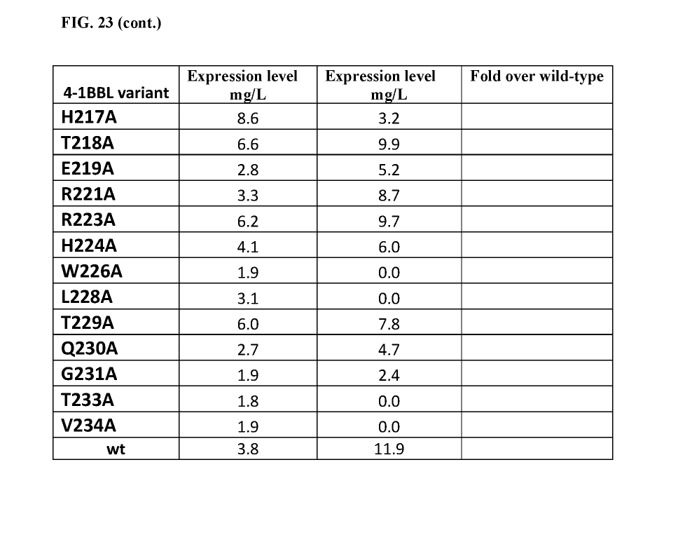

SynTacs comprising wild-type (wt) 4-1BBL, or comprising 4-1BBL with amino acid substitutions as set out in FIG. 23 were transiently expressed in CHO cells. The amount of synTac produced was determined. The amounts produced are provided in FIG. 23.

### Example 3: In Vivo Effect of a 4-1BBL synTac

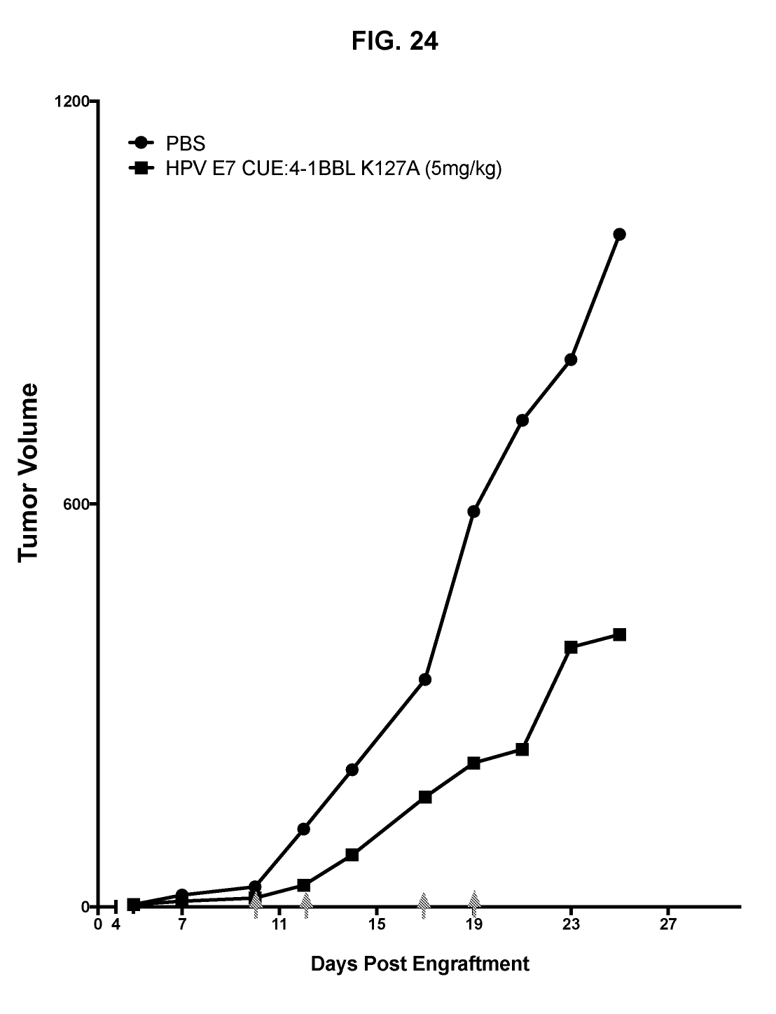

A synTac comprising a human papilloma virus (HPV) E7 antigenic peptide and a 4-1BBL K127A variant of the present disclosure (referred to as “CUE:4-1BBL (K127A)” in FIG. 24) was administered at 5 mg/kg by intraperitoneal (IP) injection into mice bearing flank engrafted HPV⁺ TC-1 lung carcinoma. As a control, phosphate buffered saline (PBS) was administered to mice bearing the same tumor. As shown in FIG. 24, tumor volume was decreased in mice treated with CUE:4-1BBL (K127A), compared to mice treated with PBS.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A variant 4-1BBL immunomodulatory polypeptide comprising an amino acid sequence having at least 85% amino acid sequence identity to the 4-1BBL amino acid sequence depicted in FIG. 2A or to the 4-1BBL amino acid sequence set forth in one of SEQ ID NOs:1-3,
wherein the variant 4-1BBL immunomodulatory polypeptide has one or more amino acid substitutions relative to the 4-1BBL amino acid sequence depicted in FIG. 2A or to the 4-1BBL amino acid sequence set forth in one of SEQ ID NOs:1-3; and
wherein the variant 4-1BBL immunomodulatory polypeptide: i) exhibits reduced binding affinity to a 4-1BB polypeptide having an amino acid sequence depicted in FIG. 3 and set forth in SEQ ID NO:91, compared to the binding affinity of the 4-1BBL amino acid sequence depicted in FIG. 2A or set forth in one of SEQ ID NOs:1-3 for the 4-1BB polypeptide; and/or ii) wherein the variant 4-1BBL immunomodulatory polypeptide exhibits increased production levels by a mammalian cell, compared to the production levels of the 4-1BBL amino acid sequence depicted in FIG. 2A or set forth in one of SEQ ID NOs:1-3.

2. The variant immunomodulatory polypeptide of claim 1, wherein the polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A.

3. The variant immunomodulatory polypeptide of claim 1, wherein the variant immunomodulatory polypeptide exhibits less than 50% of binding affinity exhibited by to the 4-1BBL amino acid sequence depicted in FIG. 2A, or as set forth in one of SEQ ID NOs:1-3, for the 4-1BB polypeptide.

4. A multimeric polypeptide comprising:
a) a first polypeptide comprising, in order from N-terminus to C-terminus:
i) an epitope;
ii) a first major histocompatibility complex (MHC) polypeptide; and
b) a second polypeptide comprising, in order from N-terminus to C-terminus:
i) a second MHC polypeptide; and
ii) optionally an immunoglobulin (Ig) Fc polypeptide or a non-Ig scaffold,
wherein the multimeric polypeptide comprises one or more immunomodulatory domains, wherein the one or more immunomodulatory domain is:
A) at the C-terminus of the first polypeptide;
B) at the N-terminus of the second polypeptide;
C) at the C-terminus of the second polypeptide; or
D) at the C-terminus of the first polypeptide and at the N-terminus of the second polypeptide,
wherein the immunomodulatory domain is a variant immunomodulatory polypeptide of any one of claims 1-3; and
wherein: i) the multimeric polypeptide exhibits reduced binding affinity to a 4-1BB polypeptide having an amino acid sequence depicted in FIG. 3 and set forth in SEQ ID NO:91, compared to the binding affinity of a control multimeric polypeptide comprising an immunomodulatory domain comprising the 4-1BBL amino acid sequence depicted in FIG. 2A or set forth in one of SEQ ID NOs:1-3 for the 4-1BB polypeptide; and/or ii) wherein the multimeric polypeptide exhibits increased production levels by a mammalian cell, compared to the production levels of a control multimeric polypeptide comprising an immunomodulatory domain comprising the 4-1BBL amino acid sequence depicted in FIG. 2A or set forth in one of SEQ ID NOs:1-3.

5. The multimeric polypeptide of claim 4, wherein the multimeric polypeptide comprises:
a) a first polypeptide comprising, in order from N-terminus to C-terminus:
i) an epitope;
ii) a first MHC polypeptide; and
iii) an immunomodulatory domain; and
b) a second polypeptide comprising, in order from N-terminus to C-terminus:
i) a second MHC polypeptide; and
ii) an Ig Fc polypeptide.

6. The multimeric polypeptide of claim 4, wherein the multimeric polypeptide comprises:
a) a first polypeptide comprising, in order from N-terminus to C-terminus:
i) an epitope; and
ii) a first MHC polypeptide; and
b) a second polypeptide comprising, in order from N-terminus to C-terminus:
i) an immunomodulatory domain;
iii) a second MHC polypeptide; and
ii) an immunoglobulin (Ig) Fc polypeptide.

7. The multimeric polypeptide of claim 4, wherein the multimeric polypeptide comprises:
a) a first polypeptide comprising, in order from N-terminus to C-terminus:
i) an epitope; and
ii) a first MHC polypeptide; and
b) a second polypeptide comprising, in order from N-terminus to C-terminus:
i) a second MHC polypeptide; and
ii) an Ig Fc polypeptide; and
iii) an immunomodulatory domain.

8. The multimeric polypeptide of claim 4, wherein the multimeric polypeptide comprises:
a) a first polypeptide comprising, in order from N-terminus to C-terminus:
i) an epitope; and
ii) a first MHC polypeptide; and
b) a second polypeptide comprising, in order from N-terminus to C-terminus:
i) a second MHC polypeptide; and
ii) an immunomodulatory domain.

9. The multimeric polypeptide of claim 4, wherein the multimeric polypeptide comprises:
a) a first polypeptide comprising, in order from N-terminus to C-terminus:
i) an epitope; and
ii) a first MHC polypeptide; and
b) a second polypeptide comprising, in order from N-terminus to C-terminus:
i) an immunomodulatory domain; and
ii) a second MHC polypeptide.

10. The multimeric polypeptide of claim 4, wherein the multimeric polypeptide comprises:
a) a first polypeptide comprising, in order from N-terminus to C-terminus:
i) an epitope;
ii) a first MHC polypeptide; and
iii) an immunomodulatory domain; and
b) a second polypeptide comprising, in order from N-terminus to C-terminus:
i) a second MHC polypeptide.

11. The multimeric polypeptide of claim 4, wherein the non-Ig scaffold is an XTEN polypeptide, a transferrin polypeptide, an elastin-like polypeptide, a silk-like polypeptide, or a silk-elastin-like polypeptide.

12. The multimeric polypeptide of any one of claims 4-11, wherein the first MHC polypeptide is a β2-microglobulin polypeptide; and wherein the second MHC polypeptide is an MHC class I heavy chain polypeptide.

13. The multimeric polypeptide of claim 12, wherein the β2-microglobulin polypeptide comprises an amino acid sequence having at least 85% amino acid sequence identity to any one of the amino acid sequences set forth in SEQ ID NO:6.

14. The multimeric polypeptide of claim 11, wherein the MHC class I heavy chain polypeptide is an HLA-A, an HLA-B, or an HLA-C heavy chain.

15. The multimeric polypeptide of claim 12, wherein the MHC class I heavy chain polypeptide comprises an amino acid sequence having at least 85% amino acid sequence identity to the amino acid sequence depicted in any one of FIG. 5A-5C.

16. The multimeric polypeptide of any one of claims 4-11, wherein the first MHC polypeptide is an MHC Class II alpha chain polypeptide; and wherein the second MHC polypeptide is an MHC class II beta chain polypeptide.

17. The multimeric polypeptide of any one of claims 4-16, wherein the epitope is a T-cell epitope.

18. The multimeric polypeptide of any one of claims 4-10 and 12-17, wherein multimeric polypeptide comprises an Fc polypeptide, and wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide.

19. The multimeric polypeptide of claim 18, wherein the Ig Fc polypeptide comprises an amino acid sequence having at least 85% amino acid sequence identity to an amino acid sequence depicted in FIG. 4A-4C.

20. The multimeric polypeptide of any one of claims 4-19, wherein the first polypeptide and the second polypeptide are non-covalently associated.

21. The multimeric polypeptide of any one of claims 4-19, wherein the first polypeptide and the second polypeptide are covalently linked.

22. The multimeric polypeptide of claim 21, wherein the covalent linkage is via a disulfide bond.

23. The multimeric polypeptide of claim 22, wherein the first MHC polypeptide or a linker between the epitope and the first MHC polypeptide comprises an amino acid substitution to provide a first Cys residue, and the second MHC polypeptide comprises an amino acid substitution to provide a second Cys residue, and wherein the disulfide linkage is between the first and the second Cys residues.

24. The multimeric polypeptide of any one of claims 4-11, comprising a first linker interposed between the epitope and the first MHC polypeptide.

25. The multimeric polypeptide of any one of claims 4-11, wherein the variant 4-1BBL immunomodulatory polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A.

26. The multimeric polypeptide of any one of claims 4-25, comprising 2 or more immunomodulatory polypeptides.

27. The multimeric polypeptide of claim 26, wherein the 2 or more immunomodulatory polypeptides are in tandem.

28. The multimeric polypeptide of any one of claims 26 and 27, wherein the multimeric polypeptide comprises a third polypeptide, wherein the third polypeptide comprises an immunomodulatory polypeptide comprising an amino acid sequence having at least 90% amino acid sequence identity to the immunomodulatory polypeptide of the first polypeptide or the second polypeptide.

29. The multimeric polypeptide of claim 28, wherein the third polypeptide is covalently linked to the first polypeptide.

30. The multimeric polypeptide of any one of claims 4-10 and 12-29, wherein the second polypeptide comprises, in order from N-terminus to C-terminus:
i) the second MHC polypeptide;
ii) the Ig Fc polypeptide; and
iii) an affinity tag.

31. A nucleic acid comprising a nucleotide sequence encoding a recombinant polypeptide,
i) wherein the recombinant polypeptide comprises, in order from N-terminus to C-terminus:
a) an epitope;
b) a first major histocompatibility complex (MHC) polypeptide;
c) an immunomodulatory polypeptide;
d) a proteolytically cleavable linker or a ribosome skipping signal;
e) a second MHC polypeptide; and
f) an immunoglobulin (Ig) Fc polypeptide;
wherein the immunomodulatory polypeptide is a variant immunomodulatory polypeptide of any one of claims 1-3; or
ii) wherein the recombinant polypeptide comprises, in order from N-terminus to C-terminus:
a) an epitope;
b) a first MHC polypeptide;
c) a proteolytically cleavable linker or a ribosome skipping signal;
d) an immunomodulatory polypeptide
e) a second MHC polypeptide; and
f) an Ig Fc polypeptide,
wherein the immunomodulatory polypeptide is a variant immunomodulatory polypeptide of any one of claims 1-3.

32. The nucleic acid of claim 31, wherein the first MHC polypeptide is a β2-microglobulin polypeptide; and wherein the second MHC polypeptide is an MHC class I heavy chain polypeptide.

33. The nucleic acid of claim 32, wherein the β2-microglobulin polypeptide comprises an amino acid sequence having at least 85% amino acid sequence identity to any one of the amino acid sequences depicted in FIG. 6

34. The nucleic acid of claim 31, wherein the MHC class I heavy chain polypeptide is an HLA-A, HLA-B, or HLA-C heavy chain.

35. The nucleic acid of claim 34, wherein the MHC class I heavy chain polypeptide comprises an amino acid sequence having at least 85% amino acid sequence identity to the amino acid sequence depicted in any one of FIG. 5A-5C.

36. The nucleic acid of claim 31, wherein the first MHC polypeptide is an MHC Class II alpha chain polypeptide; and wherein the second MHC polypeptide is an MHC class II beta chain polypeptide.

37. The nucleic acid of claim 31, wherein the epitope is a T-cell epitope.

38. The nucleic acid of claim 31, wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide.

39. The nucleic acid of claim 38, wherein the Ig Fc polypeptide comprises an amino acid sequence having at least 85% amino acid sequence identity to an amino acid sequence depicted in FIGS. 4A-4C.

40. The nucleic acid of claim 31, wherein the variant 4-1BBL immunomodulatory polypeptide comprises a substitution of one of amino acids 91, 92, 94-115, 117-126, 128-132, 144-153, 155-158, 184-187, 189-191, 193-195, 197, 210-219, 221-224, 226, 228-231, 233, and 234 based on the amino acid numbering set out in FIG. 2A.

41. The nucleic acid of claim 31, wherein the multimeric polypeptide comprises a second immunomodulatory polypeptide selected from a CD7, CD30L, CD40, CD70, CD83, HLA-G, MICA, MICB, HVEM, lymphotoxin beta receptor, 3/TR6, ILT3, ILT4, and HVEM.

42. The nucleic acid of claim 31, wherein the proteolytically cleavable linker or ribosome skipping signal comprises an amino acid sequence selected from:
a)
(SEQ ID NO: 116)
LEVLFQGP;
b)
(SEQ ID NO: 117)
ENLYTQS;
c) a furin cleavage site;
d)
(SEQ ID NO: 118)
LVPR;
e)
(SEQ ID NO: 119)
GSGATNFSLLKQAGDVEENPGP;
f)
(SEQ ID NO: 120)
GSGEGRGSLLTCGDVEENPGP;
g)
(SEQ ID NO: 121)
GSGQCTNYALLKLAGDVESNPGP;
and
h)
(SEQ ID NO: 122)
GSGVKQTLNFDLLKLAGDVESNPGP.

43. The nucleic acid of claim 31, wherein the recombinant polypeptide comprises, in order from N-terminus to C-terminus:
a) a first leader peptide;
b) the epitope;
c) the first MHC polypeptide;
d) the immunomodulatory polypeptide;
e) the proteolytically cleavable linker or ribosome skipping signal;
f) a second leader peptide;
g) the second MHC polypeptide; and
h) the immunoglobulin (Ig) Fc polypeptide.

44. The nucleic acid of claim 43, wherein the first leader peptide and the second leader peptide is a β2-M leader peptide.

45. The nucleic acid of claim 31, wherein the nucleotide sequence is operably linked to a transcriptional control element.

46. The nucleic acid of claim 45, wherein the transcriptional control element is a promoter that is functional in a eukaryotic cell.

47. The nucleic acid of claim 31, wherein the first MHC polypeptide or a linker between the epitope and the first MHC polypeptide comprises an amino acid substitution to provide a first Cys residue, and the second MHC polypeptide comprises an amino acid substitution to provide a second Cys residue, and wherein the first and the second Cys residues provide for a disulfide linkage between the first MHC polypeptide and the second MHC polypeptide.

48. A recombinant expression vector comprising the nucleic acid of any one of claims 31-47.

49. The recombinant expression vector of claim 48, wherein the vector is a viral vector or a non-viral vector.

50. A host cell genetically modified with the recombinant expression vector of claim 48 or claim 49.

51. The host cell of claim 50, wherein the host cell is in vitro.

52. The host cell of claim 50, wherein the host cell is genetically modified such that the cell does not produce an endogenous MHC β2-microglobulin polypeptide.

53. The host cell of claim 50, wherein the host cell is a T lymphocyte.

54. A composition comprising:
a) a first nucleic acid comprising a nucleotide sequence encoding a first polypeptide comprising, in order from N-terminus to C-terminus:
i) an epitope;
ii) a first MHC polypeptide; and
iii) an immunomodulatory domain,
wherein the immunomodulatory domain is a variant immunomodulatory polypeptide of any one of claims 1-3; and
b) a first nucleic acid comprising a nucleotide sequence encoding a second polypeptide comprising, in order from N-terminus to C-terminus:
i) a second MHC polypeptide; and
ii) an Ig Fc polypeptide.

55. A composition comprising:
a) a first nucleic acid comprising a nucleotide sequence encoding a first polypeptide comprising, in order from N-terminus to C-terminus:
i) an epitope; and
ii) a first MHC polypeptide; and
b) a first nucleic acid comprising a nucleotide sequence encoding a second polypeptide comprising, in order from N-terminus to C-terminus:
i) an immunomodulatory domain, wherein the immunomodulatory domain is a variant immunomodulatory polypeptide of any one of claims 1-3;
ii) a second MHC polypeptide; and
iii) an Ig Fc polypeptide.

56. The composition of claim 54 or 55, wherein the first and/or the second nucleic acid is present in a recombinant expression vector.

57. A host cell genetically modified with the composition of any one of claims 54-56.

58. A method of producing the multimeric polypeptide of any one of claims 4-30, the method comprising:
a) culturing the host cell of 48, 49, or 57 in vitro in a culture medium under conditions such that the host cell synthesizes the multimeric polypeptide; and
b) isolating the multimeric polypeptide from the host cell and/or from the culture medium.

59. The method of claim 58, wherein the second polypeptide comprises an affinity tag, and wherein said isolating comprises contacting the multimeric polypeptide produced by the cell with a binding partner for the affinity tag, wherein the binding partner is immobilized, thereby immobilizing the multimeric polypeptide.

60. The method of claim 58, comprising eluting the immobilized multimeric polypeptide.

61. A method of selectively modulating the activity of an epitope-specific T cell, the method comprising contacting the T cell with the multimeric polypeptide of any one of claims 4-30, wherein said contacting selectively modulates the activity of the epitope-specific T cell.

62. The method of claim 61, wherein the immunomodulatory polypeptide is an activating polypeptide, and wherein the multimeric polypeptide activates the epitope-specific T cell.

63. The method of claim 61, wherein the immunomodulatory polypeptide is an inhibiting polypeptide, and wherein the multimeric polypeptide inhibits the epitope-specific T cell.

64. The method of claim 61, wherein said contacting is in vitro.

65. The method of claim 61, wherein said contacting is in vivo.

66. A method of selectively modulating the activity of an epitope-specific T cell in an individual, the method comprising administering to the individual an effective amount of the multimeric polypeptide of any one of claims 4-30 effective to selectively modulate the activity of an epitope-specific T cell in an individual.

67. The method of claim 66, wherein the immunomodulatory polypeptide is an activating polypeptide, and wherein the multimeric polypeptide activates the epitope-specific T cell.

68. The method of claim 67, wherein the epitope is a cancer-associated epitope, and wherein said administering selectively increases the activity of a T cell specific for the cancer-associate epitope.

69. The method of claim 66, wherein the immunomodulatory polypeptide is an inhibitory polypeptide, and wherein the multimeric polypeptide inhibits activity of the epitope-specific T cell.

70. The method of claim 69, wherein the epitope is a self-epitope, and wherein said administering selectively inhibits the activity of a T cell specific for the self-epitope.

71. A method of treating an infection in an individual, the method comprising administering to the individual an effective amount of
a) the multimeric polypeptide of any one of claims 4-30; or
b) one or more recombinant expression vectors comprising nucleotide sequences encoding the multimeric polypeptide of any one of claims 4-30; or
c) one or more mRNAs comprising nucleotide sequences encoding the multimeric polypeptide of any one of claims 4-30.
wherein the epitope is a pathogen-associated epitope, wherein the immunomodulatory polypeptide is an activating polypeptide, and wherein said administering effective to selectively modulate the activity of a pathogen-associated epitope-specific T cell in an individual.

72. The method of claim 71, wherein the pathogen is a virus, a bacterium, or a protozoan.

73. The method of any one of claims 66-71, wherein said administering is subcutaneous.

74. The method of any one of claims 66-71, wherein said administering is intravenous.

75. The method of any one of claims 66-71, wherein said administering is intramuscular.

76. The method of any one of claims 66-71, wherein said administering is systemic.

77. The method of any one of claims 66-71, wherein said administering is distal to a treatment site.

78. The method of any one of claims 66-71, wherein said administering is local.

79. The method of any one of claims 66-71, wherein said administering is at or near a treatment site.

80. A composition comprising:
a) the multimeric polypeptide of any one of claims 4-30; and
b) a pharmaceutically acceptable excipient.

81. A composition comprising:
a) the nucleic acid of any one of claims 31-47 or the recombinant expression vector of claim 48 or 49; and
b) a pharmaceutically acceptable excipient.
